(19) United States
(12) Patent Application Publication
(10) Pub. No.: US 2021/0290597 A1
(43) Pub. Date: Sep. 23, 2021

(54) METHOD OF TREATING A CONDITION ASSOCIATED WITH NEURODEGENERATION USING INHIBITORS OF OAT3

(71) Applicant: Neuropore Therapies, Inc., San Diego, CA (US)

(72) Inventors: Douglas W. BONHAUS, San Diego, CA (US); Wolfgang J. WRASIDLO, La Jolla, CA (US); Emily M. STOCKING, Encinitas, CA (US); Srinivasa Reddy NATALA, San Diego, CA (US)

(73) Assignee: Neuropore Therapies, Inc., San Diego, CA (US)

(21) Appl. No.: 17/253,581

(22) PCT Filed: Jun. 20, 2019
(86) PCT No.: PCT/US2019/038328

§ 371 Date: Dec. 17, 2020

Related U.S. Application Data
(60) Provisional application No. 62/752,265, filed on Oct. 29, 2018.
(60) Provisional application No. 62/687,733, filed on Jun. 20, 2018.

(51) Int. Cl. A61K 31/4196; A61K 31/496; A61K 31/4439; A61K 45/06; A61K 31/335; A61P 25/28; A61K 9/16

(52) U.S. Cl. CPC A61K 31/4196; A61K 31/496; A61K 31/4439; A61K 9/16; A61K 31/335; A61P 25/28; A61K 45/06

(57) ABSTRACT

The present disclosure relates to therapeutic agents that may be useful intreatment and prophylaxis of neurodegenerative disorders and/or neural inflammation.

### CROSS-REFERENCE TO RELATED APPLICATIONS

This application claims priority to U.S. Provisional Application No. 62/687,733, filed Jun. 20, 2018, entitled “METHOD OF TREATING A CONDITION ASSOCIATED WITH NEURODEGENERATION USING AN INHIBITOR OF OAT3” and U.S. Provisional Application No. 62/752,265, filed Oct. 29, 2018, entitled “METHOD OF TREATING A CONDITION ASSOCIATED WITH NEURODEGENERATION USING AN INHIBITOR OF OAT3” the contents of which are hereby incorporated by reference in their entireties for all purposes.

### TECHNICAL FIELD

The present disclosure relates generally to therapeutic agents that may be useful in treatment and prophylaxis of neurodegenerative disorders and/or neural inflammation.

### BACKGROUND

The blood-brain barrier (BBB), formed by a tight monolayer of endothelial cells, allows passive diffusion of water, some gases and certain lipid-soluble molecules. Nevertheless, other molecules, such as organic anions, are selectively transported across the BBB. An active efflux system in the BBB controls the unbound concentrations of exogenous compounds in the brain interstitial space and inactivates neuroactive compounds by transferring them into the blood. Organic anion transporter 3 (OAT3), which is also known as “solute carrier family 22 member 8” (SLC22A8), is possibly the most abundantly expressed organic ion transport subtype in the brain (Gasser et al., 2009; Roberts et al., 2008). OAT3 mediates the active efflux from the brain of bioactive endogenous metabolites, some of which possess anti-inflammatory and neuroprotective activity.

Among the bioactive endogenous metabolites found in the brain, soluble uric acid as well as dehydroepiandrosterone (DHEA) and its conjugate ester, DHEA sulfate (DHEAS) have been shown in various animal models to demonstrate beneficial neuroprotection and anti-inflammatory actions. In some animal disease models, the beneficial actions of uric acid and DHEA are associated with upregulation of pAkt as well as downregulation of inflammation markers such as GFAP. Nevertheless, systemic administration of uric acid would elevate the plasma concentrations leading to other deleterious effects, such as gout and hyperuricemia. There exists the need to locally increase levels of neuroprotectants such as uric acids and DHEA in the brain without significant changes in their plasma levels.

OAT3 has been reported as a transporter for DHEAS (Miyajima et al.) and purportedly for uric acid (Bakhlya et al., 2003; Eraly et al., 2008). By inhibiting OAT3 selectively, neuroprotective substrates such as uric acids and DHEA normally transported out of the brain by OAT3 will remain in the brain, thereby elevating their levels in the brain interstitial space.

The present invention relates to the use of a compound that inhibits OAT3, thereby inhibiting the efflux of neuroprotectants from the brain interstitial space, hence elevating the levels of neuroprotectants in the brain to confer neuroprotection and anti-neuroinflammation. In certain embodiments, the compound (also referred to herein as the “ion transporter inhibitor”) selectively inhibits OAT3. For instance, in some embodiments, the compound displays more potent inhibitory activity against OAT3 compared with its activity against other ion transporter proteins such as OAT1, OAT2, OAT3, OAT4, OAT6, OAT7, OAT9, OAT10, OCT2, OATP1B1, OATP1B3, MATE1, MATE2-K, BCRP, PBP, and URAT1.

### SUMMARY

Provided in some aspects are methods of treating a disease or condition associated with neurodegeneration or neuroinflammation in the brain in a subject in need thereof, comprising administering to the subject an effective amount of an ion transporter inhibitor, wherein the ion transporter inhibitor modulates the efflux of one or more bioactive endogenous metabolites across the blood brain barrier (BBB) of the subject. In some embodiments, the condition associated with neurodegeneration is Alzheimer's Disease, Parkinson's Disease, fronto-temporal dementia, dementia with Lewy Bodies, PD dementia, multiple system atrophy, Huntington's disease, Amyotrophic lateral sclerosis, progressive supranuclear palsy, or neuroinflammation.

Provided in other aspects are methods of modulating efflux of one or more bioactive endogenous metabolites across the blood brain barrier (BBB) in a subject in need thereof, comprising administering to the subject in need thereof an ion transporter inhibitor.

Provided in another aspect is a method of improving neuroprotection in a subject in need thereof, comprising administering to the subject an effective amount of an ion transporter inhibitor that modulates the concentration of one or more bioactive endogenous metabolites in the brain interstitial space

In other aspects, provided are methods of decreasing neuroinflammation in a subject in need thereof, comprising administering to the subject an effective amount of an ion transporter inhibitor that modulates the concentration of one or more bioactive endogenous metabolites in the brain interstitial space.

In some embodiments of any of the methods described herein, the ion transporter inhibitor is an inhibitor of organic anion transporter 3 (OAT3). In some embodiments, the ion transporter inhibitor selectively inhibits OAT3 compared with other ion transporter proteins. In some embodiments of any of the methods described herein, the ion transporter inhibitor has an IC₅₀ for OAT3 of about 1 μM or less. In some embodiments of any of the methods described herein, the ion transporter inhibitor has an IC₅₀ for OAT3 that is at least about 10 fold lower compared with its IC₅₀ for organic anion transporter 1 (OAT1).

In some embodiments of any of the methods described herein, after administration of the ion transporter inhibitor, the efflux of the one or more bioactive endogenous metabolites across the BBB is reduced. In some embodiments of any of the methods described herein, after administration of the ion transporter inhibitor, the local concentrations of the one or more bioactive endogenous metabolites in the brain interstitial space are increased. In some embodiments of any of the methods described herein, after administration of the ion transporter inhibitor, the levels of the one or more bioactive endogenous metabolites in the brain interstitial space are increased by about 50% or more. In some embodiments of any of the methods described herein, after administration of the ion transporter inhibitor, the plasma levels of the one or more bioactive endogenous metabolites are decreased.

In some embodiments of any of the methods described herein, the plasma levels of the bioactive endogenous metabolites are modulated by 50% or less. In some embodiments, the plasma levels of the bioactive endogenous metabolites are decreased by 50% or less. In some embodiments of any of the methods described herein, the one or more bioactive endogenous metabolites is an anionic neurotransmitter metabolite of epinephrine, norepinephrine, dopamine, and/or serotonin. In some embodiments, the one or more bioactive endogenous metabolites are selected from the group consisting of: uric acid, glutathione, dehydroepianodrosterone (DHEA), and DHEA sulfate (DHEAS). In some embodiments, the one or more bioactive endogenous metabolites have neuroprotective and/or anti-neuroinflammatory properties. In some embodiments of any of the methods described herein, the anti-neuroinflammatory properties include reduction of a pro-inflammatory response in the brain of the subject. In some embodiments of any of the methods described herein, the reduction of a pro-inflammatory response comprises reduction in gene expression of one or more of TNF, IL6, IL12/23p40 or MCP1. In some embodiments, the reduction of a pro-inflammatory response is mediated by processes comprising activation of TrkA/Akt/CREB/Jmjd3 pathway in the brain of the subject. In some embodiments of any of the methods described herein, activation of the TrkA/Akt/CREB/Jmjd3 pathway comprises increase of pTrkA levels in the brain of the subject. In some embodiments, activation of the TrkA/Akt/CREB/Jmjd3 pathway comprises increase of pAkt levels in the brain of the subject. In some embodiments, activation of the TrkA/Akt/CREB/Jmjd3 pathway comprises increase of pCREB levels in the brain of the subject. In some embodiments, activation of the TrkA/Akt/CREB/Jmjd3 pathway comprises an increase in Jmjd3 expression in the brain of the subject. In some embodiments of any of the methods described herein, the anti-neuroinflammatory properties comprises induction of an anti-inflammatory phenotype of microglia in the subject. In some embodiments of any of the methods described herein, the anti-inflammatory phenotype of microglia comprises increased gene expression of one or more of M2 polarization markers comprising one or more of arginase 1, Ym1 (chitinase-like protein 3), Fizz1, Klf4 (Kruppel like factor 4) or IL10. In some embodiments, the anti-inflammatory phenotype of microglia comprises inhibition of a pro-inflammatory phenotype of microglia in the subject.

Provided in other aspects are methods of preventing aggregation or accumulation or enhancing clearance of protease-resistant protein, comprising contacting the protease-resistant protein with an effective amount of a compound that is an inhibitor of organic anion transporter 3 (OAT3), wherein the contacting is in vitro, ex vivo, or in vivo. In some embodiments, the protease-resistant protein is selected from alpha synuclein, a-beta, tau, Huntingtin, and TAR DNA binding protein 43 (TDP43) proteins.

In some embodiments of any of the methods described herein, the compound is a compound of Formula (I):

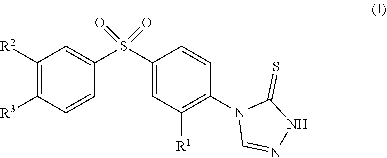

wherein
R¹, R², and R³ are each independently hydrogen, hydroxy, halogen, optionally substituted C₁₋₄ alkyl, optionally substituted C₁₋₄ alkoxy, —CN, —C(O)R^{x}, —C(O)OR^{x}, —S(O)₂R^{x}, or —NR^{y}R^{z};
R^{x}, R^{y}, and R^{z} are each independently H or optionally substituted C₁₋₄alkyl, or R^{y} and R^{z} taken together with the nitrogen to which they are attached form an optionally substituted monocyclic heterocycloalkyl ring;
or a pharmaceutically acceptable salt thereof.

In some embodiments of any of the methods described herein, the compound is a compound of Formula (IIA):

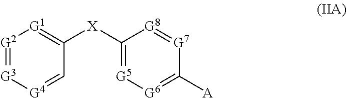

wherein

### G¹ is CH or N;

G² is CR^{2a} or N;
G³ is CR^{3a} or N;

### G⁴ is CH or N;

wherein no more than two of G¹, G², G³, and G⁴ are N;

### G⁵ is CH or N;

G⁶ is CR¹a or N;

### G⁷ is CH or N;

### G⁸ is CH or N;

wherein no more than one of G⁵, G⁶, G⁷, and G⁸ is N;
R^{1a}, R^{2a}, and R^{3a} are each independently hydrogen, hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, or an optionally substituted heterocyclyl;
   wherein R^{x} and R^{y} are each independently H or optionally substituted C₁₋₄alkyl;
or R^{1a} and R^{2a} are taken together with the carbons to which they are attached to form a 5- to 16-membered heterocyclyl ring;
   X is —CR^{4a}R^{5a}—, —O—, —S—, —S(O)—, —NR^{6a}—, —S(O)₂—, —NR^{6a}S(O)₂—, —CR^{4a}R^{5a}S(O)₂—, —C(O)—, —NR^{6a}C(O)—, or —NHNHC(O)—;

wherein R^{4a} and R^{5a} are independently hydrogen, hydroxy, halogen, substituted C₁₋₄ alkyl, C₁₋₄ alkoxy, or substituted C₁₋₄ alkoxy;

or R^{4a} and R^{5a} are taken together with the carbon to which they are attached to form a 3- to 6-membered cycloalkyl ring;

each R^{6a} is independently hydrogen or C₁₋₄ alkyl;

### A is

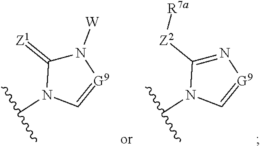

### G⁹ is CH or N;

Z¹ and Z² are independently S or O; and
W and R^{7a} are independently hydrogen or C₁₋₄ alkyl,
or a pharmaceutically acceptable salt thereof.

In some embodiments of any of the methods described herein, the compound is a compound of Formula (II):

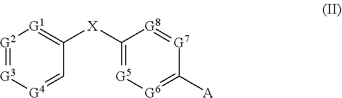

wherein

### G¹ is CH or N;

G² is CR^{2a} or N;
G³ is CR^{3a} or N;

### G⁴ is CH or N;

wherein no more than two of G¹, G², G³, and G⁴ are N;

### G⁵ is CH or N;

G⁶ is CR^{1a} or N;

### G⁷ is CH or N;

### G⁸ is CH or N;

wherein no more than one of G⁵, G⁶, G⁷, and G⁸ is N;
R^{1a}, R^{2a}, and R^{3a} are each independently hydrogen, hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, or an optionally substituted heterocyclyl;
   wherein R^{x} and R^{y} are each independently H or optionally substituted C₁₋₄alkyl;
or R^{1a} and R^{2a} are taken together with the carbons to which they are attached to form a 5- to 16-membered heterocyclyl ring;
   X is —CR^{4a}R^{5a}—, —O—, —S—, —S(O)—, —NR^{6a}—, —S(O)₂—, or —C(O)—;

wherein R^{4a} and R^{5a} are independently hydrogen, hydroxy, halogen, substituted C₁₋₄ alkyl, C₁₋₄ alkoxy, or substituted C₁₋₄ alkoxy;

or R^{4a} and R^{5a} are taken together with the carbon to which they are attached to form a 3- to 6-membered cycloalkyl ring;

R^{6a} is hydrogen or C₁₋₄ alkyl;

A is

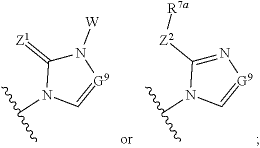

### G⁹ is CH or N;

Z¹ and Z² are independently S or O; and
W and R^{7a} are independently hydrogen or C₁₋₄ alkyl,
or a pharmaceutically acceptable salt thereof.

Provided in other aspects are compounds of Formula (IIA):

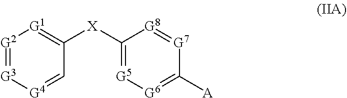

wherein

### G¹ is CH or N;

G² is CR^{2a} or N;
G³ is CR^{3a} or N;

### G⁴ is CH or N;

wherein no more than two of G¹, G², G³, and G⁴ are N;

### G⁵ is CH or N;

G⁶ is CR^{1a} or N;

### G⁷ is CH or N;

### G⁸ is CH or N;

wherein no more than one of G⁵, G⁶, G⁷, and G⁸ is N;
R^{1a}, R^{2a}, and R^{3a} are each independently hydrogen, hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, or an optionally substituted heterocyclyl;
   wherein R^{x} and R^{y} are each independently H or optionally substituted C₁₋₄alkyl; or R^{1a} and R^{2a} are taken together with the carbons to which they are attached to form a 5- to 16-membered heterocyclyl ring;
      X is —CR^{4a}R^{5a}—, —O—, —S—, —S(O)—, —NR^{6a}—, —S(O)₂—, —NR^{6a}S(O)₂—, —CR^{4a}R^{5a}S(O)₂—, —C(O)—, —NR^{6a}C(O)—, or —NHNHC(O)—;

wherein R^{4a} and R^{5a} are independently hydrogen, hydroxy, halogen, substituted C₁₋₄ alkyl, C₁₋₄ alkoxy, or substituted C₁₋₄ alkoxy;

or R^{4a} and R^{5a} are taken together with the carbon to which they are attached to form a 3- to 6-membered cycloalkyl ring;

each R^{6a} is independently hydrogen or C₁₋₄ alkyl;

A is

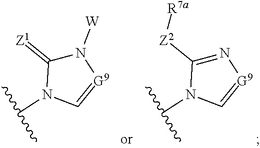

### G⁹ is CH or N;

Z¹ and Z² are independently S or O; and
W and R^{7a} are independently hydrogen or C₁₋₄ alkyl; and

one or more of the following apply:

(i) X is —CR^{4a}R^{5a}—, —O—, —S—, —S(O)—, —NR^{6a}—, —NR^{6a}S(O)₂—, —CR^{4a}R^{5a}S(O)₂—, —C(O)—, —NR^{6a}C(O)—, or —NHNHC(O)—;

(ii) one or two of G¹, G², G³, and G⁴ is N;

(iii) one of G⁵, G⁶, G⁷, and G⁸ is N;

(iv) R^{1a} is an optionally substituted heterocyclyl, wherein when the heterocyclyl is monocyclic, the point of connection is via a carbon atom;

(v) R^{2a} is an optionally substituted heterocyclyl, wherein when the heterocyclyl is monocyclic, the point of connection is via a carbon atom;

(vi) R^{3a} is an optionally substituted heterocyclyl, wherein when the heterocyclyl is monocyclic, the point of connection is via a carbon atom;

(vii) R^{2a} and R^{3a} are taken together with the carbons to which they are attached to form a 5- to 16-membered heterocyclyl ring;

(viii) A is

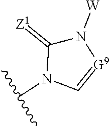

### and Z¹ is O;

(ix) A is

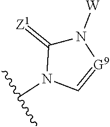

and W is C₁₋₄ alkyl;

(x) A is

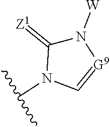

### and G⁹ is CH; and

(xii) A is

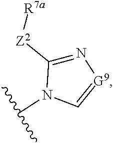

or a pharmaceutically acceptable salt thereof.

Provided in other aspects are compounds of Formula (II):

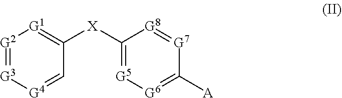

wherein

### G¹ is CH or N;

G² is CR^{2a} or N;
G³ is CR^{3a} or N;

### G⁴ is CH or N;

wherein no more than two of G¹, G², G³, and G⁴ are N;

### G⁵ is CH or N;

G⁶ is CR^{1a} or N;

### G⁷ is CH or N;

### G⁸ is CH or N;

wherein no more than one of G⁵, G⁶, G⁷, and G⁸ is N;
R^{1a}, R^{2a}, and R^{3a} are each independently hydrogen, hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, or an optionally substituted heterocyclyl;
   wherein R^{x} and R^{y} are each independently H or optionally substituted C₁₋₄alkyl;
or R^{1a} and R^{2a} are taken together with the carbons to which they are attached to form a 5- to 16-membered heterocyclyl ring;
   X is —CR^{4a}R^{5a}—, —O—, —S—, —S(O)—, —NR^{6a}—, —S(O)₂—, or —C(O)—;

wherein R^{4a} and R^{5a} are independently hydrogen, hydroxy, halogen, substituted C₁₋₄ alkyl, C₁₋₄ alkoxy, or substituted C₁₋₄ alkoxy;

or R^{4a} and R^{5a} are taken together with the carbon to which they are attached to form a 3- to 6-membered cycloalkyl ring;

R^{6a} is hydrogen or C₁₋₄ alkyl;

### A is

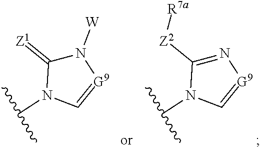

### G⁹ is CH or N;

Z¹ and Z² are independently S or O; and
W and R^{7a} are independently hydrogen or C₁₋₄ alkyl; and

one or more of the following apply:

(i) X is —CR^{4a}R^{5a}—, —O—, —S—, —S(O)—, —NR^{a}—, or —C(O)—;

(ii) one or two of G¹, G², G³, and G⁴ is N;

(iii) one of G⁵, G⁶, G⁷, and G⁸ is N;

(iv) R^{1a} is an optionally substituted heterocyclyl, wherein when the heterocyclyl is monocyclic, the point of connection is via a carbon atom;

(v) R^{2a} is an optionally substituted heterocyclyl, wherein when the heterocyclyl is monocyclic, the point of connection is via a carbon atom;

(vi) R^{3a} is an optionally substituted heterocyclyl, wherein when the heterocyclyl is monocyclic, the point of connection is via a carbon atom;

(vii) R^{2a} and R^{3a} are taken together with the carbons to which they are attached to form a 5- to 16-membered heterocyclyl ring;

(viii) A is

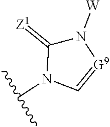

### and Z¹ is O;

(ix) A is

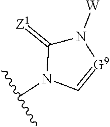

and W is C₁₋₄ alkyl;

(x) A is

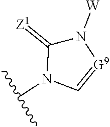

### and G⁹ is CH; and

(xii) A is

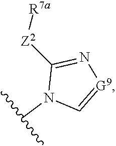

or a pharmaceutically acceptable salt thereof.

### BRIEF DESCRIPTION OF THE DRAWINGS

FIG. 1 shows a dose response curve for the activity of Compound 1 in the inhibition of organic anion transporter 3 (OAT3) in the uptake of 3H-PAH, using MDCK-II transfected with an OAT3 expression vector.

FIG. 2 shows a dose response curve for the activity of Compound 1 in the inhibition of organic anion transporter 1 (OAT1) in the uptake of 3H-PAH, using MDCK-II transfected with an OAT1 expression vector.

FIG. 3A shows the concentration of uric acid in the brain of mice administered (p.o.) with 50 mg/kg of compound 1. FIG. 3B shows the concentration of uric acid in blood plasma of mice administered (p.o.) with 50 mg/kg of compound 1.

FIG. 4A shows the concentration of DHEAS in the brain of mice administered (p.o.) with 50 mg/kg of compound 1. FIG. 4B shows the concentration of DHEAS in blood plasma of mice administered (p.o.) with 50 mg/kg of compound 1.

FIG. 5A shows the concentration of DHEA in the brain of mice administered (p.o.) with 50 mg/kg of compound 1. FIG. 5B shows the concentration of DHEA in blood plasma of mice administered (p.o.) with 50 mg/kg of compound 1.

FIG. 6A shows a ¹H NMR spectrum of Compound 1 in DMSO-d6 (400 MHz). FIG. 6B shows a 2D NOESY spectrum of Compound 1 in DMSO-d6 (400 MHz) as synthesized from Route B. FIG. 6C shows an expansion of the 2D NOESY spectrum of compound 1 in DMSO-d6 (500 mHz) as synthesized from Route B. FIG. 6D shows a 2D NOESY spectrum of Compound 1 in DMSO-d6 (400 MHz) as synthesized from Route C. FIG. 6E shows the HMBC of Compound 1 in DMSO-d6 (400 MHz) as synthesized from Route C.

FIG. 7A shows the PXRD diffractogram of Compound 1. FIG. 7B shows the overlayed PXRD diffractogram of four different spray dried formulations of Compound 1.

FIG. 8A shows the overlay of the DSC and TGA thermograms for Compound 1. FIG. 8B and FIG. 8C show the TGA and DSC thermograms, respectively, for spray dry dispersion (SDD) #1. FIG. 8D and FIG. 8E show the TGA and DSC thermograms, respectively, for spray dry dispersion (SDD) #2. FIG. 8F and FIG. 8G show the TGA and DSC thermograms, respectively, for spray dry dispersion (SDD) #3. FIG. 8H and FIG. 8I show the TGA and DSC thermograms, respectively, for spray dry dispersion (SDD) #4.

FIG. 9A shows the pharmacokinetic curves of Compound 1 in free base form (FB) and two spray dry dispersions of Compound 1 (SDD #1 and SDD #3). FIG. 9B shows the AUC vs. dose for Compound 1 in free base form (FB) and two spray dry dispersions of Compound 1 (SDD #1 and SDD #3).

FIG. 10A shows the single crystal structural analysis of Compound 1 (4-(4-((4-chloro-3-(trifluoromethyl)phenyl)sulfonyl)phenyl)-2,4-dihydro-3H-1,2,4-triazole-3-thione). FIG. 10B shows the single crystal structural analysis of an asymmetric unit of Compound 1.

FIG. 11 shows the X-ray powder diffractogram (XRPD) of Compound 1.

FIGS. 12A-C show the optical density of total alpha-synuclein deposits in the (12A) cortex, (12B) hippocampus, and (12C) striatum of L61 ASYN transgenic mice after i.p. administration of Compound 1 (1, 5, or 10 mg/kg per day) or a vehicle (5% DMSO+20% Cremphor EL+0.9% normal saline) for 1 month. Non-transgenic mice were used as a control group and were administered (i.p.) with Compound 1 (10 mg/kg per day) or a vehicle (5% DMSO+20% Cremphor EL+0.9% normal saline) for 1 month.

FIG. 13 shows the total alpha-synuclein deposits in representative images of cross-sections of the cortex, hippocampus, and striatum of L61 ASYN transgenic mice after i.p. administration of Compound 1 (1, 5, or 10 mg/kg per day) or a vehicle (5% DMSO+20% Cremphor EL+0.9% normal saline) for 1 month. Non-transgenic mice were used as a control group and were administered (i.p.) with a vehicle (5% DMSO+20% Cremphor EL+0.9% normal saline) for 1 month.

FIGS. 14A-C show the optical density of insoluble alpha-synuclein deposits (PK+resistant) in the (14A) cortex, (14B) hippocampus, and (14C) striatum of L61 ASYN transgenic mice after i.p. administration of Compound 1 (1, 5, or 10 mg/kg per day) or a vehicle (5% DMSO+20% Cremphor EL+0.9% normal saline) for 1 month. Non-transgenic mice were used as a control group and were administered (i.p.) with Compound 1 (10 mg/kg per day) or a vehicle (5% DMSO+20% Cremphor EL+0.9% normal saline) for 1 month.

FIG. 15 shows the insoluble alpha-synuclein deposits (PK+resistant) in representative images of cross-sections of the cortex, hippocampus, and striatum of L61 ASYN transgenic mice after i.p. administration of Compound 1 (1, 5, or 10 mg/kg per day) or a vehicle (5% DMSO+20% Cremphor EL+0.9% normal saline) for 1 month. Non-transgenic mice were used as a control group and were administered (i.p.) with a vehicle (5% DMSO+20% Cremphor EL+0.9% normal saline) for 1 month.

FIGS. 16A-B show the biochemical evaluation of brain levels of monomeric ASYN in the (16A) frontal cortex and (16B) hippocampus of L61 ASYN transgenic mice after i.p. administration of Compound 1 (1, 5, or 10 mg/kg per day) or a vehicle (5% DMSO+20% Cremphor EL+0.9% normal saline) for 1 month. Non-transgenic mice were used as a control group and were administered (i.p.) with a vehicle (5% DMSO+20% Cremphor EL+0.9% normal saline) for 1 month.

FIGS. 17A-C show the optical density of microtubule-associated protein 1A/1B-light chain 3 (LC3) in the (17A) cortex, (17B) hippocampus, and (17C) striatum of L61 ASYN transgenic mice after i.p. administration of Compound 1 (1, 5, or 10 mg/kg per day) or a vehicle (5% DMSO+20% Cremphor EL+0.9% normal saline) for 1 month. Non-transgenic mice were used as a control group and were administered (i.p.) with Compound 1 (10 mg/kg per day) or a vehicle (5% DMSO+20% Cremphor EL+0.9% normal saline) for 1 month.

FIG. 18 shows the levels of LC3 immunolabeling via IHC in representative images of cross-sections of the cortex, hippocampus, and striatum of L61 ASYN transgenic mice after i.p. administration of Compound 1 (1, 5, or 10 mg/kg per day) or a vehicle (5% DMSO+20% Cremphor EL+0.9% normal saline) for 1 month. Non-transgenic mice were used as a control group and were administered (i.p.) with a vehicle (5% DMSO+20% Cremphor EL+0.9% normal saline) for 1 month.

FIG. 19 shows the grip strength evaluation of L61 ASYN transgenic mice after administration with Compound 1 (5 or 10 mg/kg) or a vehicle (5% DMSO+20% Cremphor EL+0.9% normal saline) for 3 months. Non-transgenic mice were used as a control group and were administered (i.p.) with Compound 1 (10 mg/kg per day) or a vehicle (5% DMSO+20% Cremphor EL+0.9% normal saline) for 3 months.

FIG. 20A shows the levels of Translocator Protein (18 kDa) (TSPO) in representative images of cross-sections of the frontal cortex of L61 ASYN transgenic mice after administration with Compound 1 (5 or 10 mg/kg) or a vehicle (5% DMSO+20% Cremphor EL+0.9% normal saline) for 3 months. Non-transgenic mice were used as a control group and were administered (i.p.) with Compound 1 (10 mg/kg per day—data not shown) or a vehicle (5% DMSO+20% Cremphor EL+0.9% normal saline) for 3 months. FIG. 20B shows the quantification of the TSPO images from FIG. 20A.

FIG. 21 shows the IHC staining for GFAP in representative images of the hippocampus of L61 ASYN transgenic mice after i.p. administration of Compound 1 (1, 5, or 10 mg/kg per day) or a vehicle (5% DMSO+20% Cremphor EL+0.9% normal saline) for 1 month. Non-transgenic mice were used as a control group and were administered (i.p.) with Compound 1 (10 mg/kg per day—data not shown) or a vehicle (5% DMSO+20% Cremphor EL+0.9% normal saline) for 3 months.

FIG. 22 shows the optical density in IHC staining for GFAP in the hippocampus of L61 ASYN transgenic mice after i.p. administration of Compound 1 (1, 5, or 10 mg/kg per day) or a vehicle (5% DMSO+20% Cremphor EL+0.9% normal saline) for 3 months. Non-transgenic mice were used as a control group and were administered (i.p.) with Compound 1 (10 mg/kg per day—data not shown) or a vehicle (5% DMSO+20% Cremphor EL+0.9% normal saline) for 1 month.

FIG. 23 shows IHC staining of DAT in representative images of cross-sections of the striatum of L61 ASYN transgenic mice after administration with Compound 1 (5 or 10 mg/kg) or a vehicle (5% DMSO+20% Cremphor EL+0.9% normal saline) for 3 months. Non-transgenic mice were used as a control group and were administered (i.p.) with Compound 1 (10 mg/kg per day—data not shown) or a vehicle (5% DMSO+20% Cremphor EL+0.9% normal saline—data not shown) for 3 months.

FIG. 24 shows the striatal-to-reference ratio from optical density of IHC staining of DAT in representative images of cross-sections of the striatum and reference region (cortex) of L61 ASYN transgenic mice after administration with Compound 1 (5 or 10 mg/kg) or a vehicle (5% DMSO+20% Cremphor EL+0.9% normal saline) for 3 months. Non-transgenic mice were used as a control group and were administered (i.p.) with Compound 1 (10 mg/kg per day) or a vehicle (5% DMSO+20% Cremphor EL+0.9% normal saline) for 1 month.

FIG. 25 shows quantitation in TSPO immunofluorescence staining in representative brain sections of L41 APP transgenic mouse after daily i.p. injections of vehicle or Compound 1 at 5 mg/kg or vehicle for 70 days. Data for non-transgenic mouse administered with daily ip injections of vehicle was also shown.

FIG. 26 shows quantitation in immunofluorescence staining of amyloid beta using 6E10 antibodies in representative brain sections of L41 APP transgenic mouse after daily i.p. injections of vehicle or Compound 1 at 5 mg/kg or vehicle for 70 days. Data for non-transgenic mouse administered with daily ip injections of vehicle was also shown.

### DETAILED DESCRIPTION

The present disclosure relates to therapeutic agents that may be useful in the treatment and prophylaxis of neurodegenerative disorders and/or neural inflammation.

The transport of bioactive molecules across the blood-brain barrier (BBB) is tightly regulated in order to afford precise control of their levels in the brain interstitial space. The BBB is formed by a tight monolayer of endothelial cells and is an interface for exchange of compounds, allowing passive diffusion of water, some gases and certain lipid-soluble molecules. Nevertheless, other molecules, such as organic anions, are selectively transported across the BBB. An active efflux system in the BBB controls the unbound concentrations of exogenous compounds in the brain interstitial space and inactivates neuroactive compounds by transferring them into the blood. The organic anion transporter (OAT) family of proteins includes members that transport organic ions across various membranes. Organic anion transporter 3 (OAT3), possibly the most abundantly expressed organic ion transport subtype in the brain (Gasser et al., 2009; Roberts et al., 2008), mediates the active efflux from the brain of bioactive endogenous metabolites, some of which possess anti-inflammatory and neuroprotective activities.

Among the bioactive endogenous metabolites found in the brain, soluble uric acid as well as dehydroepiandrosterone (DHEA) and its conjugate ester, DHEA sulfate (DHEAS) have been shown in various animal models to demonstrate beneficial neuroprotection and anti-inflammatory actions.

Uric acid has been widely shown to have robust neuroprotective and anti-inflammatory actions in cell systems and in a wide range of animal models of neurodegenerative disorders. In cell based systems, uric acid has neuroprotective actions that are mediated via astroglial cells in co-culture with neuronal cells and directly on dopaminergic cell lines (Bakshi et al., 2015; Du et al., 2007; Zhang et al., 2014). This neuroprotective action is likely the result of Nrf2 stability and nuclear translocation with consequent upregulation of glutathione and other antioxidant gene products (Bakshi et al., 2015; Zhang et al., 2014). In particular, the beneficial effects on inflammatory and neuropathology endpoints have been demonstrated by a number of different investigators using either 6-OHDA or MPTP lesion models for Parkinson's disease (Crotty et al., 2017; Chen et al., 2013; Gong et al., 2012; Huang et al., 2017). Some of the beneficial effects are exemplified by decreased levels of inflammatory markers such as glial fibrillary acidic protein (GFAP), improved dopamine system integrity and an associated upregulation of phosphorylated Akt (pAkt) in these toxin based models of Parkinson's disease.

DHEA and DHEAS are the most abundant circulating steroid hormones in humans. In the brain, DHEA is synthesized by neurons and astrocytes, and is therefore considered a neurosteroid. Unlike the lipophilic DHEA, DHEAS is hydrophilic and does not readily cross the BBB. It has been shown that OAT3 plays a major role in the efflux of DHEAS from the brain interstitial space into the blood (Miyajima et al., 2011). Age-related decline of circulating DHEAS has been associated with declining levels of circulating DHEAS (Maninger et al., 2009, Callier et al., 2003, Belanger et al., 2006, Li et al., 2001, Charalampopoulos et al., 2004, Gravanis et al., 2012, Charalampopoulos et al., 2008). It has been shown that DHEA treatment inhibits acute microglia-mediated neuroinflammation through activation of the TrkA-Akt1/2-CREB-Jmjd3 pathway. The anti-inflammatory action conferred by DHEAS is thus associated with an increase in pAkt, activation of Jmjd3, promotion of anti-inflammatory microglia phenotype, and inhibition of pro-inflammatory microglia phenotype, among other resultant phenotypes (Alexaki et al., 2016).

Nevertheless, a systemic increase in concentration of these bioactive metabolites can introduce deleterious effects because of their bioactive properties outside of the brain interstitial space. For example, elevated plasma levels of uric acid will lead to the increased risk of gout as well as other consequences of hyperuricemia (Roddy and Choi 2015, Terkeltaub et al., 2006).

As a result, there exists the need to selectively increase the concentration of neuroprotectants in the brain interstitial space without elevating the corresponding plasma levels. Because OAT3 may be a primary efflux transporter for uric acid and DHEAS from the brain interstitial space into the blood, while in other organs its function (such as urate secretion in kidney) could be largely compensated by structurally similar anion transporters (such as OAT1) (Riedmaier et al., 2012, Wu et al., 2017, Eraly et al., 2008), the selective blockade of OAT3 would elevate the levels of neuroprotectants in the brain interstitial space without significantly changing the corresponding plasma levels. Such a selective OAT3 blockade would also avail the option of treating a subject with systemic administration instead of being restricted to targeted delivery. Therefore there exists a need to utilize a selective inhibitor that blocks OAT3-mediated efflux without significantly changing the transport mediated by other anion transporters such as OAT1. While there are molecules such as probenecid or taurocholate that block OAT3 to increase levels of DHEAS and presumably uric acid in the brain interstitial space, these molecules will also inhibit structurally similar anion transporters such as OAT1 (Yin and Wang 2016, Wu et al., 2017). Specifically, probenecid and taurocholate, in addition to blocking OAT3, can block other anion transporters (Miyajima et al., 2011), and can also alter plasma levels of certain therapeutic agents, leading to the concerns for drug-drug interactions (Yin and Wang 2016, Klatt et al., 2011). Likewise, in vitro substrates such as para-aminohippurate (PAH) that are used to competitively inhibit OAT3 (Miyajima et al., 2011), are also substrates for OAT1 (Nigam et al., 2015, Nozaki et al., 2007) and have long been used to decrease the clearance of drugs from the body by the kidney (Beyer et al., 1944).

One aspect of the current disclosure involves compounds that selectively block the efflux of bioactive endogenous metabolites from the brain interstitial space into the blood. In some embodiments, the blockade is mediated by inhibition of ion transporter-mediated efflux across the BBB. In some embodiments, the compounds in the present application selectively inhibit the ion transporters that mediate the efflux of bioactive endogenous compounds across the BBB. In some embodiments, the ion transporters comprise OAT3. In some embodiments, the bioactive endogenous metabolites comprise molecules that exhibit neuroprotective and/or anti-neuroinflammatory activities. In further embodiments, the bioactive endogenous metabolites comprise DHEA, DHEAS and/or uric acid. In some embodiments, the presently disclosed compounds are inhibitors of organic anion transporter 3 (OAT3). For instance, in some embodiments, the presently disclosed compounds have an IC₅₀ for OAT3 of about 1 μM or less. In certain embodiments, the potency of the presently disclosed compounds to inhibit OAT3 is about 20-fold higher than that for the structurally similar OAT1. In one aspect, the potent inhibitory function of the presently disclosed compounds on OAT3 are harnessed to block the OAT3-mediated efflux across the BBB, therefore increasing the local levels of bioactive molecules in the brain interstitial space which confer neuroprotection and/or anti-neuroinflammation. In some of the embodiments, the bioactive molecules that confer neuroprotection and/or anti-neuroinflammation comprise one or more of dehydroepianodrosterone (DHEAS), DHEA sulfate (DHEAS), glutathione, and uric acid. As described earlier, such benefits of neuroprotection and anti-neuroinflammation are accompanied by activation of the TrkA-Akt1/2-CREB-Jmjd3 pathway, decreased levels of inflammatory markers (GFAP), improved dopamine system integrity and an associated upregulation of pAkt. Interestingly, Line61 transgenic models when treated with certain compounds presently disclosed exhibited similar beneficial effects of reduced expression of inflammatory markers (such as GFAP) and improved dopamine system integrity, together with an associated upregulation of pAkt. These results correlate with the general effects of DHEA/DHEAS and/or uric acid administration to mouse models, and are highly similar to the phenotypes exhibited by toxin-based models of Parkinson's diseases when treated with uric acid. Moreover, by selectively blocking OAT3, the presently disclosed compounds could confer neuroprotection and anti-neuroinflammation without the harmful side effects brought on by systemic increases of uric acid or DHEA/DHEAS levels resulting from molecules that inhibit multiple ion transporters in addition to OAT3. Without being bound by theory, the compounds described herein selectively block the efflux of neuroprotectants from the brain interstitial space into the blood by selectively inhibiting the active efflux by ion transporters across the blood brain barrier (BBB), thereby increasing the local concentration of neuroprotectants in the brain interstitial space. In some embodiments, the ion transporters being inhibited comprise one or more ion transporters including OAT3. In some embodiments, the ion transporters being inhibited comprise anion transporter OAT3. In some embodiments, the ion transporter inhibitor comprises a compound of Formula (I), or a pharmaceutically acceptable salt thereof, as described herein. In one aspect, provided is a method for treating a condition associated with neurodegeneration or accumulation of proteins in the brain in a subject in need thereof, comprising administering to the subject a therapeutically effective amount of a compound that is an inhibitor of organic anion transporter 3 (OAT3). In another aspect, provided is a method of treating a disease or condition associated with neuroinflammation, comprising administering to a subject in need of such treatment an effective amount of a compound that is an inhibitor of organic anion transporter 3 (OAT3). In some embodiments, the compound selectively inhibits OAT3. In some embodiments, the compound is administered systematically. In other embodiments, the compound is administered to the brain interstitial space with targeted delivery.

One aspect of the current invention involves a compound that selectively blocks the efflux of neuroprotectants from the brain interstitial space into the blood. In some embodiments, the blockade is mediated by inhibition of OAT3-mediated efflux across the BBB. In some embodiments, the neuroprotectants comprise one or more of dehydroepianodrosterone (DHEAS), DHEA sulfate (DHEAS), glutathione, and uric acid. Certain compounds described herein are developed by the applicant, and are potent and selective inhibitors of OAT3. It has been shown that the potency of some of these compounds to inhibit OAT3 is about 20-fold higher than that for the structurally similar OAT1. The potent inhibitory function of these compounds on OAT3 is harnessed to block the OAT3-mediated efflux across the BBB, therefore increasing the local levels of bioactive molecules in the brain interstitial space which confer neuroprotection and/or anti-neuroinflammation. In some embodiments, the bioactive molecules that confer neuroprotection and/or anti-neuroinflammation comprise one or more of dehydroepianodrosterone (DHEAS), DHEA sulfate (DHEAS), glutathione, and uric acid. As described earlier, such benefits of neuroprotection and anti-neuroinflammation are accompanied by activation of the TrkA-Akt1/2-CREB-Jmjd3 pathway, decreased levels of inflammatory markers (GFAP), improved dopamine system integrity and an associated upregulation of pAkt. Interestingly, Line61 transgenic models when treated with certain compounds described herein exhibit similar beneficial effects of reduced expression of inflammatory marker (such as GFAP) and improved dopamine system integrity, together with an associated upregulation of pAkt. These results correlate with the general effects of DHEA/DHEAS and/or uric acid administration to mouse models, and are highly similar to the phenotypes exhibited by toxin-based models of Parkinson's diseases when treated with uric acid. Moreover, by selectively blocking OAT3, these compounds described herein could confer neuroprotection and anti-neuroinflammation without the harmful side effects brought on by systemic elevation of uric acid or DHEA/DHEAS levels resulting from inhibition of multiple ion transporters in addition to OAT3.

Without being bound by theory, the compounds described herein selectively block the efflux of neuroprotectants from the brain interstitial space into the blood by selectively inhibiting the active efflux by ion transporters across the blood brain barrier (BBB), thereby increasing the local concentration of neuroprotectants in the brain interstitial space. In some embodiments, the ion transporters being inhibited comprise one or more ion transporters including OAT3. In some embodiments, the ion transporters being inhibited comprise anion transporter OAT3. In some embodiments, the ion transporter inhibitor comprises a compound of Formula (I), or a pharmaceutically acceptable salt thereof. In some embodiments the compound or the pharmaceutically acceptable salt thereof comprises compound 1. In one aspect, provided is a method for treating a condition associated with neurodegeneration or accumulation of proteins in the brain in a subject in need thereof, comprising administering to the subject a therapeutically effective amount of a compound that is an inhibitor of organic anion transporter 3 (OAT3). In another aspect, provided is a method of treating a disease or condition associated with neuroinflammation, comprising administering to a subject in need of such treatment an effective amount of a compound that is an inhibitor of organic anion transporter 3 (OAT3). In some embodiments, the compound selectively inhibits OAT3. In some embodiments, the ion transporter inhibitor (e.g., a compound of Formula (I), or a pharmaceutically acceptable salt thereof) is administered systematically. In other embodiments, the compound is administered to the brain interstitial space with targeted delivery.

Another aspect of the current disclosure involves OAT3 inhibitors that enhance the action of other drugs that are eliminated by OAT3. For instance, potent and selective inhibitors of OAT3 may augment cancer chemotherapy for certain cancers, such as brain cancers. Certain therapeutic agents used to treat brain cancers or viral infections in the brain are eliminated from the brain by the OAT3 transporter located in the choroid plexus. This active transport of these therapeutic agents out of the brain limits their efficacy. Thus, blockade of OAT3 transporters in the choroid plexus will slow the efflux of these therapeutic agents from the brain and thereby increase their efficacy in treating cancers and infections in the brain. (See Li et al., Clin Cancer Res. 2017 Dec. 15; 23(24):7454-7466; Nagle et al., Neurosci Lett. 2013 Feb. 8; 534:133-8; and Ose et al., Drug Metab Dispos. 2009 February; 37(2):315-21.) In some embodiments, OAT3 inhibitors such as compounds described herein enhance the action of other drugs that are eliminated by OAT3 from the brain. In some embodiments, OAT3 inhibitors such as compounds described herein enhances the action of drugs including, but not limited to, cancer chemotherapeutic agents (e.g., methyltrexate) and anti-viral agents (e.g., HIV therapeutics).

Another aspect of the current disclosure involves OAT3 inhibitors that prevent renal toxicity of cancer chemotherapeutics and other OAT3 substrates by blocking the uptake of such agents into kidney cells. For some cancer agents and some antiviral agents, the dose limiting toxicity that precludes achieving full efficacy is renal toxicity (caused by their accumulation in the renal proximal tubule cells). Certain cytotoxic drugs (e.g., cisplatin and methotrexate) cause dose-limiting nephrotoxicity. In some cases, the uptake of these cancer therapeutic agents into the renal proximal tubule cells is mediated by OAT3. The drugs are eliminated from the blood into the urine by active transport (as opposed to passive filtration). The path from blood to urine for these drugs includes active transport across the renal tubular basolateral membrane (blood side) into the kidney cell and then transport out of the kidney cell by different transporters on the apical (urine) side. If the drugs are cytotoxic and if the drugs accumulate in the renal tubule cell (more basolateral transport than apical transport), then they can cause renal toxicity. A blocker of the basolateral transport of these drugs would slow the uptake into the renal tubule cell and so prevent its accumulation in these cells and thus prevent renal toxicity. One of the basolateral transporters for these cytotoxic drugs is OAT3. Thus, an OAT3 inhibitor may slow uptake of these toxic drugs into the kidney proximal tubule cell and thereby prevent their renal toxicity. (See Hu et al., Clin Transl Sci. 2017 September; 10(5):412-420; Xue et al., Mol Pharm. 2011 Dec. 5; 8(6):2183-92; Hagos et al., Toxins (Basel). 2010 August; 2(8):2055-82; and Jung et al., Life Sci. 2002 Mar. 8; 70(16):1861-74.) In some embodiments, OAT3 inhibitors would act to spare the kidney from damage while allowing for higher and more prolonged dosing with cytotoxic drugs (e.g., cancer chemotherapeutic agents).

### Terms

It is to be understood that the compounds, compositions, methods, and uses described herein are not limited to particular embodiments described, as such may, of course, vary. It is also to be understood that the terminology used herein is for the purpose of describing particular embodiments only, and is not intended to be limiting, since the scope of the compounds, compositions, methods, and uses described herein will be limited only by the appended claims.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

The following terms have the following meanings unless otherwise indicated. Any undefined terms have their art recognized meanings.

The term “effective amount” used herein refers to an amount of a compound or composition sufficient to treat a specified disorder, condition or disease, such as a disease or condition associated with neurodegeneration or neuroinflammation, so as to ameliorate, palliate, lessen, and/or delay one or more of its symptoms.

As used herein, “treatment” is an approach for obtaining beneficial or desired clinical results. “Treatment” as used herein, covers any administration or application of a therapeutic for disease in a mammal, including a human. For purposes of this invention, beneficial or desired clinical results include, but are not limited to, any one or more of: alleviation of one or more symptoms, diminishment of extent of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods of the invention contemplate any one or more of these aspects of treatment. As used herein, the term “prevention” is an approach that includes but is not limited to preventing or delaying the occurrence or spread of disease, preventing or delaying recurrence of disease, or preventing or delaying the progression of the disease.

As used herein, the term “inhibit” may refer to the act of blocking, reducing, eliminating, or otherwise antagonizing the presence, or an activity of, a particular target. Inhibition may refer to partial inhibition or complete inhibition. For example, inhibiting an ion transporter protein may refer to any act leading to a blockade, reduction, elimination, or any other antagonism of the activity of the ion transporter protein. In some examples, activity of a target (e.g., a protein) is inhibited by a substrate, such as a small molecule. In some embodiments, inhibition of a target by a substrate is competitive. In other embodiments, inhibition of the target by a substrate is noncompetitive. In some embodiments, inhibition of the target by a substrate is reversible. In other embodiments, inhibition of the target by a substrate is non-reversible.

As used herein, the term “modulate” may refer to the act of changing, altering, varying, or otherwise modifying the presence, or an activity of, a particular target. For example, modulating the efflux of certain molecules may refer to any act leading to changing, altering, varying, or otherwise modifying the efflux of the molecules. For example, modulating the local concentration of neuroprotectants may refer to any act leading to changing, altering, varying, or otherwise modifying the local concentration of neuroprotectants. In some examples, “modulate” refers to enhancing the presence or activity of a particular target. In other examples, “modulate” refers to suppressing the presence or activity of a particular target. In some examples, modulating the efflux of certain molecules may refer to any act leading to increasing the efflux of the molecules. In other examples, modulating the efflux of certain molecules may refer to any act leading to decreasing the efflux of the molecules.

Unless clearly indicated otherwise, a “subject” as used herein intends a mammal, including but not limited to a primate, human, bovine, horse, feline, canine, or rodent. In one variation, the subject is a human.

As used herein, “endogenous metabolites” are products of metabolism that are formed via chemical reactions that occur within the cells of a subject. For example, uric acid is an endogenous metabolite that can be produced from the chemical reactions in the breakdown of purine nucleotides within cells of the subject. For example, DHEA is an endogenous metabolite that can be produced as an intermediate metabolite in the chemical reactions in the chemical synthesis of androgen and estrogen in the adrenal glands and the brains of the subject. Other examples of endogenous metabolites are known in the art.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

In some aspects, the half maximal inhibitory concentration (IC₅₀) is a measure of the effectiveness of a substance in inhibiting a specific biological or biochemical function. In some aspects, the IC₅₀ is a quantitative measure that indicates how much of an inhibitor is needed to inhibit a given biological process or component of a process such as an enzyme, cell, cell receptor or microorganism by half. Methods of determining IC₅₀ in vitro and in vivo are known in the art.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

The term “alkyl” refers to a straight- or branched-chain alkyl (hydrocarbon) group having from 1 to 12 carbon atoms in the chain. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. In some instances, alkyl groups are C₁₋₄alkyl.

“Alkenyl” refers to an unsaturated branched or straight-chain hydrocarbon group having the indicated number of carbon atoms (e.g., 2 to 8, or 2 to 6 carbon atoms) and at least one site of olefinic unsaturation (having at least one carbon-carbon double bond). The alkenyl group may be in either the cis or trans configuration (Z or E configuration) about the double bond(s). Alkenyl groups include, but are not limited to, ethenyl, propenyl (e.g., prop-1-en-1-yl, prop-1-en-2-yl, prop-2-en-1-yl (allyl), prop-2-en-2-yl), and butenyl (e.g., but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl).

“Alkynyl” refers to an unsaturated branched or straight-chain hydrocarbon group having the indicated number of carbon atoms (e.g., 2 to 8 or 2 to 6 carbon atoms) and at least one site of acetylenic unsaturation (having at least one carbon-carbon triple bond). Alkynyl groups include, but are not limited to, ethynyl, propynyl (e.g., prop-1-yn-1-yl, prop-2-yn-1-yl) and butynyl (e.g., but-1-yn-1-yl, but-1-yn-3-yl, but-3-yn-1-yl).

“Aryl” or “Ar” as used herein refers to an unsaturated aromatic carbocyclic group having a single ring (e.g., phenyl) or multiple condensed rings (e.g., naphthyl or anthryl), which condensed rings are carbocyclic and may or may not be aromatic, provided at least one ring in the multiple condensed ring structure is aromatic. Particular aryl groups are those having from 6 to 14 annular carbon atoms (a “C₆-C₁₄ aryl”). An aryl group having more than one ring where at least one ring is non-aromatic is connected to the parent structure at either an aromatic ring position or at a non-aromatic ring position. In one variation, an aryl group having more than one ring where at least one ring is non-aromatic is connected to the parent structure at an aromatic ring position.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

“Cycloalkyl” as used herein refers to and includes, unless otherwise stated, saturated or partially unsaturated nonaromatic cyclic univalent hydrocarbon structures, having the number of carbon atoms designated (i.e., C₃-C₁₀ means three to ten carbon atoms). Cycloalkyl can consist of one ring, such as cyclohexyl, or multiple rings, such as adamantyl. A cycloalkyl comprising more than one ring may be fused, spiro or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃-C₈ cycloalkyl”), having 3 to 6 annular carbon atoms (a “C₃-C₆ cycloalkyl”), or having from 3 to 4 annular carbon atoms (a “C₃-C₄ cycloalkyl”). Examples of cycloalkyl include, but are not limited to, cyclopropyl, cyclobutyl, cyclopentyl, cyclohexyl, cycloheptyl, norbornyl, and the like.

“Cyano” or “nitrile” refers to the group —CN.

“Halo” or “halogen” refers to fluoro, chloro, bromo, and iodo.

“Hydroxy” or “hydroxyl” refers to the group —OH.

“Heterocycloalkyl” or “heterocyclyl” refers to a saturated or partially unsaturated group having a single ring or multiple condensed rings, including fused, bridged, or spiro ring systems, and having from 3 to 20 ring atoms, including 1 to 10 hetero atoms. These ring atoms are selected from the group consisting of carbon, nitrogen, sulfur, and oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are oxidized to provide for N-oxide, —S(O)—, or —S(O)₂— moieties. Examples of heterocycloalkyls include, but are not limited to, azetidine, oxetane, tetrahydrofuran, pyrrolidine, piperazine, piperidine, morpholine, thiomorpholine, 1,1-dioxothiomorpholinyl, dihydroindole, indazole, quinolizine, imidazolidine, imidazoline, indoline, 1,2,3,4-tetrahydroisoquinoline, thiazolidine, and the like. In some instances, heterocycloalkyl groups are 4-, 5-, or 6-membered rings. In some instances, the heterocycloalkyl comprises a fused phenyl ring.

“Heteroaryl” as used herein refers to an unsaturated aromatic cyclic group having from 1 to 14 annular carbon atoms and at least one annular heteroatom, including but not limited to heteroatoms such as nitrogen, oxygen and sulfur. A heteroaryl group may have a single ring (e.g., pyridyl, furyl) or multiple condensed rings (e.g., indolizinyl, benzothienyl), which condensed rings may be carbocyclic or may contain one or more annular heteroatom and which may or may not be aromatic, provided at least one ring in the multiple condensed ring structure is both aromatic and contains at least one annular heteroatom, and provided that the point of attachment is through the aromatic ring containing at least one annular heteroatom. A heteroaryl group may be connected to the parent structure at a ring carbon atom or a ring heteroatom. Particular heteroaryl groups are 5 to 14-membered rings having 1 to 12 annular carbon atoms and 1 to 6 annular heteroatoms independently selected from nitrogen, oxygen and sulfur, 5 to 10-membered rings having 1 to 8 annular carbon atoms and 1 to 4 annular heteroatoms independently selected from nitrogen, oxygen and sulfur, or 5, 6 or 7-membered rings having 1 to 5 annular carbon atoms and 1 to 4 annular heteroatoms independently selected from nitrogen, oxygen and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen and sulfur.

“Oxo” refers to the group (═O) or (O).

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —S(O)R⁷⁰, —S(O)₂R⁷⁰, —S(O)₂O⁻M⁺, —S(O)₂OR⁷⁰, —OS(O)₂R⁷⁰, —OS(O)₂O⁻ M⁺, —OS(O)₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻ M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC (O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰C(O)O⁻ M⁺, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heterocycloalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 3-, 4-, 5-, 6-, or 7-membered heterocycloalkyl which may optionally include from 1 to 4 of the same or different additional heteroatoms selected from the group consisting of O, N and S, of which N may have —H, C₁-C₄ alkyl, —C(O)C₁₋₄alkyl, —C(O)OC₁₋₄alkyl, or —S(O)₂C₁₋₄alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound provided herein and the other a typical counter ion such as chloride, or two ionized compounds disclosed herein can serve as counter ions for such divalent alkali earth ions, or a doubly ionized compound provided herein can serve as the counter ion for such divalent alkali earth ions). As specific examples, —NR⁸⁰R⁸⁰ is meant to include —NH₂, —NH-alkyl, N-pyrrolidinyl, N-piperazinyl, 4-N-methyl-piperazin-1-yl and N-morpholinyl.

In addition to the substituent groups disclosed with respect to the individual terms herein, substituent groups for hydrogens on nitrogen atoms in “substituted” heterocycloalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)R⁷⁰, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P (O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C (O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined. Where a heterocycloalkyl group is “substituted,” unless otherwise constrained by the definition for the heterocycloalkyl substituent, such groups can be substituted with 1 to 5, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, azido, cyano, halogen, hydroxyl, oxo, thioketo, carboxyl, carboxyl ester, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, sulfonylamino, —S(O)-alkyl, —S(O)-substituted alkyl, —S(O)-aryl, —S(O)-heteroaryl, —S(O)-heterocyclyl, —S(O)₂-alkyl, —S(O)₂-substituted alkyl, —S(O)₂-aryl, —S(O)₂-heteroaryl, and —S(O)₂-heterocyclyl.

It is understood that when a group is indicated as “substituted”, it may be substituted with 1 or more substituents, and that the substituents may be present at any or all of the valency-allowed position(s) on the system. In some embodiments, a group that is substituted has 1, 2, 3, or 4 substituents, 1, 2, or 3 substituents, 1 or 2 substituents, or 1 substituent.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 2 to 5, 3 to 5, 2 to 3, 2 to 4, 3 to 4, 1 to 3, 1 to 4 or 1 to 5 substituents. In one embodiment, the “optionally substituted” group is not substituted.

Unless indicated otherwise, the nomenclature of substituents that are not explicitly defined herein are arrived at by naming the terminal portion of the functionality followed by the adjacent functionality toward the point of attachment.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers. Compounds that have asymmetric centers can exist as one or more enantiomeric forms, one or more diastereomeric forms, one or more atropisomeric forms, and mixtures thereof in any ratio.

Any formula given herein is intended to refer also to any one of hydrates, solvates, and amorphous and polymorphic forms of such compounds, and mixtures thereof, even if such forms are not listed explicitly.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into the compounds described herein include isotopes of hydrogen, carbon, nitrogen, oxygen, phosphorous, fluorine, chlorine, and iodine, such as ²H, ³H, ¹¹C, ¹³C, ¹⁴C, ¹⁵N ¹⁸O, ¹⁷O, ³¹P, ³²p, ³⁵S, ¹⁸F, ³⁶Cl, and ¹²⁵I, respectively. Such isotopically labeled compounds are useful in metabolic studies (e.g., with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In some embodiments, ¹⁸F or ¹¹C labeled compounds are used for PET or SPECT studies. PET and SPECT studies may be performed as described, for example, by Brooks, D. J., “Positron Emission Tomography and Single-Photon Emission Computed Tomography in Central Nervous System Drug Development,” NeuroRx 2005, 2(2), 226-236, and references cited therein. Further, substitution with heavier isotopes such as deuterium (i.e., ²H) may afford certain therapeutic advantages resulting from greater metabolic stability, for example increased in vivo half-life or reduced dosage requirements. Isotopically labeled compounds and prodrugs thereof can generally be prepared by carrying out the procedures disclosed in the schemes or in the examples and preparations described below by substituting a readily available isotopically labeled reagent for a non-isotopically labeled reagent.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art. The methods and materials are now described; however, any methods and materials similar or equivalent to those described herein can also be used in the practice or testing of the compounds of compositions described herein. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, 4^{th} edition, New York: Oxford University Press, 2002, pp. 360-361, 1084-1085; Smith and March, March's Advanced Organic Chemistry: Reactions, Mechanisms, and Structure, 5^{th} edition, Wiley-Interscience, 2001.

The nomenclature used herein to name the subject compounds is illustrated in the Examples herein. This nomenclature has generally been derived using the commercially-available ChemBioDraw Ultra 13.0.2.3021 (CambridgeSoft, Cambridge, Mass.).

It is appreciated that certain features of the compounds, compositions, methods, and uses described herein, which are, for clarity, described in the context of separate embodiments, may also be provided in combination in a single embodiment. Conversely, various features of the compounds, compositions, methods, and uses described herein which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Methods of Use and Uses

Provided herein are methods of treating a disease or condition associated with neurodegeneration or accumulation of proteins in the brain in a subject in need thereof, comprising administering to the subject an effective amount of an ion transporter inhibitor (e.g., an inhibitor of organic anion transporter 3 (OAT3)), wherein the ion transporter inhibitor modulates the efflux of one or more bioactive endogenous metabolites across the blood brain barrier (BBB) of the subject. Neurodegenerative disorders may refer to disorders characterized by a loss of neurons and may or may not include a neuroinflammatory process. Neurodegenerative disorders include stroke, head trauma, cerebral hypoxia, spinal cord injury, senile dementia, Alzheimer's disease, amyotrophic lateral sclerosis (ALS) and other motor neuron diseases, cerebral amyloid angiopathy, HIV-related dementia, Parkinson's disease, Huntington's disease, prion diseases, myasthenia gravis, Down's syndrome, Creutzfeldt-Jakob disease, Friedreich's ataxia, Fergusson and Critchley's ataxia and other ataxias, Leber's hereditary optic neuropathy diabetic neuropathy, neuropathic pain, encephalitis, meningitis, and Duchenne's muscular dystrophy, fronto-temporal dementia, dementia with Lewy Bodies, PD dementia, multiple system atrophy, progressive supranuclear palsy, or neuroinflammation. In some embodiments, the condition associated with neurodegeneration is Alzheimer's Disease, Parkinson's Disease, fronto-temporal dementia, dementia with Lewy Bodies, PD dementia, multiple system atrophy, Huntington's disease, Amyotrophic lateral sclerosis, progressive supranuclear palsy, or neuroinflammation.

Provided in other aspects are methods of modulating efflux of one or more bioactive endogenous metabolites across the blood brain barrier (BBB) in a subject in need thereof, comprising administering to the subject in need thereof an ion transporter inhibitor (e.g., an inhibitor of organic anion transporter 3 (OAT3)).

Provided in another aspect is a method of improving neuroprotection in a subject in need thereof, comprising administering to the subject an effective amount of an ion transporter inhibitor (e.g., an inhibitor of organic anion transporter 3 (OAT3)) that modulates the concentration of one or more bioactive endogenous metabolites in the brain interstitial space. “Neuroprotection” may refer to actions, mechanisms, functions or characteristics that counteract neurodegenerative disorders or diseases and can be any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “neuroprotection” is a reduction of pathological consequence of a neurodegenerative or neurological disease. A “neuroprotectant” comprises an agent that can provide neuroprotection or possesses neuroprotective properties. The methods of the invention contemplate any one or more of these aspects of protection.

In other aspects, provided are methods of decreasing neuroinflammation in a subject in need thereof, comprising administering to the subject an effective amount of an ion transporter inhibitor (e.g., an inhibitor of organic anion transporter 3 (OAT3)) that modulates the concentration of one or more bioactive endogenous metabolites in the brain interstitial space. The term “neuroinflammation” or “neuroinflammatory diseases, disorders or conditions” may refer to diseases, disorders or conditions characterized by large numbers of reactive microglia in postmortem brain samples, indicative of an active inflammatory process (McGeer E. G. and McGeer P. L., “Neurodegeneration and the immune system”. Calne D. B., ed. Neurodegenerative Diseases, 1994:277-300). Neuroinflammation refers to inflammation which occurs in response to brain injury or autoimmune disorders, and has been shown to cause destruction of healthy neuronal and/or cerebral tissue. Neuroinflammation relates to mechanisms implicated in a broad range of acute and chronic neurodegenerative disorders, including stroke, head trauma, cerebral amyloid angiopathy, HIV-related dementia, Huntington's disease, prion diseases, meningitis, myelin degradation, epilepsy, Down's syndrome, post-ischemic brain injury, encephalopathy, Parkinson's disease, senile dementia, Alzheimer's disease, amyotrophic lateral sclerosis, multiple sclerosis and certain disorders involving the peripheral nervous system, such as myasthenia gravis and Duchenne's muscular dystrophy.

In some embodiments of any of the methods described herein, the ion transporter inhibitor has an IC₅₀ of about 1 μM or less. In some embodiments, the ion transporter inhibitor has an IC₅₀ of between about 0.1 μM and about 1 μM. In some embodiments, the ion transporter inhibitor has an IC₅₀ of between about 1 nM and about 10 μM, such as between about 0.5 μM and about 5 μM, between about 0.1 μM and about 2 μM, between about 50 nM and about 1 μM, between about 10 nM and about 500 nM, between about 5 nM and about 100 nM, or between about 1 nM and about 50 nM. In some embodiments, the ion transporter inhibitor has an IC₅₀ of less than about 1 nM. In some embodiments of any of the methods described herein, the ion transporter inhibitor selectively inhibits OAT3 compared with other ion transporter proteins. In some embodiments of any of the methods described herein, the ion transporter inhibitor has an IC₅₀ for OAT3 of about 1 μM or less. In some embodiments, the ion transporter inhibitor has an IC₅₀ for OAT3 of between about 0.1 μM and about 1 μM. In some embodiments, the ion transporter inhibitor has an IC₅₀ for OAT3 of between about 1 nM and about 10 μM, such as between about 0.5 μM and about 5 μM, between about 0.1 μM and about 2 μM, between about 50 nM and about 1 μM, between about 10 nM and about 500 nM, between about 5 nM and about 100 nM, or between about 1 nM and about 50 nM. In some embodiments, the ion transporter inhibitor has an IC₅₀ for OAT3 of less than about 1 nM.

In some embodiments of any of the methods described herein, the ion transporter inhibitor is an inhibitor of organic anion transporter 3 (OAT3). In some embodiments, the ion transporter inhibitor has an IC₅₀ for OAT3 of about 500 nM or less. In some embodiments, the ion transporter inhibitor has an IC₅₀ for OAT3 of about 1 μM or less. In some embodiments of any of the methods described herein, the ion transporter inhibitor selectively inhibits OAT3 compared with other ion transporter proteins. For instance, the ion transporter inhibitor has an IC₅₀ for OAT3 that is lower than its IC₅₀ for one or more other ion transporter proteins (e.g., OAT1, OAT2, OAT3, OAT4, OAT6, OAT7, OAT9, OAT10, OCT2, OATP1B1, OATP1B3, MATE1, MATE2-K, BCRP, PBP, or URAT1). In certain embodiments, the ion transporter inhibitor has an IC₅₀ for OAT3 that is at least about 10 fold lower compared with its IC₅₀ for one or more other ion transporter proteins (e.g., OAT1, OAT2, OAT3, OAT4, OAT6, OAT7, OAT9, OAT10, OCT2, OATP1B1, OATP1B3, MATE1, MATE2-K, BCRP, PBP, or URAT1).

In certain embodiments, the ion transporter inhibitor has an IC₅₀ for OAT3 that is at least about 10 fold lower compared with its IC₅₀ for organic anion transporter 1 (OAT1). In certain embodiments, the ion transporter inhibitor has an IC₅₀ for OAT3 that is at least about 10-20 fold lower compared with its IC₅₀ for organic anion transporter 1 (OAT1). In certain embodiments, the ion transporter inhibitor has an IC₅₀ for OAT3 that is at least about 20 fold lower compared with its IC₅₀ for organic anion transporter 1 (OAT1). In certain embodiments, the ion transporter inhibitor has an IC₅₀ for OAT3 that is at least about 20-50 fold lower compared with its IC₅₀ for organic anion transporter 1 (OAT1).

In some embodiments, the ion transporter inhibitor has an IC₅₀ for OAT3 that is at least about 10 fold lower compared with its IC₅₀ for organic cation transporter 2 (OCT2). In some embodiments, the ion transporter inhibitor has an IC₅₀ for OAT3 that is at least about 10-50 fold lower compared with its IC₅₀ for organic cation transporter 2 (OCT2). In certain embodiments, the ion transporter inhibitor has an IC₅₀ for OAT3 that is at least about 50 fold lower compared with its IC₅₀ for organic cation transporter 2 (OCT2). In some embodiments, the ion transporter inhibitor has an IC₅₀ for OAT3 that is at least about 100-500 fold lower compared with its IC₅₀ for organic cation transporter 2 (OCT2). In some embodiments, the ion transporter inhibitor has an IC₅₀ for OAT3 that is at least about 100 fold lower compared with its IC₅₀ for organic cation transporter 2 (OCT2).

In some embodiments, the ion transporter inhibitor has an IC₅₀ for OAT3 that is at least about 10 fold lower compared with its IC₅₀ for organic anion transporting polypeptide 1B1 (OATP1B1). In some embodiments, the ion transporter inhibitor has an IC₅₀ for OAT3 that is at least about 10-20 fold lower compared with its IC₅₀ for organic anion transporting polypeptide 1B1 (OATP1B1). In certain embodiments, the ion transporter inhibitor has an IC₅₀ for OAT3 that is at least about 20 fold lower compared with its IC₅₀ for organic anion transporting polypeptide 1B1 (OATP1B1). In some embodiments, the ion transporter inhibitor has an IC₅₀ for OAT3 that is at least about 20-100 fold lower compared with its IC₅₀ for organic anion transporting polypeptide 1B1 (OATP1B1). In some embodiments, the ion transporter inhibitor has an IC₅₀ for OAT3 that is at least about 100 fold lower compared with its IC₅₀ for organic anion transporting polypeptide 1B1 (OATP1B1).

In some embodiments, the ion transporter inhibitor has an IC₅₀ for OAT3 that is at least about 10 fold lower compared with its IC₅₀ for organic anion transporting polypeptide 1B3 (OATP1B3). In some embodiments, the ion transporter inhibitor has an IC₅₀ for OAT3 that is at least about 10-20 fold lower compared with its IC₅₀ for organic anion transporting polypeptide 1B3 (OATP1B3). In certain embodiments, the ion transporter inhibitor has an IC₅₀ for OAT3 that is at least about 20 fold lower compared with its IC₅₀ for organic anion transporting polypeptide 1B3 (OATP1B3). In some embodiments, the ion transporter inhibitor has an IC₅₀ for OAT3 that is at least about 20-100 fold lower compared with its IC₅₀ for organic anion transporting polypeptide 1B3 (OATP1B3). In some embodiments, the ion transporter inhibitor has an IC₅₀ for OAT3 that is at least about 100 fold lower compared with its IC₅₀ for organic anion transporting polypeptide 1B3 (OATP1B3).

In some embodiments, the ion transporter inhibitor has an IC₅₀ for OAT3 that is at least about 10 fold lower compared with its IC₅₀ for multidrug and toxic compound extrusion protein-1 (MATE1/SLC47A1). In some embodiments, the ion transporter inhibitor has an IC₅₀ for OAT3 that is at least about 10-20 fold lower compared with its IC₅₀ for multidrug and toxic compound extrusion protein-1 (MATE1/SLC47A1). In certain embodiments, the ion transporter inhibitor has an IC₅₀ for OAT3 that is at least about 20 fold lower compared with its IC₅₀ for multidrug and toxic compound extrusion protein-1 (MATE1/SLC47A1). In some embodiments, the ion transporter inhibitor has an IC₅₀ for OAT3 that is at least about 20-100 fold lower compared with its IC₅₀ for multidrug and toxic compound extrusion protein-1 (MATE1/SLC47A1). In some embodiments, the ion transporter inhibitor has an IC₅₀ for OAT3 that is at least about 100 fold lower compared with its IC₅₀ for multidrug and toxic compound extrusion protein-1 (MATE1/SLC47A1).

In some embodiments, the ion transporter inhibitor has an IC₅₀ for OAT3 that is at least about 10 fold lower compared with its IC₅₀ for multidrug and toxic compound extrusion protein 2-K (MATE2-K). In some embodiments, the ion transporter inhibitor has an IC₅₀ for OAT3 that is at least about 10-20 fold lower compared with its IC₅₀ for multidrug and toxic compound extrusion protein 2-K (MATE2-K). In certain embodiments, the ion transporter inhibitor has an IC₅₀ for OAT3 that is at least about 20 fold lower compared with its IC₅₀ for multidrug and toxic compound extrusion protein 2-K (MATE2-K). In some embodiments, the ion transporter inhibitor has an IC₅₀ for OAT3 that is at least about 20-100 fold lower compared with its IC₅₀ for multidrug and toxic compound extrusion protein 2-K (MATE2-K). In some embodiments, the ion transporter inhibitor has an IC₅₀ for OAT3 that is at least about 100 fold lower compared with its IC₅₀ for multidrug and toxic compound extrusion protein 2-K (MATE2-K).

In some embodiments, the ion transporter inhibitor has an IC₅₀ for OAT3 that is at least about 10 fold lower compared with its IC₅₀ for breast cancer resistance protein (BCRP). In some embodiments, the ion transporter inhibitor has an IC₅₀ for OAT3 that is at least about 10-20 fold lower compared with its IC₅₀ for breast cancer resistance protein (BCRP). In certain embodiments, the ion transporter inhibitor has an IC₅₀ for OAT3 that is at least about 20 fold lower compared with its IC₅₀ for breast cancer resistance protein (BCRP). In some embodiments, the ion transporter inhibitor has an IC₅₀ for OAT3 that is at least about 20-100 fold lower compared with its IC₅₀ for breast cancer resistance protein (BCRP). In some embodiments, the ion transporter inhibitor has an IC₅₀ for OAT3 that is at least about 100 fold lower compared with its IC₅₀ for breast cancer resistance protein (BCRP).

In some embodiments, the ion transporter inhibitor has an IC₅₀ for OAT3 that is at least about 10 fold lower compared with its IC₅₀ for p-glycoprotein (PGP). In some embodiments, the ion transporter inhibitor has an IC₅₀ for OAT3 that is at least about 10-20 fold lower compared with its IC₅₀ for p-glycoprotein (PGP). In certain embodiments, the ion transporter inhibitor has an IC₅₀ for OAT3 that is at least about 20 fold lower compared with its IC₅₀ for p-glycoprotein (PGP). In some embodiments, the ion transporter inhibitor has an IC₅₀ for OAT3 that is at least about 20-100 fold lower compared with its IC₅₀ for p-glycoprotein (PGP). In some embodiments, the ion transporter inhibitor has an IC₅₀ for OAT3 that is at least about 100 fold lower compared with its IC₅₀ for p-glycoprotein (PGP).

In some embodiments, the ion transporter inhibitor has an IC₅₀ for OAT3 that is at least about 10 fold lower compared with its IC₅₀ for uric acid transporter 1 (URAT1). In some embodiments, the ion transporter inhibitor has an IC₅₀ for OAT3 that is at least about 2-10 fold lower compared with its IC₅₀ for uric acid transporter 1 (URAT1). In some embodiments, the ion transporter inhibitor has an IC₅₀ for OAT3 that is at least about 10-20 fold lower compared with its IC₅₀ for uric acid transporter 1 (URAT1). In certain embodiments, the ion transporter inhibitor has an IC₅₀ for OAT3 that is at least about 20 fold lower compared with its IC₅₀ for uric acid transporter 1 (URAT1). In some embodiments, the ion transporter inhibitor has an IC₅₀ for OAT3 that is at least about 20-100 fold lower compared with its IC₅₀ for uric acid transporter 1 (URAT1). In some embodiments, the ion transporter inhibitor has an IC₅₀ for OAT3 that is at least about 100 fold lower compared with its IC₅₀ for uric acid transporter 1 (URAT1).

In some embodiments, the ion transporter inhibitor has an IC₅₀ for OAT3 that is at least about 5 fold, at least about 10 fold, at least about 20 fold, at least about 30 fold, at least about 40 fold, at least about 50 fold, at least about 60 fold, at least about 70 fold, at least about 80 fold, at least about 90 fold, or at least about 100 fold, at least about 200 fold, at least about 300 fold, at least about 400 fold, at least about 500 fold, at least about 600 fold, at least about 700 fold, at least about 10 fold, at least about 800 fold, at least about 900 fold, at least about 10000 fold, at least about 5000 fold, or at least about 10000 fold lower compared with its IC₅₀ for one or more other ion transporter proteins (e.g., OAT1, OAT2, OAT3, OAT4, OAT6, OAT7, OAT9, OAT10, OCT2, OATP1B1, OATP1B3, MATE1, MATE2-K, BCRP, PBP, or URAT1).

In some embodiments of any of the methods described herein, after administration of the ion transporter inhibitor, the local concentrations of one or more bioactive endogenous metabolites in the brain interstitial space are increased. For instance, in some embodiments, administration of an OAT3 inhibitor blocks the transport of bioactive endogenous metabolites (e.g., OAT3 substrates) from the cerebrospinofluid (CSF) into the bloodstream. As used herein, “bioactive” molecules may refer to molecules that exhibit biological activity. For instance, a bioactive molecule can inhibit the interaction between an enzyme or receptor and its respective substrate(s) or endogenous ligand(s), or inhibit cell growth of a microorganism, by at least 15% at a solution concentration of 10⁻³ molar or lower (i.e., it has inhibitory activity). In some embodiments, the bioactive molecule will inhibit such processes at solution concentrations of any one of 10⁻⁴ molar or lower, 10⁻⁵ molar or lower, 10 ⁻⁶ molar or lower, or 10⁻⁷ molar or lower. In any of the embodiments described herein, the one or more bioactive endogenous metabolites are selected from an anionic neurotransmitter metabolite of epinephrine, norepinephrine, dopamine, and/or serotonin. In certain embodiments, the one or more bioactive endogenous metabolites are selected from the group consisting of: uric acid, glutathione, dehydroepianodrosterone (DHEA), and DHEA sulfate (DHEAS).

Certain OAT3 inhibitors are known in the art. For example, probenecid and para-aminohippurate (PAH) have long been used in blocking OAT3 (Dantzler et al. 1995). In some embodiments, the OAT 3 inhibitor comprises probenecid (Takeda et al., J Pharmacol Exp Ther. 2002 August; 302(2):666-71; Takeda et al., Eur J Pharmacol. 2001 May 11; 419(2-3):113-20; Khamdang et al., J Pharmacol Sci. 2004 February; 94(2):197-202; Jung et al., Life Sci. 2001 Sep. 21; 69(18):2123-35). In some embodiments, the OAT 3 inhibitor comprises para-aminohippurate (PAH). In some embodiments, the OAT 3 inhibitor comprises one or more of Ciprofloxacin, linezolid, para-aminosalicylic acid (PAS), and rifampin (Parvez et al. 2016). In some embodiments, the OAT 3 inhibitor comprises one or more antituberculosis drugs and their derivatives (Parvez et al. 2016). In some embodiments, the OAT3 inhibitor comprises one or more of novobiocin, steviol, and the HIV integrase inhibitor cabotegravir (Duan and You 2009, Srimaroeng 2005, Chatsudthipong and Jutabha 2001). In further embodiments, the OAT 3 inhibitor comprises one or more of mefenamic acid, meclofenamic acid, pioglitazone, oxaprozin, nateglinide, amlexanox, ketorolac tromethamine, diflunisal, nitazoxanide, irbesartan, valsartan, telmisartan, balsalazide, and ethacrynic acid (Duan et al. 2012). In some embodiments, the OAT 3 inhibitor comprises one or more of stiripentol, cortisol succinate, demeclocycline, penciclovir, ornidazole, benazepril, chlorpropamide, and artesunate (Duan et al. 2012). In some embodiments, the OAT3 inhibitor comprises one or more of cephalothin (also known as keflin or cefalotin), cefamandole, cefotaxime (also known as cefotax, claforan, or kefotex), cefazolin, cefoperazone (also known as cefobid), cephaloridine (also known as aliporina, cefaloridine, ceporine, kefloridin, lloncefal, loridine, or sefacin), and ceftriaxone (also known as rocefin, rocephin, or ceftriaxone) (Takeda et al. Eur J Pharmacol. 2002 Mar. 8; 438(3):137-42). In some embodiments, the OAT3 inhibitor comprises rolofylline (Takeda et al. Eur J Pharmacol 2002 Apr. 26; 441(3):215). In some embodiments, the OAT3 inhibitor comprises one or more of probenecid (also known as benemid, probalan, probampicin, or probecid), piroxicam (also known as feldene), octanoic acid (also known as caprylic acid), citrinin, aminohippuric acid (also known as aminohippurate) (Jung et al. Life Sci. 2001 Sep. 21; 69(18):2123-35). In some embodiments, the OAT3 inhibitor comprises indomethacin (also known as Indocin, Indocin SR, Indo-Lemmon, indometacin, or indometacin farnesil) (Takeda et al. J Pharmacol Exp Ther. 2002 August; 302(2):666-71). In some embodiments, the OAT3 inhibitor comprises one or more of (1r,4r)-4-((5-(2-((4-fluorobenzyl)carbamoyl)-6-methylpyridin-4-yl)-2H-tetrazol-2-yl)methyl)cyclohexane-1-carboxylic acid (CHEMBL603656) and 4-((1-methyl-2,4-dioxo-6-(3-phenylprop-1-yn-1-yl)-1,4-dihydroquinazolin-3(2H)-yl)methyl)benzoic acid (Ruminski, et al. J Med Chem. 2016 Jan. 14; 59(1):313-27). In some embodiments, the OAT3 inhibitor is zonampanel (CHEMBL119625) (Mattes et al., J Med Chem. 2010 Aug. 12; 53(15):5367-82). In some embodiments, the OAT3 inhibitor comprises one or more of cefadroxil (also known as cefadrops, cefatabs, or CHEMBL1644) and cefadroxil hemihydrate (Wolman et al., Drug Metab Dispos. 2013 April; 41(4):791-800). In some embodiments, the OAT3 inhibitor comprises one or more of betamipron (CHEMBL1231530) and pravastatin (also known as pravachol or CHEMBL1144) (Khamdang et al., J Pharmacol Sci. 2004 February; 94(2):197-202). In some embodiments, the OAT3 inhibitor is hippuric acid (CHEMBL461) (Deguchi et al., Kidney Int. 2004 January; 65(1):162-74). In certain embodiments, the OAT 3 inhibitor comprises an OAT3-inhibitory antibody or a binding protein specific to OAT3.

In some embodiments of any of the methods described herein, after administration of the ion transporter inhibitor, the efflux of the one or more bioactive endogenous metabolites across the blood brain barrier is reduced. For example, in some embodiments, the efflux of the one or more bioactive endogenous metabolites from the brain interstitial space across the blood brain barrier to the blood stream is reduced.

In some embodiments according to any one of the methods above, the ion transporter inhibitor increases the concentration of one or more bioactive endogenous metabolites in the brain. In some embodiments, the ion transporter inhibitor increases the concentration of one or more bioactive endogenous metabolites in the brain by at least about 50%. In some embodiments, the ion transporter inhibitor increases the concentration of one or more bioactive endogenous metabolites in the brain by at least about 50%, such as by at least about 75%, by at least about 100%, by at least about 150%, by at least about 200%, by at least about 300%, by at least about 400%, by at least about 500%, by at least about 600%, by at least about 700%, by at least about 800%, by at least about 900%, by at least about 1000%, or by at least about 2000%.

In some embodiments, the ion transporter inhibitor increases the concentration of one or more bioactive endogenous metabolites in the brain by at least 5 fold. In some embodiments, the ion transporter inhibitor increases the concentration of one or more bioactive endogenous metabolites in the brain by at least about 0.5 fold, by at least about 0.75 fold, by at least about 1 fold, by at least about 1.5 fold, by at least about 1.75 fold, by at least about 2 fold, by at least about 3 fold, by at least about 4 fold, by at least about 5 fold, by at least about 6 fold, by at least about 7 fold, by at least about 8 fold, by at least about 9 fold, by at least about 10 fold, by at least about 20 fold, by at least about 30 fold, by at least about 40 fold, by at least about 50 fold, by at least about 100 fold, by at least about 500 fold, or by at least about 1000 fold.

In some embodiments, the ion transporter inhibitor does not alter (e.g., does not increase or decrease) the plasma level of the bioactive endogenous metabolites by more than 5 fold. In some embodiments, the compound does not alter the plasma level of the bioactive endogenous metabolites by more than 0.5 fold, by more than 0.75 fold, by more than 1 fold, by more than 2 fold, by more than 3 fold, by more than 4 fold, by more than 5 fold, by more than 6 fold, by more than 7 fold, by more than 8 fold, by more than 9 fold, by more than 10 fold, by more than 20 fold, by more than 30 fold, by more than 40 fold, by more than 50 fold, by more than 100 fold, by more than 500 fold, or by more than 1000 fold. In some embodiments, the ion transporter inhibitor does not alter (e.g., does not increase or decrease) the plasma level of the bioactive endogenous metabolites by more than 500%. In some embodiments, the compound does not alter the plasma level of the bioactive endogenous metabolites by more than 50%, by more than 75%, by more than 100%, by more than 200%, by more than 300%, by more than 400%, by more than 500%, by more than 600%, by more than 700%, by more than 800%, by more than 900%, by more than 1000%, by more than 2000%, by more than 3000%, by more than 4000%, by more than 5000%, by more than 10,000%, by more than 50,000%, or by more than 100,000%.

In some embodiments, the compound does not increase the plasma level of the bioactive endogenous metabolites by more than 5 fold. In some embodiments, the compound does not increase the plasma level of the bioactive endogenous metabolites by more than 0.5 fold, by more than 0.75 fold, by more than 1 fold, by more than 2 fold, by more than 3 fold, by more than 4 fold, by more than 5 fold, by more than 6 fold, by more than 7 fold, by more than 8 fold, by more than 9 fold, by more than 10 fold, by more than 20 fold, by more than 30 fold, by more than 40 fold, by more than 50 fold, by more than 100 fold, by more than 500 fold, or by more than 1000 fold. In some embodiments, the compound does not increase the plasma level of the bioactive endogenous metabolites by more than 500%. In some embodiments, the compound does not alter the plasma level of the bioactive endogenous metabolites by more than 50%, by more than 75%, by more than 100%, by more than 200%, by more than 300%, by more than 400%, by more than 500%, by more than 600%, by more than 700%, by more than 800%, by more than 900%, by more than 1000%, by more than 2000%, by more than 3000%, by more than 4000%, by more than 5000%, by more than 10,000%, by more than 50,000%, or by more than 100,000%.

In some embodiments according to any one of the methods above, the ion transporter inhibitor increases or elevates one or more bioactive endogenous metabolites in the brain. In some embodiments, the ion transporter inhibitor alters the plasma level of the bioactive metabolites by about 5 fold or less. In some embodiments, the ion transporter inhibitor alters the plasma level of the bioactive endogenous metabolites by more than 0.5 fold, by more than 0.75 fold, by more than 1 fold, by more than 2 fold, by more than 3 fold, by more than 4 fold, by more than 5 fold, by more than 6 fold, by more than 7 fold, by more than 8 fold, by more than 9 fold, by more than 10 fold, by more than 20 fold, by more than 30 fold, by more than 40 fold, by more than 50 fold, by more than 100 fold, by more than 500 fold, or by more than 1000 fold. In some embodiments, the ion transporter inhibitor does not increase the plasma level of the bioactive endogenous metabolites by more than 5 fold. In some embodiments, the compound does not increase the plasma level of the bioactive endogenous metabolites by more than 0.5 fold, by more than 0.75 fold, by more than 1 fold, by more than 2 fold, by more than 3 fold, by more than 4 fold, by more than 5 fold, by more than 6 fold, by more than 7 fold, by more than 8 fold, by more than 9 fold, by more than 10 fold, by more than 20 fold, by more than 30 fold, by more than 40 fold, by more than 50 fold, by more than 100 fold, by more than 500 fold, or by more than 1000 fold. In some embodiments according to any one of the methods above, the ion transporter inhibitor increases or elevates one or more bioactive endogenous metabolites in the brain. In some embodiments, the ion transporter inhibitor alters the plasma level of the bioactive metabolites by about 5000% or less. In some embodiments, the ion transporter inhibitor alters the plasma level of the bioactive endogenous metabolites by more than 50%, by more than 75%, by more than 100%, by more than 200%, by more than 300%, by more than 400%, by more than 500%, by more than 600%, by more than 700%, by more than 800%, by more than 900%, by more than 1000%, by more than 2000%, by more than 3000%, by more than 4000%, by more than 5000%, by more than 10,000%, by more than 50,000%, or by more than 100,000%. In some embodiments, the ion transporter inhibitor does not increase the plasma level of the bioactive endogenous metabolites by more than 500%. In some embodiments, the compound does not increase the plasma level of the bioactive endogenous metabolites by more than 50%, by more than 75%, by more than 100%, by more than 200%, by more than 300%, by more than 400%, by more than 500%, by more than 600%, by more than 700%, by more than 800%, by more than 900%, by more than 1000%, by more than 2000%, by more than 3000%, by more than 4000%, by more than 5000%, by more than 10,000%, by more than 50,000%, or by more than 100,000%.

In some embodiments, the compound does not alter uric acid secretion from blood into urine by more than 5 fold. In some embodiments, the compound does not alter uric acid secretion from blood into urine by more than 0.5 fold, by more than 0.75 fold, by more than 1 fold, by more than 2 fold, by more than 3 fold, by more than 4 fold, by more than 5 fold, by more than 6 fold, by more than 7 fold, by more than 8 fold, by more than 9 fold, by more than 10 fold, by more than 20 fold, by more than 30 fold, by more than 40 fold, by more than 50 fold, by more than 100 fold, by more than 500 fold, or by more than 1000 fold. In some embodiments, the compound does not reduce uric acid secretion from blood into urine by more than 5 fold. In some embodiments, the compound does not reduce uric acid secretion from blood into urine by more than 0.5 fold, by more than 0.75 fold, by more than 1 fold, by more than 2 fold, by more than 3 fold, by more than 4 fold, by more than 5 fold, by more than 6 fold, by more than 7 fold, by more than 8 fold, by more than 9 fold, by more than 10 fold, by more than 20 fold, by more than 30 fold, by more than 40 fold, by more than 50 fold, by more than 100 fold, by more than 500 fold, or by more than 1000 fold.

In some embodiments, the compound does not alter uric acid secretion from blood into urine by more than 500%. In some embodiments, the compound does not alter uric acid secretion from blood into urine by more than 50%, by more than 75%, by more than 100%, by more than 200%, by more than 300%, by more than 400%, by more than 500%, by more than 600%, by more than 700%, by more than 800%, by more than 900%, by more than 1000%, by more than 2000%, by more than 3000%, by more than 4000%, by more than 5000%, by more than 10,000%, by more than 50,000%, or by more than 100,000%. In some embodiments, the compound does not reduce uric acid secretion from blood into urine by more than 500%. In some embodiments, the compound does not reduce uric acid secretion from blood into urine by more than 50%, by more than 75%, by more than 100%, by more than 200%, by more than 300%, by more than 400%, by more than 500%, by more than 600%, by more than 700%, by more than 800%, by more than 900%, by more than 1000%, by more than 2000%, by more than 3000%, by more than 4000%, by more than 5000%, by more than 10,000%, by more than 50,000%, or by more than 100,000%.

In one aspect, provided is a method for treating a condition associated with neurodegeneration or accumulation of proteins in the brain in a subject in need thereof, comprising administering to the subject a therapeutically effective amount of a compound that upregulates one or more bioactive endogenous metabolites in the brain, wherein the concentration of the one or more bioactive endogenous metabolites is increased or elevated in the brain.

In some embodiments according to any one of the methods above, the one or more bioactive endogenous metabolite is selected from the group consisting of uric acid, glutathione, and dehydroepianodrosterone (DHEA), and DHEA sulfate (DHEAS).

In some embodiments according to any one of the methods above, the condition associated with neurodegeneration is Alzheimer's Disease, Parkinson's Disease, fronto-temporal dementia, dementia with Lewy Bodies, PD dementia, multiple system atrophy, Huntington's disease, Amyotrophic lateral sclerosis, progressive supranuclear palsy, or neuroinflammation.

In one aspect, provided is a method of decreasing neuroinflammation in a subject, comprising administering to the subject an effective amount of a compound that is an inhibitor of organic anion transporter 3 (OAT3).

In one aspect, provided is a method of treating a disease or condition associated with neuroinflammation, comprising administering to a subject in need of such treatment an effective amount of a compound that is an inhibitor of organic anion transporter 3 (OAT3). In some embodiments, the compound increases the expression of anti-inflammatory markers. In some embodiments, the compound reduces pro-inflammatory response. In some embodiments, the reduction in pro-inflammatory response comprises reduction in gene expression one or more of TNF, IL6, IL12/23p40 or MCP1. In some embodiments, the compound activates TrkA/Akt/CREB/Jmjd3 pathway. In some embodiments, the compound increases the level of pTrkA. In some embodiments, the compound increases the level of pAkt. In some embodiments, the compound increases the activation of CREB. In some embodiments, the compound increases the expression of Jmjd3. In some embodiments, the compound promotes anti-inflammatory phenotype of microglia. In some embodiments, the anti-inflammatory phenotype of microglia comprises increased gene expression of one or more of M2 polarization markers comprising arginase 1, Ym1 (chitinase-like protein 3), Fizz1, Klf4 (Kruppel like factor 4) or IL10. In some embodiments, the compound inhibits pro-inflammatory phenotype of microglia.

One aspect of the invention provides a method of preventing aggregation or accumulation or enhancing clearance of protease-resistant protein, comprising contacting the protease-resistant protein with an effective amount of a compound that is an inhibitor of organic anion transporter 3 (OAT3), wherein the contacting is in vitro, ex vivo, or in vivo. In some embodiments, the protease-resistant protein is selected from alpha synuclein, a-beta, tau, Huntingtin, and TAR DNA binding protein 43 (TDP43) proteins.

Provided in other embodiments are methods of preventing aggregation or accumulation or enhancing clearance of protease-resistant protein, comprising contacting the protease-resistant protein with an effective amount of a compound that is an inhibitor of organic anion transporter 3 (OAT3), wherein the contacting is in vitro, ex vivo, or in vivo. In some embodiments, the protease-resistant protein is selected from alpha synuclein, a-beta, tau, Huntingtin, and TAR DNA binding protein 43 (TDP43) proteins.

Also provided are ion transporter inhibitors (e.g., a compound of Formula (I), or a pharmaceutically acceptable salt thereof, as described herein) for use in a method of treating a disease or condition associated with neurodegeneration or accumulation of proteins in the brain in a subject in need thereof. In some embodiments, the ion transporter inhibitor modulates the efflux of one or more bioactive endogenous metabolites across the blood brain barrier (BBB) of the subject. In some embodiments, the disease or condition associated with neurodegeneration is Alzheimer's Disease, Parkinson's Disease, fronto-temporal dementia, dementia with Lewy Bodies, PD dementia, multiple system atrophy, Huntington's disease, Amyotrophic lateral sclerosis, progressive supranuclear palsy, or neuroinflammation.

Provided in some embodiments are ion transporter inhibitors (e.g., a compound of Formula (I), or a pharmaceutically acceptable salt thereof, as described herein) for use in a method of modulating efflux of one or more bioactive endogenous metabolites across the blood brain barrier (BBB) in a subject in need thereof.

Provided in some embodiments are ion transporter inhibitors (e.g., a compound of Formula (I), or a pharmaceutically acceptable salt thereof, as described herein) for use in a method of improving neuroprotection in subject in need thereof. Provided in other embodiments are ion transporter inhibitors (e.g., a compound of Formula (I), or a pharmaceutically acceptable salt thereof, as described herein) for use in a method of decreasing neuroinflammation in a subject in need thereof. In some embodiments, the ion transporter inhibitor modulates the concentration of one or more bioactive endogenous metabolites in the brain interstitial space.

Also provided is the use of ion transporter inhibitors (e.g., a compound of Formula (I), or a pharmaceutically acceptable salt thereof, as described herein) in the manufacture of a medicament for the treatment of a disease or condition associated with neurodegeneration or accumulation of proteins in the brain in a subject in need thereof. In some embodiments, the ion transporter inhibitor modulates the efflux of one or more bioactive endogenous metabolites across the blood brain barrier (BBB) of the subject. In some embodiments, the disease or condition associated with neurodegeneration is Alzheimer's Disease, Parkinson's Disease, fronto-temporal dementia, dementia with Lewy Bodies, PD dementia, multiple system atrophy, Huntington's disease, Amyotrophic lateral sclerosis, progressive supranuclear palsy, or neuroinflammation.

Provided in some embodiments is the use of ion transporter inhibitors (e.g., a compound of Formula (I), or a pharmaceutically acceptable salt thereof, as described herein) in the manufacture of a medicament for improving neuroprotection in subject. Provided in other embodiments is the use of ion transporter inhibitors (e.g., a compound of Formula (I), or a pharmaceutically acceptable salt thereof, as described herein) in the manufacture of a medicament for decreasing neuroinflammation in a subject. In some embodiments, the ion transporter inhibitor modulates the concentration of one or more bioactive endogenous metabolites in the brain interstitial space.

### Compounds

Compounds and salts thereof (such as pharmaceutically acceptable salts) that may be useful in the presently described methods are detailed herein, including in the Summary and in the appended claims. Also provided are the use of all of the compounds described herein, including salts and solvates of the compounds described herein, as well as methods of making such compounds. Any compound described herein may also be referred to as a drug.

In one aspect, the present disclosure provides a compound of Formula (I):

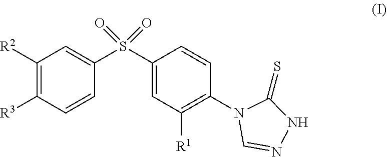

wherein

R¹, R², and R³ are each independently hydrogen, hydroxy, halogen, optionally substituted C₁₋₄ alkyl, optionally substituted C₁₋₄ alkoxy, —CN, —C(O)R^{x}, —C(O)OR^{x}, —S(O)₂R^{x}, or —NR^{y}R^{z};

R^{x}, R^{y}, and R^{z} are each independently H or optionally substituted C₁₋₄alkyl, or R^{y} and R^{z} taken together with the nitrogen to which they are attached form an optionally substituted monocyclic heterocycloalkyl ring;

or a pharmaceutically acceptable salt thereof.

In some embodiments, when a group is described as being optionally substituted, the indicated group is unsubstituted or substituted by one or more substituents selected from the group consisting of oxo, C₁-C₆ alkyl, C₂-C₆ alkenyl, C₂-C₆ alkynyl, halogen, —CN, —OR⁴, —SR⁴, —NR⁵R⁶, —NO₂, —C═NH(OR⁴), —C(O)R⁴, —OC(O)R⁴, —C(O)OR⁴, —C(O)NR⁵R⁶, —OC(O)NR⁵R⁶, —NR⁴C(O)R⁵, —NR⁴C(O)OR⁵, —NR⁴C(O)NR⁵R⁶, —S(O)R⁴, —S(O)₂R⁴, —NR⁴S(O)R⁵, —C(O)NR⁴S(O)R⁵, —NR⁴S(O)₂R⁵, —C(O)NR⁴S(O)₂R⁵, —S(O)NR⁵R⁶, —S(O)₂NR⁵R⁶, —P(O)(OR⁵) (OR⁶), C₃-C₆ cycloalkyl, 3-12-membered heterocyclyl, 5- to 10-membered heteroaryl, C₆-C₁₄ aryl, —(C₁-C₃ alkylene)CN, —(C₁-C₃ alkylene)OR⁴, —(C₁-C₃ alkylene)SR⁴, —(C₁-C₃ alkylene)NR⁵R⁶, —(C₁-C₃ alkylene)CF₃, —(C₁-C₃ alkylene)NO₂, —C═NH(OR⁴), —(C₁-C₃ alkylene)C(O)R⁴, —(C₁-C₃ alkylene)OC(O)R⁴, —(C₁-C₃ alkylene)C(O)OR⁴, —(C₁-C₃ alkylene)C(O)NR⁵R⁶, —(C₁-C₃ alkylene)OC(O)NR⁵R⁶, —(C₁-C₃ alkylene)NR⁴C(O)R⁵, —(C₁-C₃ alkylene)NR⁴C(O)OR⁵, —(C₁-C₃ alkylene)NR⁴C(O)NR⁵R⁶, —(C₁-C₃ alkylene)S(O)R⁴, —(C₁-C₃ alkylene)S(O)₂R⁴, —(C₁-C₃ alkylene)NR⁴S(O)R⁵, —C(O)(C₁-C₃ alkylene)NR⁴S(O)R⁵, —(C₁-C₃ alkylene)NR⁴S(O)₂R⁵, —(C₁-C₃ alkylene)C(O)NR⁴S(O)₂R⁵, —(C₁-C₃ alkylene)S(O)NR⁵R⁶, —(C₁-C₃ alkylene)S(O)₂NR⁵R⁶, —(C₁-C₃ alkylene)P(O)(OR⁵)(OR⁶), —(C₁-C₃ alkylene)(C₃-C₆ cycloalkyl), —(C₁-C₃ alkylene)(3-12-membered heterocyclyl), —(C₁-C₃ alkylene)(5-10-membered heteroaryl) and —(C₁-C₃ alkylene)(C₆-C₁₄ aryl), wherein the one or more substituents are each independently unsubstituted or substituted with one or more further substituents selected from the group consisting of halogen, oxo, —OR⁷, —NR⁷R⁸, —C(O)R⁷, —CN, —S(O)R⁷, —S(O)₂R⁷, —P(O)(OR⁷)(OR⁸), —(C₁-C₃ alkylene)OR⁷, —(C₁-C₃ alkylene)NR⁷R⁸, —(C₁-C₃ alkylene)C(O)R⁷, —(C₁-C₃ alkylene)S(O)R⁷, —(C₁-C₃ alkylene)S(O)₂R⁷, —(C₁-C₃ alkylene)P(O)(OR⁷)(OR⁸), C₃-C₈ cycloalkyl, C₁-C₆ alkyl, and C₁-C₆ alkyl substituted by oxo, —OH or halogen; wherein each R⁴ is independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl or 3-6-membered heterocyclyl, wherein the C₁-C₆ alkyl, C₂-C₆ alkenyl, C₂-C₆ alkynyl, C₃-C₆ cycloalkyl, C₆-C₁₄ aryl, 5-6-membered heteroaryl and 3-6-membered heterocyclyl are independently unsubstituted or substituted by halogen, oxo, —CN, —OR⁹, —NR⁹R¹⁰, —P(O)(OR⁹)(OR¹⁰), phenyl, phenyl substituted by halogen, C₁-C₆ alkyl, or C₁-C₆ alkyl substituted by halogen, —OH or oxo; R⁵ and R⁶ are each independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl or 3-6 membered heterocyclyl, wherein the C₁-C₆ alkyl, C₂-C₆ alkenyl, C₂-C₆ alkynyl, C₃-C₆ cycloalkyl, C₆-C₁₄ aryl, 5-6-membered heteroaryl and 3-6 membered heterocyclyl are each independently unsubstituted or substituted by halogen, oxo, —CN, —OR⁹, —NR⁹R¹⁰, C₁-C₆ alkyl, or C₁-C₆ alkyl substituted by halogen, —OH or oxo; and R⁷, R⁸, R⁹ and R¹⁰ are each independently hydrogen, C₁-C₆ alkyl, C₂-C₆ alkenyl, C₂-C₆ alkynyl, C₁-C₆ alkyl substituted by one or more halogen, C₂-C₆ alkenyl substituted by one or more halogen, or C₂-C₆ alkynyl substituted by one or more halogen.

In some embodiments of Formula (I), R¹, R², and R³ are each independently hydrogen, hydroxy, halogen, optionally substituted C₁₋₄ alkyl, optionally substituted C₁₋₄ alkoxy, or —NR^{y}R^{z}. In certain instances, for each of R¹, R², and R³, the C₁₋₄ alkyl or C₁₋₄ alkoxy groups are substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄alkyl, —C(O)C₁₋₄alkyl, —C(O)OC₁₋₄alkyl, or —S(O)₂C₁₋₄alkyl.

In some embodiments, one or more of R¹, R², or R³ is C₁₋₄ alkyl, which is unsubstituted or substituted with one or more substituents selected from the group consisting of halogen, —CN, —OR⁴, —SR⁴, —NR⁵R⁶, —NO₂, —C═NH(OR⁴), —C(O)R⁴, —OC(O)R⁴, —C(O)OR⁴, —C(O)NR⁵R⁶, —OC(O)NR⁵R⁶, —NR⁴C(O)R⁵, —NR⁴C(O)OR⁵, —NR⁴C(O)NR⁵R⁶, —S(O)R⁴, —S(O)₂R⁴, —NR⁴S(O)R⁵, —C(O)NR⁴S(O)R⁵, —NR⁴S(O)₂R⁵, —C(O)NR⁴S(O)₂R⁵, —S(O)NR⁵R⁶, —S(O)₂NR⁵R⁶, —P(O)(OR⁵) (OR⁶), C₃-C₆ cycloalkyl, 3-12-membered heterocyclyl, 5- to 10-membered heteroaryl, and C₆-C₁₄ aryl; wherein R⁴ is independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl or 3-6-membered heterocyclyl, wherein the C₁-C₆ alkyl, C₂-C₆ alkenyl, C₂-C₆ alkynyl, C₃-C₆ cycloalkyl, C₆-C₁₄ aryl, 5-6-membered heteroaryl and 3-6-membered heterocyclyl are independently optionally substituted by halogen, oxo, —CN, —OR⁹, —NR⁹R¹⁰, —P(O)(OR⁹)(OR¹⁰), phenyl optionally substituted by halogen, or C₁-C₆ alkyl optionally substituted by halogen, —OH or oxo; R⁵ and R⁶ are each independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl or 3-6 membered heterocyclyl, wherein the C₁-C₆ alkyl, C₂-C₆ alkenyl, C₂-C₆ alkynyl, C₃-C₆ cycloalkyl, C₆-C₁₄ aryl, 5-6-membered heteroaryl and 3-6 membered heterocyclyl are independently optionally substituted by halogen, oxo, —CN, —OR⁹, —NR⁹R¹⁰ or C₁-C₆ alkyl optionally substituted by halogen, —OH or oxo; and R⁹ and R¹⁰ are each independently hydrogen, C₁-C₆ alkyl, C₂-C₆ alkenyl, C₂-C₆ alkynyl, C₁-C₆ alkyl substituted by one or more halogen, C₂-C₆ alkenyl substituted by one or more halogen, or C₂-C₆ alkynyl substituted by one or more halogen.

In some embodiments, one or more of R¹, R², or R³ is C₁₋₄ alkoxy, which is unsubstituted or substituted with one or more substituents selected from the group consisting of C₁-C₆ alkyl, C₂-C₆ alkenyl, C₂-C₆ alkynyl, halogen, —CN, —OR⁴, —SR⁴, —NR⁵R⁶, —NO₂, —C═NH(OR⁴), —C(O)R⁴, —OC(O)R⁴, —C(O)OR⁴, —C(O)NR⁵R⁶, —OC(O)NR⁵R⁶, —NR⁴C(O)R⁵, —NR⁴C(O)OR⁵, —NR⁴C(O)NR⁵R⁶, —S(O)R⁴, —S(O)₂R⁴, —NR⁴S(O)R⁵, —C(O)NR⁴S(O)R⁵, —NR⁴S(O)₂R⁵, —C(O)NR⁴S(O)₂R⁵, —S(O)NR⁵R⁶, —S(O)₂NR⁵R⁶, —P(O)(OR⁵) (OR⁶), C₃-C₆ cycloalkyl, 3-12-membered heterocyclyl, 5- to 10-membered heteroaryl, and C₆-C₁₄ aryl; wherein R⁴ is independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl or 3-6-membered heterocyclyl, wherein the C₁-C₆ alkyl, C₂-C₆ alkenyl, C₂-C₆ alkynyl, C₃-C₆ cycloalkyl, C₆-C₁₄ aryl, 5-6-membered heteroaryl and 3-6-membered heterocyclyl are independently optionally substituted by halogen, oxo, —CN, —OR⁹, —NR⁹R¹⁰, —P(O)(OR⁹)(OR¹⁰), phenyl optionally substituted by halogen, or C₁-C₆ alkyl optionally substituted by halogen, —OH or oxo; and R⁵ and R⁶ are each independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl or 3-6 membered heterocyclyl, wherein the C₁-C₆ alkyl, C₂-C₆ alkenyl, C₂-C₆ alkynyl, C₃-C₆ cycloalkyl, C₆-C₁₄ aryl, 5-6-membered heteroaryl and 3-6 membered heterocyclyl are independently optionally substituted by halogen, oxo, —CN, —OR⁹, —NR⁹R¹⁰ or C₁-C₆ alkyl optionally substituted by halogen, —OH or oxo; and R⁹ and R¹⁰ are each independently hydrogen, C₁-C₆ alkyl, C₂-C₆ alkenyl, C₂-C₆ alkynyl, C₁-C₆ alkyl substituted by one or more halogen, C₂-C₆ alkenyl substituted by one or more halogen, or C₂-C₆ alkynyl substituted by one or more halogen.

In some embodiments, R¹ is hydrogen, hydroxy, halogen, optionally substituted C₁₋₄ alkyl, optionally substituted C₁₋₄ alkoxy, or —NR^{y}R^{z}. In some embodiments, R¹ is hydrogen. In some embodiments, R¹ is hydroxyl. In some embodiments, R¹ is halogen. In some embodiments, R¹ is chloro. In some embodiments, R¹ is fluoro. In other embodiments, R¹ is bromo or iodo. In some embodiments, R¹ is optionally substituted C₁₋₄ alkyl. In some embodiments, R¹ is C₁₋₄ alkyl substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄alkyl, —C(O)C₁₋₄ alkyl, —C(O)OC₁₋₄alkyl, or —S(O)₂C₁₋₄alkyl. In some embodiments, R¹ is C₁₋₄ alkyl substituted with one or more halogen groups. In some embodiments, R¹ is —CF₃, —(CH₂)F, —CHF₂, CH₂Br, —CH₂CF₃, —CH₂CHF₂, or —CH₂CH₂F. In some embodiments, R¹ is CF₃. In some embodiments, R¹ is unsubstituted C₁₋₄ alkyl. For instance, in some embodiments, R¹ is methyl, ethyl, propyl, isopropyl, butyl, isobutyl, secbutyl, or tertbutyl.

In other embodiments, R¹ is —NR^{y}R^{z}, wherein R^{y} and R^{z} taken together with the nitrogen to which they are attached form an optionally substituted monocyclic heterocycloalkyl ring. In some embodiments, R¹ is —NR^{y}R^{z}, wherein R^{y} and R^{z} taken together with the nitrogen to which they are attached form an optionally substituted 5- to 12-membered heterocycloalkyl ring. In some embodiments, R¹ is —NR^{y}R^{z}, wherein R^{y} and R^{z} taken together with the nitrogen to which they are attached form an optionally substituted 5- to 6-membered heterocycloalkyl ring. In some embodiments, R¹ is morpholinyl. In some embodiments, R¹ is morpholinyl substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄alkyl. In some embodiments, R¹ is piperazinyl. In some embodiments, R¹ is piperazinyl substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄ alkyl. In some embodiments, R¹ is piperadinyl. In some embodiments, R¹ is piperadinyl substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄ alkyl, —C(O)OC₁₋₄alkyl, or —S(O)₂C₁₋₄alkyl. In some embodiments, R¹ is pyrrolidinyl. In some embodiments, R¹ is pyrrolidinyl substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄alkyl.

In some embodiments, R¹ is —NR^{y}R^{z}, wherein R^{y} and R^{z} are each independently H or optionally substituted C₁₋₄alkyl. In some embodiments, R¹ is —NR^{y}R^{z}, wherein R^{y} and R^{z} are each H. In some embodiments, R¹ is —NR^{y}R^{z}, wherein R^{y} and R^{z} are each optionally substituted C₁₋₄alkyl. In some embodiments, R¹ is —NR^{y}R^{z}, wherein R^{y} and R^{z} are each optionally C₁₋₄alkyl substituted with one or more substituents selected from the group consisting of hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄ alkyl and R^{f} and R^{g} are each independently H, C₁₋₄alkyl, —C(O)C₁₋₄alkyl, —C(O)OC₁₋₄alkyl, or —S(O)₂C₁₋₄alkyl. In some embodiments, R¹ is —NR^{y}R^{z}, wherein R^{y} and R^{z} are each optionally unsubstituted C₁₋₄alkyl. In certain embodiments, R¹ is —N(CH₂)₂ or —N(CH₂CH₃)₂. In some embodiments, R¹ is —NR^{y}R^{z}, wherein R^{y} and R^{z} are each unsubstituted C₁₋₄alkyl or C₁₋₄alkyl substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄alkyl, —C(O)C₁₋₄ alkyl, —C(O)OC₁₋₄alkyl, or —S(O)₂C₁₋₄alkyl. In some embodiments, R¹ is —NR^{y}R^{z}, wherein one of R^{y} and R^{z} is H and the other is unsubstituted C₁₋₄alkyl. In other embodiments, R¹ is —NR^{y}R^{z}, wherein one of R^{y} and R^{z} is H and the other is C₁₋₄alkyl substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄ alkyl, —C(O)C₁₋₄alkyl, —C(O)OC₁₋₄alkyl, or —S(O)₂C₁₋₄alkyl. In some embodiments, R¹ is —NR^{y}R^{z}, wherein one or R^{y} and R^{z} is H and the other is C₁₋₄alkyl unsubstituted or substituted with hydroxyl. In certain embodiments, R¹ is —NH(CH₂)₂OH.

In some embodiments, R¹ is optionally substituted C₁₋₄ alkoxy. In some embodiments, R¹ is unsubstituted C₁₋₄ alkoxy. In other embodiments, R¹ is C₁₋₄ alkoxy substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄ alkyl, —C(O)OC₁₋₄alkyl, or —S(O)₂C₁₋₄alkyl. In certain embodiments, R¹ is C₁₋₄ alkoxy further substituted with C₁₋₄ alkoxy. For instance, in some embodiments, R¹ is —OCH₂CH₂OCH₂CH₃ or —OCH₂CH₂OCH₃. In other embodiments, R¹ is C₁₋₄ alkoxy substituted with optionally substituted C₁₋₄ alkoxy. In some embodiments, R¹ is —(OCH₂CH₂)ₚ—O—CH₂CH₃, wherein p is 0-10. In other embodiments, R¹ is —(OCH₂CH₂)ₚ—O—CH₃, wherein p is 0-10.

In some embodiments, R² is hydrogen, hydroxy, halogen, optionally substituted C₁₋₄ alkyl, optionally substituted C₁₋₄ alkoxy, or —NR^{x}R^{y}. In some embodiments, R² is hydrogen. In some embodiments, R² is hydroxyl. In some embodiments, R² is halogen. In some embodiments, R² is chloro. In some embodiments, R² is fluoro. In other embodiments, R² is bromo or iodo. In some embodiments, R² is optionally substituted C₁₋₄ alkyl. In some embodiments, R² is C₁₋₄ alkyl substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄alkyl, —C(O)C₁₋₄ alkyl, —C(O)OC₁₋₄alkyl, or —S(O)₂C₁₋₄alkyl. In some embodiments, R² is C₁₋₄ alkyl substituted with one or more halogen groups. In some embodiments, R² is —CF₃, —(CH₂)F, —CHF₂, CH₂Br, —CH₂CF₃, —CH₂CHF₂, or —CH₂CH₂F. In some embodiments, R² is CF₃. In some embodiments, R² is unsubstituted C₁₋₄ alkyl. For instance, in some embodiments, R² is methyl, ethyl, propyl, isopropyl, butyl, isobutyl, secbutyl, or tertbutyl.

In some embodiments, R² is —NR^{y}R^{z}, wherein R^{y} and R^{z} taken together with the nitrogen to which they are attached form an optionally substituted monocyclic heterocycloalkyl ring. In some embodiments, R² is —NR^{y}R^{z}, wherein R^{y} and R^{z} taken together with the nitrogen to which they are attached form an optionally substituted 5- to 12-membered heterocycloalkyl ring. In some embodiments, R² is —NR^{y}R^{z}, wherein R^{y} and R^{z} taken together with the nitrogen to which they are attached form an optionally substituted 5- to 6-membered heterocycloalkyl ring. In some embodiments, R² is morpholinyl. In some embodiments, R² is morpholinyl substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄alkyl. In some embodiments, R² is piperazinyl. In some embodiments, R² is piperazinyl substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄ alkyl. In some embodiments, R² is piperadinyl. In some embodiments, R² is piperadinyl substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄ alkyl, —C(O)OC₁₋₄alkyl, or —S(O)₂C₁₋₄alkyl. In some embodiments, R² is pyrrolidinyl. In some embodiments, R² is pyrrolidinyl substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄alkyl.

In some embodiments, R² is —NR^{y}R^{z}, wherein R^{y} and R^{z} are each independently H or optionally substituted C₁₋₄alkyl. In some embodiments, R² is —NR^{y}R^{z}, wherein R^{y} and R^{z} are each H. In some embodiments, R² is —NR^{y}R^{z}, wherein R^{y} and R^{z} are each optionally substituted C₁₋₄alkyl. In some embodiments, R² is —NR^{y}R^{z}, wherein R^{y} and R^{z} are each optionally C₁₋₄alkyl substituted with one or more substituents selected from the group consisting of hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄ alkyl and R^{f} and R^{g} are each independently H, C₁₋₄alkyl, —C(O)C₁₋₄alkyl, —C(O)OC₁₋₄alkyl, or —S(O)₂C₁₋₄alkyl. In some embodiments, R² is —NR^{y}R^{z}, wherein R^{y} and R^{z} are each optionally unsubstituted C₁₋₄alkyl. In certain embodiments, R² is —N(CH₂)₂ or —N(CH₂CH₃)₂. In some embodiments, R² is —NR^{y}R^{z}, wherein R^{y} and R^{z} are each unsubstituted C₁₋₄alkyl or C₁₋₄alkyl substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄alkyl, —C(O)C₁₋₄ alkyl, —C(O)OC₁₋₄alkyl, or —S(O)₂C₁₋₄alkyl. In some embodiments, R² is —NR^{y}R^{z}, wherein one of R^{y} and R^{z} is H and the other is unsubstituted C₁₋₄alkyl. In other embodiments, R² is —NR^{y}R^{z}, wherein one of R^{y} and R^{z} is H and the other is C₁₋₄alkyl substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄ alkyl, —C(O)C₁₋₄alkyl, —C(O)OC₁₋₄alkyl, or —S(O)₂C₁₋₄alkyl. In some embodiments, R² is —NR^{y}R^{z}, wherein one or R^{y} and R^{z} is H and the other is C₁₋₄alkyl unsubstituted or substituted with hydroxyl. In certain embodiments, R² is —NH(CH₂)₂OH.

In some embodiments, R² is optionally substituted C₁₋₄ alkoxy. In some embodiments, R² is unsubstituted C₁₋₄ alkoxy. In other embodiments, R² is C₁₋₄ alkoxy substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄ alkyl, —C(O)OC₁₋₄alkyl, or —S(O)₂C₁₋₄alkyl. In certain embodiments, R² is C₁₋₄ alkoxy further substituted with C₁₋₄ alkoxy. For instance, in some embodiments, R² is —OCH₂CH₂OCH₂CH₃ or —OCH₂CH₂OCH₃. In other embodiments, R² is C₁₋₄ alkoxy substituted with optionally substituted C₁₋₄ alkoxy. In some embodiments, R² is —(OCH₂CH₂)ₚ—O—CH₂CH₃, wherein p is 0-10. In other embodiments, R² is —(OCH₂CH₂)ₚ—O—CH₃, wherein p is 0-10.

In some embodiments, R³ is hydrogen, hydroxy, halogen, optionally substituted C₁₋₄ alkyl, optionally substituted C₁₋₄ alkoxy, or —NR^{x}R^{y}. In certain instances, the C₁₋₄ alkyl or C₁₋₄ alkoxy groups are substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄alkyl, —C(O)C₁₋₄alkyl, —C(O)OC₁₋₄alkyl, or —S(O)₂C₁₋₄alkyl. In some embodiments, R³ is hydrogen. In some embodiments, R³ is hydroxyl. In some embodiments, R³ is halogen. In some embodiments, R³ is chloro. In some embodiments, R³ is fluoro. In other embodiments, R³ is bromo or iodo. In some embodiments, R³ is optionally substituted C₁₋₄ alkyl. In some embodiments, R³ is C₁₋₄ alkyl substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄alkyl, —C(O)C₁₋₄alkyl, —C(O)OC₁₋₄alkyl, or —S(O)₂C₁₋₄alkyl. In some embodiments, R³ is C₁₋₄ alkyl substituted with one or more halogen groups. In some embodiments, R³ is —CF₃, —(CH₂)F, —CHF₂, CH₂Br, —CH₂CF₃, —CH₂CHF₂, or —CH₂CH₂F. In some embodiments, R³ is CF₃. In some embodiments, R³ is unsubstituted C₁₋₄ alkyl. For instance, in some embodiments, R³ is methyl, ethyl, propyl, isopropyl, butyl, isobutyl, secbutyl, or tertbutyl.

In some embodiments, R³ is —NR^{y}R^{z}, wherein R^{y} and R^{z} taken together with the nitrogen to which they are attached form an optionally substituted monocyclic heterocycloalkyl ring. In some embodiments, R³ is —NR^{y}R^{z}, wherein R^{y} and R^{z} taken together with the nitrogen to which they are attached form an optionally substituted 5- to 12-membered heterocycloalkyl ring. In some embodiments, R³ is —NR^{y}R^{z}, wherein R^{y} and R^{z} taken together with the nitrogen to which they are attached form an optionally substituted 5- to 6-membered heterocycloalkyl ring. In some embodiments, R³ is morpholinyl. In some embodiments, R³ is morpholinyl substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄alkyl. In some embodiments, R³ is piperazinyl. In some embodiments, R³ is piperazinyl substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄ alkyl. In some embodiments, R³ is piperadinyl. In some embodiments, R³ is piperadinyl substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄ alkyl, —C(O)OC₁₋₄alkyl, or —S(O)₂C₁₋₄alkyl. In some embodiments, R³ is pyrrolidinyl. In some embodiments, R³ is pyrrolidinyl substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄alkyl.

In some embodiments, R³ is —NR^{y}R^{z}, wherein R^{y} and R^{z} are each independently H or optionally substituted C₁₋₄alkyl. In some embodiments, R³ is —NR^{y}R^{z}, wherein R^{y} and R^{z} are each H. In some embodiments, R³ is —NR^{y}R^{z}, wherein R^{y} and R^{z} are each optionally substituted C₁₋₄alkyl. In some embodiments, R³ is —NR^{y}R^{z}, wherein R^{y} and R^{z} are each optionally C₁₋₄alkyl substituted with one or more substituents selected from the group consisting of hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄ alkyl and R^{f} and R^{g} are each independently H, C₁₋₄alkyl, —C(O)C₁₋₄alkyl, —C(O)OC₁₋₄alkyl, or —S(O)₂C₁₋₄alkyl. In some embodiments, R³ is —NR^{y}R^{z}, wherein R^{y} and R^{z} are each optionally unsubstituted C₁₋₄alkyl. In certain embodiments, R³ is —N(CH₂)₂ or —N(CH₂CH₃)₂. In some embodiments, R³ is —NR^{y}R^{z}, wherein R^{y} and R^{z} are each unsubstituted C₁₋₄alkyl or C₁₋₄alkyl substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄alkyl, —C(O)C₁₋₄ alkyl, —C(O)OC₁₋₄alkyl, or —S(O)₂C₁₋₄alkyl. In some embodiments, R³ is —NR^{y}R^{z}, wherein one of R^{y} and R^{z} is H and the other is unsubstituted C₁₋₄alkyl. In other embodiments, R³ is —NR^{y}R^{z}, wherein one of R^{y} and R^{z} is H and the other is C₁₋₄alkyl substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄ alkyl, —C(O)C₁₋₄alkyl, —C(O)OC₁₋₄alkyl, or —S(O)₂C₁₋₄alkyl. In some embodiments, R³ is —NR^{y}R^{z}, wherein one or R^{y} and R^{z} is H and the other is C₁₋₄alkyl unsubstituted or substituted with hydroxyl. In certain embodiments, R³ is —NH(CH₂)₂OH.

In some embodiments, R³ is optionally substituted C₁₋₄ alkoxy. In some embodiments, R³ is unsubstituted C₁₋₄ alkoxy. In other embodiments, R³ is C₁₋₄ alkoxy substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄ alkyl, —C(O)OC₁₋₄alkyl, or —S(O)₂C₁₋₄alkyl. In certain embodiments, R³ is C₁₋₄ alkoxy further substituted with C₁₋₄ alkoxy. For instance, in some embodiments, R³ is —OCH₂CH₂OCH₂CH₃ or —OCH₂CH₂OCH₃. In other embodiments, R³ is C₁₋₄ alkoxy substituted with optionally substituted C₁₋₄ alkoxy. In some embodiments, R³ is —(OCH₂CH₂)ₚ—O—CH₂CH₃, wherein p is 0-10. In other embodiments, R³ is —(OCH₂CH₂)ₚ—O—CH₃, wherein p is 0-10.

In some embodiments, R¹, R², and R³ are independently selected from the group consisting of H, —Cl, —CN, —CF₃, methyl, methoxy, —NHCH₂CH₂OH, —N(CH₂CH₃)₂, —N(CH₃)₂, —OCH₂CH₂—O—CH₂CH₃, —OCH₂CH₂OCH₃, morpholinyl, 4-methyl-piperazin-1-yl, piperidinyl, and pyrrolidinyl. In some embodiments, R¹ is selected from the group consisting of H, —NHCH₂CH₂OH, —N(CH₂CH₃)₂, morpholinyl, 4-methyl-piperazin-1-yl, piperidinyl, pyrrolidinyl, —OCH₂CH₂—O—CH₂CH₃, and —OCH₂CH₂OCH₃. In some embodiments, R² is selected from the group consisting of H, —CF₃, —CN, methyl, methoxy, —OCH₂CH₂—O—CH₂CH₃, —N(CH₃)₂, and morpholinyl. In some embodiments, R³ is selected from the group consisting of H, —C₁, —CN, methyl, methoxy, and morpholinyl.

In some embodiments, R² is optionally substituted C₁₋₄ alkyl and R³ is halogen or C₁₋₄ alkyl. In some embodiments, R² is C₁₋₄ alkyl substituted with one or more halogen, and R³ is halogen. In certain embodiments, R² is —CF₃ and R³ is Cl. In certain embodiments, R² is —CF₃ and R³ is methyl. In some embodiments, R³ is optionally substituted C₁₋₄ alkyl and R² is halogen or C₁₋₄ alkyl. In some embodiments, R³ is C₁₋₄ alkyl substituted with one or more halogen, and R² is halogen. In some embodiments, R³ is —CF₃ and R² is Cl. In some embodiments, R³ is —CF₃ and R² is methyl. In some embodiments, R² and R³ are each H. In some embodiments, R² is H and R³ is halogen, —CN, optionally substituted C₁₋₄ alkyl, optionally substituted C₁₋₄ alkoxy, or —NR^{y}R^{z}, wherein R^{y} and R^{z} are each independently H or optionally substituted C₁₋₄alkyl, or R^{y} and R^{z} taken together with the nitrogen to which they are attached form an optionally substituted monocyclic heterocycloalkyl ring. In some embodiments, R² is H and R³ is halogen. For instance, in some embodiments, R² is H and R³ is Cl. In other some embodiments, R² is H and R³ is F. In some embodiments, R² is H and R³ is —CN. In some embodiments, R² is H and R³ is optionally substituted C₁₋₄ alkyl. For instance, in some embodiments, R² is H and R³ is methyl. In some embodiments, R² is H and R³ is —CF₃. In some embodiments, R² is H and R³ is optionally substituted C₁₋₄ alkoxy. For instance, in some embodiments, R² is H and R³ is methoxy. In some embodiments, R² is H and R³ is —NR^{y}R^{z}, wherein R^{y} and R^{z} are each independently H or optionally substituted C₁₋₄alkyl. In certain embodiments, R² is H and R³ is —N(CH₃)₂. In some embodiments, R² is H and R³ is —NR^{y}R^{z}, wherein R^{y} and R^{z} taken together with the nitrogen to which they are attached form an optionally substituted monocyclic heterocycloalkyl ring. In certain embodiments, R² is H and R³ is morpholinyl. In some embodiments, R² is C₁₋₄ alkyl and R³ is halogen. For instance, in some embodiments, R² is methyl and R³ is Cl. In other embodiments, R² is H and R³ is —CN. In some embodiments, R³ is H and R² is halogen, —CN, optionally substituted C₁₋₄ alkyl, optionally substituted C₁₋₄ alkoxy, or —NR^{y}R^{z}, wherein R^{y} and R^{z} are each independently H or optionally substituted C₁₋₄alkyl, or R^{y} and R^{z} taken together with the nitrogen to which they are attached form an optionally substituted monocyclic heterocycloalkyl ring. In some embodiments, R³ is H and R² is halogen. For instance, in some embodiments, R³ is H and R² is Cl. In other some embodiments, R³ is H and R² is F. In some embodiments, R³ is H and R² is —CN. In some embodiments, R³ is H and R² is optionally substituted C₁₋₄ alkyl. For instance, in some embodiments, R³ is H and R² is methyl. In some embodiments, R³ is H and R² is —CF₃. In some embodiments, R³ is H and R² is optionally substituted C₁₋₄ alkoxy. For instance, in some embodiments, R³ is H and R² is methoxy. In some embodiments, R³ is H and R² is —NR^{y}R^{z}, wherein R^{y} and R^{z} are each independently H or optionally substituted C₁₋₄alkyl. In certain embodiments, R³ is H and R² is —N(CH₃)₂. In some embodiments, R³ is H and R² is —NR^{y}R^{z}, wherein R^{y} and R^{z} taken together with the nitrogen to which they are attached form an optionally substituted monocyclic heterocycloalkyl ring. In certain embodiments, R³ is H and R² is morpholinyl. In some embodiments, R³ is C₁₋₄ alkyl and R² is halogen. For instance, in some embodiments, R³ is methyl and R² is Cl. In other embodiments, R³ is H and R² is —CN.

It is understood that the descriptions of any variable of Formula (I) may, where applicable, be combined with one or more descriptions of any other variable, the same as if each and every combination of variables were specifically and individually listed. For example, every description of R¹ may be combined with every description of R² and R³ the same as if each and every combination were specifically and individually listed. Likewise, every description of R² may be combined with every description of R¹ and R³ the same as if each and every description were specifically and individually listed, and every description of R³ may be combined with every description of R¹ and R² the same as if each and every description were specifically and individually listed.

In some embodiments, the compound of Formula (I) is a compound shown in the following table.

| Compound | | |
|---|---|---|
| No. | Structure | Name |
| 1 | 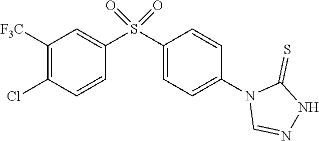 [SMILES: O=S(=O)(c1ccc(-n2cn[nH]c2=S)cc1)c1ccc(Cl)c(C(F)(F)F)c1] | 4-(4-((4-chloro-3- (trifluoromethyl)phenyl)sulfonyl)phenyl)- 2,4-dihydro-3H-1,2,4-triazole-3-thione |
| 2 | 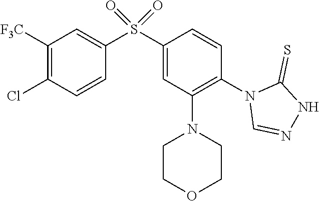 [SMILES: O=S(=O)(c1ccc(-n2cn[nH]c2=S)c(N2CCOCC2)c1)c1ccc(Cl)c(C(F)(F)F)c1] | 4-(4-((4-chloro-3- (trifluoromethyl)phenyl)sulfonyl)-2- morpholinophenyl)-2,4-dihydro-3H- 1,2,4-triazole-3-thione |
| 3 | 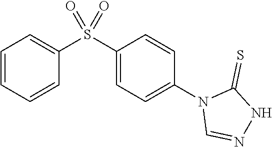 [SMILES: O=S(=O)(c1ccccc1)c1ccc(-n2cn[nH]c2=S)cc1] | 4-(4-(phenylsulfonyl)phenyl)-2,4- dihydro-3H-1,2,4-triazole-3-thione |
| 4 | 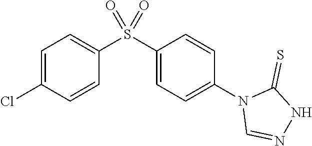 [SMILES: O=S(=O)(c1ccc(Cl)cc1)c1ccc(-n2cn[nH]c2=S)cc1] | 4-(4-((4-chlorophenyl)sulfonyl)phenyl)- 2,4-dihydro-3H-1,2,4-triazole-3-thione |
| 5 | 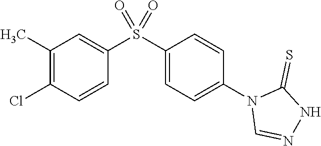 [SMILES: Cc1cc(S(=O)(=O)c2ccc(-n3cn[nH]c3=S)cc2)ccc1Cl] | 4-(4-((4-chloro-3- methylphenyl)sulfonyl)phenyl)-2,4- dihydro-3H-1,2,4-triazole-3-thione |
| 6 | 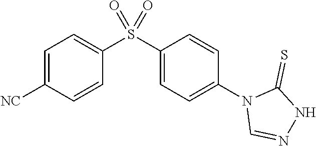 [SMILES: N#Cc1ccc(S(=O)(=O)c2ccc(-n3cn[nH]c3=S)cc2)cc1] | 4-((4-(5-thioxo-1,5-dihydro-4H-1,2,4- triazol-4-yl)phenyl)sulfonyl)benzonitrile |
| 7 | 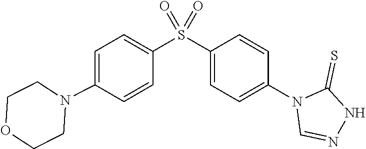 [SMILES: O=S(=O)(c1ccc(N2CCOCC2)cc1)c1ccc(-n2cn[nH]c2=S)cc1] | 4-(4-((4- morpholinophenyl)sulfonyl)phenyl)-2,4- dihydro-3H-1,2,4-triazole-3-thione |
| 8 | 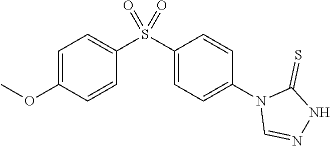 [SMILES: COc1ccc(S(=O)(=O)c2ccc(-n3cn[nH]c3=S)cc2)cc1] | 4-(4-((4- methoxyphenyl)sulfonyl)phenyl)-2,4- dihydro-3H-1,2,4-triazole-3-thione |
| 9 | 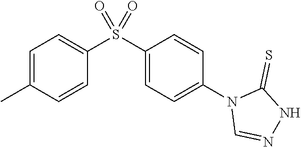 [SMILES: Cc1ccc(S(=O)(=O)c2ccc(-n3cn[nH]c3=S)cc2)cc1] | 4-(4-tosylphenyl)-2,4-dihydro-3H-1,2,4- triazole-3-thione |
| 10 | 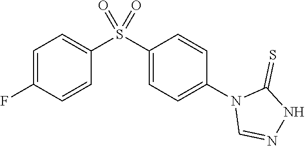 [SMILES: O=S(=O)(c1ccc(F)cc1)c1ccc(-n2cn[nH]c2=S)cc1] | 4-(4-((4-fluorophenyl)sulfonyl)phenyl)- 2,4-dihydro-3H-1,2,4-triazole-3-thione |
| 11 | 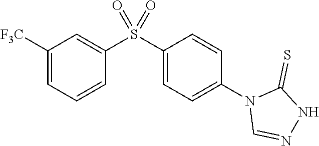 [SMILES: O=S(=O)(c1ccc(-n2cn[nH]c2=S)cc1)c1cccc(C(F)(F)F)c1] | 4-(4-((3- (trifluoromethyl)phenyl)sulfonyl)phenyl)- 2,4-dihydro-3H-1,2,4-triazole-3-thione |
| 12 | 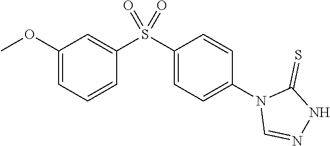 [SMILES: COc1cccc(S(=O)(=O)c2ccc(-n3cn[nH]c3=S)cc2)c1] | 4-(4-((3- methoxyphenyl)sulfonyl)phenyl)-2,4- dihydro-3H-1,2,4-triazole-3-thione |
| 13 | 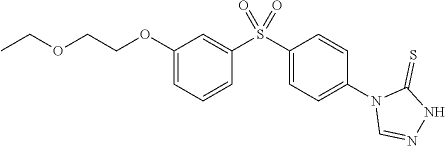 [SMILES: CCOCCOc1cccc(S(=O)(=O)c2ccc(-n3cn[nH]c3=S)cc2)c1] | 4-(4-((3-(2- ethoxyethoxy)phenyl)sulfonyl)phenyl)- 2,4-dihydro-3H-1,2,4-triazole-3-thione |
| 14 | 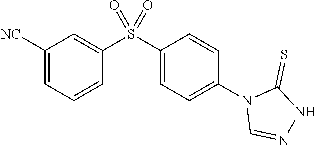 [SMILES: N#Cc1cccc(S(=O)(=O)c2ccc(-n3cn[nH]c3=S)cc2)c1] | 3-((4-(5-thioxo-1,5-dihydro-4H-1,2,4- triazol-4-yl)phenyl)sulfonyl)benzonitrile |
| 15 | 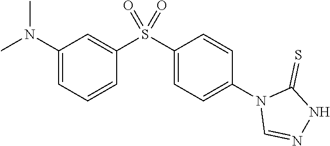 [SMILES: CN(C)c1cccc(S(=O)(=O)c2ccc(-n3cn[nH]c3=S)cc2)c1] | 4-(4-((3- (dimethylamino)phenyl)sulfonyl)phenyl)- 2,4-dihydro-3H-1,2,4-triazole-3-thione |
| 16 | 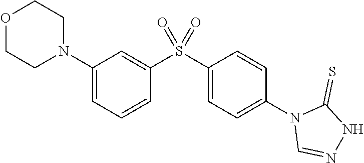 [SMILES: O=S(=O)(c1ccc(-n2cn[nH]c2=S)cc1)c1cccc(N2CCOCC2)c1] | 4-(4-((3- morpholinophenyl)sulfonyl)phenyl)-2,4- dihydro-3H-1,2,4-triazole-3-thione |
| 17 | 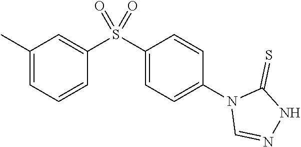 [SMILES: Cc1cccc(S(=O)(=O)c2ccc(-n3cn[nH]c3=S)cc2)c1] | 4-(4-(m-tolylsulfonyl)phenyl)-2,4- dihydro-3H-1,2,4-triazole-3-thione |
| 18 | 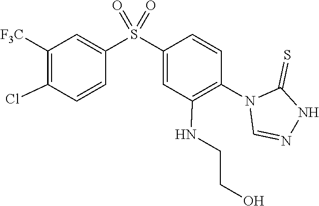 [SMILES: O=S(=O)(c1ccc(-n2cn[nH]c2=S)c(NCCO)c1)c1ccc(Cl)c(C(F)(F)F)c1] | 4-(4-((4-chloro-3- (trifluoromethyl)phenyl)sulfonyl)-2-((2- hydroxyethyl)amino)phenyl)-2,4- dihydro-3H-1,2,4-triazole-3-thione |
| 19 | 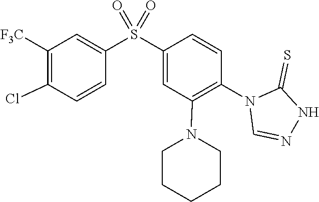 [SMILES: O=S(=O)(c1ccc(-n2cn[nH]c2=S)c(N2CCCCC2)c1)c1ccc(Cl)c(C(F)(F)F)c1] | 4-(4-((4-chloro-3- (trifluoromethyl)phenyl)sulfonyl)-2- (piperidin-1-yl)phenyl)-2,4-dihydro-3H- 1,2,4-triazole-3-thione |
| 20 | 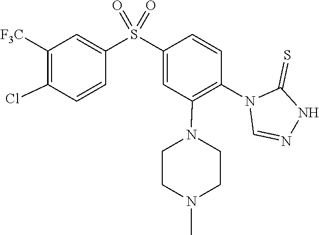 [SMILES: CN1CCN(c2cc(S(=O)(=O)c3ccc(Cl)c(C(F)(F)F)c3)ccc2-n2cn[nH]c2=S)CC1] | 4-(4-((4-chloro-3- (trifluoromethyl)phenyl)sulfonyl)-2-(4- methylpiperazin-1-yl)phenyl)-2,4- dihydro-3H-1,2,4-triazole-3-thione |
| 21 | 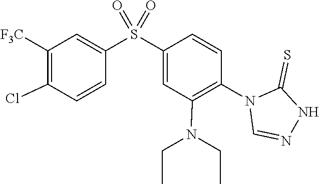 [SMILES: CCN(CC)c1cc(S(=O)(=O)c2ccc(Cl)c(C(F)(F)F)c2)ccc1-n1cn[nH]c1=S] | 4-(4-((4-chloro-3- (trifluoromethyl)phenyl)sulfonyl)-2- (diethylamino)phenyl)-2,4-dihydro-3H- 1,2,4-triazole-3-thione |
| 22 | 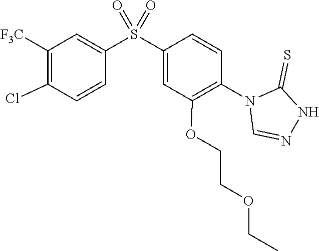 [SMILES: CCOCCOc1cc(S(=O)(=O)c2ccc(Cl)c(C(F)(F)F)c2)ccc1-n1cn[nH]c1=S] | 4-(4-((4-chloro-3- (trifluoromethyl)phenyl)sulfonyl)-2-(2- ethoxyethoxy)phenyl)-2,4-dihydro-3H- 1,2,4-triazole-3-thione |
| 23 | 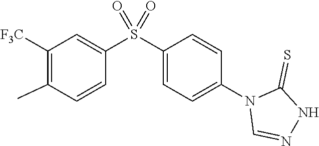 [SMILES: Cc1ccc(S(=O)(=O)c2ccc(-n3cn[nH]c3=S)cc2)cc1C(F)(F)F] | 4-(4-((4-methyl-3- (trifluoromethyl)phenyl)sulfonyl)phenyl)- 2,4-dihydro-3H-1,2,4-triazole-3-thione |
| 24 | 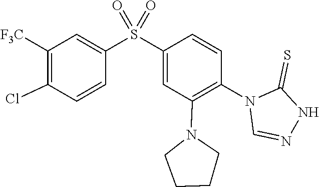 [SMILES: O=S(=O)(c1ccc(-n2cn[nH]c2=S)c(N2CCCC2)c1)c1ccc(Cl)c(C(F)(F)F)c1] | 4-(4-((4-chloro-3- (trifluoromethyl)phenyl)sulfonyl)-2- (pyrrolidin-1-yl)phenyl)-2,4-dihydro-3H- 1,2,4-triazole-3-thione |
| 25 | 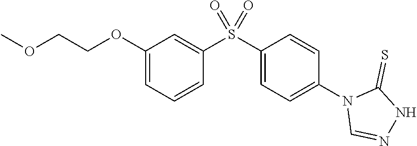 [SMILES: COCCOc1cccc(S(=O)(=O)c2ccc(-n3cn[nH]c3=S)cc2)c1] | 4-(4-((3-(2- methoxyethoxy)phenyl)sulfonyl)phenyl)- 2,4-dihydro-3H-1,2,4-triazole-3-thione |

or a pharmaceutically acceptable salt thereof.

In one aspect, the present disclosure provides a compound of Formula (IIA):

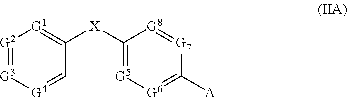

wherein

### G¹ is CH or N;

G² is CR^{2a} or N;
G³ is CR^{3a} or N;

### G⁴ is CH or N;

wherein no more than two of G¹, G², G³, and G⁴ are N;

### G⁵ is CH or N;

G⁶ is CR^{1a} or N;

### G⁷ is CH or N;

### G⁸ is CH or N;

wherein no more than one of G⁵, G⁶, G⁷, and G⁸ is N;
R^{1a}, R^{2a}, and R^{3a} are each independently hydrogen, hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, or an optionally substituted heterocyclyl;
   wherein R^{x} and R^{y} are each independently H or optionally substituted C₁₋₄alkyl;
or R^{1a} and R^{2a} are taken together with the carbons to which they are attached to form a 5- to 16-membered heterocyclyl ring;
   X is —CR^{4a}R^{5a}—, —O—, —S—, —S(O)—, —NR^{6a}—, —S(O)₂—, —NR^{6a}S(O)₂—, —CR^{4a}R^{5a}S(O)₂—, —C(O)—, —NR^{6a}C(O)—, or —NHNHC(O)—;

wherein R^{4a} and R^{5a} are independently hydrogen, hydroxy, halogen, substituted C₁₋₄ alkyl, C₁₋₄ alkoxy, or substituted C₁₋₄ alkoxy;

or R^{4a} and R^{5a} are taken together with the carbon to which they are attached to form a 3- to 6-membered cycloalkyl ring;

each R^{6a} is independently hydrogen or C₁₋₄ alkyl;

A is

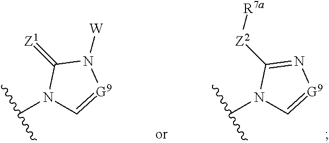

### G⁹ is CH or N;

Z¹ and Z² are independently S or O; and
W and R^{7a} are independently hydrogen or C₁₋₄ alkyl,

or a pharmaceutically acceptable salt thereof.

In one aspect, the present disclosure provides a compound of Formula (II):

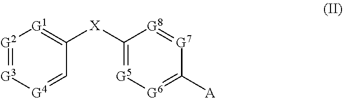

wherein

### G¹ is CH or N;

G² is CR^{2a} or N;
G³ is CR^{3a} or N;

### G⁴ is CH or N;

wherein no more than two of G¹, G², G³, and G⁴ are N;

### G⁵ is CH or N;

G⁶ is CR^{1a} or N;

### G⁷ is CH or N;

### G⁸ is CH or N;

wherein no more than one of G⁵, G⁶, G⁷, and G⁸ is N;
R^{1a}, R^{2a}, and R^{3a} are each independently hydrogen, hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, or an optionally substituted heterocyclyl;
   wherein R^{x} and R^{y} are each independently H or optionally substituted C₁₋₄alkyl;
or R^{1a} and R^{2a} are taken together with the carbons to which they are attached to form a 5- to 16-membered heterocyclyl ring;
   X is —CR^{4a}R^{5a}—, —O—, —S—, —S(O)—, —NR^{6a}—, —S(O)₂—, or —C(O)—;

wherein R^{4a} and R^{5a} are independently hydrogen, hydroxy, halogen, substituted C₁₋₄ alkyl, C₁₋₄ alkoxy, or substituted C₁₋₄ alkoxy;

or R^{4a} and R^{5a} are taken together with the carbon to which they are attached to form a 3- to 6-membered cycloalkyl ring;

R^{6a} is hydrogen or C₁₋₄ alkyl;

A is

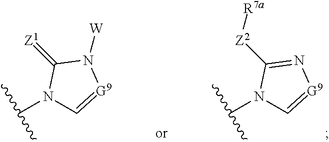

### G⁹ is CH or N;

Z¹ and Z² are independently S or O; and
W and R^{7a} are independently hydrogen or C₁₋₄ alkyl,

or a pharmaceutically acceptable salt thereof.

In some embodiments of Formula (IIA), one or more of the following apply: (i) X is —CR^{4a}R^{5a}—, —O—, —S—, —S(O)—, —NR^{6a}—, —NR^{6a}S(O)₂—, —CR^{4a}R^{5a}S(O)₂—, —C(O)—, —NR^{6a}C(O)—, or —NHNHC(O)—; (ii) one or two of G¹, G², G³, and G⁴ is N; (iii) one of G⁵, G⁶, G⁷, and G⁸ is N; (iv) R^{1a} is an optionally substituted heterocyclyl, wherein when the heterocyclyl is monocyclic, the point of connection is via a carbon atom; (v) R^{2a} is an optionally substituted heterocyclyl, wherein when the heterocyclyl is monocyclic, the point of connection is via a carbon atom; (vi) R^{3a} is an optionally substituted heterocyclyl, wherein when the heterocyclyl is monocyclic, the point of connection is via a carbon atom; (vii) R^{2a} and R^{3a} are taken together with the carbons to which they are attached to form a 5- to 16-membered heterocyclyl ring; (viii) A is

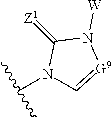

### and Z is O; (ix) A is

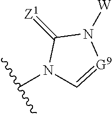

and W is C₁₋₄ alkyl; (x) A is

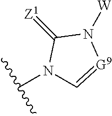

and G⁹ is CH; and (xii) A is

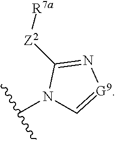

In some embodiments of Formula (II), one or more of the following apply: (i) X is —CR^{4a}R^{5a}—, —O—, —S—, —S(O)—, —NR^{6a}—, or —C(O)—; (ii) one or two of G¹, G², G³, and G⁴ is N; (iii) one of G⁵, G⁶, G⁷, and G⁸ is N; (iv) R^{1a} is an optionally substituted heterocyclyl, wherein when the heterocyclyl is monocyclic, the point of connection is via a carbon atom; (v) R^{2a} is an optionally substituted heterocyclyl, wherein when the heterocyclyl is monocyclic, the point of connection is via a carbon atom; (vi) R^{3a} is an optionally substituted heterocyclyl, wherein when the heterocyclyl is monocyclic, the point of connection is via a carbon atom; (vii) R^{2a} and R^{3a} are taken together with the carbons to which they are attached to form a 5- to 16-membered heterocyclyl ring; (viii) A is

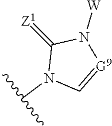

### and Z¹ is O; (ix) A is

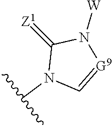

and W is C₁₋₄ alkyl; (x) A is

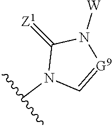

and G⁹ is CH; and (xii) A is

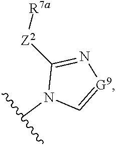

In some embodiments of Formula (IIA) or Formula (II), G¹ is CH. In some embodiments, G¹ is N.

In some embodiments of Formula (IIA) or Formula (II), G² is CR^{2a}. In some embodiments, G² is N. In some embodiments, G² is CR^{2a} and R^{2a} is selected from the group consisting of hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, and an optionally substituted heterocyclyl. In some embodiments, G² is CH. In some embodiments, G² is CR^{2a} and R^{2a} is hydroxy. In some embodiments, G² is CR^{2a} and R^{2a} is halogen. In certain embodiments, G² is CR^{2a} and R^{2a} is Cl. In certain embodiments, G² is CR^{2a} and R^{2a} is F. In other embodiments, G² is CR^{2a} and R^{2a} is Br or I. In some embodiments, G² is CR^{2a} and R^{2a} is C₁₋₄ alkyl. For example, in some embodiments, G² is CR^{2a} and R^{2a} is methyl. In some embodiments, G² is CR^{2a} and R^{2a} is ethyl. In some embodiments, G² is CR^{2a} and R^{2a} is n-propyl or isopropyl. In other embodiments, G² is CR^{2a} and R^{2a} is n-butyl, isobutyl, secbutyl, or tertbutyl. In some embodiments, G² is CR^{2a} and R^{2a} is C₁₋₄ alkyl substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄ alkyl, —C(O)C₁₋₄alkyl, —C(O)OC₁₋₄alkyl, or —S(O)₂C₁₋₄alkyl. In some embodiments, G² is CR^{2a} and R^{2a} is C₁₋₄ alkoxy. For example, in some embodiments, G² is CR^{2a} and R^{2a} is methoxy. In some embodiments, G² is CR^{2a} and R^{2a} is ethoxy, propoxy, isopropoxy, butoxy, isobutoxy, secbutoxy, or tertbutoxy. In some embodiments, G² is CR^{2a} and R^{2a} is C₁₋₄ alkoxy substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄alkyl, —C(O)C₁₋₄alkyl, —C(O)OC₁₋₄alkyl, or —S(O)₂C₁₋₄ alkyl. In some embodiments, G² is CR^{2a} and R^{2a} is —CN. In some embodiments, G² is CR^{2a} and R^{2a} is —C(O)R^{x}, wherein R^{x} is H or optionally substituted C₁₋₄alkyl. In some embodiments, In some embodiments, G² is CR^{2a} and R^{2a} is —C(O)H, —C(O)CH₃, —C(O)CH₂CH₃, —C(O)CH₂CH₂CH₃, —C(O)CH(CH₃)₂, —C(O)CH₂CH₂CH₂CH₃, —C(O)CH(CH₃)CH₂CH₃, —C(O)CH₂CH(CH₃)₂, or —C(O)C(CH₃)₃. In some embodiments, G² is CR^{2a} and R^{2a} is —C(O)OR^{x}, wherein R^{x} is H or optionally substituted C₁₋₄alkyl. In some embodiments, In some embodiments, G² is CR^{2a} and R^{2a} is —C(O)OH, —C(O)OCH₃, —C(O)OCH₂CH₃, —C(O)OCH₂CH₂CH₃, —C(O)OCH(CH₃)₂, —C(O)OCH₂CH₂CH₂CH₃, —C(O)OCH(CH₃)CH₂CH₃, —C(O)OCH₂CH(CH₃)₂, or —C(O)OC(CH₃)₃. In some embodiments, G² is CR^{2a} and R^{2a} is —S(O)₂R^{x}, wherein R^{x} is H or optionally substituted C₁₋₄ alkyl. In some embodiments, In some embodiments, G² is CR^{2a} and R^{2a} is —S(O)₂H, —S(O)₂CH₃, —S(O)₂CH₂CH₃, —S(O)₂CH₂CH₂CH₃, —S(O)₂CH(CH₃)₂, —S(O)₂CH₂CH₂CH₂CH₃, —S(O)₂CH(CH₃)CH₂CH₃, —S(O)₂CH₂CH(CH₃)₂, or —S(O)₂C(CH₃)₃. In some embodiments, G² is CR^{2a} and R^{2a} is —NR^{x}R^{y}, wherein R^{x} and R^{y} are each independently H or optionally substituted C₁₋₄alkyl. In some embodiments, G² is CR^{2a} and R^{2a} is —NH₂. In some embodiments, G² is CR^{2a} and R^{2a} is —NH(C₁₋₄alkyl), such as —NHCH₃, —NHCH₂CH₃, —NHCH₂CH₂CH₃, —NHCH(CH₃)₂, —NHCH₂CH₂CH₂CH₃, —NHCH(CH₃)CH₂CH₃, —NHCH₂CH(CH₃)₂, or —NHC(CH₃)₃. In other embodiments, G² is CR^{2a} and R^{2a} is —N(C₁₋₄ alkyl)₂, including, but not limited to —N(CH₃)₂, —N(CH₂CH₃)₂, —N(CH(CH₃)₂)₂, and —N(CH(CH₃)₂)₂. In some embodiments, G² is CR^{2a} and R^{2a} is an optionally substituted heterocyclyl containing one or more heteroatoms selected from N, O, and S. In some embodiments, G² is CR^{2a} and R^{2a} is an optionally substituted 5- to 12-membered heterocycloalkyl ring. In some embodiments, G² is CR^{2a} and R^{2a} is an optionally substituted monocyclic heterocycloalkyl ring. In some embodiments, G² is CR^{2a} and R^{2a} is an optionally substituted bicyclic heterocycloalkyl ring. In some embodiments, G² is CR^{2a} and R^{2a} is an optionally substituted 5- to 6-membered heterocycloalkyl ring.

In some embodiments of Formula (IIA) or Formula (II), G³ is CR³a. In some embodiments, G³ is N. In some embodiments, G³ is CR^{3a} and R^{3a} is selected from the group consisting of hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, and an optionally substituted heterocyclyl. In some embodiments, G³ is CH. In some embodiments, G³ is CR^{3a} and R^{3a} is hydroxy. In some embodiments, G³ is CR^{3a} and R^{3a} is halogen. In certain embodiments, G³ is CR^{3a} and R^{3a} is Cl. In certain embodiments, G³ is CR^{3a} and R^{3a} is F. In other embodiments, G³ is CR^{3a} and R^{3a} is Br or I. In some embodiments, G³ is CR^{3a} and R^{3a} is C₁₋₄ alkyl. For example, in some embodiments, G³ is CR^{3a} and R^{3a} is methyl. In some embodiments, G³ is CR^{3a} and R^{3a} is ethyl. In some embodiments, G³ is CR^{3a} and R^{3a} is n-propyl or isopropyl. In other embodiments, G³ is CR^{3a} and R^{3a} is n-butyl, isobutyl, secbutyl, or tertbutyl. In some embodiments, G³ is CR^{3a} and R^{3a} is C₁₋₄ alkyl substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄ alkyl, —C(O)C₁₋₄alkyl, —C(O)OC₁₋₄alkyl, or —S(O)₂C₁₋₄alkyl. In some embodiments, G³ is CR^{3a} and R^{3a} is C₁₋₄ alkoxy. For example, in some embodiments, G³ is CR^{3a} and R^{3a} is methoxy. In some embodiments, G³ is CR^{3a} and R^{3a} is ethoxy, propoxy, isopropoxy, butoxy, isobutoxy, secbutoxy, or tertbutoxy. In some embodiments, G³ is CR^{3a} and R^{3a} is C₁₋₄ alkoxy substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄alkyl, —C(O)C₁₋₄alkyl, —C(O)OC₁₋₄alkyl, or —S(O)₂C₁₋₄ alkyl. In some embodiments, G³ is CR^{3a} and R^{3a} is —CN. In some embodiments, G³ is CR^{3a} and R^{3a} is —C(O)R^{x}, wherein R^{x} is H or optionally substituted C₁₋₄alkyl. In some embodiments, In some embodiments, G³ is CR^{3a} and R^{3a} is —C(O)H, —C(O)CH₃, —C(O)CH₂CH₃, —C(O)CH₂CH₂CH₃, —C(O)CH(CH₃)₂, —C(O)CH₂CH₂CH₂CH₃, —C(O)CH(CH₃)CH₂CH₃, —C(O)CH₂CH(CH₃)₂, or —C(O)C(CH₃)₃. In some embodiments, G³ is CR^{3a} and R^{3a} is —C(O)OR^{x}, wherein R^{x} is H or optionally substituted C₁₋₄alkyl. In some embodiments, In some embodiments, G³ is CR^{3a} and R^{3a} is —C(O)OH, —C(O)OCH₃, —C(O)OCH₂CH₃, —C(O)OCH₂CH₂CH₃, —C(O)OCH(CH₃)₂, —C(O)OCH₂CH₂CH₂CH₃, —C(O)OCH(CH₃)CH₂CH₃, —C(O)OCH₂CH(CH₃)₂, or —C(O)OC(CH₃)₃. In some embodiments, G³ is CR^{3a} and R^{3a} is —S(O)₂R^{x}, wherein R^{x} is H or optionally substituted C₁₋₄ alkyl. In some embodiments, In some embodiments, G³ is CR^{3a} and R^{3a} is —S(O)₂H, —S(O)₂CH₃, —S(O)₂CH₂CH₃, —S(O)₂CH₂CH₂CH₃, —S(O)₂CH(CH₃)₂, —S(O)₂CH₂CH₂CH₂CH₃, —S(O)₂CH(CH₃)CH₂CH₃, —S(O)₂CH₂CH(CH₃)₂, or —S(O)₂C(CH₃)₃. In some embodiments, G³ is CR^{3a} and R^{3a} is —NR^{x}R^{y}, wherein R^{x} and R^{y} are each independently H or optionally substituted C₁₋₄alkyl. In some embodiments, G³ is CR^{3a} and R^{3a} is —NH₂. In some embodiments, G³ is CR^{3a} and R^{3a} is —NH(C₁₋₄alkyl), such as —NHCH₃, —NHCH₂CH₃, —NHCH₂CH₂CH₃, —NHCH(CH₃)₂, —NHCH₂CH₂CH₂CH₃, —NHCH(CH₃)CH₂CH₃, —NHCH₂CH(CH₃)₂, or —NHC(CH₃)₃. In other embodiments, G³ is CR^{3a} and R^{3a} is —N(C₁₋₄ alkyl)₂, including, but not limited to —N(CH₃)₂, —N(CH₂CH₃)₂, —N(CH(CH₃)₂)₂, and —N(CH(CH₃)₂)₂. In some embodiments, G³ is CR^{3a} and R^{3a} is an optionally substituted heterocyclyl containing one or more heteroatoms selected from N, O, and S. In some embodiments, G³ is CR^{3a} and R^{3a} is an optionally substituted 5- to 12-membered heterocycloalkyl ring. In some embodiments, G³ is CR^{3a} and R^{3a} is an optionally substituted monocyclic heterocycloalkyl ring. In some embodiments, G³ is CR^{3a} and R^{3a} is an optionally substituted bicyclic heterocycloalkyl ring. In some embodiments, G³ is CR^{3a} and R^{3a} is an optionally substituted 5- to 6-membered heterocycloalkyl ring.

In some embodiments of Formula (IIA) or Formula (II), G⁴ is CH. In some embodiments, G⁴ is N.

In some embodiments of Formula (IIA) or Formula (II), G² is CR^{2a}, G³ is CR^{3a}, and R^{2a} and R^{3a} are taken together with the carbons to which they are attached to form a 5- to 16-membered heterocyclyl ring. In some embodiments, G² is CR^{2a}, G³ is CR^{3a}, and R^{2a} and R^{3a} are taken together with the carbons to which they are attached to form a 5- to 16-membered heterocyclyl ring comprising one or more heteroatoms selected from N, O, and S. In some embodiments, G² is CR^{2a}, G³ is CR^{3a}, and R^{2a} and R^{3a} are taken together with the carbons to which they are attached to form a 6- to 12-membered heterocyclyl ring comprising one or more O atoms. In some embodiments, G² is CR^{2a}, G³ is CR^{3a}, and R^{2a} and R^{3a} are taken together with the carbons to which they are attached to form a 9- to 12-membered heterocyclyl ring comprising one or more O atoms.

In some embodiments, the compound of Formula (IIA) or Formula (II) is a compound of Formula (II-1):

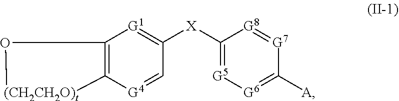

or a pharmaceutically acceptable salt thereof, wherein G¹, G⁴, G⁵, G⁶, G⁷, G⁸, X and A are as defined for Formula (IIA) or Formula (II), and t is 1, 2, or 3.

In some embodiments, the compound of Formula (IIA) or Formula (II) is a compound of Formula (II-2):

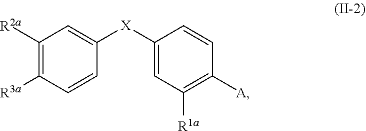

or a pharmaceutically acceptable salt thereof, wherein R^{1a}, R^{2a}, R^{3a}, X and A are as defined for Formula (IIA) or Formula (II).

In some embodiments of Formula (IIA) or Formula (II), one of G¹, G², G³, and G⁴ is N. In some embodiments, G¹ is N, G² is CR^{2a}, G³ is CR^{3a}, and G⁴ is CH. In some embodiments, G¹ is CH, G² is N, G³ is CR^{3a}, and G⁴ is CH. In some embodiments, G¹ is CH, G² is CR^{2a}, G³ is N, and G⁴ is CH. In some embodiments, G¹ is CH, G² is CR^{2a}, G³ is CR^{3a}, and G⁴ is N. In some embodiments, two of G¹, G², G³, and G⁴ are N. In some embodiments, G¹ is N, G² is N, G³ is CR^{3a}, and G⁴ is CH. In some embodiments, G¹ is N, G² is CR^{2a}, G³ is N, and G⁴ is CH. In some embodiments, G¹ is N, G² is CR^{2a}, G³ is CR^{3a}, and G⁴ is N. In some embodiments, G¹ is CH, G² is N, G³ is N, and G⁴ is CH. In some embodiments, G¹ is CH, G² is N, G³ is CR^{3a}, and G⁴ is N. In some embodiments, G¹ is CH, G² is CR^{2a}, G³ is N, and G⁴ is N. In some embodiments, G¹ is CH, G² is CR^{2a}, G³ is CR^{3a}, and G⁴ is CH. In some embodiments, no more than one of G¹, G², G³, and G⁴ is N. In some embodiments, no more than two of G¹, G², G³, and G⁴ is N.

In some embodiments of Formula (IIA) or Formula (II), G⁵ is CH. In some embodiments, G⁵ is N. In some embodiments of Formula (IIA) or Formula (II), G⁶ is CR^{1a} In some embodiments, G⁶ is CR^{1a} and R^{1a} is selected from the group consisting of hydrogen, hydroxy, halogen, C₁₋₄ alkyl, C₁₋₄ haloalkyl, C₁₋₄ alkoxy, and C₁₋₄ haloalkoxy. In some embodiments, G⁶ is N. In some embodiments of Formula (IIA) or Formula (II), G⁷ is CH. In some embodiments, G⁷ is N. In some embodiments of Formula (IIA) or Formula (II), G⁸ is CH. In some embodiments, G⁸ is N.

In some embodiments of Formula (IIA) or Formula (II), one of G⁵, G⁶, G⁷, and G⁸ is N. In some embodiments, G⁵ is N, G⁶ is CR^{1a}, and G⁷ is CH, and G⁸ is CH. In some embodiments, G⁵ is CH, G⁶ is N, G⁷ is CH, and G⁸ is CH. In other embodiments, G⁵ is CH, G⁶ is CR^{1a}, G⁷ is N, and G⁸ is CH. In some embodiments, G⁵ is CH, G⁶ is CR^{1a}, G⁷ is CH, and G⁸ is N. In some embodiments, G⁵ is CH, G⁶ is CR^{1a}, G⁷ is CH, and G⁸ is CH. In some embodiments, no more than one of G⁵, G⁶, G⁷, and G⁸ is N.

In some embodiments of Formula (IIA) or Formula (II), In some embodiments, G⁶ is CR^{1a} and R^{1a} is selected from the group consisting of hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, and an optionally substituted heterocyclyl. In some embodiments, G⁶ is CH. In some embodiments, G⁶ is CR^{1a} and R^{1a} is hydroxy. In some embodiments, G⁶ is CR^{1a} and R^{1a} is halogen. In certain embodiments, G⁶ is CR^{1a} and R^{1a} is Cl. In certain embodiments, G⁶ is CR^{1a} and R^{1a} is F. In other embodiments, G⁶ is CR^{1a} and R^{1a} is Br or I. In some embodiments, G⁶ is CR^{1a} and R^{1a} is C₁₋₄ alkyl. For example, in some embodiments, G⁶ is CR^{1a} and R^{1a} is methyl. In some embodiments, G⁶ is CR^{1a} and R^{1a} is ethyl. In some embodiments, G⁶ is CR^{1a} and R^{1a} is n-propyl or isopropyl. In other embodiments, G⁶ is CR^{1a} and R^{1a} is n-butyl, isobutyl, secbutyl, or tertbutyl. In some embodiments, G⁶ is CR^{1a} and R^{1a} is C₁₋₄ alkyl substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄alkyl, —C(O)C₁₋₄alkyl, —C(O)OC₁₋₄alkyl, or —S(O)₂C₁₋₄ alkyl. In some embodiments, G⁶ is CR^{1a} and R^{1a} is C₁₋₄ alkoxy. For example, in some embodiments, G⁶ is CR^{1a} and R^{1a} is methoxy. In some embodiments, G⁶ is CR^{1a} and R^{1a} is ethoxy, propoxy, isopropoxy, butoxy, isobutoxy, secbutoxy, or tertbutoxy. In some embodiments, G⁶ is CR^{1a} and R^{1a} is C₁₋₄ alkoxy substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄alkyl, —C(O)C₁₋₄ alkyl, —C(O)OC₁₋₄alkyl, or —S(O)₂C₁₋₄alkyl. In some embodiments, G⁶ is CR^{1a} and R^{1a} is —CN. In some embodiments, G⁶ is CR^{1a} and R^{1a} is —C(O)R^{x}, wherein R^{x} is H or optionally substituted C₁₋₄alkyl. In some embodiments, In some embodiments, G⁶ is CR^{1a} and R^{1a} is —C(O)H, —C(O)CH₃, —C(O)CH₂CH₃, —C(O)CH₂CH₂CH₃, —C(O)CH(CH₃)₂, —C(O)CH₂CH₂CH₂CH₃, —C(O)CH(CH₃)CH₂CH₃, —C(O)CH₂CH(CH₃)₂, or —C(O)C(CH₃)₃. In some embodiments, G⁶ is CR^{1a} and R^{1a} is —C(O)OR^{x}, wherein R^{x} is H or optionally substituted C₁₋₄alkyl. In some embodiments, In some embodiments, G⁶ is CR^{1a} and R^{1a} is —C(O)OH, —C(O)OCH₃, —C(O)OCH₂CH₃, —C(O)OCH₂CH₂CH₃, —C(O)OCH(CH₃)₂, —C(O)OCH₂CH₂CH₂CH₃, —C(O)OCH(CH₃)CH₂CH₃, —C(O)OCH₂CH(CH₃)₂, or —C(O)OC(CH₃)₃. In some embodiments, G⁶ is CR^{1a} and R^{1a} is —S(O)₂R^{x}, wherein R^{x} is H or optionally substituted C₁₋₄alkyl. In some embodiments, In some embodiments, G⁶ is CR^{1a} and R^{1a} is —S(O)₂H, —S(O)₂CH₃, —S(O)₂CH₂CH₃, —S(O)₂CH₂CH₂CH₃, —S(O)₂CH(CH₃)₂, —S(O)₂CH₂CH₂CH₂CH₃, —S(O)₂CH(CH₃)CH₂CH₃, —S(O)₂CH₂CH(CH₃)₂, or —S(O)₂C(CH₃)₃. In some embodiments, G⁶ is CR^{1a} and R^{1a} is —NR^{x}R^{y}, wherein R^{x} and R^{y} are each independently H or optionally substituted C₁₋₄alkyl. In some embodiments, G⁶ is CR^{1a} and R^{1a} is —NH₂. In some embodiments, G⁶ is CR^{1a} and R^{1a} is —NH(C₁₋₄alkyl), such as —NHCH₃, —NHCH₂CH₃, —NHCH₂CH₂CH₃, —NHCH(CH₃)₂, —NHCH₂CH₂CH₂CH₃, —NHCH(CH₃)CH₂CH₃, —NHCH₂CH(CH₃)₂, or —NHC(CH₃)₃. In other embodiments, G⁶ is CR^{1a} and R^{1a} is —N(C₁₋₄alkyl)₂, including, but not limited to —N(CH₃)₂, —N(CH₂CH₃)₂, —N(CH(CH₃)₂)₂, and —N(CH(CH₃)₂)₂. In some embodiments, G⁶ is CR^{1a} and R^{1a} is an optionally substituted heterocyclyl containing one or more heteroatoms selected from N, O, and S. In some embodiments, G⁶ is CR^{1a} and R^{1a} is an optionally substituted 5- to 12-membered heterocycloalkyl ring. In some embodiments, G⁶ is CR^{1a} and R^{1a} is an optionally substituted monocyclic heterocycloalkyl ring. In some embodiments, G⁶ is CR^{1a} and R^{1a} is an optionally substituted bicyclic heterocycloalkyl ring. In some embodiments, G⁶ is CR^{1a} and R^{1a} is an optionally substituted 5- to 6-membered heterocycloalkyl ring.

In some embodiments, one or more of R^{1a}, R^{2a}, and R^{3a} is C₁₋₄ alkyl, which is unsubstituted or substituted with one or more substituents selected from the group consisting of halogen, —CN, —OR⁴, —SR⁴, —NR⁵R⁶, —NO₂, —C═NH(OR⁴), —C(O)R⁴, —OC(O)R⁴, —C(O)OR⁴, —C(O)NR⁵R⁶, —OC(O)NR⁵R⁶, —NR⁴C(O)R⁵, —NR⁴C(O)OR⁵, —NR⁴C(O)NR⁵R⁶, —S(O)R⁴, —S(O)₂R⁴, —NR⁴S(O)R⁵, —C(O)NR⁴S(O)R⁵, —NR⁴S(O)₂R⁵, —C(O)NR⁴S(O)₂R⁵, —S(O)NR⁵R⁶, —S(O)₂NR⁵R⁶, —P(O)(OR⁵) (OR⁶), C₃-C₆ cycloalkyl, 3-12-membered heterocyclyl, 5- to 10-membered heteroaryl, and C₆-C₁₄ aryl; wherein R⁴ is independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl or 3-6-membered heterocyclyl, wherein the C₁-C₆ alkyl, C₂-C₆ alkenyl, C₂-C₆ alkynyl, C₃-C₆ cycloalkyl, C₆-C₁₄ aryl, 5-6-membered heteroaryl and 3-6-membered heterocyclyl are independently optionally substituted by halogen, oxo, —CN, —OR⁹, —NR⁹R¹⁰, —P(O)(OR⁹)(OR¹⁰), phenyl optionally substituted by halogen, or C₁-C₆ alkyl optionally substituted by halogen, —OH or oxo; R⁵ and R⁶ are each independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl or 3-6 membered heterocyclyl, wherein the C₁-C₆ alkyl, C₂-C₆ alkenyl, C₂-C₆ alkynyl, C₃-C₆ cycloalkyl, C₆-C₁₄ aryl, 5-6-membered heteroaryl and 3-6 membered heterocyclyl are independently optionally substituted by halogen, oxo, —CN, —OR⁹, —NR⁹R¹⁰ or C₁-C₆ alkyl optionally substituted by halogen, —OH or oxo; and R⁹ and R¹⁰ are each independently hydrogen, C₁-C₆ alkyl, C₂-C₆ alkenyl, C₂-C₆ alkynyl, C₁-C₆ alkyl substituted by one or more halogen, C₂-C₆ alkenyl substituted by one or more halogen, or C₂-C₆ alkynyl substituted by one or more halogen.

In some embodiments, one or more of R^{1a}, R^{2a}, and R^{3a} is C₁₋₄ alkoxy, which is unsubstituted or substituted with one or more substituents selected from the group consisting of C₁-C₆ alkyl, C₂-C₆ alkenyl, C₂-C₆ alkynyl, halogen, —CN, —OR⁴, —SR⁴, —NR⁵R⁶, —NO₂, —C═NH(OR⁴), —C(O)R⁴, —OC(O)R⁴, —C(O)OR⁴, —C(O)NR⁵R⁶, —OC(O)NR⁵R⁶, —NR⁴C(O)R⁵, —NR⁴C(O)OR⁵, —NR⁴C(O)NR⁵R⁶, —S(O)R⁴, —S(O)₂R⁴, —NR⁴S(O)R⁵, —C(O)NR⁴S(O)R⁵, —NR⁴S(O)₂R⁵, —C(O)NR⁴S(O)₂R⁵, —S(O)NR⁵R⁶, —S(O)₂NR⁵R⁶, —P(O)(OR⁵) (OR⁶), C₃-C₆ cycloalkyl, 3-12-membered heterocyclyl, 5- to 10-membered heteroaryl, and C₆-C₁₄ aryl; wherein R⁴ is independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl or 3-6-membered heterocyclyl, wherein the C₁-C₆ alkyl, C₂-C₆ alkenyl, C₂-C₆ alkynyl, C₃-C₆ cycloalkyl, C₆-C₁₄ aryl, 5-6-membered heteroaryl and 3-6-membered heterocyclyl are independently optionally substituted by halogen, oxo, —CN, —OR⁹, —NR⁹R¹⁰, —P(O)(OR⁹)(OR¹⁰), phenyl optionally substituted by halogen, or C₁-C₆ alkyl optionally substituted by halogen, —OH or oxo; and R⁵ and R⁶ are each independently hydrogen, C₁-C₆ alkyl, C₂-C₆ alkenyl, C₂-C₆ alkynyl, C₃-C₆ cycloalkyl, C₆-C₁₄ aryl, 5-6-membered heteroaryl or 3-6 membered heterocyclyl, wherein the C₁-C₆ alkyl, C₂-C₆ alkenyl, C₂-C₆ alkynyl, C₃-C₆ cycloalkyl, C₆-C₁₄ aryl, 5-6-membered heteroaryl and 3-6 membered heterocyclyl are independently optionally substituted by halogen, oxo, —CN, —OR⁹, —NR⁹R¹⁰ or C₁-C₆ alkyl optionally substituted by halogen, —OH or oxo; and R⁹ and R¹⁰ are each independently hydrogen, C₁-C₆ alkyl, C₂-C₆ alkenyl, C₂-C₆ alkynyl, C₁-C₆ alkyl substituted by one or more halogen, C₂-C₆ alkenyl substituted by one or more halogen, or C₂-C₆ alkynyl substituted by one or more halogen.

In some embodiments of Formula (IIA) or Formula (II), A is

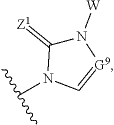

wherein Z¹ is S or O, G⁹ is N, and W is H or C₁₋₄ alkyl. In some embodiments, Z¹ is S, G⁹ is N, and W is H. In some embodiments, Z¹ is O, G⁹ is N, and W is H. In some embodiments, Z¹ is S, G⁹ is N, and W is C₁₋₄ alkyl. In some embodiments, Z¹ is O, G⁹ is N, and W is C₁₋₄ alkyl. In some embodiments, A is

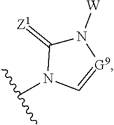

wherein Z¹ is S or O, G⁹ is CH, and W is H or C₁₋₄ alkyl. In some embodiments, Z¹ is S, G⁹ is CH, and W is H. In some embodiments, Z¹ is O, G⁹ is CH, and W is H. In some embodiments, Z¹ is S, G⁹ is CH, and W is C₁₋₄ alkyl. In some embodiments, Z¹ is O, G⁹ is CH, and W is C₁₋₄ alkyl. In some embodiments, A is

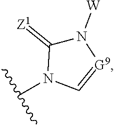

wherein Z¹ is S, G⁹ is CH or N, and W is H or C₁₋₄ alkyl. In some embodiments, A is

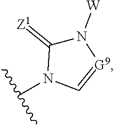

wherein Z¹ is O, G⁹ is CH or N, and W is H or C₁₋₄ alkyl. In some embodiments, A is

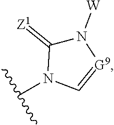

wherein Z¹ is S or O, G⁹ is CH or N, and W is H. In some embodiments, A is

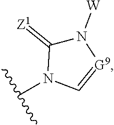

wherein Z¹ is S or O, G⁹ is CH or N, and W is C₁₋₄ alkyl.

In some embodiments of Formula (IIA) or Formula (II), A is

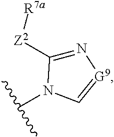

wherein Z² is S or O, G⁹ is N, and R^{7a} is H or C₁₋₄ alkyl. In some embodiments, Z² is S, G⁹ is N, and R^{7a} is H. In some embodiments, Z² is O, G⁹ is N, and R^{7a} is H. In some embodiments, Z² is S, G⁹ is N, and R^{7a} is C₁₋₄ alkyl. In some embodiments, Z² is O, G⁹ is N, and R^{7a} is C₁₋₄ alkyl. In some embodiments, A is

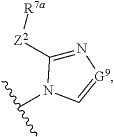

wherein Z² is S or O, G⁹ is CH, and R^{7a} is H or C₁₋₄ alkyl. In some embodiments, Z² is S, G⁹ is CH, and R^{7a} is H. In some embodiments, Z² is O, G⁹ is CH, and R^{7a} is H. In some embodiments, Z² is S, G⁹ is CH, and R^{7a} is C₁₋₄ alkyl. In some embodiments, Z² is O, G⁹ is CH, and R^{7a} is C₁₋₄ alkyl. In some embodiments, A is

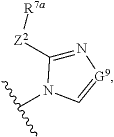

wherein Z² is S, G⁹ is CH or N, and R^{7a} is H or C₁₋₄ alkyl. In some embodiments, A is

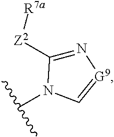

wherein Z² is O, G⁹ is CH or N, and R^{7a} is H or C₁₋₄ alkyl. In some embodiments, A is

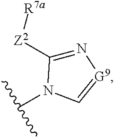

wherein Z² is S or O, G⁹ is CH or N, and R^{7a} is H. In some embodiments, A is

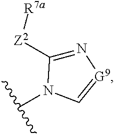

wherein Z² is S or O, G⁹ is CH or N, and R^{7a} is C₁₋₄ alkyl.

In some embodiments of Formula (IIA) or Formula (II), X is —CR^{4a}R^{5a}—, wherein R^{4a} and R^{5a} are each independently hydrogen, hydroxy, halogen, substituted C₁₋₄ alkyl, C₁₋₄ alkoxy, or substituted C₁₋₄ alkoxy. In some embodiments, X is —CR^{4a}R^{5a}—; wherein R^{4a} and R^{5a}, are each independently hydrogen, hydroxy, halogen, or R^{4a} and R^{5a} are taken together with the carbon to which they are attached to form a 3- to 6-membered cycloalkyl ring. In some embodiments, X is —CH₂—. In some embodiments, X is —CR^{4a}R^{5a}—, wherein one of R^{4a} and R^{5a} is hydrogen and the other is hydroxy, halogen, substituted C₁₋₄ alkyl, C₁₋₄ alkoxy, or substituted C₁₋₄ alkoxy. In some embodiments, X is —CR^{4a}R^{5a}—, wherein each of R^{4a} and R^{5a} is independently hydroxy, halogen, substituted C₁₋₄ alkyl, C₁₋₄ alkoxy, or substituted C₁₋₄ alkoxy. In some embodiments, X is —CR^{4a}R^{5a}—, wherein R^{4a} and R^{5a} are each independently hydrogen, hydroxy, halogen, C₁₋₄ alkyl substituted with one or more halogen, C₁₋₄ alkoxy, or C₁₋₄ alkoxy substituted with one or more halogen. In some embodiments, X is —CR^{4a}R^{5a}—, and one of R^{4a} and R^{5a} is hydroxy. For instance, in some embodiments, X is —CH(OH)—. In some embodiments, X is —CR^{4a}R^{5a}—, and one or both R^{4a} and R^{5a} is F, Cl, Br, or I. For instance, in some embodiments, X is —C(F)₂—. In some embodiments, X is —CR^{4a}R^{5a}—, and one or both R^{4a} and R^{5a} is C₁₋₄ alkyl substituted with halogen, including, but not limited to —CF₃, —(CH₂)F, —CHF₂, CH₂Br, —CH₂CF₃, —CH₂CHF₂, and —CH₂CH₂F. In some embodiments, X is —CR^{4a}R^{5a}—, and one or both R^{4a} and R^{5a} is C₁₋₄ alkoxy, including, but not limited to methoxy, ethoxy, n-propoxy, isopropoxy, n-butoxy, t-butoxy, and sec-butoxy. In some embodiments, X is —CR^{4a}R^{5a}—, and one or both R^{4a} and R^{5a} is C₁₋₄ alkoxy substituted with halogen, including, but not limited to —OCF₃, —O(CH₂)F, —OCHF₂, —OCH₂Br, —OCH₂CF₃, —OCH₂CHF₂, and —OCH₂CH₂F.

In some embodiments, X is —CR^{4a}R^{5a}—, wherein R^{4a} and R^{5a} are taken together with the carbon to which they are attached to form a 3- to 6-membered cycloalkyl ring. In some embodiments, X is —CR^{4a}R^{5a}—, wherein R^{4a} and R^{5a} are taken together with the carbon to which they are attached to form a cyclopropyl ring. In some embodiments, X is —CR^{4a}R^{5a}—, wherein R^{4a} and R^{5a} are taken together with the carbon to which they are attached to form a cyclobutyl ring. In some embodiments, X is —CR^{4a}R^{5a}—, wherein R^{4a} and R^{5a} are taken together with the carbon to which they are attached to form a cyclopentyl ring. In some embodiments, X is —CR^{4a}R^{5a}—, wherein R^{4a} and R^{5a} are taken together with the carbon to which they are attached to form a cyclohexyl ring.

In some embodiments, X is —O—. In some embodiments, X is —S—. In some embodiments, X is —S(O)—. In some embodiments, X is —NR^{6a}—, wherein R^{6a} is hydrogen or C₁₋₄ alkyl. For instance, in some embodiments, X is —NH—. In some embodiments, X is —NR^{6a}—, wherein R^{6a} is C₁₋₄ alkyl. For instance, in some embodiments, X is —N(CH₃)—. In some embodiments, X is —S(O)₂—. In other embodiments, X is or —C(O)—. In some embodiments, X is —NR^{6a}S(O)₂—, wherein R^{6a} is hydrogen or C₁₋₄ alkyl. For instance, in some embodiments, X is —NHS(O)₂—. In some embodiments, X is —CR^{4a}R^{5a}S(O)₂—, wherein R^{4a} and R^{5a} are each independently hydrogen, hydroxy, halogen. For instance, in some embodiments, X is —CF₂S(O)₂—. In other embodiments, X is —CH₂S(O)₂—. In some embodiments, X is so —NR^{6a}C(O)—, wherein R^{6a} is hydrogen or C₁₋₄ alkyl. For instance, in some embodiments, X is —NHC(O)—. In other embodiments, X is —NHNHC(O)—.

In some embodiments, the compound of Formula (IIA) or Formula (II) is a compound of Formula (IIa) or (IIb):

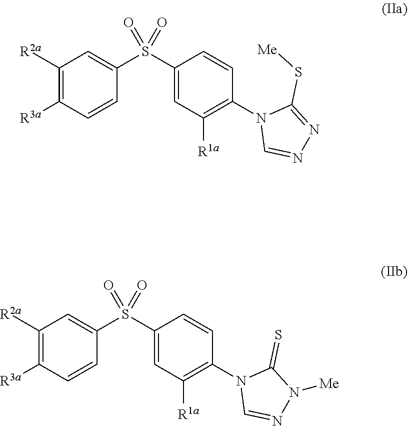

or a pharmaceutically acceptable salt thereof, wherein R^{1a}, R^{2a}, and R^{3a} are as defined for Formula (IIA) or Formula (II).

In some embodiments, the compound of Formula (IIA) or Formula (II) is a compound of Formula (IIc), (IId), (IIe), (IIf), or (IIg):

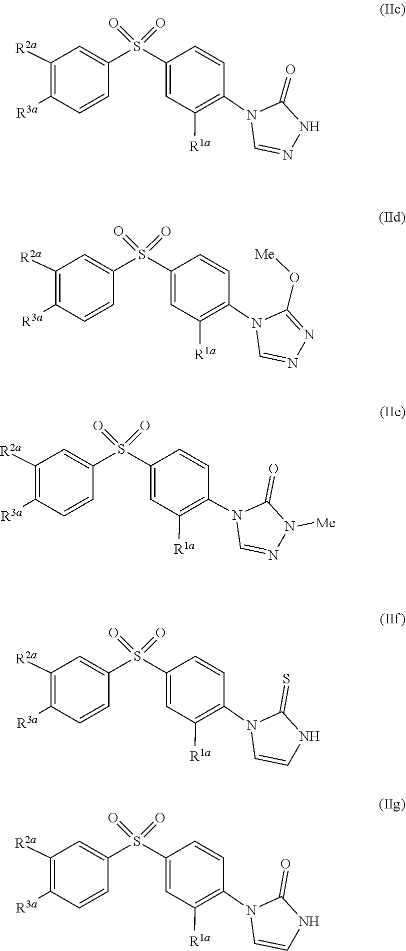

or a pharmaceutically acceptable salt thereof, wherein R^{1a}, R^{2a}, and R^{3a} are as defined for Formula (IIA) or Formula (II).

In some embodiments, the compound of Formula (IIA) or Formula (II) is a compound of Formula (IIh), (IIi), (IIj), (Ilk), (IIl), (IIm), (IIn), (IIo), or (IIp):

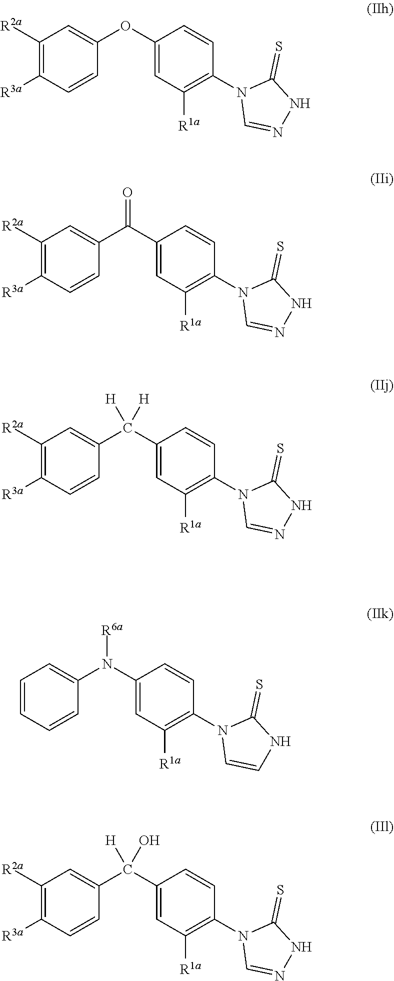

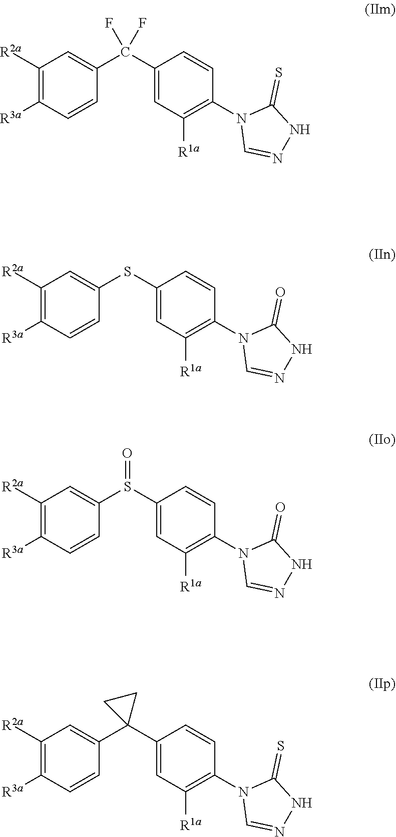

or a pharmaceutically acceptable salt thereof, wherein R^{1a}, R^{2a}, R^{3a}, and R^{6a} are as defined for Formula (IIA) or Formula (II).

In some embodiments, the compound of Formula (IIA) or Formula (II) is a compound of Formula (IIq), (IIr), (IIs), (IIt), (IIu), (IIv), or (IIw):

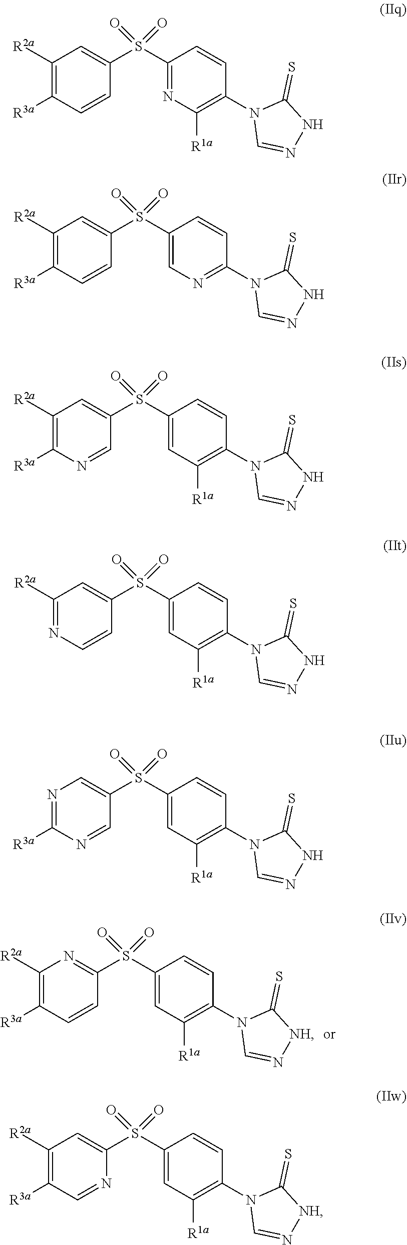

or a pharmaceutically acceptable salt thereof, wherein R^{1a}, R^{2a}, and R^{3a} are as defined for Formula (IIA) or Formula (II).

In some embodiments of Formula (IIA) or Formula (II), or Formulae (II-1), (II-2), (IIa), (IIb), (IIc), (IId), (IIe), (IIf), (IIg), (IIh), (IIi), (IIj), (IIk), (IIl), (IIm), (IIn), (IIo), (IIp), (IIq), (IIr), (IIs), (lit), (IIv), or (IIw), R^{2a} is selected from the group consisting of hydrogen, hydroxy, halogen, C₁₋₄ alkyl, substituted C₁₋₄ alkyl, C₁₋₄ alkoxy, substituted C₁₋₄ alkoxy, —CN, —NR^{x}R^{y}, and optionally substituted heterocyclyl. In some embodiments, R^{2a} is selected from the group consisting of hydrogen, C₁₋₄ alkyl, C₁₋₄haloalkyl, C₁₋₄ alkoxy, and substituted C₁₋₄ alkoxy. In some embodiments, R^{2a} is hydrogen. In some embodiments, R^{2a} is methyl, ethyl, n-propyl, isopropyl, n-butyl, secbutyl, or tertbutyl. In some embodiments, R^{2a} is methyl, ethyl, n-propyl, isopropyl, n-butyl, secbutyl, or tertbutyl. In some embodiments, R^{2a} is methyl. In some embodiments, R^{2a} is CF₃. In some embodiments, R^{2a} is methoxy, ethoxy, propoxy, isopropoxy, butoxy, or tertbutoxy. In some embodiments, R^{2a} is a 5- to 12-membered heterocyclyl. In some embodiments, R^{2a} is a 5- to 6-membered heterocyclyl.

In some embodiments of Formula (IIA) or Formula (II), or Formulae (II-1), (II-2), (IIa), (IIb), (IIc), (IId), (IIe), (IIf), (IIg), (IIh), (IIi), (IIj), (Ilk), (IIl), (IIm), (IIn), (IIo), (IIp), (IIq), (IIr), (IIs), (IIu), (IIv), or (IIw), R^{3a} is selected from the group consisting of hydrogen, hydroxy, halogen, C₁₋₄ alkyl, substituted C₁₋₄ alkyl, C₁₋₄ alkoxy, substituted C₁₋₄ alkoxy, —CN, —NR^{x}R^{y}, and optionally substituted heterocyclyl. In some embodiments, R^{3a} is selected from the group consisting of hydrogen, C₁₋₄ alkyl, C₁₋₄haloalkyl, C₁₋₄ alkoxy, and substituted C₁₋₄ alkoxy. In some embodiments, R^{3a} is hydrogen. In some embodiments, R^{3a} is methyl, ethyl, n-propyl, isopropyl, n-butyl, secbutyl, or tertbutyl. In some embodiments, R^{3a} is methyl, ethyl, n-propyl, isopropyl, n-butyl, secbutyl, or tertbutyl. In some embodiments, R^{3a} is methyl. In some embodiments, R^{3a} is CF₃. In some embodiments, R^{3a} is methoxy, ethoxy, propoxy, isopropoxy, butoxy, or tertbutoxy. In some embodiments, R^{3a} is a 5- to 12-membered heterocyclyl. In some embodiments, R^{3a} is a 5- to 6-membered heterocyclyl.

In some embodiments of Formula (IIA) or Formula (II), or Formulae (II-1), (II-2), (IIa), (IIb), (IIc), (IId), (IIe), (IIf), (IIg), (IIh), (IIi), (IIj), (IIk), (IIl), (IIm), (IIn), (IIo), (IIp), (IIq), (IIr), (IIs), (IIv), or (IIw), R^{2a} and R^{3a} are taken together with the carbons to which they are attached to form a 5- to 16-membered heterocyclyl ring. In some embodiments, R^{2a} and R^{3a} are taken together with the carbons to which they are attached to form a 5- to 16-membered heterocyclyl ring comprising one or more heteroatoms selected from N, O, and S. In some embodiments, R^{2a} and R^{3a} are taken together with the carbons to which they are attached to form a 9- to 12-membered heterocyclyl ring comprising one or more O atoms.

In some embodiments of Formula (IIA) or Formula (II), or Formulae (II-1), (II-2), (IIa), (IIb), (IIc), (IId), (IIe), (IIf), (IIg), (IIh), (IIi), (IIj), (IIk), (IIl), (IIm), (IIn), (IIo), (IIp), (IIq), (IIr), (IIs), (IIv), or (IIw), R^{2a} is C₁₋₄ alkyl or substituted C₁₋₄ alkyl and R^{3a} is halogen or C₁₋₄ alkyl. In some embodiments, R^{2a} is C₁₋₄ alkyl substituted with one or more halogen, and R^{3a} is halogen. In certain embodiments, R^{2a} is —CF₃ and R^{3a} is Cl. In certain embodiments, R^{2a} is —CF₃ and R^{3a} is methyl. In some embodiments, R^{3a} is C₁₋₄ alkyl or substituted C₁₋₄ alkyl and R^{2a} is halogen or C₁₋₄ alkyl. In some embodiments, R^{3a} is C₁₋₄ alkyl substituted with one or more halogen, and R^{2a} is halogen. In some embodiments, R^{3a} is —CF₃ and R^{2a} is Cl. In some embodiments, R^{3a} is —CF₃ and R^{2a} is methyl. In some embodiments, R^{2a} and R^{3a} are each H. In some embodiments, R^{2a} is H and R^{3a} is halogen, —CN, C₁₋₄ alkyl, substituted C₁₋₄ alkyl, C₁₋₄ alkoxy, substituted C₁₋₄ alkoxy, —NR^{x}R^{y}, wherein R^{x} and R^{y} are each independently H or optionally substituted C₁₋₄alkyl, or an optionally substituted heterocyclyl. In some embodiments, R^{2a} is H and R^{3a} is halogen. For instance, in some embodiments, R^{2a} is H and R^{3a} is Cl. In other some embodiments, R^{2a} is H and R^{3a} is F. In some embodiments, R^{2a} is H and R^{3a} is —CN. In some embodiments, R^{2a} is H and R^{3a} is optionally substituted C₁₋₄ alkyl. For instance, in some embodiments, R^{2a} is H and R^{3a} is methyl. In some embodiments, R^{2a} is H and R^{3a} is —CF₃. In some embodiments, R^{2a} is H and R^{3a} is optionally substituted C₁₋₄ alkoxy. For instance, in some embodiments, R^{2a} is H and R^{3a} is methoxy. In some embodiments, R^{2a} is H and R^{3a} is —N(CH₃)₂. In some embodiments, R^{2a} is H and R^{3a} is an optionally substituted heterocyclyl. In certain embodiments, R^{2a} is H and R^{3a} is morpholinyl. In some embodiments, R^{2a} is C₁₋₄ alkyl and R^{3a} is halogen. For instance, in some embodiments, R^{2a} is methyl and R^{3a} is Cl. In other embodiments, R^{2a} is H and R^{3a} is —CN. In some embodiments, R^{3a} is H and R^{2a} is halogen, —CN, C₁₋₄ alkyl, substituted C₁₋₄ alkyl, C₁₋₄ alkoxy, substituted C₁₋₄ alkoxy, —NR^{x}R^{y}, wherein R^{x} and R^{y} are each independently H or optionally substituted C₁₋₄alkyl, or an optionally substituted heterocyclyl. In some embodiments, R^{3a} is H and R^{2a} is halogen. For instance, in some embodiments, R^{3a} is H and R^{2a} is Cl. In other some embodiments, R^{3a} is H and R^{2a} is F. In some embodiments, R^{3a} is H and R^{2a} is —CN. In some embodiments, R^{3a} is H and R^{2a} is optionally substituted C₁₋₄ alkyl. For instance, in some embodiments, R^{3a} is H and R^{2a} is methyl. In some embodiments, R^{3a} is H and R^{2a} is —CF₃. In some embodiments, R^{3a} is H and R^{2a} is optionally substituted C₁₋₄ alkoxy. For instance, in some embodiments, R^{3a} is H and R^{2a} is methoxy. In some embodiments, R^{3a} is H and R^{2a} is —N(CH₃)₂. In some embodiments, R^{3a} is H and R^{2a} is an optionally substituted heterocyclyl. In certain embodiments, R^{3a} is H and R^{2a} is morpholinyl. In some embodiments, R^{3a} is C₁₋₄ alkyl and R^{2a} is halogen. For instance, in some embodiments, R^{3a} is methyl and R^{2a} is Cl. In other embodiments, R^{3a} is H and R^{2a} is —CN.

In some embodiments of Formula (IIA) or Formula (II), or Formulae (II-1), (II-2), (IIa), (IIb), (IIc), (IId), (IIe), (IIf), (IIg), (IIh), (IIi), (IIj), (Ilk), (IIl), (IIm), (IIn), (IIo), (IIp), (IIq), (IIs), (IIt), (IIu), (IIv), or (IIw), R^{1a} is selected from the group consisting of hydrogen, hydroxy, halogen, C₁₋₄ alkyl, substituted C₁₋₄ alkyl, C₁₋₄ alkoxy, substituted C₁₋₄ alkoxy, —CN, —NR^{x}R^{y}, and optionally substituted heterocyclyl. In some embodiments, R^{1a} is selected from the group consisting of hydrogen, C₁₋₄ alkyl, C₁₋₄haloalkyl, C₁₋₄ alkoxy, and substituted C₁₋₄ alkoxy. In some embodiments, R^{1a} is hydrogen. In some embodiments, Ria is methyl, ethyl, n-propyl, isopropyl, n-butyl, secbutyl, or tertbutyl. In some embodiments, R^{1a} is methyl, ethyl, n-propyl, isopropyl, n-butyl, secbutyl, or tertbutyl. In some embodiments, R^{1a} is methyl. In some embodiments, R^{1a} is CF₃. In some embodiments, Ria is methoxy, ethoxy, propoxy, isopropoxy, butoxy, or tertbutoxy. In some embodiments, R^{1a} is a 5- to 12-membered heterocyclyl. In some embodiments, R^{1a} is a 5- to 6-membered heterocyclyl.

It is understood that the descriptions of any variable of Formula (IIA) or Formula (II) may, where applicable, be combined with one or more descriptions of any other variable, the same as if each and every combination of variables were specifically and individually listed. For example, every description of A may be combined with every description of G¹, G², G³, G⁴, G⁵, G⁶, G⁷, G⁸, G⁸, G⁹, R^{1a}, R^{2a}, R^{3a}, R^{4a}, R^{5a}, R^{6a}, R^{7a}, X, Z, and W the same as if each and every combination were specifically and individually listed. Likewise, every description of X may be combined with every description of A, G¹, G², G³, G⁴, G⁵, G⁶, G⁷, G⁸, G⁸, G⁹, R^{1a}, R^{2a}, R^{3a}, R^{4a}, R^{5a}, R^{6a}, R^{7a}, Z, and W the same as if each and every description were specifically and individually listed, and every description of G¹ may be combined with every description of A, G², G³, G⁴, G⁵, G⁶, G⁷, G⁸, G⁸, G⁹, R^{1a}, R^{2a}, R^{3a}, R^{4a}, R^{5a}, R^{6a}, R^{7a}, X, Z, and W the same as if each and every description were specifically and individually listed.

In some embodiments, the compound of Formula (IIA) or Formula (II) is a compound shown in the following table.

| Compound | | |
|---|---|---|
| No. | Structure | Name |
| 26 | 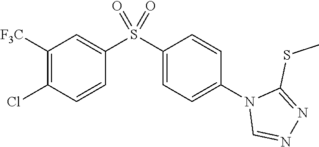 [SMILES: CSc1nncn1-c1ccc(S(=O)(=O)c2ccc(Cl)c(C(F)(F)F)c2)cc1] | 4-(4-((4-chloro-3- (trifluoromethyl)phenyl)sulfonyl)phenyl)- 3-(methylthio)-4H-1,2,4-triazole |
| 27 | 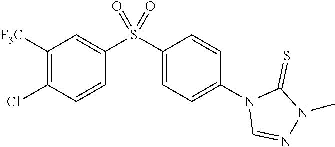 [SMILES: Cn1ncn(-c2ccc(S(=O)(=O)c3ccc(Cl)c(C(F)(F)F)c3)cc2)c1=S] | 4-(4-((4-chloro-3- (trifluoromethyl)phenyl)sulfonyl)phenyl)- 2-methyl-2,4-dihydro-3H-1,2,4-triazole- 3-thione |
| 28 | 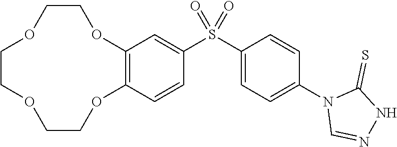 [SMILES: O=S(=O)(c1ccc(-n2cn[nH]c2=S)cc1)c1ccc2c(c1)OCCOCCOCCO2] | 4-(4-((2,3,5,6,8,9- hexahydrobenzo[b][1,4,7,10]tetraoxacyclo- dodecin-12-yl)sulfonyl)phenyl)-2,4- dihydro-3H-1,2,4-triazole-3-thione |
| 29 | 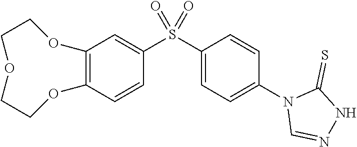 [SMILES: O=S(=O)(c1ccc(-n2cn[nH]c2=S)cc1)c1ccc2c(c1)OCCOCCO2] | 4-(4-((2,3,5,6- tetrahydrobenzo[b][1,4,7]trioxonin-9- yl)sulfonyl)phenyl)-2,4-dihydro-3H- 1,2,4-triazole-3-thione |
| 30 | 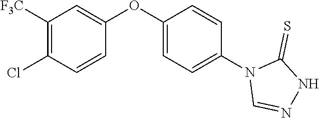 [SMILES: FC(F)(F)c1cc(Oc2ccc(-n3cn[nH]c3=S)cc2)ccc1Cl] | 4-(4-(4-chloro-3-(trifluoro-methyl)- phenoxy)-phenyl)-3,4-dihydro-2H-1,2,4- triazole-3-thione |
| 31 | 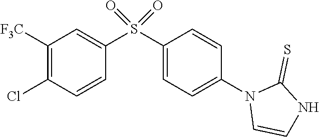 [SMILES: O=S(=O)(c1ccc(-n2cc[nH]c2=S)cc1)c1ccc(Cl)c(C(F)(F)F)c1] | 1-(4-(4-chloro-3-(trifluoro-methyl)- phenylsulfonyl)-phenyl)-2,3-dihydro- 1H-imidazole-2-thione |
| 32 | 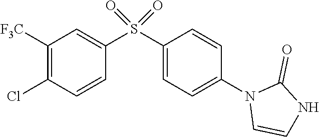 [SMILES: O=c1[nH]ccn1-c1ccc(S(=O)(=O)c2ccc(Cl)c(C(F)(F)F)c2)cc1] | 1-(4-(4-chloro-3-(trifluoro-methyl)- phenylsulfonyl)-phenyl)-2,3-dihydro- 1H-imidazol-2-one |
| 33 | 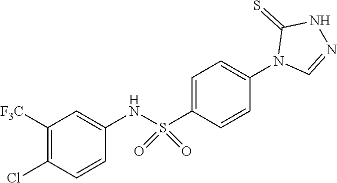 [SMILES: O=S(=O)(Nc1ccc(Cl)c(C(F)(F)F)c1)c1ccc(-n2cn[nH]c2=S)cc1] | N-(4-chloro-3-(trifluoromethyl)phenyl)- 4-(5-thioxo-1,5-dihydro-4H-1,2,4- triazol-4-yl)benzenesulfonamide |
| 34 | 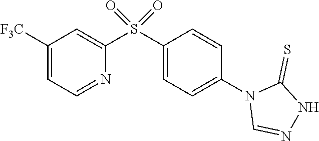 [SMILES: O=S(=O)(c1ccc(-n2cn[nH]c2=S)cc1)c1cc(C(F)(F)F)ccn1] | 4-(4-((4-(trifluoromethyl)pyridin-2- yl)sulfonyl)phenyl)-2,4-dihydro-3H- 1,2,4-triazole-3-thione |
| 35 | 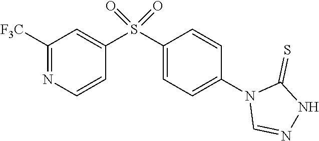 [SMILES: O=S(=O)(c1ccc(-n2cn[nH]c2=S)cc1)c1ccnc(C(F)(F)F)c1] | 4-(4-((2-(trifluoromethyl)pyridin-4- yl)sulfonyl)phenyl)-2,4-dihydro-3H- 1,2,4-triazole-3-thione |
| 36 | 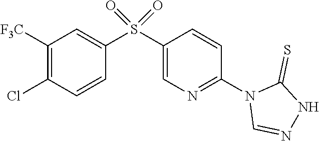 [SMILES: O=S(=O)(c1ccc(-n2cn[nH]c2=S)nc1)c1ccc(Cl)c(C(F)(F)F)c1] | 4-(5-((4-chloro-3- (trifluoromethyl)phenyl)sulfonyl)pyridin- 2-yl)-2,4-dihydro-3H-1,2,4-triazole-3- thione |
| 37 | 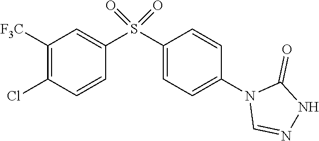 [SMILES: O=c1[nH]ncn1-c1ccc(S(=O)(=O)c2ccc(Cl)c(C(F)(F)F)c2)cc1] | 4-(4-((4-chloro-3- (trifluoromethyl)phenyl)sulfonyl)phenyl)- 2,4-dihydro-3H-1,2,4-triazol-3-one |
| 38 | 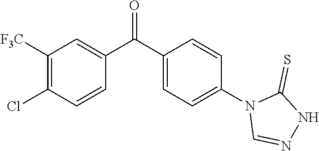 [SMILES: O=C(c1ccc(-n2cn[nH]c2=S)cc1)c1ccc(Cl)c(C(F)(F)F)c1] | (4-chloro-3-(trifluoromethyl)phenyl)(4- (5-thioxo-1,5-dihydro-4H-1,2,4-triazol- 4-yl)phenyl)methanone |
| 39 | 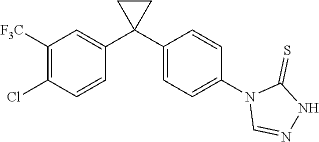 [SMILES: FC(F)(F)c1cc(C2(c3ccc(-n4cn[nH]c4=S)cc3)CC2)ccc1Cl] | 4-(4-(1-(4-chloro-3- (trifluoromethyl)phenyl)cyclopropyl) phenyl)-2,4-dihydro-3H-1,2,4-triazole-3- thione |
| 40 | 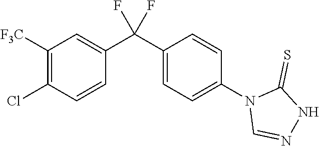 [SMILES: FC(F)(F)c1cc(C(F)(F)c2ccc(-n3cn[nH]c3=S)cc2)ccc1Cl] | 4-(4-((4-chloro-3- (trifluoromethyl)phenyl)difluoromethyl) phenyl)-2,4-dihydro-3H-1,2,4-triazole-3- thione |
| 41 | 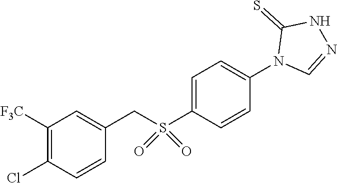 [SMILES: O=S(=O)(Cc1ccc(Cl)c(C(F)(F)F)c1)c1ccc(-n2cn[nH]c2=S)cc1] | 4-(4-((4-chloro-3- (trifluoromethyl)benzyl)sulfonyl)phenyl)- 2,4-dihydro-3H-1,2,4-triazole-3-thione |
| 42 | 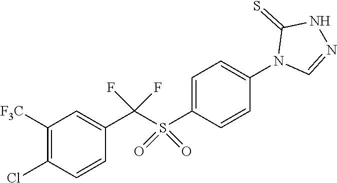 [SMILES: O=S(=O)(c1ccc(-n2cn[nH]c2=S)cc1)C(F)(F)c1ccc(Cl)c(C(F)(F)F)c1] | 4-(4-(((4-chloro-3- (trifluoromethyl)phenyl)difluoromethyl) sulfonyl)phenyl)-2,4-dihydro-3H-1,2,4- triazole-3-thione |
| 43 | 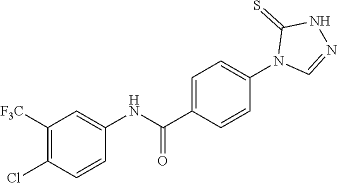 [SMILES: O=C(Nc1ccc(Cl)c(C(F)(F)F)c1)c1ccc(-n2cn[nH]c2=S)cc1] | N-(4-chloro-3-(trifluoromethyl)phenyl)- 4-(5-thioxo-1,5-dihydro-4H-1,2,4- triazol-4-yl)benzamide |
| 44 | 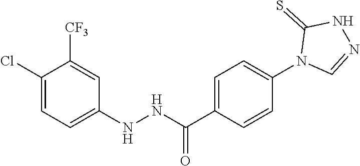 [SMILES: O=C(NNc1ccc(Cl)c(C(F)(F)F)c1)c1ccc(-n2cn[nH]c2=S)cc1] | N′-(4-chloro-3-(trifluoromethyl)phenyl)- 4-(5-thioxo-1,5-dihydro-4H-1,2,4- triazol-4-yl)benzohydrazide |

Additional compounds are shown in the following table.

| Ex. | Structure | Name |
|---|---|---|
| A1 | 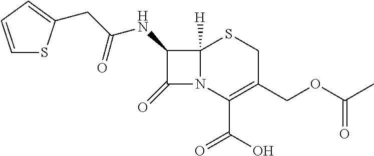 [SMILES: CC(=O)OCC1=C(C(=O)O)N2C(=O)[C@@H](NC(=O)Cc3cccs3)[C@H]2SC1] | Cephalothin |
| A2 | 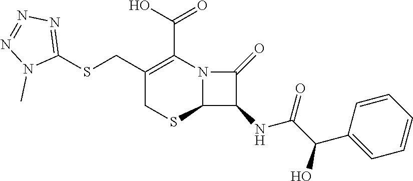 [SMILES: Cn1nnnc1SCC1=C(C(=O)O)N2C(=O)[C@@H](NC(=O)[C@H](O)c3ccccc3)[C@H]2SC1] | Cefamandole |
| A3 | 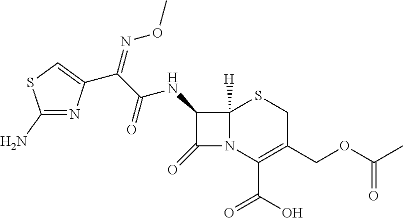 [SMILES: CO/N=C(\C(=O)N[C@@H]1C(=O)N2C(C(=O)O)=C(COC(C)=O)CS[C@H]12)c1csc(N)n1] | Cefotaxime |
| A4 | 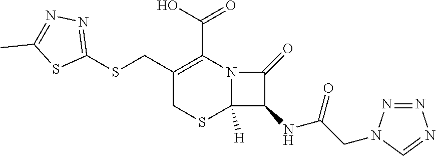 [SMILES: Cc1nnc(SCC2=C(C(=O)O)N3C(=O)[C@@H](NC(=O)Cn4cnnn4)[C@H]3SC2)s1] | Cefazolin |
| A5 | 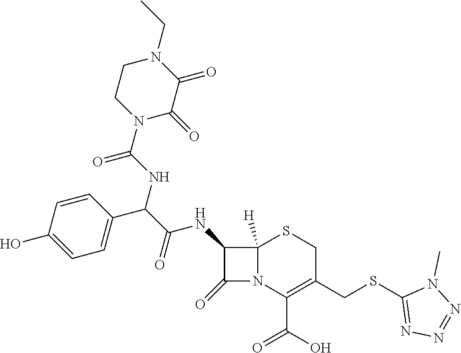 [SMILES: CCN1CCN(C(=O)NC(C(=O)N[C@@H]2C(=O)N3C(C(=O)O)=C(CSc4nnnn4C)CS[C@H]23)c2ccc(O)cc2)C(=O)C1=O] | Cefoperazone |
| A6 | 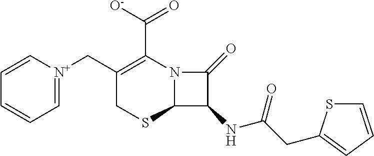 [SMILES: O=C(Cc1cccs1)N[C@@H]1C(=O)N2C(C(=O)[O-])=C(C[n+]3ccccc3)CS[C@H]12] | Cephaloridine |
| A7 | 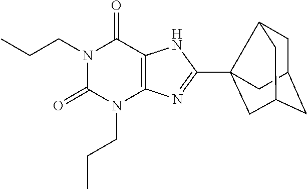 [SMILES: CCCn1c(=O)c2[nH]c(C34CC5CC(CC3C5)C4)nc2n(CCC)c1=O] | Rolofylline |
| A8 | 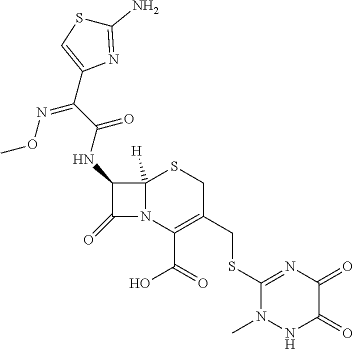 | Ceftriaxone |
| A9 | 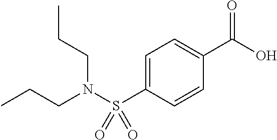 [SMILES: CCCN(CCC)S(=O)(=O)c1ccc(C(=O)O)cc1] | Probenecid |
| A10 | 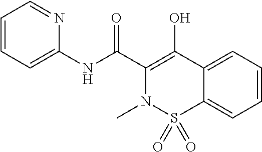 [SMILES: CN1C(C(=O)Nc2ccccn2)=C(O)c2ccccc2S1(=O)=O] | Piroxicam |
| A11 | 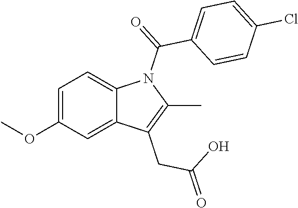 [SMILES: COc1ccc2c(c1)c(CC(=O)O)c(C)n2C(=O)c1ccc(Cl)cc1] | Indomethacin |
| A12 | 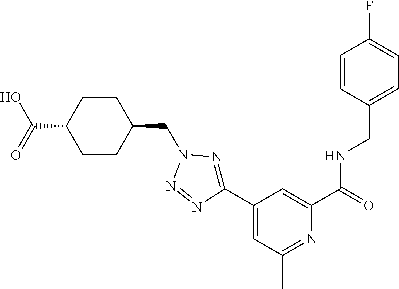 [SMILES: Cc1cc(-c2nnn(C[C@H]3CC[C@H](C(=O)O)CC3)n2)cc(C(=O)NCc2ccc(F)cc2)n1] | (1r,4r)-4-((5-(2-((4-fluorobenzyl)carbamoyl)- 6-methylpyridin-4-yl)-2H-tetrazol-2- yl)methyl)cyclohexane-1-carboxylic acid (CHEMBL603656) |
| A13 | 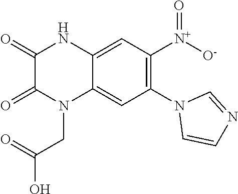 [SMILES: O=C(O)Cn1c(=O)c(=O)[nH]c2cc([N+](=O)[O-])c(-n3ccnc3)cc21] | Zonampanel |
| A14 | 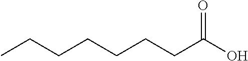 | Octanoic Acid |
| A15 | 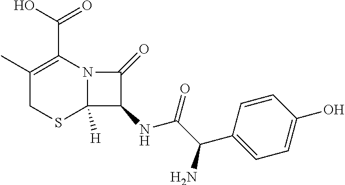 [SMILES: CC1=C(C(=O)O)N2C(=O)[C@@H](NC(=O)[C@H](N)c3ccc(O)cc3)[C@H]2SC1] | Cefadroxil |
| A16 | 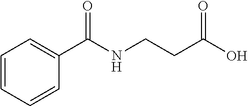 [SMILES: O=C(O)CCNC(=O)c1ccccc1] | Betamipron |
| A17 | 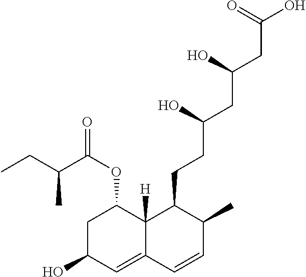 [SMILES: CC[C@H](C)C(=O)O[C@H]1C[C@H](O)C=C2C=C[C@H](C)[C@H](CC[C@@H](O)C[C@@H](O)CC(=O)O)[C@H]21] | Pravastatin |
| A18 | 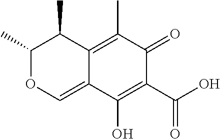 [SMILES: CC1=C2C(=CO[C@H](C)[C@H]2C)C(O)=C(C(=O)O)C1=O] | Citrinin |
| A19 | 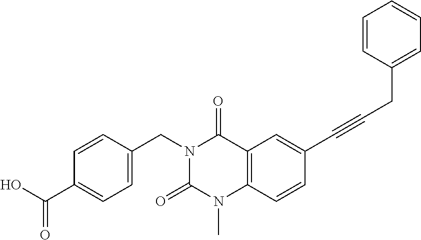 [SMILES: Cn1c(=O)n(Cc2ccc(C(=O)O)cc2)c(=O)c2cc(C#CCc3ccccc3)ccc21] | 4-((1-methyl-2,4-dioxo-6-(3-phenylprop-1-yn- 1-yl)-1,4-dihydroquinazolin-3(2H)- yl)methyl)benzoic acid |
| A20 | 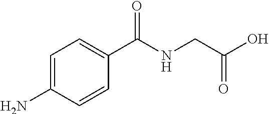 [SMILES: Nc1ccc(C(=O)NCC(=O)O)cc1] | Aminohippuric Acid |
| A21 | 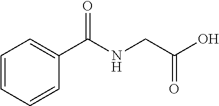 [SMILES: O=C(O)CNC(=O)c1ccccc1] | Hippuric Acid |

or a pharmaceutically acceptable salt thereof.

The compounds of Formula (I), Formula (IIA), and Formula (II) may be prepared and/or formulated as pharmaceutically acceptable salts. In some embodiments, pharmaceutically acceptable salts include acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like. These salts may be derived from inorganic or organic acids. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. In some embodiments, pharmaceutically acceptable salts are formed when an acidic proton present in the parent compound either is replaced by a metal ion, e.g., an alkali metal ion, an alkaline earth ion, or an aluminum ion; or coordinates with an organic base. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, trimetharnine, dicyclohexylamine, caffeine, procaine, hydrabamine, choline, betaine, ethylenediamine, glucosamine, N-ethylglucamine, N-methylglucamine, theobromine, purines, piperazine, piperidine, N-ethylpiperidine, polyamine resins, amino acids such as lysine, arginine, histidine, and the like. Examples of pharmaceutically acceptable base addition salts include those derived from inorganic bases such as sodium, potassium, lithium, ammonium, calcium, magnesium, iron, zinc, copper, manganese, aluminum salts and the like. In some embodiments, the organic non-toxic bases are L-amino acids, such as L-lysine and L-arginine, tromethamine, N-ethylglucamine and N-methylglucamine. Acceptable inorganic bases include aluminum hydroxide, calcium hydroxide, potassium hydroxide, sodium carbonate, sodium hydroxide, and the like. Lists of other suitable pharmaceutically acceptable salts are found in Remington's Pharmaceutical Sciences, 17th Edition, Mack Publishing Company, Easton, Pa., 1985.

For a compound described herein that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, benzenesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The embodiments also relate to pharmaceutically acceptable prodrugs of the compounds described herein, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I), Formula (IIA), or Formula (II). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The embodiments also relate to pharmaceutically active metabolites of compounds described herein, and uses of such metabolites in the methods provided herein. A “pharmaceutically active metabolite” means a pharmacologically active product of metabolism in the body of a compound described herein or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

In some embodiments, the ion transporter inhibitor comprises one or more of compounds 1-23, or a pharmaceutically acceptable salt thereof, as described herein. In some embodiments, the ion transporter inhibitor does not comprise compounds 1-23, or a pharmaceutically acceptable salt thereof.

### Pharmaceutical Compositions

For treatment purposes, a pharmaceutical composition according to the present disclosure comprises at least one compound of Formula (I), Formula (IIA), or Formula (II), or a pharmaceutically acceptable salt thereof. The pharmaceutical compositions may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In some embodiments, pharmaceutical compositions according to the embodiments are sterile compositions. Pharmaceutical compositions may be prepared using compounding techniques known or that become available to those skilled in the art.

Sterile compositions are also contemplated by the embodiments, including compositions that are in accord with national and local regulations governing such compositions.

The pharmaceutical compositions and compounds described herein may be formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions provided herein may be administered by a suitable route of delivery, such as oral, parenteral, rectal, nasal, topical, or ocular routes, or by inhalation. In some embodiments, the compositions are formulated for intravenous or oral administration.

For oral administration, the compounds the embodiments may be provided in a solid form, such as a tablet or capsule, or as a solution, emulsion, or suspension. To prepare the oral compositions, the compounds provided herein may be formulated to yield a dosage of, e.g., from about 0.01 to about 50 mg/kg daily, or from about 0.05 to about 20 mg/kg daily, or from about 0.1 to about 10 mg/kg daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinylpyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compositions described herein may be formulated for rectal administration as a suppository. For parenteral use, including intravenous, intramuscular, intraperitoneal, intranasal, or subcutaneous routes, the agents provided herein may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the compounds or pharmaceutical compositions described herein may be administered using, for example, a spray formulation also containing a suitable carrier.

In some embodiments, for topical applications, the compounds of the present embodiments are formulated as creams or ointments or a similar vehicle suitable for topical administration. For topical administration, the compounds or pharmaceutical compositions described herein may be mixed with a pharmaceutical carrier at a concentration of about 0.1% to about 10% of drug to vehicle. Another mode of administering the agents provided herein may utilize a patch formulation to effect transdermal delivery.

As used herein, “treat”, “treatment”, or “treating” is an approach for obtaining beneficial or desired results including clinical results. For purposes of the compositions and methods provided herein, beneficial or desired clinical results include, but are not limited to, one or more of the following: decreasing one or more symptoms resulting from the condition, diminishing the extent of the condition, stabilizing the condition (e.g., preventing or delaying the worsening of the condition), ameliorating a disease state, providing a remission (whether partial or total) of a disease, decreasing the dose of one or more other medications required to treat the condition, enhancing the effect of another medication used to treat the condition, increasing the quality of life of an individual having the condition, and/or prolonging survival. A method of treating a disease or condition encompasses a reduction of the pathological consequence of the disease or condition. The methods described herein contemplate any one or more of these aspects of treatment.

As used herein, the term “prevent,” “preventing” or “prevention” of a condition, disease, or disorder refers in one embodiment, to delay or avoidance of onset of the disease or disorder (i.e., slowing or preventing the onset of the disease or disorder in a patient susceptible to development of the disease or disorder). In some embodiments, “prevent,” “preventing” or “prevention” refers in to delaying or slowing the progression of the condition, disease, or disorder.

The term “subject” refers to a mammalian patient in need of such treatment, such as a human.

Exemplary diseases that may be therapeutic targets for such compounds include, but are not limited to, central neurodegenerative disorders such as Alzheimer's Disease, Parkinson's Disease, Huntington Disease and other central neurodegenerative disorders and peripheral degenerative disorders where there is evidence of accumulated neurotoxic proteins.

In one aspect, the compounds and pharmaceutical compositions of the present disclosure specifically target the accumulation of neurotoxic proteins or their aggregated species. Thus, these compounds and pharmaceutical compositions can treat degenerative neurological diseases related to or caused by mis-regulation of protein homeostasis (proteostasis) e.g., such as inadequate clearance of protein aggregates and/or damaged organelles, insufficient activation of a survival pattern of gene expression, and/or deficiencies in cell energetics. In some embodiments, the methods of the present disclosure target neurodegenerative diseases associated with the accumulation of neurotoxic misfolded and aggregated proteins. In some embodiments, methods of treatment target Parkinson's disease, Alzheimer's disease, Lewy body disease, multiple system atrophy, or Huntington's disease. The compounds, compositions, and methods of the present disclosure are also used to mitigate deleterious effects of impaired protein homeostasis including impairments of various forms of macro autophagy and other protein clearance mechanisms. While the present disclosure is not limited by any particular mechanism of action, dysregulation of autophagy is thought to be caused by alpha synuclein beta amyloid and other proteins that accumulate and aggregate in neurodegenerative disorders. Many pathologies in Parkinson's disease including oxidative stress, mitochondrial dysfunction, and protein aggregation (such as alpha-synuclein aggregation) are linked to autophagy, which is also dysregulated in Parkinson's disease.

In treatment methods according to the embodiments, an “effective amount” means an amount or dose sufficient to generally bring about the desired therapeutic benefit in subjects needing such treatment. Effective amounts or doses of the compounds provided herein may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about 1 μg to 2 mg of active agent per kilogram of subject's body weight per day, such as about 0.05 to 100 mg/kg/day, or about 1 to 35 mg/kg/day, or about 0.1 to 10 mg/kg/day. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

Drug Combinations

The compounds described herein may be used in pharmaceutical compositions or methods in combination with one or more additional active ingredients in the treatment of neurodegenerative disorders. For example, additional active ingredients are those that are known or discovered to be effective in treating neurodegenerative disorders, including those active against another target associated with the disease, such as but not limited to, a) compounds that address protein misfolding (such as drugs which reduce the production of these proteins, which increase their clearance or which alter their aggregation and/or propagation); b) compounds that treat symptoms of such disorders (e.g., dopamine replacement therapies, cholinesterase inhibitors and precognitive glutamatergic drugs); and c) drugs that act as neuroprotectants by complementary mechanisms (e.g., those targeting autophagy, those that are anti-oxidants, and those acting by other mechanisms such as adenosine A2A antagonists).

For example, additional active ingredients are those that are known or discovered to be effective in treating neurodegenerative disorders, including those active against another target associated with the disease, such as but not limited to, a) compounds that target different mechanisms of protein misfolding (such as aggregation and/or propagation); b) compounds that treat symptoms of such disorders (e.g., dopamine replacement therapies); and c) drugs that act as neuroprotectants by complementary mechanisms (e.g., those targeting autophagy, anti-oxidants, and adenosine A2A antagonists).

For example, compositions and formulations provided herein, as well as methods of treatment, can further comprise other drugs or pharmaceuticals, e.g., other active agents useful for treating or palliative for a degenerative neurological disease related to or caused by protein aggregation, e.g., synuclein, beta-amyloid, tau, Huntingtin, or TDP43 protein aggregation, e.g., Parkinson's disease, Alzheimer's Disease (AD), Lewy body disease (LBD) and multiple system atrophy (MSA), or related symptoms or conditions. In this regard, compositions and formulations of the generic and specific compounds described herein are useful in methods of treatment for Alzheimer's Disease, Parkinson's Disease, fronto-temporal dementia, dementia with Lewy Bodies, PD dementia, multiple system atrophy, Huntington's disease, Amyotrophic lateral sclerosis, cancer, infection, Crohn's disease, heart disease, aging, or traumatic brain injury (TBI). The pharmaceutical compositions provided herein may additionally comprise one or more of such active agents, and methods of treatment may additionally comprise administering an effective amount of one or more of such active agents. In some embodiments, the one or more additional active agents is a compound that is used to treat the symptoms or progression of a neurodegenerative disorder (e.g., Alzheimer's Disease, Parkinson's Disease, Huntington's disease). In certain embodiments, additional active agents may be cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof. In some embodiments, the additional active agent is an anti-inflammatory agent. Additional active agents include those useful in such compositions and methods include dopamine therapy drugs, catechol-O-methyl transferase (COMT) inhibitors, monoamine oxidase inhibitors, cognition enhancers (such as acetylcholinesterase inhibitors or memantine), adenosine 2A receptor antagonists, beta-secretase inhibitors, or gamma-secretase inhibitors. In particular embodiments, at least one compound of the present embodiments may be combined in a pharmaceutical composition or a method of treatment with one or more drugs selected from the group consisting of: tacrine (Cognex), donepezil (Aricept), rivastigmine (Exelon) galantamine (Reminyl), physostigmine, neostigmine, Icopezil (CP-118954, 5,7-dihydro-3-[2-[1-(phenylmethyl)-4-piperidinyl]ethyl]-6H-pyrrolo-[4,5-f-]-1,2-benzisoxazol-6-one maleate), ER-127528 (4-[(5,6-dimethoxy-2-fluoro-1-indanon)-2-yl]methyl-1-(3-fluorobenzyl)piperidine hydrochloride), zanapezil (TAK-147; 3-[1-(phenylmethyl)piperidin-4-yl]-1-(2,3,4,5-tetrahydro-1H-1-benzazepin-8-yl)-1-propane fumarate), Metrifonate (T-588; (−)-R-alpha-[[2-(dimethylamino)ethoxy]methyl] benzo[b]thiophene-5-methanol hydrochloride), FK-960 (N-(4-acetyl-1-piperazinyl)-p-fluorobenzamide-hydrate), TCH-346 (N-methyl-N-2-pyropinyldibenz[b,f]oxepine-10-methanamine), SDZ-220-581 ((S)-alpha-amino-5-(phosphonomethyl)-[1,1′-biphenyl]-3-propionic acid), memantine (Namenda/Exiba) and 1,3,3,5,5-pentamethylcyclohexan-1-amine (Neramexane), tarenflurbil (Flurizan), tramiprosate (Alzhemed), clioquinol, PBT-2 (an 8-hydroxyquinilone derivative), 1-(2-(2-Naphthyl)ethyl)-4-(3-trifluoromethylphenyl)-1, 2,3,6-tetrahydropyr-idine, Huperzine A, posatirelin, leuprolide or derivatives thereof, ispronicline, (3-aminopropyl)(n-butyl)phosphinic acid (SGS-742), N-methyl-5-(3-(5-isopropoxypyridinyl))-4-penten-2-amine (ispronicline), 1-decanaminium, N-(2-hydroxy-3-sulfopropyl)-N-methyl-N-octyl-, inner salt (zt-1), salicylates, aspirin, amoxiprin, benorilate, choline magnesium salicylate, diflunisal, faislamine, methyl salicylate, magnesium salicylate, salicyl salicylate, diclofenac, aceclofenac, acemetacin, bromfenac, etodolac, indometacin, nabumetone, sulindac, tolmetin, ibuprofen, carprofen, fenbufen, fenoprofen, flurbiprofen, ketoprofen, ketorolac, loxoprofen, naproxen, tiaprofenic acid, suprofen, mefenamic acid, meclofenamic acid, phenylbutazone, azapropazone, metamizole, oxyphenbutazone, sulfinprazone, piroxicam, lornoxicam, meloxicam, tenoxicam, celecoxib, etoricoxib, lumiracoxib, parecoxib, rofecoxib, valdecoxib, nimesulide, arylalkanoic acids, 2-arylpropionic acids (profens), N-arylanthranilic acids (fenamic acids), pyrazolidine derivatives, oxicams, COX-2 inhibitors, sulphonanilides, essential fatty acids, and Minozac (2-(4-(4-methyl-6-phenylpyridazin-3-yl)piperazin-1-yl)pyrimidine dihydrochloride hydrate). Such a combination may serve to increase efficacy, ameliorate other disease symptoms, decrease one or more side effects, or decrease the required dose of the compounds or compositions described herein. The additional active ingredients may be administered in a separate pharmaceutical composition from a compound provided herein or may be included with a compound provided herein in a single pharmaceutical composition. The additional active ingredients may be administered simultaneously with, prior to, or after administration of a compound of Formula (I), Formula (IIA), or Formula (II).

### Kits and Articles of Manufacture

Provided herein are articles of manufacture or kits comprising an ion transporter inhibitor (e.g., an OAT inhibitor). In some embodiments, the kits further include instructions for use, e.g. for administering an effective amount of the ion transporter inhibitor for treatment of disease or condition associated with neurodegeneration to an subject in need thereof according to a method as described herein. In some embodiments, the disease or condition associated with neurodegeneration is Alzheimer's Disease, Parkinson's Disease, fronto-temporal dementia, dementia with Lewy Bodies, PD dementia, multiple system atrophy, Huntington's disease, Amyotrophic lateral sclerosis, progressive supranuclear palsy, or neuroinflammation.

In one aspect, provided herein are kits containing a compound or composition described herein (e.g., compounds of Formula (I), (IIA), or (II), or a pharmaceutically acceptable salt thereof) and instructions for use. The kits may contain instructions for use in the treatment of a condition in an individual in need thereof. In some embodiments, the condition is a neurodegenerative disease or condition.

A person skilled in the art knows that various chemical and polymorphic forms of a compound exist and any form is contemplated for the ion transporter inhibitor in the kits and articles of manufacture provided herein. In some embodiments, the ion transporter inhibitor can be in its free form or in the form of a stereoisomer, pharmaceutically acceptable salt, solvate, hydrate, co-crystal, polymorph, or prodrug. In some embodiments, the ion transporter inhibitor can be in its free form or in the form of a stereoisomer, pharmaceutically acceptable salt, solvate, hydrate, co-crystal, or polymorph. In some embodiments, the hydrate is a monohydrate form, dihydrate form, or trihydrate form.

A kit may additionally contain any materials or equipment that may be used in the administration of the compound or composition, such as vials, syringes, or IV bags. A kit may also contain sterile packaging.

### Chemical Synthesis

The embodiments are also directed to processes and intermediates useful for preparing subject compounds or a salt or solvate thereof.

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001.)

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd ed., ed. L. R. Snyder and J. J. Kirkland, John Wiley and Sons, 1979; and Thin Layer Chromatography, E. Stahl (ed.), Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as T. W. Greene and P. G. M. Wuts, “Protective Groups in Organic Synthesis,” 4^{th} ed., Wiley, New York 2006. The protecting groups may be removed at a convenient subsequent stage using methods known from the art.

Exemplary chemical entities useful in methods provided herein will now be described by reference to illustrative synthetic schemes for their general preparation herein and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups. Each of the reactions depicted in the general schemes is run at a temperature from about 0° C. to the reflux temperature of the organic solvent used. Unless otherwise specified, the variables are as defined above in reference to Formula (I), Formula (IIA), or Formula (II). Isotopically labeled compounds as described herein are prepared according to the methods described below, using suitably labeled starting materials. Such materials are generally available from commercial suppliers of radiolabeled chemical reagents.

Representative syntheses for compounds of Formula (I) are described in Schemes 1 and 2.

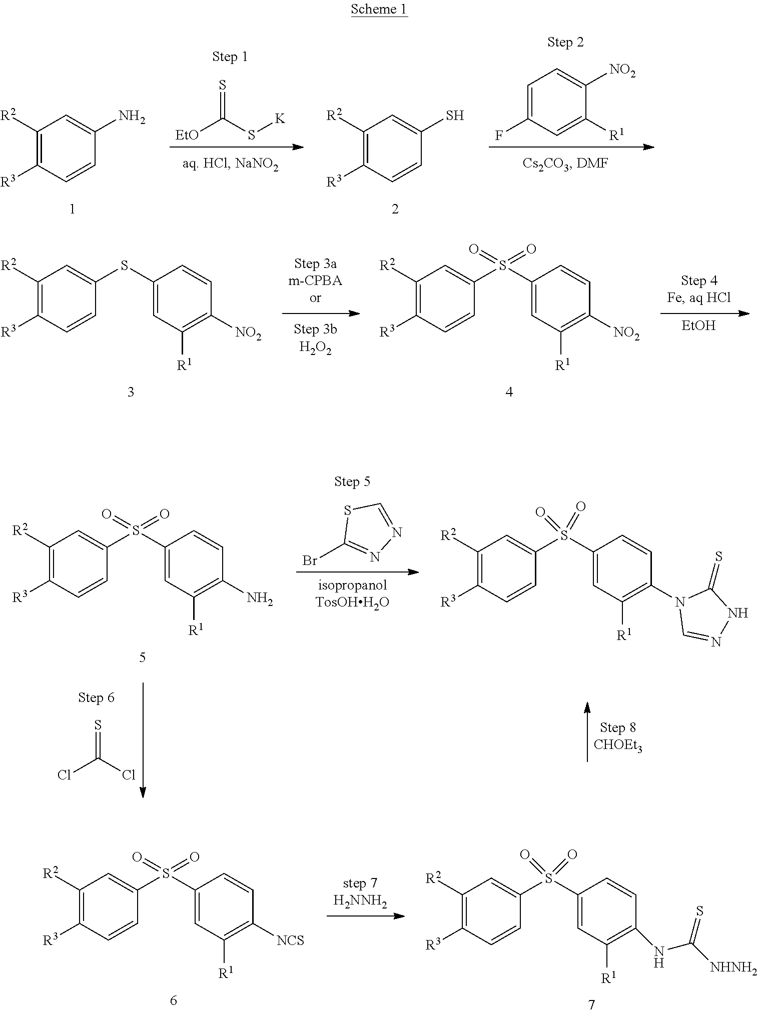

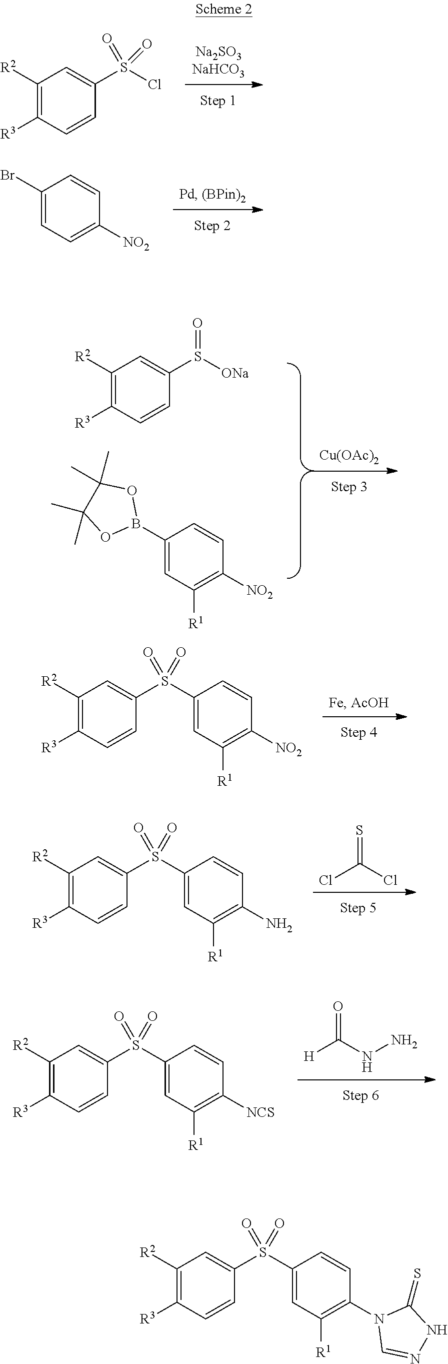

In Schemes 1 and 2, R¹, R², and R³ are as defined herein. Starting materials may be obtained from commercial sources or via well-established synthetic procedures.

Scheme 3 shows the general synthesis for compounds of an embodiment of Formula (I).

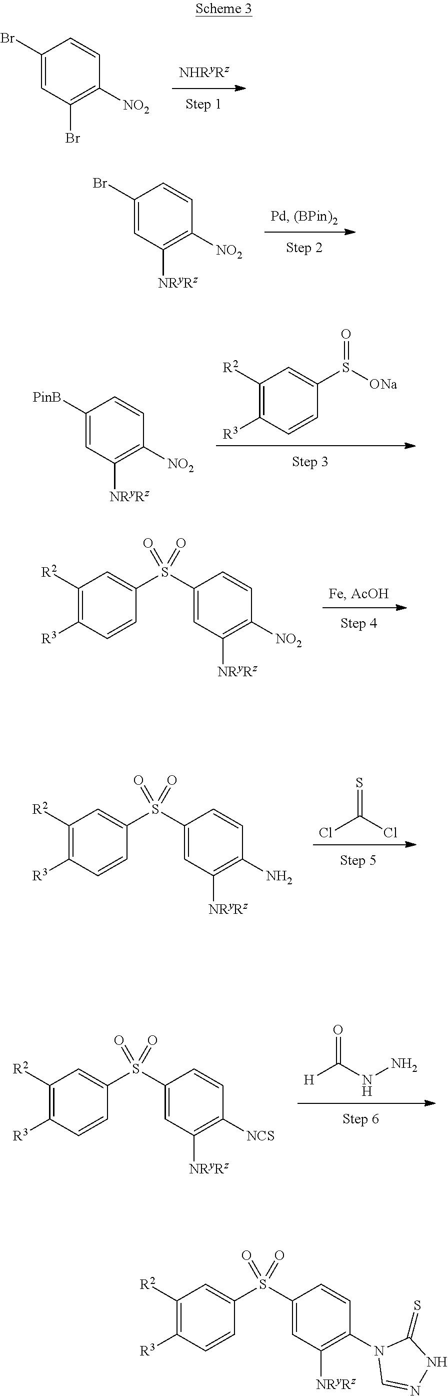

In Scheme 3, R¹, R², R³, R^{y}, and R^{z} are as defined herein.

In certain instances, the above processes further involving the step of forming a salt of a compound of the present disclosure. Embodiments are directed to the other processes described herein; and to the product prepared by any of the processes described herein.

### EXAMPLES

The following examples are offered to illustrate but not to limit the present disclosure. The compounds are prepared using the general methods described above.

The following chemical abbreviations are used throughout the Examples: ACN (acetonitrile), (BPin)₂ (bis(pinacolato)diboron), DCM (dichloromethane), DMF (dimethylformamide), DMSO (dimethyl sulfoxide), EDTA (ethylenediaminetetraacetic acid), EtOH (ethanol), HPLC (high-performance liquid chromatography), IPA (isopropyl alcohol), IPAc (isopropyl acetate), LCMS (liquid chromatography-mass spectrometry), mCPBA (meta-chloroperoxybenzoic acid), MeOH (methanol), MTBE (methyl terbutyl ether), THF (tetrahydrofuran), 2-MeTHF (2-methyltetrahydrofuran), and p-TSA or TsOH (p-toluenesulfonic acid).

### Example 1: In Vitro Ion Transport Assays

To determine the effect of the ion transporter inhibitors on the uptake activity of solute carrier (SLC) ion transporter proteins such as OAT1 and OAT3, an ion transport assay utilizing a polarized monolayer of MDCK-II cells grown on permeable supports was used. The MDCK-II cells were treated to express the transporter of interest (e.g., OAT3 or OAT 1) or were treated with a control vector. 96-well culture plates with both donor and receiver cells containing a monolayer of MDCK-II cells were maintained at 37° C. in 5% CO₂ atmosphere. Radio-labeled para-aminohippurate ([3H]-PAH) was used as a substrate for measuring OAT1 and OAT3 mediated transport, where the transport of the substrate was determined by radiometric detection. OAT1-mediated transport was assayed using 2 μM of [3H]-PAH; OAT3-mediated transport was assayed using 10 μM of [3H]-PAH. The probe substrate for OAT1 was 2 μM [3H]-p-aminohippurate. The probe substrate for OAT3 was 10 μM [3H]p-aminohippurate. The reference inhibitor for both assays was 100 μM probenecid. Following pre incubation of the transfected MDCK-II cells in HBSS, [3H]-PAH was added at the concentrations described above. Wells were then treated with either reference inhibitor probeneic acid or with the test compound. Cells transfected with empty vector served as an additional control. Experiments were performed under the same conditions for the cells expressing the transporter or those treated with the control vector.

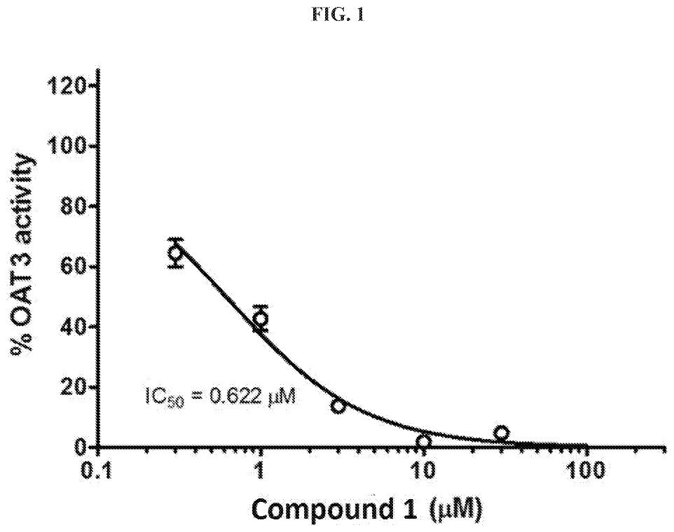

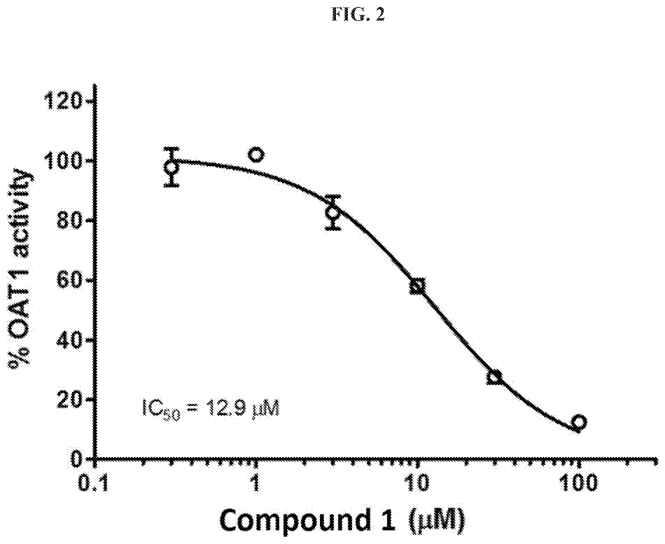

FIG. 1 shows the dose response curve of Compound 1 in its inhibition against organic anion transporter 3 (OAT3) in the uptake of [3H]-PAH. FIG. 2 shows the dose response curve of Compound 1 in its inhibition against organic anion transporter 1 (OAT1) in the uptake of [3H]-PAH. In this assay, the potency of compound 1 against OAT3 was observed to be about 20-fold lower compared with its potency against OAT1. While Compound 1 inhibited OAT3 with an IC₅₀ value of 0.62 μM (FIG. 1), its IC₅₀ against OAT1 was 12.9 μM (FIG. 2).

Compound 1 was also assessed against several other ion transporter proteins, including organic anion transporter 1 (OAT1), organic cation transporter 2 (OCT2), organic anion transporting polypeptide 1B1 (OATP1B1), organic anion transporting polypeptide 1B3 (OATP3B3), multidrug and toxic compound extrusion protein-1 (MATE1/SLC47A1), multidrug and toxic compound extrusion protein 2-K (MATE-2K), breast cancer resistance protein (BCRP), p-glycoprotein (PGP), and uric acid transporter 1 (URAT1).

### Assay 1:

Compound 1 was studied in the concentration range of 0.1-100 μM for its potency against transport mediated by various transporter proteins. The experimental conditions for the in vitro transport assay 1 are summarized in the table below.

| | |
|---|---|
| Test System | 1. MDCK-II cells expressing human transporter MATE1, MATE2-K, |
| | OAT1, OAT3, OCT2, OATP1B1, OATP1B3, or BCRP |
| | 2. MDCK-II control cells transfected with a control vector (GFP) |
| | 3. MDCK-II stable cells expressing P-gp (MDR1) |
| Probe Substrate | 1. MATE1: 10 μM [¹⁴C]-metformin |
| (positive control | 2. MATE2-K: 10 μM [¹⁴C]-metformin |
| for transport) | 3. OCT2: 10 μM [¹⁴C]-metformin |
| | 4. OAT1: 2 μM [³H]-p-aminohippurate |
| | 5. OAT3: 10 μM [³H]-p-aminohippurate |
| | 6. OATP1B1: 2 μM [³H]-estradiol-17β-D-glucuronide |
| | 7. OATP1B3: 2 μM [³H]-CCK-8 |
| | 8. BCRP: 2 μM [³H]-prazosin |
| | 9. P-gp: 100 nM [³H]-quinidine |
| Reference | 1. MATE1: 10 μM cimetidine |
| Inhibitor (Positive | 2. MATE2-K: 10 μM cimetidine |
| Control for | 3. OCT2: 1000 μM quinidine |
| Inhibition) | 4. OAT1: 100 μM probenecid |
| | 5. OAT3: 100 μM probenecid |
| | 6. OATP1B1: 100 μM rifampicin |
| | 7. OATP1B3: 100 μM rifampicin |
| | 8. BCRP: 1 μM Kol43 |
| | 9. P-gp: 3 μM elacridar |
| Test Article | 0, 0.3, 1, 3, 10, 30, & 100 μM, solubility permitting |
| Concentration(s) | |
| Pre-incubation | 15 for OAT1, OAT3, OCT2, OATP1B1, and OATP1B3; |
| Time (min) | 20 for MATE1, MATE2-K; |
| | 30 for BCRP and P-gp |
| Incubation Time | 5 for MATE1, MATE2-K, OAT1, OAT3, OCT2, OATP1B1, and |
| (min) | OATP1B3 |
| | 90 for BCRP and P-gp |

Table 1A summarizes the inhibitory activity of Compound 1 against each of these ion transporter proteins in assay 1.

TABLE 1A

| Transporter | IC₅₀ (μM) |
|---|---|
| OAT1 | 12.9 |
| OAT3 | 0.622 |
| OCT2 | >100 |
| OATP1B1 | 14.6 |
| OATP1B3 | 51.2 |
| MATE1 | 30.3 |
| MATE2-K | 75.8 |
| BCRP | 51.5 |
| PGP | 37.1 |

### Assay 2:

Compound 1 was studied in the concentration range of 0.1-10 μM for its potency against transport mediated by various transporter proteins. The experimental conditions for the in vitro transport assay 2 are summarized in the table below.

| | |
|---|---|
| Test System | 1. MDCK-II cells expressing human transporter OAT3 or URAT1 |
| | 2. MDCK-II transfected with a control vector (GFP) as control cells |
| | for transporters listed in #1. |
| Probe Substrate | 1. OAT3: 10 μM [³H]-p-aminohippurate |
| (positive control | 2. OAT3: 100 nM [³H]-estrone-3-sulfate |
| for transport) | 3. URAT1: 20 μM [¹⁴C]-uric acid |
| Reference | 1. OAT3: 100 μM probenecid |
| Inhibitor (Positive | 2. OAT3: 100 μM probenecid |
| Control for | 3. URAT1: 10 μM benzbromarone |
| Inhibition) | |
| Test Article | For OAT3 evaluations (1 & 2 above) 0, 0.03, 0.1, 0.3, 1, 3, & 10 μM |
| Concentration(s) | For URAT1 evaluation (3 above): 0, 0.03, 0.1, 0.3, 1, 3, & 10 μM |
| Pre-incubation | 30 |
| Time (min) | |
| Incubation Time | 5 |
| (min) | |

Table 1B summarizes the inhibitory activity of Compound 1 against each of these ion transporter proteins in assay 2.

TABLE 1B

| Transporter | IC₅₀ (μM) |
|---|---|
| OAT3 | 0.252 |
| (Substrate: PAH) | |
| OAT3 | 0.891 |
| (Substrate: E3S) | |
| URAT1 | >10 |

In a further assay, the effects of several other compounds (including compounds 2, 3, and 4) were assessed against OAT3. The assay was carried out using a similar procedure as described above, using para-aminohippurate (PAH) and estrone-3-sulfate (E3S) as the substrates for measuring OAT3-mediated transport. Transport studies were conducted using cells expressing the transporter of interest (MDCK-II cells expressing human transporter OAT3) and control cells which do not express the transporter (MDCK-II transfected with a control vector (GFP)). MDCK-II cells were grown in 96-well cell culture plates, and the cell plates are maintained at 37° C. in 5% CO₂ atmosphere prior to initiation of the transport experiment. MDCK-II cells were maintained in DMEM with low glucose and 10% FBS. Cells passages up to 40 are seeded at 60K±10K cells/well on 96-well, transwell membrane plates approximately 24 hours before transfection. Transport assays were carried out approximately 48 hours after transfection. Radio-labeled para-aminohippurate ([3H]-PAH) and estrone-3-sulfate (E3S) were used as substrates for measuring OAT3-mediated transport, where the transport of the substrate was determined by radiometric detection. Probenecid was used as a reference inhibitor. Transport study samples were run in triplicate.

For uptake assays, the net transporter-mediated uptake rate (V) of the substrate by each SLC transporter was calculated as follows:

Transporter-mediated uptake rate(pmol/min/cm²)=((Cellular accumulation in cells expressing the transporter)−(Mean cellular accumulation in control cells))/Incubation time.

Percent inhibition was calculated by dividing the transporter-mediated uptake rate in presence of the test article or the reference inhibitor by the transporter-mediated uptake rate in presence of vehicle control:

Percent inhibition=100−(100×(transporter-mediated uptake rate)_{with inhibitor}/(transporter-mediated uptake rate)_{vehicle control})

For uptake assays, statistical analyses using an unpaired t test was performed between transporter-mediated uptake rate or ATP-dependent transport rate of probe substrate with vehicle control and the inhibitor. A p value of <0.05 was considered statistically significant.

Table 2 summarizes the inhibitory activity of these compounds against OAT3 in the uptake of probe substrate PAH (p-aminohippurate) or E3S (estrone-3-sulfate). The percent inhibition values were determined by testing compounds at a single concentration of 1 μM with a PAH concentration of 10 μM. Data represent the mean and standard deviation of triplicate samples. The IC₅₀ values were calculated by means of a concentration response curve with a compound concentration range of 0.03-10 μM and a probe substrate concentration of 10 μM PAH or 0.1 μM E3S. The percent inhibition at the various concentrations was run in triplicate and the IC₅₀ value was determined by non-linear regression using GraphPad Prism.

TABLE 2

| | | OAT3 inhibition | | |
|---|---|---|---|---|
| | | | | % |
| | | substrate = | substrate = | inhibition |
| | | PAH | E3S | @ 1 μM |
| Compound | Structure | (IC₅₀) | (IC₅₀) | (PAH) |
| 1 | 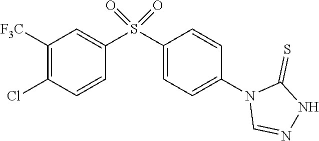 [SMILES: O=S(=O)(c1ccc(-n2cn[nH]c2=S)cc1)c1ccc(Cl)c(C(F)(F)F)c1] | 0.252 μM | 0.891 μM | 80.3% ± 5.91 |
| 2 | 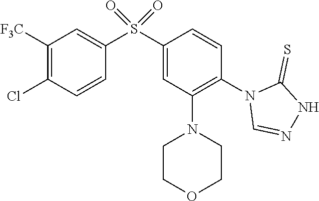 [SMILES: O=S(=O)(c1ccc(-n2cn[nH]c2=S)c(N2CCOCC2)c1)c1ccc(Cl)c(C(F)(F)F)c1] | 1.36 μM | 1.90 μM | 48.0% ± 7.53 |
| 3 | 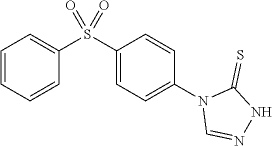 [SMILES: O=S(=O)(c1ccccc1)c1ccc(-n2cn[nH]c2=S)cc1] | N.D. | N.D. | 33.1% ± 12.9 |
| 4 | 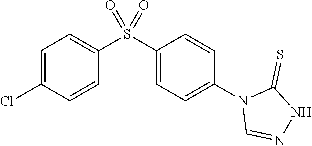 [SMILES: O=S(=O)(c1ccc(Cl)cc1)c1ccc(-n2cn[nH]c2=S)cc1] | N.D. | N.D. | 76.3% ± 5.68 |
| 5 | 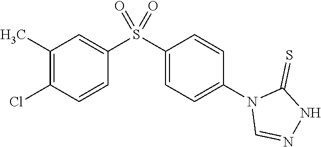 [SMILES: Cc1cc(S(=O)(=O)c2ccc(-n3cn[nH]c3=S)cc2)ccc1Cl] | N.D. | N.D. | 86.5% ± 7.89 |
| 11 | 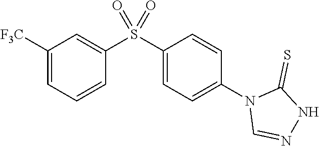 [SMILES: O=S(=O)(c1ccc(-n2cn[nH]c2=S)cc1)c1cccc(C(F)(F)F)c1] | N.D. | N.D. | 58.6% ± 4.77 |
| 12 | 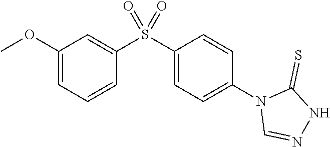 [SMILES: COc1cccc(S(=O)(=O)c2ccc(-n3cn[nH]c3=S)cc2)c1] | N.D. | N.D. | 48.4% ± 9.05 |
| 15 | 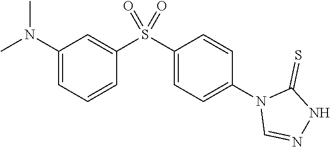 [SMILES: CN(C)c1cccc(S(=O)(=O)c2ccc(-n3cn[nH]c3=S)cc2)c1] | N.D. | N.D. | 60.1% ± 1.58 |
| 19 | 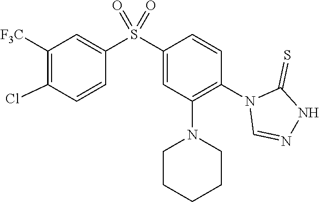 [SMILES: O=S(=O)(c1ccc(-n2cn[nH]c2=S)c(N2CCCCC2)c1)c1ccc(Cl)c(C(F)(F)F)c1] | N.D. | N.D. | 43.0% ± 2.49 |
| 20 | 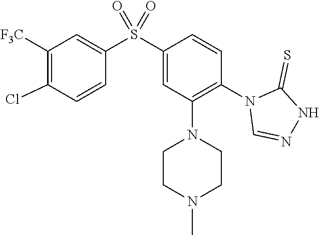 [SMILES: CN1CCN(c2cc(S(=O)(=O)c3ccc(Cl)c(C(F)(F)F)c3)ccc2-n2cn[nH]c2=S)CC1] | N.D. | N.D. | 17.2% ± 16.7 |
| 21 | 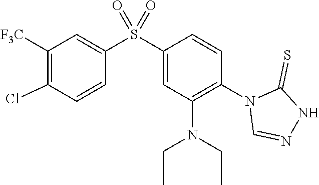 [SMILES: CCN(CC)c1cc(S(=O)(=O)c2ccc(Cl)c(C(F)(F)F)c2)ccc1-n1cn[nH]c1=S] | N.D. | N.D. | 48.2% ± 2.01 |
| 22 | 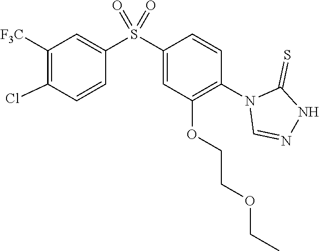 [SMILES: CCOCCOc1cc(S(=O)(=O)c2ccc(Cl)c(C(F)(F)F)c2)ccc1-n1cn[nH]c1=S] | N.D. | N.D. | 79.5% ± 1.25 |
| 26 | 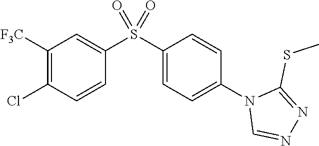 [SMILES: CSc1nncn1-c1ccc(S(=O)(=O)c2ccc(Cl)c(C(F)(F)F)c2)cc1] | 7.67 μM | 8.63 μM | 9.78% ± 3.81 |
| 30 | 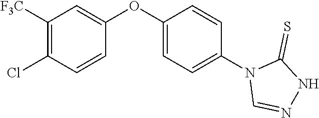 [SMILES: FC(F)(F)c1cc(Oc2ccc(-n3cn[nH]c3=S)cc2)ccc1Cl] | N.D. | N.D. | 86.3% ± 11.1 |
| 31 | 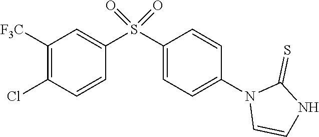 [SMILES: O=S(=O)(c1ccc(-n2cc[nH]c2=S)cc1)c1ccc(Cl)c(C(F)(F)F)c1] | N.D. | N.D. | −1.05% ± 5.92 |
| 32 | 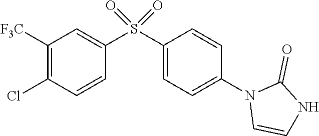 [SMILES: O=c1[nH]ccn1-c1ccc(S(=O)(=O)c2ccc(Cl)c(C(F)(F)F)c2)cc1] | N.D. | N.D. | −9.18% ± 7.51 |
| 33 | 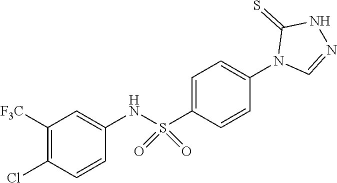 [SMILES: O=S(=O)(Nc1ccc(Cl)c(C(F)(F)F)c1)c1ccc(-n2cn[nH]c2=S)cc1] | N.D. | N.D. | 94.1% ± 4.23 |
| X1 | 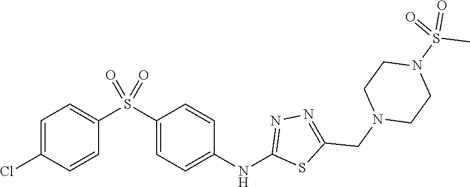 [SMILES: CS(=O)(=O)N1CCN(Cc2nnc(Nc3ccc(S(=O)(=O)c4ccc(Cl)cc4)cc3)s2)CC1] | >30 μM | >30 μM | N.D. |
| X2 | 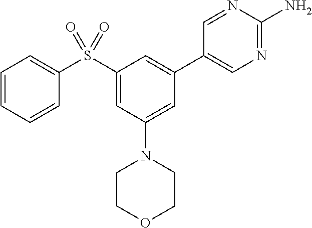 [SMILES: Nc1ncc(-c2cc(N3CCOCC3)cc(S(=O)(=O)c3ccccc3)c2)cn1] | >30 μM | >30 μM | N.D. |

N.D. = not determined

### Example 2: Effect of OAT3 Inhibitor on Levels of Bioactive Endogenous Metabolites in Brain and Plasma

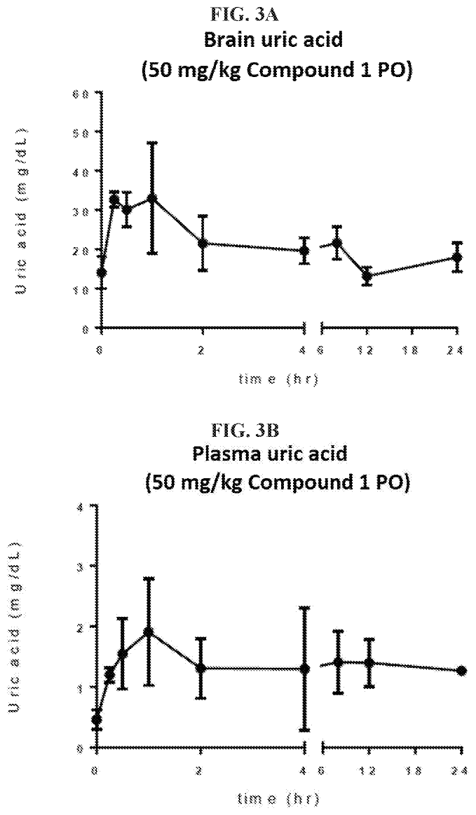

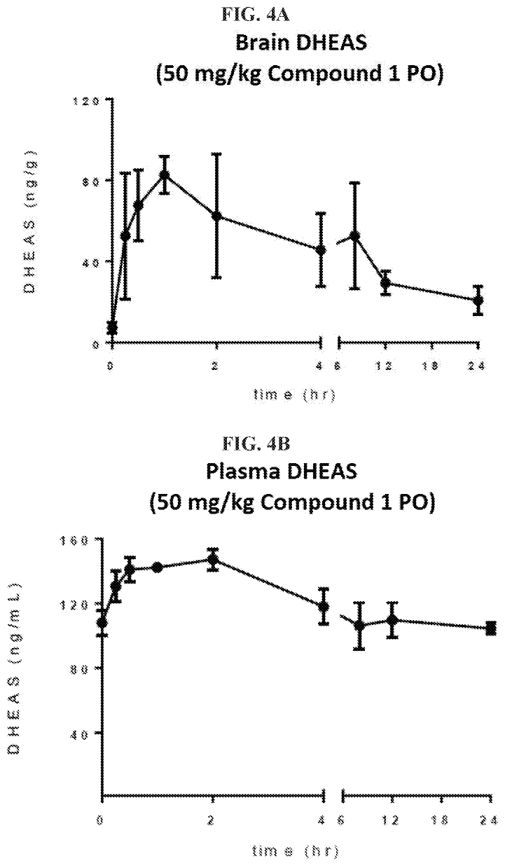

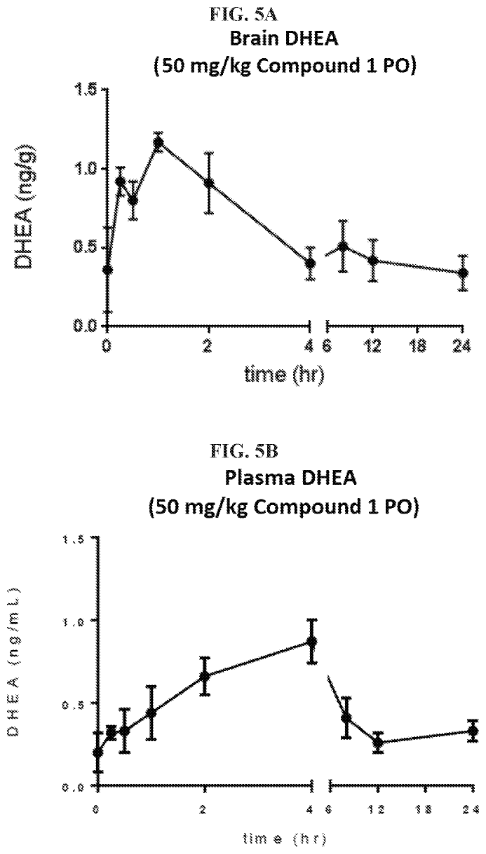

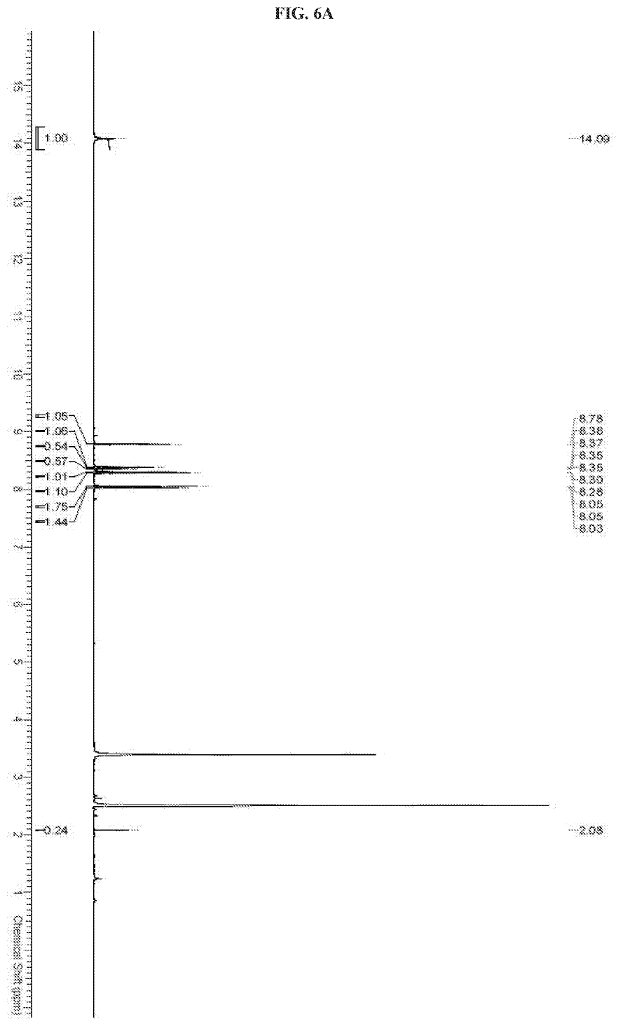

The effect of compound 1 on the levels of bioactive endogenous metabolites in the brain and plasma was assessed. FIG. 3A shows the concentration of uric acid in whole brain homogenates of mice administered (p.o.) with 50 mg/kg of compound 1. FIG. 3B shows the concentration of uric acid in blood plasma of mice administered (p.o.) with 50 mg/kg of compound 1. FIG. 4A shows the concentration of DHEAS in the brain of mice administered (p.o.) with 50 mg/kg of compound 1. FIG. 4B shows the concentration of DHEAS in blood plasma of mice administered (p.o.) with 50 mg/kg of compound 1. FIG. 5A shows the concentration of DHEA in the brain of mice administered (p.o.) with 50 mg/kg of compound 1. FIG. 5B shows the concentration of DHEA in blood plasma of mice administered (p.o.) with 50 mg/kg of compound 1.

### Example 3: Synthesis of 4-(4-((4-chloro-3-(trifluoromethyl)phenyl)sulfonyl)phenyl)-2,4-dihydro-3H-1,2,4-triazole-3-thione (Compound 1)

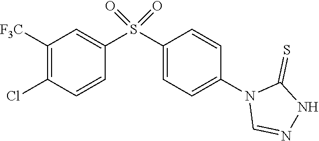 [SMILES: O=S(=O)(c1ccc(-n2cn[nH]c2=S)cc1)c1ccc(Cl)c(C(F)(F)F)c1]

### (i.) Synthesis Route A

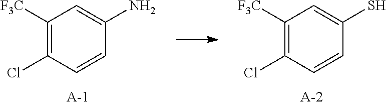

Step 1:

To a mixture of 4-chloro-3-(trifluoromethyl)aniline (500 g, 2.56 mol) in HCl (750 mL) and H₂O (750 mL) was added a solution of NaNO₂ (194 g, 2.81 mol) in 250 mL water, dropwise while keeping the temperature below 5° C. The mixture was stirred at 0-5° C. for 30 min. A solution of ethoxycarbothioyl-sulfanyl potassium (492 g, 3.07 mol) in 1 L water was added dropwise at 0-5° C., and the mixture was stirred at 20° C. for 12 hrs. The mixture was extracted with ethyl acetate (1 L, 3 times). The organic layers were washed with brine (1 L), dried over Na₂SO₄, and evaporated to give o-ethyl [4-chloro-3 (trifluoromethyl)phenyl]-sulfanylmethanethioate (600 g, crude) as a brown oil, which was used directly in the next step.

To the mixture of o-ethyl [4-chloro-3-(trifluoromethyl)phenyl]-sulfanylmethanethioate (600 g, 2 mol) in EtOH (2 L) and H₂O (200 mL) was added KOH (470 g, 8.38 mol). The mixture was stirred at 80° C. for 12 hrs. LCMS showed the desired compound. EtOH was evaporated to give a brown residue which was dissolved in H₂O (2 L) and extracted with 1:1 MTBE/petroleum ether (1 L, 3 times). The aqueous layer was adjusted to pH=1 with concentrated HCl and extracted with ethyl acetate (1 L, 2 times). The organic layers were washed with brine (1 L), dried over Na₂SO₄, and evaporated to give 4-chloro-3-(trifluoromethyl)benzenethiol (480 g, crude) as a brown oil.

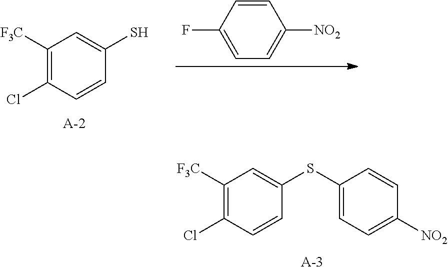

Step 2:

To the mixture of 4-chloro-3-(trifluoromethyl)benzenethiol (480 g, 2.26 mol) in DMF (3 L) was added Cs₂CO₃ (1.15 kg, 3.53 mol) and 1-fluoro-4-nitro-benzene (300 g, 2.12 mol). The mixture was stirred at 80° C. for 3 hrs. The mixture was filtered and the solvent was added to 3 L water and extracted with ethyl acetate (1 L×3). The organic layer was washed with 2 L brine, dried over Na₂SO₄, and evaporated to give (4-chloro-3-(trifluoromethyl)phenyl)(4-nitrophenyl)sulfane (640 g, crude) as a brown solid. ¹H NMR: (CDCl₃, 400 MHz) δ 8.13-8.16 (m, 2H), 7.83 (d, J=0.8 Hz, 1H), 7.58-7.60 (m, 2H), 7.27-7.29 (m, 2H).

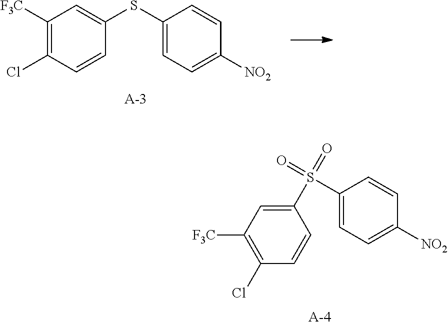 [SMILES: O=[N+]([O-])c1ccc(Sc2ccc(Cl)c(C(F)(F)F)c2)cc1 ; "A-3" ; O=[N+]([O-])c1ccc(S(=O)(=O)c2ccc(Cl)c(C(F)(F)F)c2)cc1 ; "A-4"]

Step 3a:

To the mixture of A-3 (640 g, 1.93 mol) in DCM (3.5 L) was added mCPBA (822 g, 4.05 mol, 80% purity) at 20° C. The mixture was stirred at 20° C. for 12 hrs. The mixture was added to a solution of Na₂SO₃ (100 g, 0.79 mol) and Na₂CO₃ (250 g, 2.36 mol) in 4 L H₂O, and stirred at 20° C. for 2 hrs. The mixture was filtered, and the solid was collected as the desired compound. Additionally, the aqueous layer was extracted with DCM (2 L×2), and the combined organic layers were evaporated to give a brown solid which was made a slurry with ethyl acetate (2 L) to give A-4 (475 g, 67% yield) as a white solid. ¹H NMR (DMSO, 400 MHz) δ 8.33-8.42 (m, 6H), 8.03-8.05 (m, 1H).

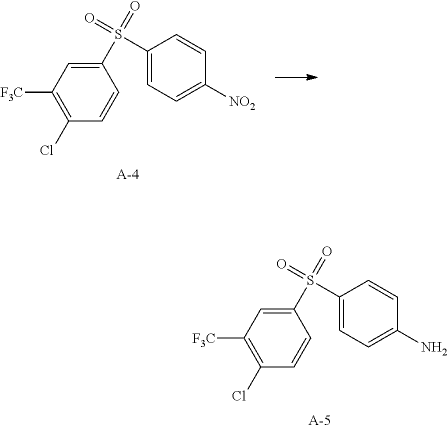 [SMILES: O=[N+]([O-])c1ccc(S(=O)(=O)c2ccc(Cl)c(C(F)(F)F)c2)cc1 ; "A-4" ; Nc1ccc(S(=O)(=O)c2ccc(Cl)c(C(F)(F)F)c2)cc1 ; "A-5"]

Step 4:

To the mixture of A-4 (450 g, 1.23 mol) in EtOH (1.25 L) and H₂O (1.25 L) was added HCl (15 mL). The mixture was heated to 70° C. Fe (140 g, 2.46 mol) was added, and the mixture was stirred at 70° C. for 3 hrs. The mixture was filtered, and EtOH was evaporated. The remaining aqueous solution was extracted with DCM (0.5 L×3), and the organic layers were evaporated to give a solid (the crude product). The solid was dissolved in DCM (1 L×3) and filtered. The solvent was evaporated to give the desired compound. The combined A-5 (200 g, 48% yield) was obtained as an earth yellow solid. ¹H NMR (CDCl₃, 400 MHz): δ 8.20 (s, 1H), 7.98 (d, J=7.2 Hz, 1H), 7.70 (d, J=8.8 Hz, 2H), 7.61 (d, J=8.4 Hz, 1H), 6.68 (d, J=8.4 Hz, 2H), 4.27 (s, 2H).

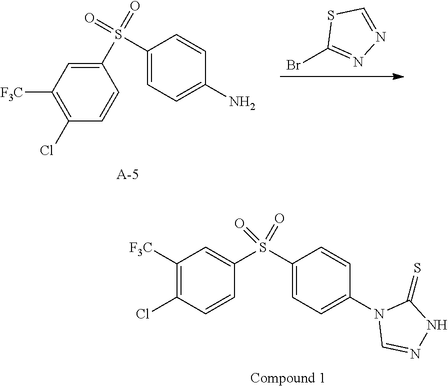 [SMILES: Nc1ccc(S(=O)(=O)c2ccc(Cl)c(C(F)(F)F)c2)cc1 ; Brc1nncs1 ; "A-5" ; O=S(=O)(c1ccc(-n2cn[nH]c2=S)cc1)c1ccc(Cl)c(C(F)(F)F)c1 ; "Compound 1"]

Step 5:

To the mixture of A-5 (100 g, 298 mmol) in isopropanol (1.20 L) was added 2-bromo-1,3,4-thiadiazole (49.2 g, 298 mmol) and TsOH-H₂O (8.50 g, 44.7 mmol). The mixture was stirred at 80° C. for 4 hrs. The mixture was filtered, and the filtrate was evaporated to give a crude product. The crude product was purified by column chromatography on silica gel (petroleum ether/ethyl acetate=3/1˜0/1, 0-10% 0.5M NH₃.H₂O/MeOH in DCM) to give a yellow solid which was made a slurry from MeOH (300 mL), MTBE (500 mL), and H₂O (500 mL), then dried in vacuum to give Compound 1 (20 g, 8% yield) as a white solid. 1H NMR (500 MHz, DMSO-d6) δ 14.07 (s, 1H), 8.77 (s, 1H), 8.42-8.32 (m, 2H), 8.32-8.25 (m, 2H), 8.03 (dd, J=8.8, 2.3 Hz, 3H). LCMS ES+(m/z), 420.0 (M+1)+, Cl pattern found.

### (ii.) Synthesis Route B

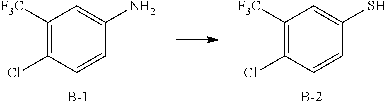

Step 1:

In a 1 L round-bottom flask equipped with a mechanical stirrer and thermometer was added 60 mL of concentrated hydrochloric acid, 60 mL of water, and 4-chloro-3-(trifluoromethyl)benzene amine (19.5 g, 0.1 mol). The mixture was heated to promote dissolution and then cooled down to below 0° C. in an ice-water bath. A solution of sodium nitrite (7.6 g, 0.11 mol) in 10 mL of water was added in dropwise while the internal temperature was kept below 5° C., and the mixture was stirred at 5° C. for 30 min. The mixture was then added into a mixture of potassium ethyl xanthate (19.2 g, 0.12 mol) in 30 mL of water over 2 hours. Upon the completion of reaction (about 30 min), the organic phase in the reaction mixture was separated, and the aqueous layer was extracted twice with diethyl ether. The combined organic layers were washed with 30 mL of 10% sodium hydroxide solution followed by several portions of water until the aqueous phase that separated was pH neutral. The organic phase was dried over Na₂SO₄ and concentrated, and the crude residue was dissolved in 95% ethanol (100 mL). The solution heated to reflux to aid dissolution. To this hot solution was added potassium hydroxide pellets (23.5 g, 0.42 mol) slowly so that the solution kept gentle refluxing until all the material was completely dissolved in water (about 8 hours). Approximately 80 mL of ethanol was then removed by distillation on a steam bath, and the residue was taken up in the minimum amount of water (about 100 mL). The aqueous solution was extracted with diethyl ether (50 mL×3). The pH of aqueous layer was adjusted to 1 with 6 N sulfuric acid. Extraction with diethyl ether (50 mL×3) was performed, and the combined organic layers were dried over Na₂SO₄ and concentrated to give the crude product, which was purified by column chromatography (0 to 2% ethyl acetate/petroleum ether) to give 4-chloro-3-(trifluoromethyl)benzenethiol (16.1 g, 75%) as a yellow solid.

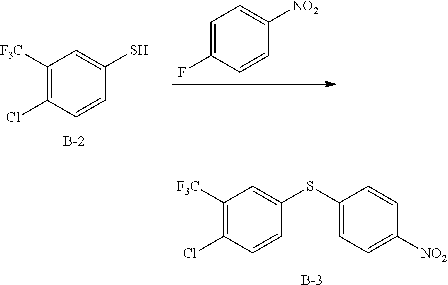

Step 2:

To a solution of 4-chloro-3-(trifluoromethyl)benzenethiol (19.2 g, 0.091 mol) in N,N-dimethylformamide (250 mL) was added 1-fluoro-4-nitrobenzene (12.8 g, 0.091 mol) and Cs₂CO₃ (59.4 g, 0.182 mol), and the reaction mixture was stirred at 80° C. under thin layer chromatography monitoring (1:30 ethyl acetate/petroleum ether). Upon the completion of the reaction, the mixture was cooled to room temperature and diluted with water (500 mL). The aqueous layer was extracted with ethyl acetate (200 mL×3), and the combined organic layers were washed with brine, dried over Na₂SO₄, filtered and concentrated to give crude 4-chloro-3-(trifluoromethyl)phenyl)(4-nitrophenyl)sulfane (25 g, 82%) as a yellow oil, which was used in the next step without further purification.

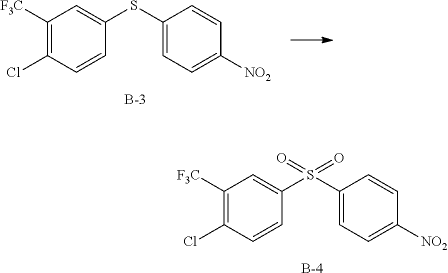

Step 3b:

To a solution of 4-chloro-3-(trifluoromethyl)phenyl)(4-nitrophenyl)sulfane (25 g, 0.075 mol) in acetic acid (100 mL) was added 30% H₂O₂ dropwise (20 g, 0.3 mol) at room temperature. The reaction mixture was stirred at 85° C. with thin layer chromatography monitoring (1:5 ethyl acetate/petroleum ether). Upon the completion of reaction, water was added to quench the reaction. The aqueous layer was extracted with ethyl acetate (100 mL×3), and the combined organic layers were washed with brine, dried over Na₂SO₄, filtered and concentrated to give the crude product, which was purified by flash chromatography (0 to 10% ethyl acetate/petroleum ether) to give 1-chloro-4-(4-nitrophenylsulfonyl)-2-(trifluoromethyl) benzene (20.8 g, 76%) as a white solid.

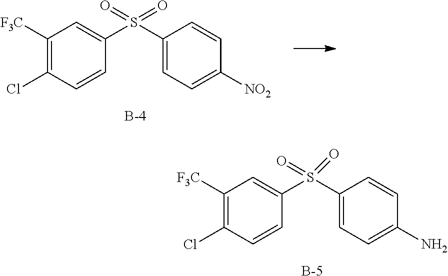

Step 4:

Five drops of concentrated HCl was added into a mixture of iron power (16 g, 0.29 mol) in water (100 mL) and ethanol (100 mL). The mixture was heated to reflux while 1-chloro-4-(4-nitrophenylsulfonyl)-2-(trifluoromethyl)benzene (26.4 g, 0.072 mol) was added. The reaction mixture was kept under reflux for an additional hour with thin layer chromatography monitoring (1:5 ethyl acetate/petroleum ether). Upon the completion of reaction, the hot mixture was filtered, and the filter cake was washed with ethanol. The pH of filtrate was adjusted to 10 with 2 N NaOH, and the aqueous phase was extracted with ethyl acetate (100 mL×3). The combined organic layers were washed with brine, dried over Na₂SO₄, filtered and concentrated to give the crude product, which was purified by flash chromatography (0 to 15% ethyl acetate/petroleum ether) to give 4-((4-chloro-3-(trifluoromethyl)phenyl)sulfonyl) aniline as a white solid (19.4 g, 79%).

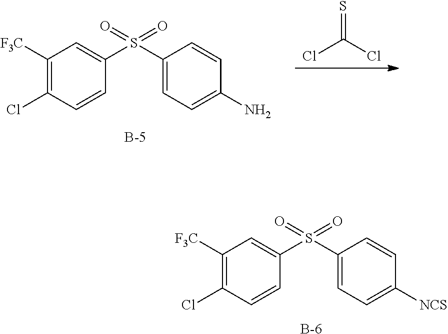

Step 6:

Thiophosgene (6.6 g, 0.057 mol) was added into a two phase solution of 4-((4-chloro-3-(trifluoromethyl)phenyl)sulfonyl) aniline (19.2 g, 0.057 mol) in dichloromethane and water containing sodium bicarbonate (13.4 g, 0.13 mol) at 0° C. The reaction mixture was stirred at 0° C. for 2 hours. Upon the completion of reaction, the organic layer was separated, dried over Na₂SO₄, filtered and concentrated to dryness. The residue was purified by column chromatography (0 to 50% ethyl acetate/petroleum ether) to give 1-chloro-4-(4-isothiocyanatophenylsulfonyl)-2-(trifluoromethyl)benzene (11.5 g, 53%) as a yellow solid.

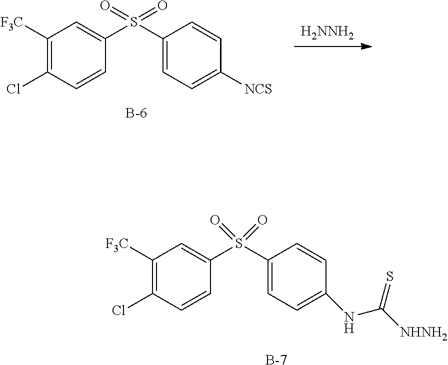

Step 7:

Hydrazine monohydrate (5.2 g, 0.058 mol) was added into a solution of 1-chloro-4-(4-isothiocyanatophenylsulfonyl)-2-(trifluoromethyl)benzene (11 g, 0.029 mol) in ethanol (60 mL) dropwise at 0° C. After 4 hours, the reaction mixture was diluted with water (100 mL) and extracted with dichloromethane (50 mL×3). The combined organic layers were washed with brine, dried over Na₂SO₄, filtered and concentrated to give crude N-(4-((4-chloro-3-(trifluoromethyl)phenyl)sulfonyl) phenyl)hydrazinecarbothioamide (8.4 g, 70%), which was used in the next step without further purification.

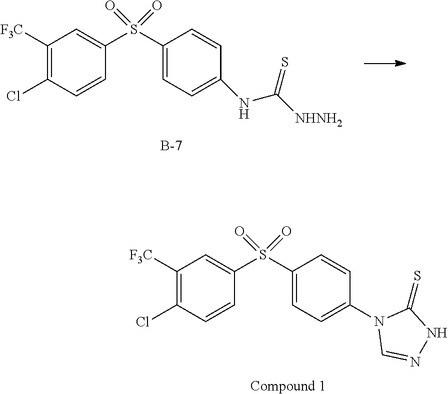

Step 8:

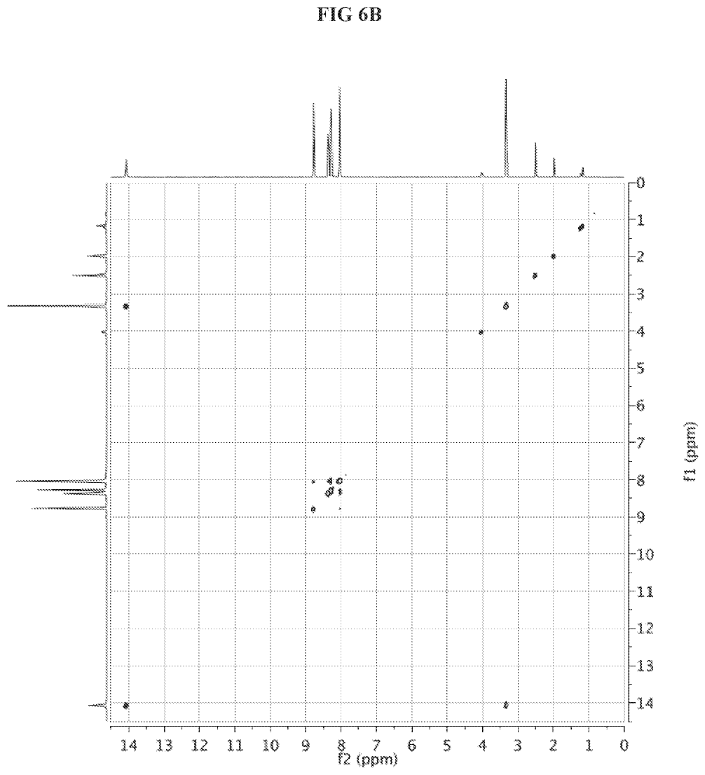

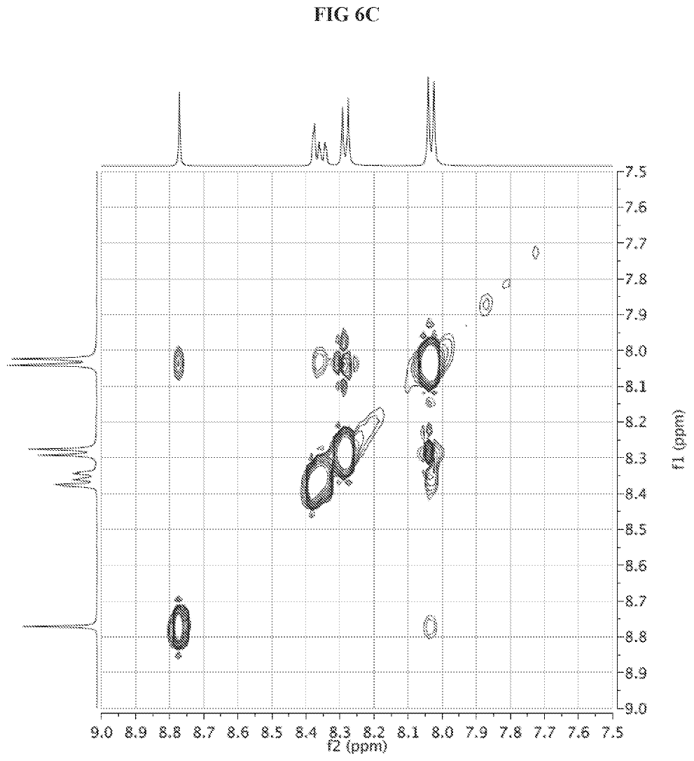

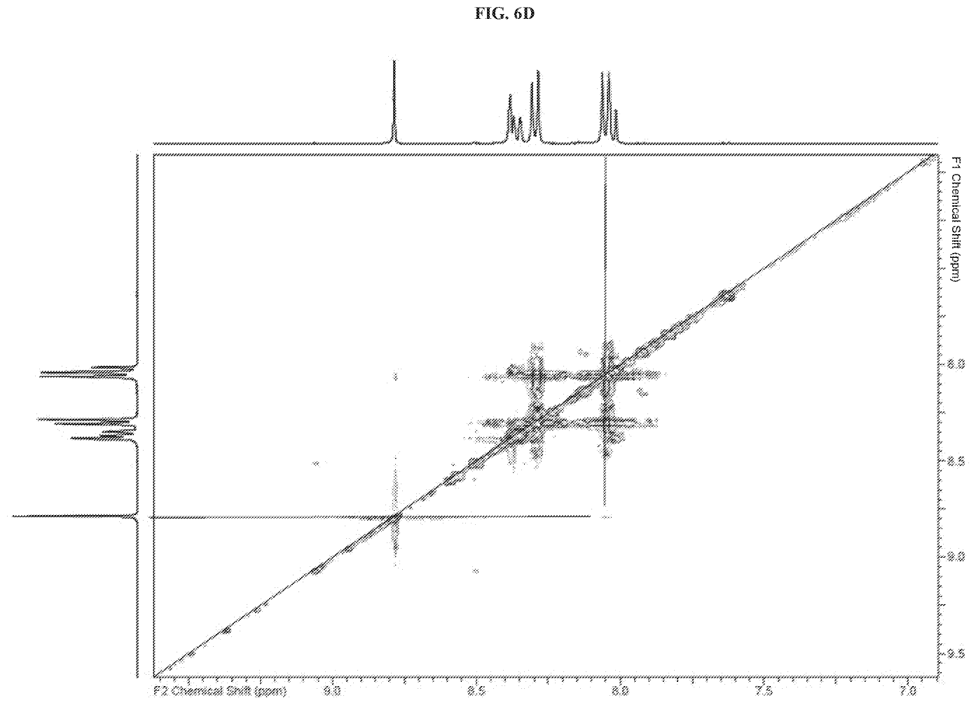

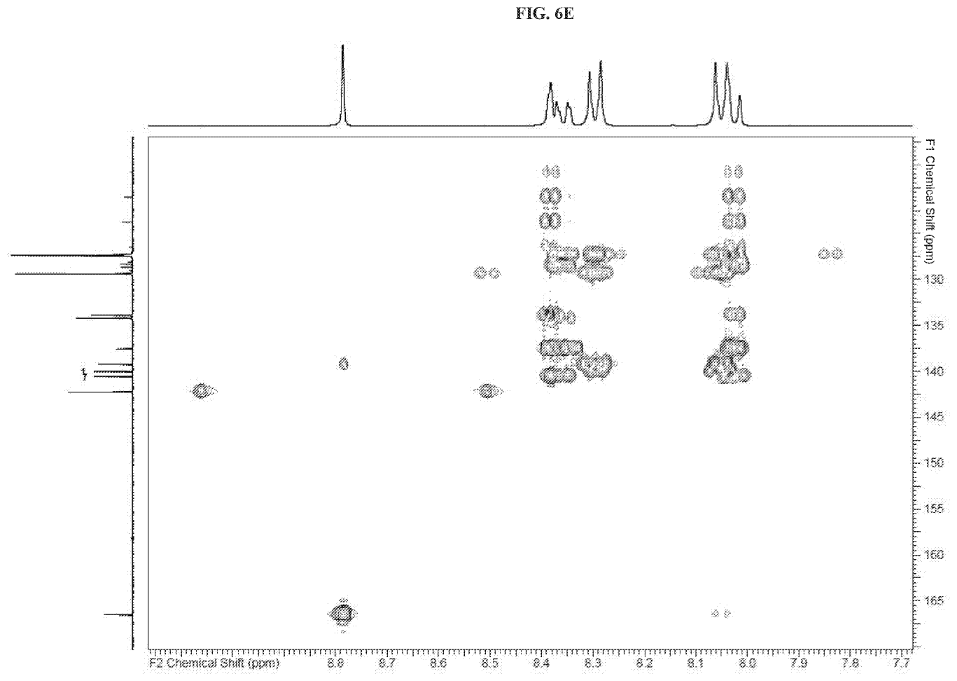

N-(4-((4-Chloro-3-(trifluoromethyl)phenyl)sulfonyl) phenyl)hydrazinecarbothioamide (8.2 g, 0.02 mol) was treated with triethoxymethane (50 mL) at 145° C. for 3 hours. Water (100 mL) was added, and the mixture was extracted with dichloromethane (50 mL×3). The combined organic layers were washed with brine, dried over Na₂SO₄, filtered and concentrated to give the crude product, which was purified by column chromatography (0 to 10% ethyl acetate/petroleum ether) to give the title compound (5.4 g, 64%) as a white solid. ¹H NMR (300 MHz, DMSO-d₆) δ 14.07 (s, 1H), 8.77 (s, 1H), 8.41-8.32 (m, 2H), 8.32-8.25 (m, 2H), 8.06-8.00 (m, 3H). LCMS ES+(m/z), 420.0 (M+1)⁺, Cl pattern found. FIG. 6B shows a 2D NOESY spectrum of Compound 1 in DMSO-d6 (400 MHz) as synthesized via Route B. FIG. 6C shows an expansion of the 2D NOESY spectrum of compound 1 in DMSO-d6 (500 mHz) as synthesized via Route B. The NOESY spectra show nOe coupling between the triazole thione CH and the phenyl CH, corresponding to R¹ in Formula 1.

### (iii.) Synthesis Route C

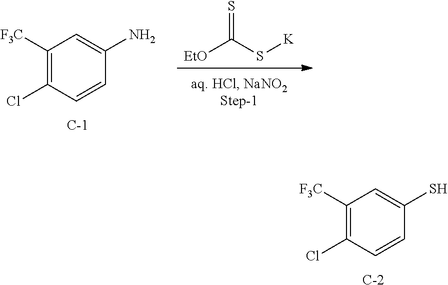

Step 1:

A 250 mL jacketed flask was equipped with a magnetic stirrer. The flask was charged with concentrated HCl (25 mL, 0.30 mol, 3.0 eq) and water (98.2 mL). 4-Chloro-3-(trifluoromethyl)aniline (20.0 g, 0.10 mol, 1.0 eq) was melted and added to the flask at 25° C. The mixture was heated to 50° C. and stirred at 50° C. for 30 min. After cooling the mixture to 0-5° C., a solution of NaNO₂ (7.6 g, 0.11 mol, 1.1 eq) in 12 mL water was added dropwise over 30 min while maintaining a temperature between 0-5° C. After completing addition of NaNO₂, the mixture was stirred at 0-5° C. for 1 h.

A second reaction flask was charged with potassium ethyl xanthate (20.8 g, 0.13 mol, 1.3 eq) followed by water (80 mL). After stirring for 20 minutes, toluene (80 mL) was added followed by dropwise addition of the diazonium salt from the first reaction flask at 19-23° C. over 3 h. After complete addition, the mixture was stirred at 20° C. for 2 h. The aqueous phase was separated from the organic phase and extracted with 20 mL toluene, three times. The organic phases were combined and washed with water (10 mL, 4 times) and then degassed by bubbling nitrogen through for 30 min.

A third flask was charged with EtOH (63.2 g), water (10 mL) and KOH (23.0 g, 0.41 mol, 4.1 eq). The ethanolic KOH solution was degassed by bubbling nitrogen through the mixture 30 minutes. The KOH solution was heated to 75-82° C. under and inert nitrogen atmosphere. The toluene solution from the second reaction vessel was added to the degassed ethanolic KOH solution at 75-82° C. over the course of 2 hours under an inert nitrogen atmosphere. After addition, the mixture was stirred at 78° C. for 3.5 hours.

The mixture was distilled to 1.5-2 V at 45° C. Additional toluene was added (60 mL, N₂ purged) to the mixture before distilling again to 1.5-2 V at 45° C. and adding toluene (20 mL, N₂ purged). Water (80 mL, N₂ purged) was added into the reaction flask and the aqueous phase was separated from the toluene. The aqueous phase was washed with 20 mL toluene 3 times. The aqueous phase was cooled to 10° C. and the pH was adjusted pH<1 with conc. HCl (32.0 mL) at 10-15° C. The mixture was purged with nitrogen for 20 minutes and warmed to 20° C. MTBE (40 mL, N₂ purged) was added under nitrogen atmosphere. The organic and aqueous phases were separated. The aqueous phase was extracted with MTBE (40 mL, N₂ purged) 3 times. The organic MTBE phases were combine and washed with water (10 mL, N₂ purged) 3 times. By HPLC, the assay yield of 4-chloro-3-(trifluoromethyl)benzenethiol was 64.5%. The product was then phase transferred from MTBE to Acetonitrile by distilling at 60° C. under atmospheric pressure. Acetonitrile (50 mL) was added, and the mixture was distilled at 80° C. under atmospheric pressure. Additional acetonitrile (40 mL) was added to give 4-chloro-3-(trifluoromethyl)benzenethiol with no residual MTBE.

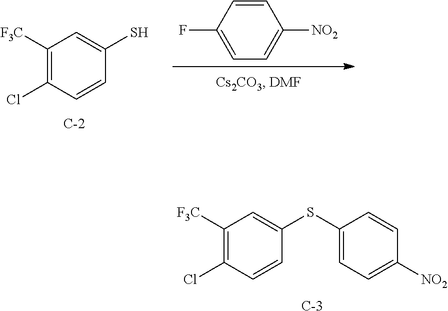

Step 2:

To a mixture of 60.0 g of 4-chloro-3-(trifluoromethyl)benzenethiol (0.285 mol, 1.0 eq.) in MeCN (1116 mL) was added Cs₂CO₃ (195.0 g, 0.60 mol, 2.1 eq.) and 1-fluoro-4-nitro-benzene (52.3 g, 0.37 mol, 1.3 eq.). The mixture was stirred at 80° C. for 11 h, cooled to 25-30° C. and filtered. The filter cake was rinsed with acetonitrile (120 mL×2). The acetonitrile solution was concentrated to 60-120 mL under reduced pressure, keeping the temperature below 45° C. Dichloromethane (1116 mL) and 15% NaCl (1600 mL) were added to the solution. The mixture was stirred at 20-30° C. for 30 minutes and the organic layer was separated. The organic layer was washed with 5 wt % NaCl solution 2 more times. The organic layer was concentrated to 480-600 mL under reduced pressure while keeping the temperature below 45° C. Dichloromethane (560 mL) was added to the solution and the organic layer was concentrated again to 480-600 mL to give a solution of (4-chloro-3-(trifluoromethyl)phenyl)(4-nitrophenyl)sulfane in DCM that was used directly in the next step.

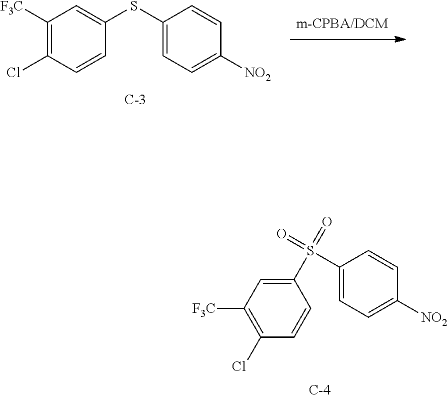

Step 3:

Additional DCM (340 mL, 20 vol.) was added to a DCM (8.5 vol.) solution of (4-chloro-3-(trifluoromethyl)phenyl)(4-nitrophenyl)sulfane (17.0 g, 50.9 mmol, 1.0 eq.) from step 2. The mixture was heated to 33-37° C. and stirred for 0.5 h before portion wise addition of m-CPBA (31.0 g, 152.8 mmol, 3.0 eq, 85 wt %) at 33-37° C. The mixture was stirred at 33-37° C. for 4 h and then cooled to 20-30° C. To the mixture, 16% wt Na₂SO₃ aq. (146.2 g, 8.6 X) and 16% Na₂CO₃ aq. (146.2 g, 8.6 X) were added while keeping the temperature below 30° C. The mixture was stirred at 20-30° C. for 1 h. The organic layer was separated, washed with 10 wt % NaCl solution (51.0 g, 3 X), and concentrated to 3-5 vol. under reduced pressure below 45° C. IPAc (15 vol.) was added and the solution was concentrated to 6-8 vol. under reduced pressure below 45° C. IPAc (15 vol.) was added to the mixture a second time before again concentrating the solution to 6-8 vol. under reduced pressure below 45° C. IPAc (28 vol.) was added and the mixture was heated to 60° C. with stirring to provide a clear solution. The solution was cooled to 55° C. with stirring for 1-2 h. The solution was distilled to 3-5 vol. under reduced pressure below 55° C. The mixture was cooled down to 45° C. for 2 h. MTBE (11 vol.) was added to the mixture and the mixture stirred at 45° C. for an additional 1-2 h. The mixture was cooled to −10° C. in 11 h and aged at −10° C. for an additional 4.5 h. The mixture was filtered, and the wet cake was washed twice with IPAc/MTBE=1/4 (4 vol.). The wet cake was dried for 1 h under reduced pressure below 45° C. to give 1-chloro-4-((4-nitrophenyl)sulfonyl)-2-(trifluoromethyl)benzene (19.5 g, 99.7% assay yield) as an off-white solid (97.5% purity).

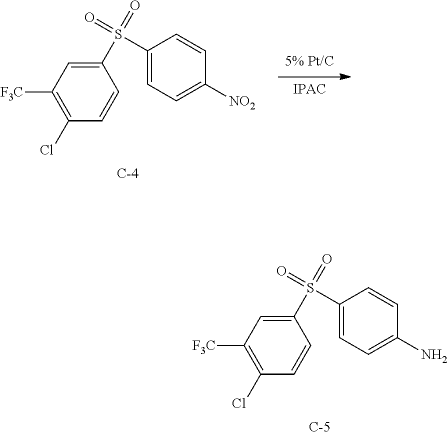

Step 4:

1-Chloro-4-((4-nitrophenyl)sulfonyl)-2-(trifluoromethyl)benzene (20.0 g, 54.7 mmol) and IPAc (200 mL) were added to a 1.0 L high-pressure vessel. The vessel was purged and degassed with Ar₂, charged with 5% Pt/C (800 mg) under N₂ protection, purged and degassed with H₂ and the mixture was stirred at 0.5 MPa (72.5 psi) H₂ atmosphere at 65° C. for 18 h. Over that period the hydrogen pressure was depleted to 0 MPa, so the vessel was recharged with H₂ to 0.5 MPa and kept at 65° C. for 14 h. The mixture was cooled, filtered through celite, washed with IPAc (50 mL×2) and the solvent was distilled to obtain a light yellow solid (18.0 g, 98.5% crude yield).

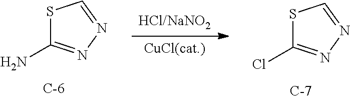

Step 5:

To a flask containing 1,3,4-thiadiazol-2-amine (5.0 g, 49.4 mmol) at 30° C. was added 30 mL of HCl (30 g, 36.5% aq, 300 mmol) followed by 25 mL of H₂O. The solution was cooled to 0° C. to give a suspension. CuCl (0.5 g, 4.9 mmol) was added at 0° C. A solution of NaNO₂ (3.4 g, 49.4 mmol) in H₂O (50 mL) was added slowly at 0° C. over a period of 30 min. and the reaction mixture was stirred for 2.5 h at 0-5° C. IPAc (100 mL) was added and the reaction was quenched with 10% NaHSO₃ (60 mL). NaHCO₃ (25 g, solid) was added slowly to pH=6-7 and the organic layer was separated. The aqueous layer was extracted with IPAc (100 mL×2). The organic layers were combined and washed with 10% EDTA (50 mL×4) and H₂O (100 mL). The combined EDTA aqueous and H₂O layers were extracted with IPAc (100 mL). The combined organic IPAc extracts were dried over Na₂SO₄, filtered, concentrated in vacuo, redissolved in IPAc (100 mL) and evaporated in vacuo (2×) to give crude product (4.0 g) as an light yellow oil. The oil was stored at 5° C. for up to 12 h.

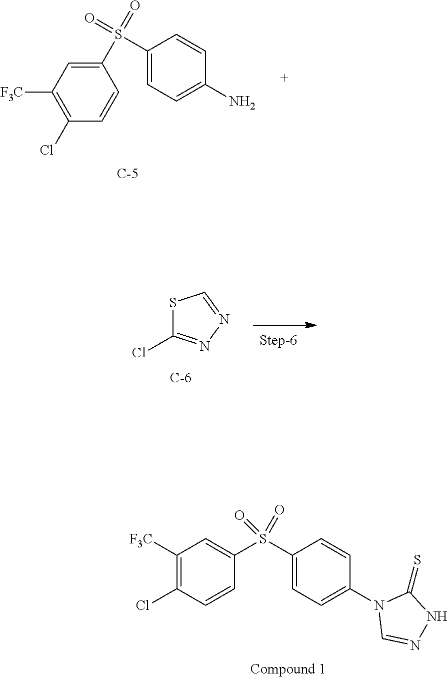

Step 6:

4-((4-Chloro-3-(trifluoromethyl)phenyl)sulfonyl)aniline (7.0 g, 20.9 mmol) and IPA (93 mL) was added to a reaction vessel at 30° C. to give a suspension. p-TSA.H2O (595 mg) was added and the reaction mixture was heated to 80-85° C. 2-Chloro-1,3,4-thiadiazole (4.6 g, 38.2 mmol) in IPA (20 mL) was added at 80-85° C. over a period of 5 h and the mixture was stirred for 1 h after the addition was complete. The mixture was cooled to 30° C. and stood for 15 h. The reaction mixture was concentrated to dryness. MTBE (50 mL) was added and the mixture was stirred for 2 h at 30° C. and filtered. The MTBE layer was retained and contained 1.0 g by assay yield (3%, 32 g×3%=1.0 g) of 4-(4-((4-chloro-3-(trifluoromethyl)phenyl)sulfonyl)phenyl)-2,4-dihydro-3H-1,2,4-triazole-3-thione. The filter cake was poured into 100 mL 2-MeTHF and saturated NaHCO₃ was added to pH=7-8. Assay yield of the 2-MeTHF layer indicted 4.1 g grams (102 g×4%=4.1 g) of 4-(4-((4-chloro-3-(trifluoromethyl)phenyl)sulfonyl)phenyl)-2,4-dihydro-3H-1,2,4-triazole-3-thione. Total weight by assay yield=5.1 g, 58% yield. 1H NMR (400 MHz, DMSO-d₆) δ 14.09 (s, 1H), 8.78 (s, 1H), 8.38-8.35 (m, 2H), 8.30-8.28 (m, 2H), 8.03 (m, 3H). LCMS ES+(m/z), 420.0 (M+1)+, Cl pattern found. The ¹H NMR spectrum of Compound 1 is shown in FIG. 6A. FIG. 6D shows a 2D NOESY spectrum of Compound 1 in DMSO-d6 (400 MHz) as synthesized from Route C. The NOESY spectrum shows nOe coupling between the triazole thione CH and the phenyl CH, corresponding to R¹ in Formula 1. FIG. 6E shows the HMBC of Compound 1 in DMSO-d6 (400 MHz) showing a correlation between the triazole thione CH, and the aromatic carbon connected to the triazole thione.

### (iv.) Synthesis Route D

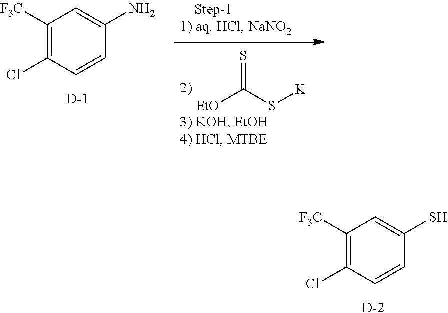

Step 1:

Purified water (178 kg) was charged into a reaction vessel followed by concentrated HCl (216 kg) and 4-chloro-3-(trifluoromethyl)aniline (60.55 kg, 1.0 eq). The mixture was heated to 45-55° C., stirred for 5 h and then cooled −5˜5° C. A solution of NaNO₂ (25.65 kg) in 38 kg water was added drop-wise over 1-2 h at −5˜5° C. After addition, the mixture was stirred at 0-5° C. for 2 h. The solution (528.2 kg) and an aqueous solution of potassium O-ethyl carbonodithioate (63.5 kg potassium O-ethyl carbonodithioate and 242 kg purified water) were added at 15-25° C. simultaneously over 2-6 h into a reactor containing toluene (211.6 kg, 4V) and 0.5 volumes of purified water. The resultant mixture was stirred at 20° C. for 5-12 h. The layers were separated, and the aqueous phase was extracted with toluene (112 kg). The organic layers were combined and washed with purified water 3 times.

Ethanol (208 kg) and water (32 kg) were charged into a second reaction vessel followed by KOH (71 kg). The mixture was heated to 75-82° C. under N₂ protection. The toluene solution from the extraction was added at 75-82° C. under N₂ protected over 5 h. The mixture was stirred at 78° C. for 5 h. The mixture was then distilled to 2-4 volumes at an inner temperature not more than 45° C. and distilled again with toluene (169 kg) to remove EtOH. Purified water (250 kg) was charged into the vessel with stirring; the toluene phase was separated and the aqueous layer was washed with 2 volumes of toluene 2 times to give a product rich aqueous layer.

The aqueous layer was cooled to 0-10° C. and purged with N₂ for 2 h at which time nitrogen-purged 6N HCl (2.0-5.0×) was added dropwise at 0-10° C. until the pH was between 1 and 2. The mixture was stirred for 1 h at 0-10° C. The resulting mixture was stirred for 1 h at 0-10° C. and was then extracted with MTBE (250 kg), which had also been purged with N₂ for 2 h. The organic layer was separated and washed with purified water twice (2×268 kg) and the resulting organic layer was stored for further processing. 36.6 kg of 4-chloro-3-(trifluoromethyl)benzenethiol (D-2) was obtained as a solution in MTBE. The product was a mixture of monomer and dimer with a yield of 55.5%.

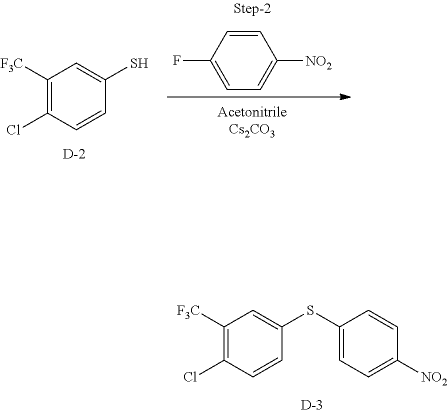

Step 2:

The mixture of D-2 and dimer (34.1 kg, 158.8 kg×21.5 wt %, 1.0 eq.) in MTBE (3 vol.) was charged into a reaction vessel. Acetonitrile (482 kg, 18.6 vol.) was added followed by Cs₂CO₃ (157 kg, 3.0 eq.) and 1-fluoro-4-nitro-benzene (29.6 kg, 1.3 eq.). The mixture was heated to 60-65° C. and stirred at that temperature for 57 h. The mixture was cooled to 20-30° C. Celite (37 kg) was added and, after stirring for 1-3 h, the mixture was filtered and washed with acetonitrile (163 kg). The acetonitrile solution was concentrated to 6-7 volumes below 45° C. under vacuum. The mixture was then stirred at 40-45° C. for 0.5-1 h until a clear solution was achieved. The mixture was cooled to 25-30° C. over 1-2 hours and then stirred for an additional 0.5-1 h. Seed crystals of D-3 (96 g) were added, and the mixture was stirred for 1-2 h. Water (136 kg) was added dropwise over 7 hours, and the mixture continued to stir at 25-30° C. for 10-20 hours. The mixture was centrifuged and the resultant cake was washed twice with 104 kg of ACN/H₂O (6:4 by volume). The wet cake was dried at 50-60° C. for 24 h to give 40.4 kg of (4-chloro-3-(trifluoromethyl)phenyl)(4-nitrophenyl)sulfane (D-3) in 74.4% isolated yield.

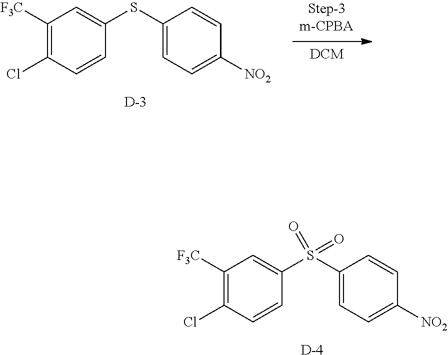

Step 3:

DCM (1480 kg) was charged into a reaction vessel followed by 40.4 kg of D-3. The mixture was heated to 33-37° C. MCPBA (3×20.6 kg) was added portion-wise at 33-37° C. and stirred for 20-30 minutes between additions. After the addition was complete, the reaction was stirred for 3-5 hours at 33-37° C. After cooling to 20-30° C., 16 wt % Na₂SO₃ aq. (344 kg) and 16% Na₂CO₃ aq. (342 kg) were added. The mixture was stirred for 1-2 h and then extracted with DCM (342 kg). The organic layer was separated and washed with an aqueous solution of 7 wt % Na₂SO₄ (134 kg) 2 times. The organic layer was concentrated to 3-4 vol. under reduced pressure below 35° C., while keeping the walls of the reaction vessel clean by rinsing down the sides with DCM (114 kg). MTBE (322 kg) was added, and the mixture was stirred at 40-50° C. for 1-2 h, cooled to 5-10° C., and stirred at 5-10° C. for 4-6 h. The precipitate was filtered and washed with solvent (DCM:MTBE=1:3,118 kg) and re-suspended in MTBE (156 kg) and DCM (66 kg). After stirring at 5-10° C. for 1-2 h, the precipitate was filtered and washed with solvent (DCM:MTBE=1:3.38 kg). The filter cake was dried under vacuum at 40-45° C. for 8-12 h to give 39.87 kg (91.4% yield) of 1-chloro-4-((4-nitrophenyl)sulfonyl)-2-(trifluoromethyl)benzene (D-4).

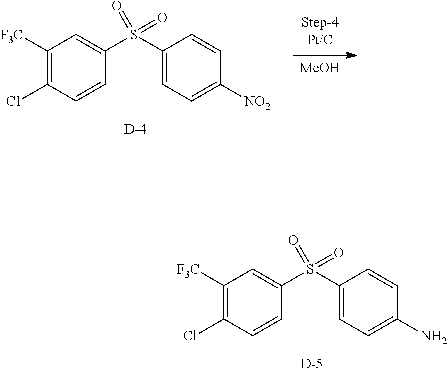

Step 4:

Pt/V/C (2.9 kg) was added to a reaction vessel containing D-4 (38.4 kg) in THF (198 kg) and MeOH (126 kg). The reaction vessel was evacuated and back-filled with nitrogen 3 times and then evacuated and back-filled with hydrogen 3 times. The temperature was adjusted to 60° C., and the reaction was stirred under H₂ (0.3-0.4 MPa) for 17 hours. The reaction mixture was filtered and washed with THF (97 kg). The filtrate was concentrated to 2-3 volumes. The solvent was exchanged by methanol addition (120 kg) and concentrated to 2-3 volumes (repeated 3 times). Methanol (64 kg) was added to the reaction vessel and the temperature was adjusted to 60° C. with stirring for 0.5-1 hour. The temperature was lowered to 55° C., and seed crystals of D-5 (0.04 kg) were added. The mixture was stirred at 50-60° C. for 5 hours and then lowered to 20° C. over 6 hours. Water (100 kg) was added over 5 h, and then the suspension was stirred for 7 h. The precipitate was filtered and washed with a MeOH:H₂O solution (3:1, 98 kg). The filter cake was dried under vacuum at 45° C. for 16 h to give 4-((4-chloro-3-(trifluoromethyl)phenyl)sulfonyl)aniline, D-5, (31.9 kg) in 90.6% yield.

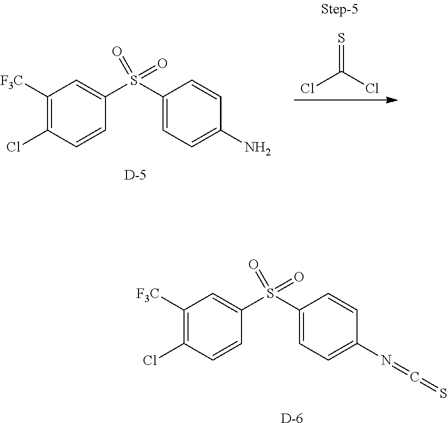

Step 5:

To a reaction vessel containing a solution of NaHCO₃ (23.4 kg) and water (293 kg), D-5 (28.5 kg) was added followed by 361 kg of DCM. After stirring at 15-25° C. for 0.5 h, the reaction vessel was cooled to −5˜5° C. Sequential addition of thiophosgene (12.3 kg, 6 kg) added dropwise with stirring at −5˜5° C. for 4 hours followed by NaHCO₃ (3.7 kg, 2.9) was repeated twice. A final portion of thiophosgene (6.0 kg) was added, and the reaction was stirred at −5˜5° C. for 2-10 h, warmed to 15-25° C. and stirred for an additional 1-2 h. The organic layer was separated and washed with water (112 kg). The organic layer was concentrated to 2-3 volumes under vacuum below 25° C. DCM (185 kg) addition and concentration (to 2-3 volumes under vacuum below 25° C.) was repeated 3 times with a final DCM concentration of 4-5 volumes. Solvent exchange was accomplished by portion-wise addition of the DCM solution of D-6 to a second reaction vessel charged with 180 kg of methylcyclohexane with stirring at 20-25° C. for 2-4 hours and concentrated to 7.5-8.5 volumes under vacuum at a temperature below 25° C. between additions. Methylcyclohexane (2×100 kg) was added to the vessel, and the mixture was concentrated to 4.0-4.5 volumes under vacuum below 35° C. twice. Additional methylcyclohexane (135 kg) was added, and the mixture was stirred at 55-65° C. for 3-4 h, cooled slowly (10-12 h) to 0-5° C. and stirred for 6-10 h. The suspension was filtered, washed with 68 kg methylcyclohexane and dried at 40-50° C. for 24 h to give 29.9 kg of 1-chloro-4-((4-isothiocyanatophenyl)sulfonyl)-2-(trifluoromethyl)benzene (D-6) in 93.2% yield.

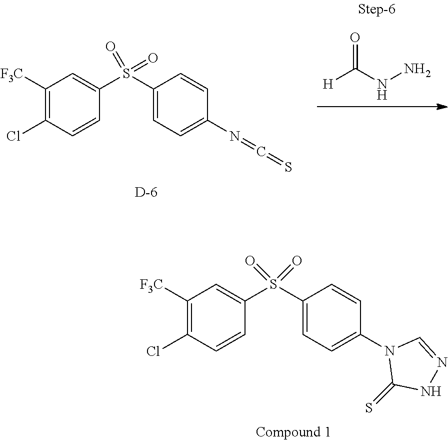

Step 6:

To a reaction vessel charged with D-6 (30.95 kg) and DABCO (11.4 k g) was added THF (268 kg) under nitrogen. The reaction vessel was cooled to 10˜20° C. and stirred for 30-60 min and before adding formohydrazide (5.6 kg) under nitrogen. The reaction was stirred at 10˜20° C. for 1.5 h, warmed to 35˜45° C., then stirred for 17 hours, and then warmed to 45˜55° C. and stirred 9 hours. The reaction was cooled to 20˜40° C. and transferred to a second reaction vessel through a fine filter. The mixture was concentrated to ˜2 volumes while keeping the temperature below 40° C. Water (251 kg) was added under N₂ followed by the addition of 6N HCl (30.9 kg) until a pH of 4 was reached. The reaction was warmed to 40-50° C., stirred for 3 hours, and then cooled to 15-25° C. and stirred for 4 hours. The mixture was centrifuged, and the precipitate was washed with water:THF (3:1, 72 kg) and water (94 kg). The solid product was dried at 40˜50° C. for 27 h to give Compound 1 in 93.7% yield and 97% purity.

4-(4-((4-chloro-3-(trifluoromethyl)phenyl)sulfonyl)phenyl)-2,4-dihydro-3H-1,2,4-triazole-3-thione was further purified by polish filtration and recrystallization. 17.4 kg of the 4-(4-((4-chloro-3-(trifluoromethyl)phenyl)sulfonyl)phenyl)-2,4-dihydro-3H-1,2,4-triazole-3-thione dissolved in acetone (158 kg) and stirred at 20-30° C. until a clear solution was obtained. The solution was filtered through a fine filter and concentrated to 7-9 volumes under vacuum while keeping the temperature below 40° C. The mixture was cooled to 30° C., charged with seed crystals (21 g), stirred 7 h, then concentrated to 3-5 volumes under vacuum while keeping the temperature below 40° C.

Solvent exchange was performed two times with ethanol by sequential addition of ethanol (56 kg, 52 kg), stirring, and concentrating to 3-5 volumes under vacuum at a temperature below 40° C. The compound was recrystallized in ethanol (88 kg) by heating to 75-82° C., stirring the mixture for 10 h, cooling the mixture to 15-25° C. over 5 h, and stirring the mixture at 15-25° C. for 8 h. The mixture was filtered, washed with 160 g ethanol, and dried at 40-50° C. for 10-16 h to give 16.64 kg of Compound 1 in 99% purity.

### Example 4: 4-(4-((4-chloro-3-(trifluoromethyl)phenyl)sulfonyl)-2-morpholinophenyl)-2,4-dihydro-3H-1,2,4-triazole-3-thione (Compound 2)

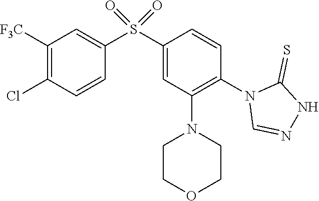 [SMILES: O=S(=O)(c1ccc(-n2cn[nH]c2=S)c(N2CCOCC2)c1)c1ccc(Cl)c(C(F)(F)F)c1]

The synthesis of 4-(4-((4-chloro-3-(trifluoromethyl)phenyl)sulfonyl)-2-morpholinophenyl)-2,4-dihydro-3H-1,2,4-triazole-3-thione was accomplished in a similar manner as described in Synthesis Route B of Example 3 from 4-(5-fluoro-2-nitrophenyl)morpholine. ¹H NMR (300 MHz, DMSO-d₆) δ 14.00 (1H, s), 8.41 (2H, m), 8.67 (1H, s), 8.04 (1H, d, J=6 Hz), 7.90 (1H, dd, J=3, 6 Hz), 7.83 (1H, d, J=3 Hz), 7.71 (1H, d, J=6 Hz), 3.55 (4H, m), 2.82 (4H, m). LCMS ES+(m/z), 505.0 (M+1)+, Cl pattern found.

### Example 5: 4-(4-(phenylsulfonyl)phenyl)-2,4-dihydro-3H-1,2,4-triazole-3-thione (Compound 3)

### Step 1: Synthesis of Sodium Benzenesulfinate

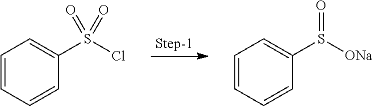

Phenyl sulfonyl chloride (3.5 g, 19.9 mmol, 1 eq.) was added to a solution of sodium sulfite (5 g, 39.8 mmol, 2 eq.) and sodium bicarbonate (3.3 g, 39.8 mmol, 2 eq.) in water (50 mL). The reaction was stirred for 2 hours at rt. The water was removed in vacuo and the residue was suspended in methanol and filtered. The residue was washed with methanol 3 more times and filtered. The methanol filtrates were combined and concentrated. The resultant solid was re-suspended in methanol and filtered. The filtrate was concentrated to give crude sodium benzenesulfinate, which was used for next reaction without further purification. Neg. LC-MS: 141.14 (M−H)⁻, C₆H₅NaO₂S.

### Step 2: Synthesis of 4,4,5,5-Tetramethyl-2-(4-nitrophenyl)-1,3,2-dioxaborolane

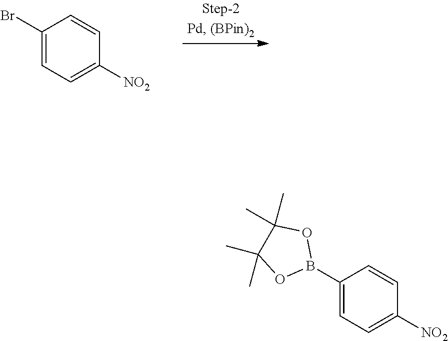 [SMILES: O=[N+]([O-])c1ccc(Br)cc1 ; CC1(C)OB(c2ccc([N+](=O)[O-])cc2)OC1(C)C]

A mixture of 1-bromo-4-nitrobenzene (2.02 g, 0.01 mol, 1 eq.), 4,4,4′,4′,5,5,5′,5′-octamethyl-2,2′-bi(1,3,2-dioxaborolane) (2.54 g, 0.01 mol, 1 eq.), potassium acetate (2.88 g, 0.03 mol, 1 eq.), and PdCl₂(dppf) (0.82 g, 10.0 mmol, 0.1 eq.) in dioxane (35 mL) was refluxed overnight. The mixture was cooled to rt, diluted with water (100 mL), and extracted with ethyl acetate (100 mL×3). The organic extracts were combined, washed with brine (50 mL), dried over anhydrous sodium sulfate, and concentrated. The residue was purified by silica gel column chromatography (petroleum ether/ethyl acetate=20:1 to 5:1) to give the product (1.83 g, 73% yield).

### Step 3: Synthesis of 1-nitro-4-(phenylsulfonyl)benzene

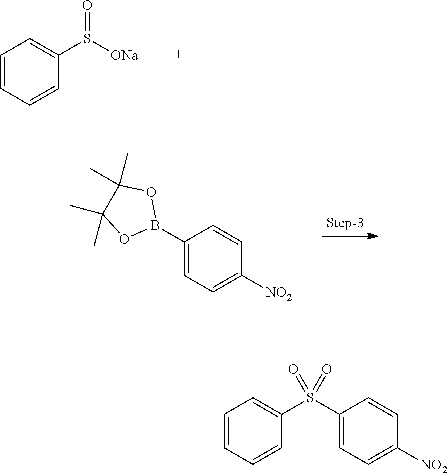

Potassium carbonate (2.01 g, 14.6 mmol, 2 eq.), 4 Å MS, and Cu(OAc)₂ (1.49 g, 8.0 mmol, 1.1 eq.) were added successively to a solution of compound 4,4,5,5-Tetramethyl-2-(4-nitrophenyl)-1,3,2-dioxaborolane (1.82 g, 7.3 mmol, 1 eq.) and crude sodium benzenesulfinate (2.39 g, 14.6 mmol, 2 eq.) in DMSO (50 mL). The reaction was stirred overnight at 45° C. under the atmosphere of an oxygen balloon. The reaction mixture was poured into water and extracted with ethyl acetate. The organic extracts were combined, washed with brine, dried over anhydrous sodium sulfate, and concentrated. The residue was purified by silica gel column chromatography to give 1-nitro-4-(phenylsulfonyl)benzene, 0.71 g, 37% yield.

### Step 4: Synthesis of 4-(phenylsulfonyl)aniline

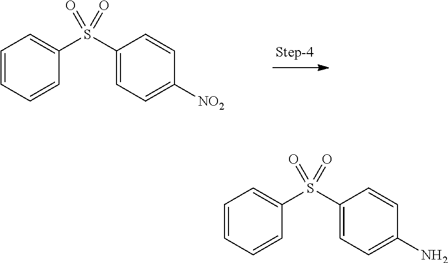 [SMILES: O=[N+]([O-])c1ccc(S(=O)(=O)c2ccccc2)cc1 ; Nc1ccc(S(=O)(=O)c2ccccc2)cc1]

1-Nitro-4-(phenylsulfonyl)benzene (0.7 g, 2.66 mmol, 1 eq.) was dissolved in acetic acid (10 mL) and Fe (1.49 g, 26.6 mmol, 10 eq.) was added. The reaction was heated at 60° C. for 2 h. The mixture was cooled to rt, diluted with ethyl acetate, filtered, and the cake was washed with ethyl acetate. The filtrate was washed with brine. The organic extract was concentrated and the residue was purified by silica gel column chromatography to give 4-(phenylsulfonyl)aniline. (0.52 g, 2.23 mmol, 84% yield). Pos. LC-MS: 233.92 (M+H)⁺, C₁₂H₁₁NO₂S.

### Step 5: Synthesis of 1-isothiocyanato-4-(phenylsulfonyl)benzene

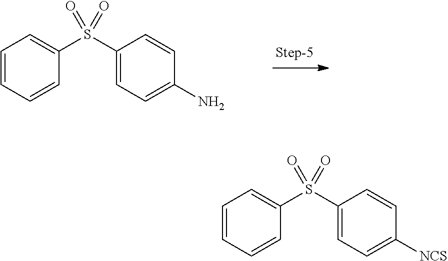

Thiophosgene (308 mg, 2.68 mmol, 1.2 eq.) was added to a mixture of 4-(phenylsulfonyl)aniline. (520 mg, 2.23 mmol, 1 eq.) and saturated sodium bicarbonate-water solution (10 mL) in chloroform (10 mL). The reaction was stirred for 2 h at rt under nitrogen protection. The mixture was extracted with dichloromethane twice. The organic extracts were combined, washed with brine, dried over anhydrous sodium sulfate, and concentrated to afford crude 1-isothiocyanato-4-(phenylsulfonyl)benzene, which was used for next reaction without further purification.

### Step 6: Synthesis of 4-(4-(phenylsulfonyl)phenyl)-2,4-dihydro-3H-1,2,4-triazole-3-thione

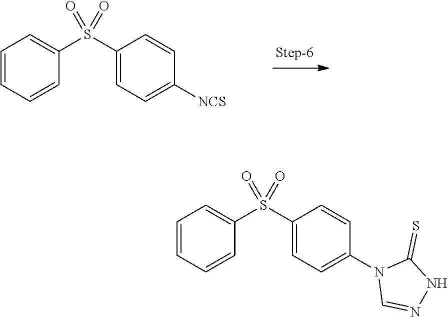

A solution of crude 1-isothiocyanato-4-(phenylsulfonyl)benzene (275 mg, 1.0 mmol, 1 eq.) and formohydrazide (60 mg, 1.0 mmol, 1 eq.) in ethanol (5 mL) was refluxed for 30 min. The solvent was removed and the residue was dissolved in 2% NaOH (5 mL). The reaction was heated at 100° C. for another 2 h. The mixture was cooled to rt and acidified to pH=3-4 by HCl. The resulting precipitate was extracted with dichloromethane two times. The organic extracts were combined, washed with brine, dried over anhydrous sodium sulfate, and concentrated. The residue was re-crystallized in ethanol to give 4-(4-(phenylsulfonyl)phenyl)-2,4-dihydro-3H-1,2,4-triazole-3-thione (48 mg, 0.15 mmol, 15% yield) as an off-white solid. Neg. LC-MS: 316.1 (M−H)⁻, C₁₄H₁₁N₃O₂S₂. ¹H NMR (DMSO-d6, 400 MHz) δ: 14.07 (br, 1H), 8.77 (s, 1H), 8.16 (d, J=8.4 Hz, 2H), 7.96-8.15 (m, 4H), 7.64-7.75 (m, 3H).

### Example 6: 4-(4-((4-chlorophenyl)sulfonyl)phenyl)-2,4-dihydro-3H-1,2,4-triazole-3-thione (Compound 4)

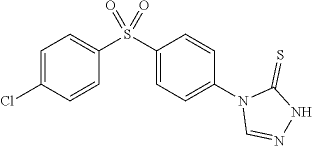 [SMILES: O=S(=O)(c1ccc(Cl)cc1)c1ccc(-n2cn[nH]c2=S)cc1]

4-(4-((4-Chlorophenyl)sulfonyl)phenyl)-2,4-dihydro-3H-1,2,4-triazole-3-thione was synthesized in a similar manner as described for 4-(4-(phenylsulfonyl)phenyl)-2,4-dihydro-3H-1,2,4-triazole-3-thione. Yield for step 6: 22%, off-white solid. Neg. LC-MS: 350.0 (M−H)⁻, C₁₄H₁₀ClN₃O₂S₂. ¹H NMR (DMSO-d6, 400 MHz) δ: 14.08 (br, 1H), 8.77 (s, 1H), 8.18 (d, J=8.4 Hz, 2H), 7.98-8.06 (m, 4H), 8.79 (d, J=8.4 Hz, 2H).

### Example 7: 4-(4-((4-Chloro-3-methylphenyl)sulfonyl)phenyl)-2,4-dihydro-3H-1,2,4-triazole-3-thione (Compound 5)

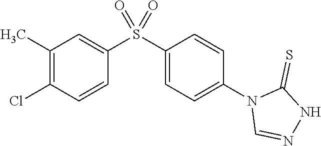 [SMILES: Cc1cc(S(=O)(=O)c2ccc(-n3cn[nH]c3=S)cc2)ccc1Cl]

4-(4-((4-Chloro-3-methylphenyl)sulfonyl)phenyl)-2,4-dihydro-3H-1,2,4-triazole-3-thione was synthesized in a similar manner as described for 4-(4-(phenylsulfonyl)phenyl)-2,4-dihydro-3H-1,2,4-triazole-3-thione. Yield for Step 6: 12%, pale yellow solid. LC-MS: 364.0 (M−H)⁻, C₁₅H₁₂ClN₃O₂S₂. ¹H NMR (DMSO-d6, 400 MHz) δ: 14.09 (br, 1H), 8.77 (s, 1H), 8.17 (d, J=8.4 Hz, 2H), 8.07 (s, 1H), 7.99 (d, J=8.4 Hz, 2H), 7.86 (d, J=7.6 Hz, 1H), 7.71 (d, J=8.4 Hz, 1H), 2.42 (s, 3H).

### Example 8: 4-((4-(5-thioxo-1,5-dihydro-4H-1,2,4-triazol-4-yl)phenyl)sulfonyl)benzonitrile (Compound 6)

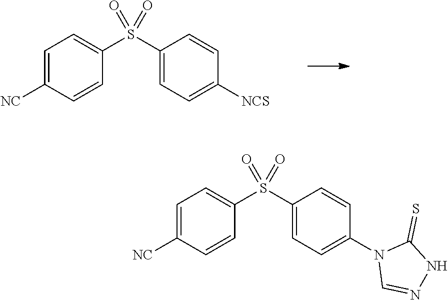

4-(4-Isothiocyanatophenylsulfonyl)benzonitrile was synthesized following the procedure as described for Compound 3 in Example 5. A solution of crude 4-(4-Isothiocyanatophenylsulfonyl)benzonitrile (200 mg, 0.67 mmol, 1 eq.) and formohydrazide (401 mg, 0.67 mmol, 1 eq.) in ethanol (5 mL) was refluxed for 30 min. Triethylamine (202 mg, 2.00 mmol, 3 eq.) was added, and the reaction was refluxed for another 2 h. The solvent was removed in vacuo, and the residue was diluted with water. The mixture was extracted with ethyl acetate three times. The organic extracts were combined, washed with brine, dried over anhydrous sodium sulfate, and concentrated. The residue was stirred with DCM and filtered to give 4-((4-(5-thioxo-1,5-dihydro-4H-1,2,4-triazol-4-yl)phenyl)sulfonyl)benzonitrile (110 mg, 0.32 mmol, 48% yield) as an off-white solid. Pos. LC-MS: 343.0 (M+H)⁺, C₁₅H₁₀N₄O₂S₂. ¹H NMR (DMSO-d6, 400 MHz) δ: 14.09 (br, 1H), 8.78 (s, 1H), 8.23 (m, 4H), 8.15 (m, 2H), 8.02 (m, 2H).

### Example 9: 4-(4-((4-morpholinophenyl)sulfonyl)phenyl)-2,4-dihydro-3H-1,2,4-triazole-3-thione (Compound 7)

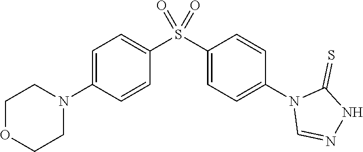 [SMILES: O=S(=O)(c1ccc(N2CCOCC2)cc1)c1ccc(-n2cn[nH]c2=S)cc1]

4-(4-(4-morpholinophenylsulfonyl)phenyl)-2,4-dihydro-3H-1,2,4-triazole-3-thione was synthesized in a similar manner as described for 4-(4-(3-(dimethylamino)phenylsulfonyl)phenyl)-2,4-dihydro-3H-1,2,4-triazole-3-thione (Compound 15) in Example 13 starting from 4-(morpholin-4-yl)benzenethiol. Yield for step 5: 10%, off-white solid. Pos. LC-MS: 402.9 (M+H)⁺, C₁₈H₁₈N₄O₃S₂. ¹H NMR (499 MHz, DMSO-d₆) δ 14.03 (s, 1H), 8.74 (d, J=1.6 Hz, 1H), 8.08-8.03 (m, 2H), 7.93-7.89 (m, 2H), 7.79-7.76 (m, 2H), 7.09-7.05 (m, 2H), 3.70 (t, J=5 Hz, 4H), 3.28 (t, J=5 Hz, 4H).

### Example 10: 4-(4-((3-(Trifluoromethyl)phenyl)sulfonyl)phenyl)-2,4-dihydro-3H-1,2,4-triazole-3-thione (Compound 11)

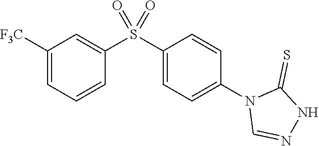 [SMILES: O=S(=O)(c1ccc(-n2cn[nH]c2=S)cc1)c1cccc(C(F)(F)F)c1]

4-(4-((3-(Trifluoromethyl)phenyl)sulfonyl)phenyl)-2,4-dihydro-3H-1,2,4-triazole-3-thione was synthesized in a similar manner as described for 4-(4-(phenylsulfonyl)phenyl)-2,4-dihydro-3H-1,2,4-triazole-3-thione. Yield for step 6: 13%, off-white solid. Neg. LC-MS: 384.1 (M−H)⁻, C₁₅H₁₀F₃N₃O₂S₂. ¹H NMR (DMSO-d6, 400 MHz) δ: 14.08 (br, 1H), 8.78 (s, 1H), 8.37 (m, 2H), 8.28 (d, J=8.4 Hz, 2H), 8.14 (d, J=7.6 Hz, 1H), 8.02 (d, J=8.4 Hz, 2H), 7.93 (m, 1H).

### Example 11: 4-(4-((3-methoxyphenyl)sulfonyl)phenyl)-2,4-dihydro-3H-1,2,4-triazole-3-thione (Compound 12)

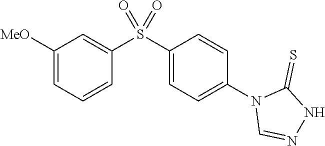 [SMILES: COc1cccc(S(=O)(=O)c2ccc(-n3cn[nH]c3=S)cc2)c1]

4-(4-((3-Methoxyphenyl)sulfonyl)phenyl)-2,4-dihydro-3H-1,2,4-triazole-3-thione was synthesized in a similar manner as described for 4-(4-(phenylsulfonyl)phenyl)-2,4-dihydro-3H-1,2,4-triazole-3-thione. Yield for step 6: 57%, off-white solid. Neg. LC-MS: 346.0 (M−H)⁻, C₁₅H₁₃N₃O₃S₂. ¹H NMR (DMSO-d6, 400 MHz) δ: 14.09 (br, 1H), 8.78 (s, 1H), 8.19 (d, J=8.4 Hz, 2H), 7.98 (d, J=8.4 Hz, 2H), 7.56 (m, 2H), 7.52 (s, 1H), 7.28 (m, 1H), 3.85 (s, 3H).

### Example 12: 3-((4-(5-thioxo-1,5-dihydro-4H-1,2,4-triazol-4-yl)phenyl)sulfonyl)benzonitrile (Compound 14)

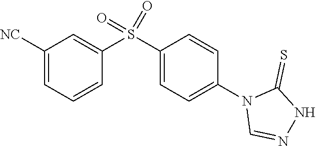 [SMILES: N#Cc1cccc(S(=O)(=O)c2ccc(-n3cn[nH]c3=S)cc2)c1]

3-((4-(5-thioxo-1,5-dihydro-4H-1,2,4-triazol-4-yl)phenyl)sulfonyl)benzonitrile was synthesized in a similar manner as described for 4-((4-(5-thioxo-1,5-dihydro-4H-1,2,4-triazol-4-yl)phenyl)sulfonyl)benzonitrile (Compound 6) in Example 8. Yield for final step: 44% as a yellow solid. Pos. LC-MS: 343.0 (M+H)⁺, C₁₅H₁₀N₄O₂S₂. ¹H NMR (DMSO-d6, 400 MHz) δ: 14.09 (br, 1H), 8.78 (s, 1H), 8.62 (s, 1H), 8.35 (m, 1H), 8.25 (m, 2H), 8.20 (m, 1H), 8.01 (m, 2H), 7.85 (m, 1H).

### Example 13: 4-(4-(3-(dimethylamino)phenylsulfonyl)phenyl)-2,4-dihydro-3H-1,2,4-triazole-3-thione (Compound 15)

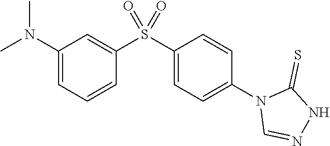 [SMILES: CN(C)c1cccc(S(=O)(=O)c2ccc(-n3cn[nH]c3=S)cc2)c1]

### Step 1: N,N-dimethyl-3-(4-nitrophenylthio)aniline

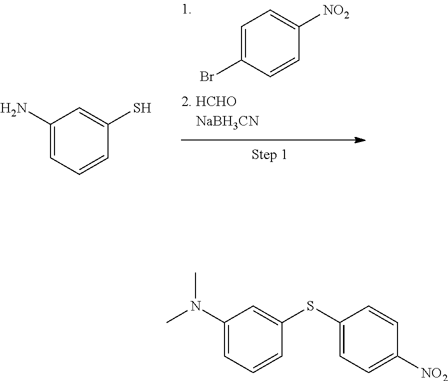 [SMILES: O=[N+]([O-])c1ccc(Br)cc1 ; Nc1cccc(S)c1 ; CN(C)c1cccc(Sc2ccc([N+](=O)[O-])cc2)c1]

3-Aminobenzenethiol (2 g, 16.0 mmol, 1 eq.) was added to a mixture of 4-bromonitrobenzene (3.5 g, 16.0 mmol, 1 eq.) and potassium carbonate (4.4 g, 32.0 mmol, 2 eq.) in DMF (30 mL). The reaction was stirred for 2 hours at rt. The mixture was poured into water and extracted with ethyl acetate three times. The organic extracts were combined, washed with brine, dried over anhydrous sodium sulfate, and concentrated. The residue was purified by silica gel column chromatography (petroleum ether/ethyl acetate=50:1 to 10:1) to give 3-(4-nitrophenylthio)aniline (2.74 g, 70% yield). Pos. LC-MS: 246.7 (M+H)⁺, C₁₂H₁₀N₂O₂S. ¹H NMR (DMSO-d6, 400 MHz) δ: 8.12 (d, J=8.4 Hz, 2H), 7.31 (d, J=8.4 Hz, 2H), 7.16 (m, 1H), 6.75 (s, 1H), 6.68 (m, 2H), 5.45 (br, 2H). 3-(4-Nitrophenylthio)aniline (1 g, 4.1 mmol, 1 eq.) was dissolved in acetonitrile (20 mL). Acetic acid (1 ml) and formaldehyde water solution (2.5 mL, 32.0 mmol, 8 eq.) were added. The solution was stirred for 10 min and NaBH₃CN (1.42 g, 20.0 mmol, 5 eq.) was added. The reaction was stirred for another 2 h. The mixture was diluted with water and extracted with ethyl acetate three times. The organic extracts were combined, washed with brine, dried over anhydrous sodium sulfate, and concentrated. The residue was purified by silica gel column chromatography (petroleum ether/ethyl acetate=200:1 to 100:1) to give N,N-dimethyl-3-(4-nitrophenylthio)aniline (340 mg, 31% yield). Pos. LC-MS: 274.7 (M+H)⁺, C₁₄H₁₄N₂O₂S.

### Step 2: N,N-dimethyl-3-(4-nitrophenylsulfonyl)aniline

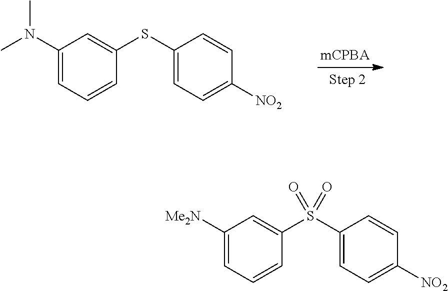 [SMILES: CN(C)c1cccc(Sc2ccc([N+](=O)[O-])cc2)c1 ; CN(C)c1cccc(S(=O)(=O)c2ccc([N+](=O)[O-])cc2)c1]

A mixture of N,N-dimethyl-3-(4-nitrophenylthio)aniline (340 mg, 1.24 mmol, 1 eq.) and mCPBA (917 mg, 3.72 mmol, 3 eq.) in dichloromethane (15 mL) was stirred overnight at rt. 4,4,4′,4′,5,5,5′,5′-octamethyl-2,2′-bi(1,3,2-dioxaborolane) (1.3 g, 4.96 mmol, 4 eq.) was added. And the reaction was stirred for another 30 min. The mixture was poured into sat. sodium bicarbonate and extracted with dichloromethane. The organic extracts were combined, washed with brine, dried over anhydrous sodium sulfate, and concentrated to give crude N,N-dimethyl-3-(4-nitrophenylsulfonyl)aniline (400 mg, quantitative yield), which was used for next reaction without further purification.

### Step 3: 3-(4-aminophenylsulfonyl)-N,N-dimethylaniline

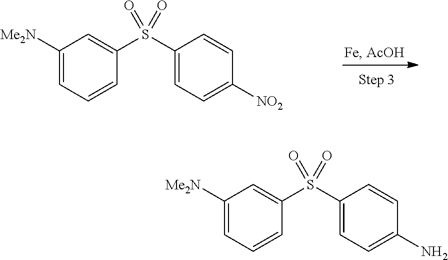 [SMILES: CN(C)c1cccc(S(=O)(=O)c2ccc([N+](=O)[O-])cc2)c1 ; CN(C)c1cccc(S(=O)(=O)c2ccc(N)cc2)c1]

N,N-dimethyl-3-(4-nitrophenylsulfonyl)aniline (400 mg, 1.3 mmol, 1 eq.) was dissolved in acetic acid (10 mL) and Fe (728 mg, 13.0 mmol, 10 eq.) was added. The reaction was heated at 60° C. for 2 h. The mixture was cooled to rt, diluted with ethyl acetate, filtered, and the cake was washed with ethyl acetate. The filtrate was washed with brine. The organic extract was concentrated and the residue was purified by silica gel column chromatography (petroleum ether/ethyl acetate=6:1 to 3:1) to give 3-(4-aminophenylsulfonyl)-N,N-dimethylaniline (240 mg, 67% yield). Pos. LC-MS: 276.9 (M+H)⁺, C₁₄H₁₆N₂O₂S.

### Step 4: 3-(4-isothiocyanatophenylsulfonyl)-N,N-dimethylaniline

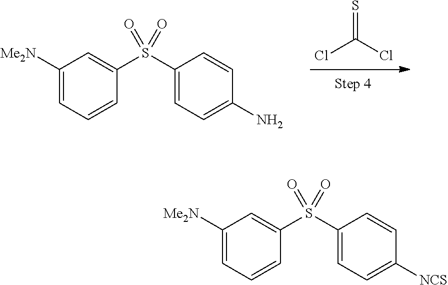

Thiophosgene (105 mg, 0.91 mmol, 1.1 eq.) was added to a mixture of 3-(4-aminophenylsulfonyl)-N,N-dimethylaniline (230 mg, 0.83 mmol, 1 eq.) and saturated sodium bicarbonate-water solution (10 mL) in chloroform (10 mL). The reaction was stirred for 2 h at rt under nitrogen protection. The mixture was extracted with dichloromethane twice. The organic extracts were combined, washed with brine, dried over anhydrous sodium sulfate, and concentrated to afford crude 3-(4-isothiocyanatophenylsulfonyl)-N,N-dimethylaniline (280 mg, quantitative yield), which was used for next reaction without further purification.

### Step 5: 4-(4-(3-(dimethylamino)phenylsulfonyl)phenyl)-1H-1,2,4-triazole-5(4H)-thione

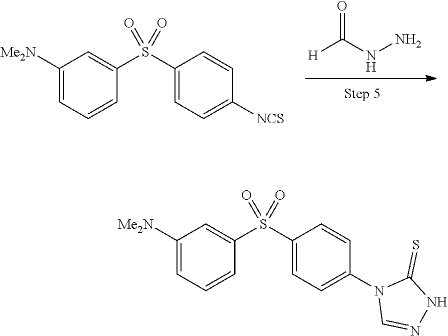

A solution of crude 3-(4-isothiocyanatophenylsulfonyl)-N,N-dimethylaniline (280 mg, 0.9 mmol, 1 eq.) and formohydrazide (54 mg, 0.9 mmol, 1 eq.) in ethanol (10 mL) was refluxed for 30 min. The solvent was removed and the residue was dissolve in 2% NaOH (10 mL). The reaction was heated at 100° C. for another 2 h. The mixture was cooled to rt and acidified to pH=3-4 by HCl. The resulting precipitate was extracted with dichloromethane two times. The organic extracts were combined, washed with brine, dried over anhydrous sodium sulfate, and concentrated. The residue was re-crystallized in ethanol to give 4-(4-(3-(dimethylamino)phenylsulfonyl)phenyl)-2,4-dihydro-3H-1,2,4-triazole-3-thione (30 mg, 9% yield). Pos. LC-MS: 360.70 (M+H)⁺, C₁₆H₁₆N₄O₂S₂. ¹H NMR (DMSO-d6, 400 MHz) δ: 14.08 (br, 1H), 8.77 (s, 1H), 8.16 (d, J=7.2 Hz, 2H), 7.95 (d, J=7.2 Hz, 2H), 7.40 (m, 1H), 7.19 (m, 2H), 6.99 (m, 1H), 2.97 (s, 6H).

### Example 14: 4-(4-((4-chloro-3-(trifluoromethyl)phenyl)sulfonyl)-2-(piperidin-1-yl)phenyl)-2,4-dihydro-3H-1,2,4-triazole-3-thione (Compound 19)

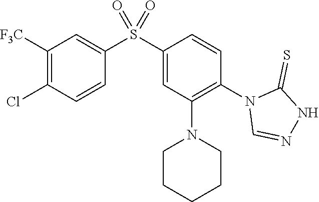 [SMILES: O=S(=O)(c1ccc(-n2cn[nH]c2=S)c(N2CCCCC2)c1)c1ccc(Cl)c(C(F)(F)F)c1]

### Step 1: Synthesis of 1-(5-bromo-2-nitrophenyl)piperidine

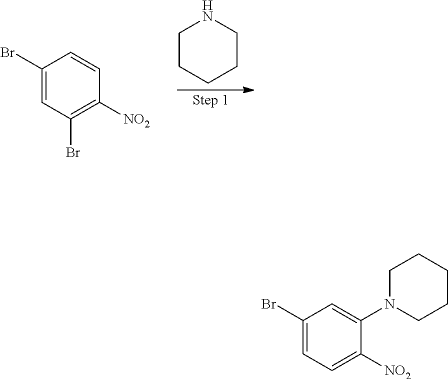 [SMILES: O=[N+]([O-])c1ccc(Br)cc1Br ; C1CCNCC1 ; O=[N+]([O-])c1ccc(Br)cc1N1CCCCC1]

A mixture of 2,4-dibromo-1-nitrobenzene (2.81 g, 10.0 mmol), piperidine (0.94 g, 11.0 mmol), and potassium carbonate (2.76 g, 20.0 mmol) in DMF (20 mL) was heated at 80° C. for 3 h. The mixture was cooled to rt, diluted with water (100 mL), and extracted with ethyl acetate (100 mL×3). The organic extracts were combined, washed with brine (50 mL×2), dried over anhydrous sodium sulfate, and concentrated. The residue was purified by silica gel column chromatography (petroleum ether/ethyl acetate=300:1 to 200:1) to give 1-(5-bromo-2-nitrophenyl)piperidine (2.2 g, 77% yield) as yellow solid. ¹H NMR (CDCl3, 400 MHz) δ: 7.66 (d, J=8.8 Hz, 1H), 7.23 (s, 1H), 7.05 (d, J=8.4 Hz, 1H), 3.03 (m, 4H), 1.71 (m, 4H), 1.62 (m, 2H).

### Step 2: Synthesis of 1-(2-Nitro-5-(4,4,5,5-tetramethyl-1,3,2-dioxaborolan-2-yl)phenyl)piperidine

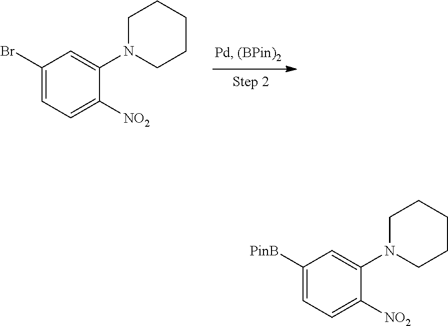

A mixture of 1-(5-bromo-2-nitrophenyl)piperidine (2.2 g, 7.8 mmol), 4,4,4′,4′,5,5,5′,5′-octamethyl-2,2′-bi(1,3,2-dioxaborolane) (1.97 g, 7.8 mmol), potassium acetate (2.23 g, 23.3 mmol), and PdCl₂(dppf) (0.63 g, 0.8 mmol) in dioxane (100 mL) was refluxed overnight. The mixture was cooled to rt, diluted with water (200 mL), and extracted with ethyl acetate (200 mL×3). The organic extracts were combined, washed with brine (50 mL×2), dried over anhydrous sodium sulfate, and concentrate. The residue was purified by silica gel column chromatography (petroleum ether/ethyl acetate=100:1 to 20:1) to give 1-(2-nitro-5-(4,4,5,5-tetramethyl-1,3,2-dioxaborolan-2-yl)phenyl)piperidine (1.1 g, 43% yield). Pos. LC-MS: 333.22 (M+H)⁺, C₁₇H₂₅BN₂O₄.

### Step 3: Synthesis of 1-(5-((4-chloro-3-(trifluoromethyl)phenyl)sulfonyl)-2-nitrophenyl)piperidine

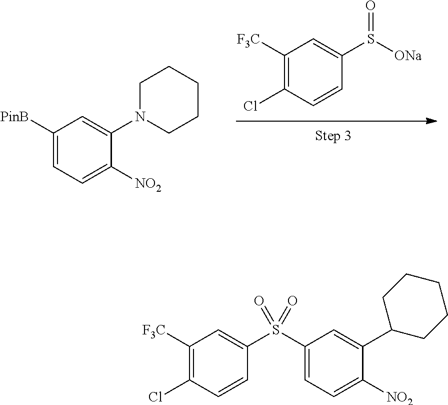

Potassium carbonate (828 mg, 6.0 mmol), 4A MS (2 g), and Cu(OAc)₂ (610 mg, 3.3 mmol) were added successively to a solution of compound 2 (1 g, 3.0 mmol) and sodium 4-chloro-3-(trifluoromethyl)benzenesulfinate (1.46 g, 6.0 mmol) in DMSO (25 mL). The reaction was stirred overnight at 60° C. in the presence of an oxygen balloon. The reaction mixture was poured into water (100 mL) and extracted with ethyl acetate (100 mL×3). The organic extracts were combined, washed with brine (50 mL×2), dried over anhydrous sodium sulfate, and concentrated. The residue was purified by silica gel column chromatography (petroleum ether/ethyl acetate=200:1 to 80:1) to 1-(5-((4-chloro-3-(trifluoromethyl)phenyl)sulfonyl)-2-nitrophenyl)piperidine (110 mg, 8% yield).

### Step 4: Synthesis of 4-(4-Chloro-3-(trifluoromethyl)phenylsulfonyl)-2-(piperidin-1-yl)aniline

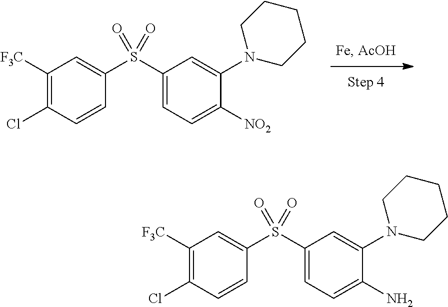 [SMILES: O=[N+]([O-])c1ccc(S(=O)(=O)c2ccc(Cl)c(C(F)(F)F)c2)cc1N1CCCCC1 ; Nc1ccc(S(=O)(=O)c2ccc(Cl)c(C(F)(F)F)c2)cc1N1CCCCC1]

1-(5-((4-chloro-3-(trifluoromethyl)phenyl)sulfonyl)-2-nitrophenyl)piperidine (110 mg, 0.24 mmol) was dissolved in acetic acid (10 mL) and Fe (137 mg, 2.4 mmol) was added. The reaction was heated at 60° C. for 2 h. The mixture was cooled to rt, diluted with ethyl acetate (30 mL), filtered, and the cake was washed with ethyl acetate (10 mL). The filtrate and wash were washed with brine (20 mL). The organic extract was concentrated and the residue was purified by silica gel column chromatography (petroleum ether/ethyl acetate=100:1 to 50:1) to give 4-(4-Chloro-3-(trifluoromethyl)phenylsulfonyl)-2-(piperidin-1-yl)aniline (100 mg, quantitative yield). LC-MS: 418.76 (M+H)⁺, C₁₈H₁₈ClF₃N₂O₂S.

### Step 5: Synthesis of 1-(5-(4-Chloro-3-(trifluoromethyl)phenylsulfonyl)-2-isothiocyanatophenyl)piperidine

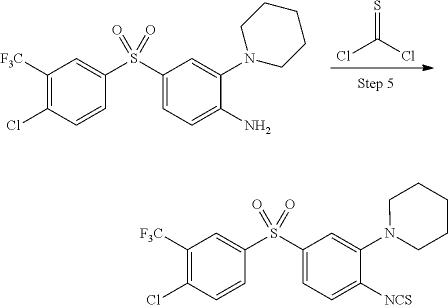

Thiophosgene (30 mg, 0.26 mmol) was added to a mixture of 4-(4-Chloro-3-(trifluoromethyl)phenylsulfonyl)-2-(piperidin-1-yl)aniline (100 mg, 0.24 mmol) and sat. sodium bicarbonate-water solution (10 mL) in chloroform (10 mL). The reaction was stirred for 2 h at rt under nitrogen protection. The mixture was extracted with dichloromethane (10 mL×2). The organic extracts were combined, washed with brine (10 mL), dried over anhydrous sodium sulfate, and concentrated to afford crude 1-(5-(4-chloro-3-(trifluoromethyl)phenylsulfonyl)-2-isothiocyanatophenyl)piperidine (80 mg, 67% yield), which was used for next reaction without further purification.

### Step 6: Synthesis of 4-(4-((4-chloro-3-(trifluoromethyl)phenyl)sulfonyl)-2-(piperidin-1-yl)phenyl)-2,4-dihydro-3H-1,2,4-triazole-3-thione

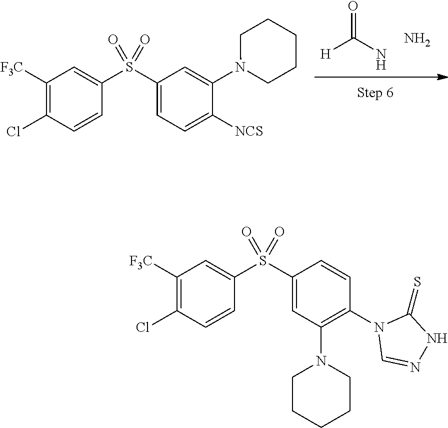

A solution of 1-(5-(4-chloro-3-(trifluoromethyl)phenylsulfonyl)-2-isothiocyanatophenyl)piperidine (80 mg, 0.17 mmol) and formohydrazide (10 mg, 0.17 mmol) in ethanol (10 mL) was refluxed for 30 min. The solvent was removed and the residue was dissolve in 2% NaOH. The reaction was heated at 100° C. for another 2 h. The mixture was cooled to rt and acidified to pH=3-4 by HCl. The resulting precipitate was extracted with dichloromethane for two times. The organic extracts were combined, washed with brine, dried over anhydrous sodium sulfate, and concentrated. The residue was re-crystallized in ethanol to give desired 4-(4-((4-chloro-3-(trifluoromethyl)phenyl)sulfonyl)-2-(piperidin-1-yl)phenyl)-2,4-dihydro-3H-1,2,4-triazole-3-thione (27 mg, 31% yield) as off-white solid. Pos. LC-MS: 502.88 (M+H)⁺, C₂₀H₁₈ClF₃N₄O₂S₂. ¹H NMR (DMSO-d6, 400 MHz) δ: 14.01 (br, 1H), 8.64 (s, 1H), 8.42 (m, 2H), 8.04 (d, J=8.0 Hz, 1H), 7.84 (d, J=8.4 Hz, 1H), 7.77 (s, 1H), 7.67 (d, J=8.4 Hz, 1H), 2.77 (m, 4H), 1.44 (m, 6H).

### Example 15: 4-(4-((4-chloro-3-(trifluoromethyl)phenyl)sulfonyl)-2-(4-methylpiperazin-1-yl)phenyl)-2,4-dihydro-3H-1,2,4-triazole-3-thione (Compound 20)

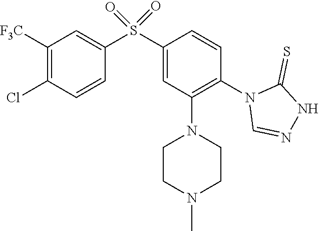 [SMILES: CN1CCN(c2cc(S(=O)(=O)c3ccc(Cl)c(C(F)(F)F)c3)ccc2-n2cn[nH]c2=S)CC1]

4-(4-(4-chloro-3-(trifluoro-methyl)-phenylsulfonyl)-2-(4-methyl-piperazin-1-yl)-phenyl)-3,4-dihydro-2H-1,2,4-triazole-3-thione was synthesized in a similar manner as described for Compound 19 in Example 14, 4-(4-((4-chloro-3-(trifluoromethyl)phenyl)sulfonyl)-2-(piperidin-1-yl)phenyl)-2,4-dihydro-3H-1,2,4-triazole-3-thione. Yield for Step 6: 14%, yellow solid. LC-MS: 517.9 (M+H)⁺, C₂₀H₁₉ClF₃N₅O₂S₂. ¹H NMR (DMSO-d6, 400 MHz) δ: 14.01. (bs, 1H), 8.62 (s, 1H), 8.43 (m, 2H), 8.04 (d, J=8.0 Hz, 1H), 7.87 (d, J=8.4 Hz, 1H), 7.80 (s, 1H), 7.69 (d, J=8.0 Hz, 1H), 2.81 (m, 4H), 2.28 (m, 4H), 2.16 (s, 3H).

### Example 16: 4-(4-((4-chloro-3-(trifluoromethyl)phenyl)sulfonyl)-2-(diethylamino)phenyl)-2,4-dihydro-3H-1,2,4-triazole-3-thione (Compound 21)

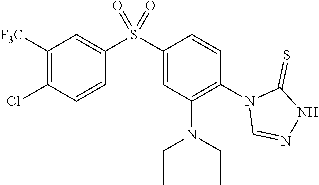 [SMILES: CCN(CC)c1cc(S(=O)(=O)c2ccc(Cl)c(C(F)(F)F)c2)ccc1-n1cn[nH]c1=S]

4-(4-((4-chloro-3-(trifluoromethyl)phenyl)sulfonyl)-2-(diethylamino)phenyl)-2,4-dihydro-3H-1,2,4-triazole-3-thione was synthesized in a similar manner as described for 4-(4-((4-chloro-3-(trifluoromethyl)phenyl)sulfonyl)-2-(piperidin-1-yl)phenyl)-2,4-dihydro-3H-1,2,4-triazole-3-thione. Yield for Step 6: 12%, off-white solid. LC-MS: 489.0 (M−H)⁻, C₁₉H₁₈ClF₃N₄O₂S₂. ¹H NMR (DMSO-d6, 300 MHz) δ: 14.01 (br, 1H), 8.47 (s, 1H), 8.39 (m, 2H), 8.05 (m, 1H), 7.77 (m, 2H), 7.62 (m, 1H), 2.93 (m, 4H), 0.84 (t, J=6.75 Hz, 6H).

### Example 17: 4-(4-((4-chloro-3-(trifluoromethyl)phenyl)sulfonyl)-2-(2-ethoxyethoxy)phenyl)-2,4-dihydro-3H-1,2,4-triazole-3-thione (Compound 22)

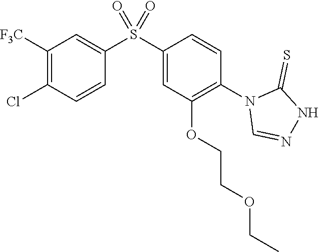 [SMILES: CCOCCOc1cc(S(=O)(=O)c2ccc(Cl)c(C(F)(F)F)c2)ccc1-n1cn[nH]c1=S]

4-(4-((4-Chloro-3-(trifluoromethyl)phenyl)sulfonyl)-2-(2-ethoxyethoxy)phenyl)-2,4-dihydro-3H-1,2,4-triazole-3-thione was synthesized in a similar manner as described for Compound 19 in Example 14, 4-(4-((4-chloro-3-(trifluoromethyl)phenyl)sulfonyl)-2-(piperidin-1-yl)phenyl)-2,4-dihydro-3H-1,2,4-triazole-3-thione. Yield for Step 6: 11%, white solid. LC-MS: 507.9 (M+H)⁺, C₁₉H₁₇ClF₃N₃O₄S₂. ¹H NMR (500 MHz, DMSO-d6) δ: 13.95 (s, 1H), 8.50 (d, J=1.7 Hz, 1H), 8.46-8.40 (m, 2H), 8.17-7.98 (m, 1H), 7.93 (d, J=1.7 Hz, 1H), 7.90-7.71 (m, 2H), 4.39-4.22 (m, 2H), 3.71-3.60 (m, 2H), 3.41 (q, J=7.0 Hz, 2H), 1.05 (t, J=7.0 Hz, 3H).

### Example 18: 4-(4-((4-chloro-3-(trifluoromethyl)phenyl)sulfonyl)phenyl)-3-(methylthio)-4H-1,2,4-triazole (Compound 26)

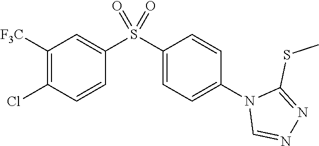 [SMILES: CSc1nncn1-c1ccc(S(=O)(=O)c2ccc(Cl)c(C(F)(F)F)c2)cc1]

A solution of 4-(4-((4-chloro-3-(trifluoromethyl)phenyl)sulfonyl)phenyl)-2,4-dihydro-3H-1,2,4-triazole-3-thione (209.9 mg, 0.5 mmol, 1 eq.), methyl iodide (37 μL, 0.6 mmol, 1.2 eq.), and potassium carbonate (70 mg, 0.5 mmol, 1 eq.) in DMF (5 mL) was heated at 100° C. for 1 h then cooled with stirring to room temperature for 2 h. The mixture was poured into water and extracted with dichloromethane three times. The organic extracts were combined, washed with brine, dried over anhydrous sodium sulfate, and concentrated. The residue was purified by silica gel column chromatography using a gradient of 0-10% 0.5 M NH₃ in MeOH/DCM to give 4-(4-((4-chloro-3-(trifluoromethyl)phenyl)sulfonyl)phenyl)-3-(methylthio)-4H-1,2,4-triazole. Yield: 77%, off-white solid. MS ES+: 434.6 (M+H)⁺, 456.6 (M⁺Na)⁺ C16H11ClF3N3O2S₂. ¹H NMR (500 MHz, DMSO-d₆) δ 8.92 (s, 1H), 8.38 (d, J=2.2 Hz, 1H), 8.35 (dd, J=8.4, 2.4 Hz, 1H), 8.33-8.27 (m, 2H), 8.04 (d, J=8.4 Hz, 1H), 7.85-7.79 (m, 2H), 2.63 (s, 3H).

### Example 19: 4-(4-(4-chloro-3-(trifluoromethyl)phenoxy)phenyl)-2,4-dihydro-3H-1,2,4-triazole-3-thione (Compound 30)

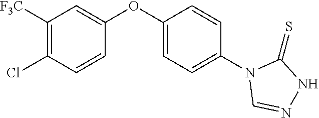 [SMILES: FC(F)(F)c1cc(Oc2ccc(-n3cn[nH]c3=S)cc2)ccc1Cl]

### Step 1: 1-Chloro-4-(4-nitrophenoxy)-2-(trifluoromethyl)benzene

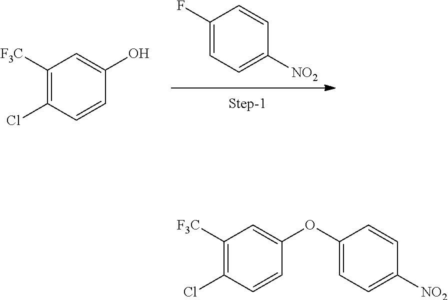 [SMILES: Oc1ccc(Cl)c(C(F)(F)F)c1 ; O=[N+]([O-])c1ccc(F)cc1 ; O=[N+]([O-])c1ccc(Oc2ccc(Cl)c(C(F)(F)F)c2)cc1]

A mixture of 4-chloro-3-(trifluoromethyl)phenol (0.98 g, 5 mmol.), 1-fluoro-4-nitrobenzene (1.06 g, 5.25 mmol), and potassium carbonate (1.38 g, 10 mmol) in DMF (20 mL) was heated for 2 h at 100° C. The mixture was cooled to rt and filtered. The filtrate was poured into water (100 mL) and extracted with ethyl acetate (100 mL×3). The organic extracts were combined, washed with brine (50 mL×2), dried over anhydrous sodium sulfate, and concentrated. The residue was purified by silica gel column chromatography (petroleum ether/ethyl acetate=300:1 to 100:1) to give 1-chloro-4-(4-nitrophenoxy)-2-(trifluoromethyl)benzene (1.4 g, 88% yield) as yellow solid.

### Step 2: 4-(4-Chloro-3-(trifluoromethyl)phenoxy)aniline

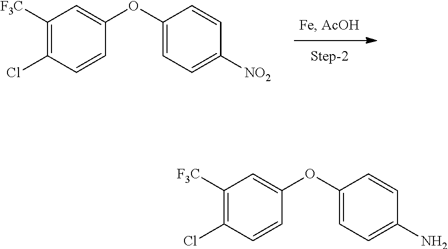 [SMILES: O=[N+]([O-])c1ccc(Oc2ccc(Cl)c(C(F)(F)F)c2)cc1 ; Nc1ccc(Oc2ccc(Cl)c(C(F)(F)F)c2)cc1]

1-chloro-4-(4-nitrophenoxy)-2-(trifluoromethyl)benzene (0.7 g, 2.21 mmol) was dissolved in acetic acid (10 mL) and Fe (1.24 g, 22.1 mmol) was added. The reaction was heated at 60° C. for 2 h. The mixture was cooled to rt, diluted with ethyl acetate, filtered, and the cake was washed with ethyl acetate. The filtrate was washed with brine. The organic extract was concentrated and the residue was purified by silica gel column chromatography (petroleum ether/ethyl acetate=300:1 to 100:1) to give 4-(4-chloro-3-(trifluoromethyl)phenoxy)aniline (510 mg, 80% yield).

### Step 3: 1-Chloro-4-(4-isothiocyanatophenoxy)-2-(trifluoromethyl)benzene

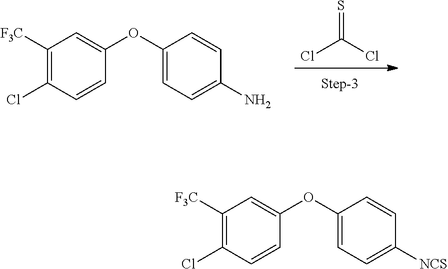

Thiophosgene (240 mg, 2.1 mmol) was added to a mixture of 4-(4-chloro-3-(trifluoromethyl)phenoxy)aniline (500 mg, 1.7 mmol) and saturated sodium bicarbonate-water solution (10 mL) in chloroform (10 mL). The reaction was stirred for 2 h at rt under nitrogen protection. The mixture was extracted with dichloromethane (10 mL×2). The organic extracts were combined, washed with brine (10 mL×2), dried over anhydrous sodium sulfate, and concentrated to afford crude 1-chloro-4-(4-isothiocyanatophenoxy)-2-(trifluoromethyl)benzene, which was used for next reaction without further purification.

### Step 4: 4-(4-(4-chloro-3-(trifluoromethyl)phenoxy)phenyl)-2,4-dihydro-3H-1,2,4-triazole-3-thione (Compound 30)

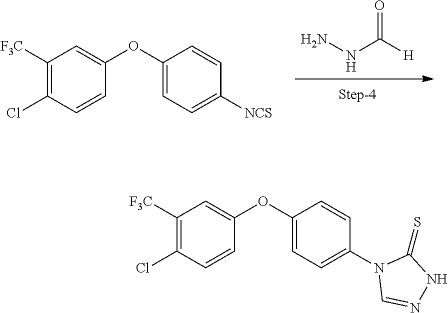

A solution of 1-chloro-4-(4-isothiocyanatophenoxy)-2-(trifluoromethyl)benzene (200 mg, 0.6 mmol) and formohydrazide (34 mg, 0.6 mmol) in ethanol (5 mL) was refluxed for 30 min. The solvent was removed and the residue was dissolve in 2% NaOH (5 mL). The reaction was heated at 100° C. for another 2 h. The mixture was cooled to rt and acidified to pH=3-4 by HCl. The resulting precipitate was extracted with dichloromethane for two times. The organic extracts were combined, washed with brine, dried over anhydrous sodium sulfate, and concentrated. The residue was re-crystallized in ethanol to give 4-(4-(4-chloro-3-(trifluoromethyl)phenoxy)phenyl)-2,4-dihydro-3H-1,2,4-triazole-3-thione (105 mg, 47% yield) as white solid. Neg. LC-MS: 370.1 (M−H)⁻, C₁₅H₉ClF₃N₃OS. ¹H NMR (DMSO-d6, 400 MHz) δ: 13.97 (br, 1H), 8.72 (s, 1H), 7.78 (d, J=8.8 Hz, 1H), 7.72 (d, J=8.8 Hz, 2H), 7.56 (s, 1H), 7.38 (m, 1H), 7.29 (d, J=8.4 Hz, 2H).

### Example 20: 1-(4-(4-chloro-3-(trifluoromethyl)phenylsulfonyl)phenyl)-1H-imidazole-2(3H)-thione (Compound 31)

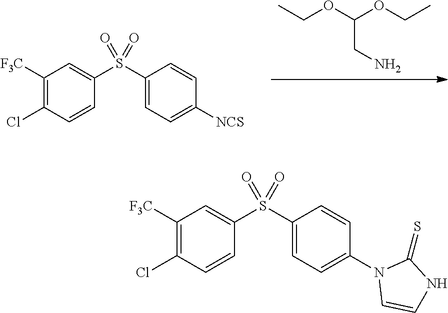

A solution of crude 1-chloro-4-(4-isothiocyanatophenylsulfonyl)-2-(trifluoromethyl)benzene (100 mg, 0.27 mmol, 1 eq.) and 2,2-diethoxyethanamine (36 mg, 0.27 mmol, 1 eq.) in ethanol (10 mL) was refluxed for 30 min. The solvent was removed and the residue was dissolve in acetic acid (10 mL) and sulfuric acid (0.5 mL). The reaction was heated at 120° C. for another 1 h. The mixture was cooled to rt, diluted with water (10 mL), and extracted with ethyl acetate (10 mL×3). The organic extracts were combined, washed with brine (10 mL), dried over anhydrous sodium sulfate, and concentrated. The residue was re-crystallized in ethanol to give 1-(4-(4-chloro-3-(trifluoromethyl)phenylsulfonyl)phenyl)-1H-imidazole-2(3H)-thione (40 mg, 0.10 mmol, 35% yield) as an off-white solid. Neg. LC-MS: 417.0 (M−H)⁻, C₁₆H₁₀ClF₃N₂O₂S₂. ¹H NMR (DMSO-d6, 300 MHz) δ: 12.57 (br, 1H), 8.36 (m, 2H), 8.22 (d, J=8.7 Hz, 2H), 8.03 (m, 3H), 7.42 (d, J=2.1 Hz, 1H), 7.14 (d, J=1.8 Hz, 1H).

### Example 21: 1-(4-((4-chloro-3-(trifluoromethyl)phenyl)sulfonyl)phenyl)-1,3-dihydro-2H-imidazol-2-one (Compound 32)

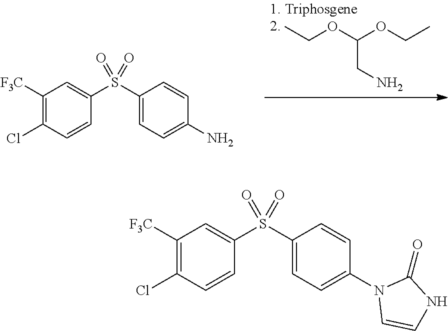

A solution of 4-(4-chloro-3-(trifluoromethyl)phenylsulfonyl)aniline (170 mg, 0.51 mmol, 1 eq.) in DCM (3 mL) was cooled to 0° C. TEA (125 mg, 1.22 mmol, 2.4 eq.) and triphosgene (301 mg, 1.02 mmol, 2 eq.) were added. The reaction was stirred for 30 min at 0° C. and then overnight at rt. 2,2-diethoxyethanamine (305 mg, 1.02 mmol, 2 eq.) was added and the reaction was stirred for another 12 h at rt. The solvent was removed and the residue was purified by prep-TLC (dichloromethane/methanol=15:1) to give 1-(4-((4-chloro-3-(trifluoromethyl)phenyl)sulfonyl)phenyl)-1,3-dihydro-2H-imidazol-2-one (30 mg, 0.07 mmol, 14% yield) as an off-white solid. Neg. LC-MS: 400.86 (M−H)⁻, C₁₆H₁₀ClF₃N₂O₃S. ¹H NMR (DMSO-d6, 300 MHz) δ: 10.52 (br, 1H), 8.28 (m, 2H), 8.09 (m, 4H), 8.00 (m, 1H), 7.14 (s, 1H), 6.70 (s, 1H).

### Example 22: N-(4-chloro-3-(trifluoromethyl)phenyl)-4-(5-thioxo-1H-1,2,4-triazol-4(5H)-yl)benzenesulfonamide (Compound 33)

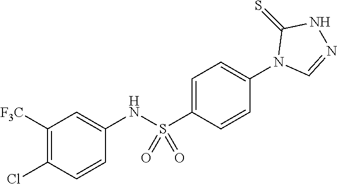 [SMILES: O=S(=O)(Nc1ccc(Cl)c(C(F)(F)F)c1)c1ccc(-n2cn[nH]c2=S)cc1]

### Step 1: N-(4-chloro-3-(trifluoromethyl)phenyl)-4-nitrobenzenesulfonamide

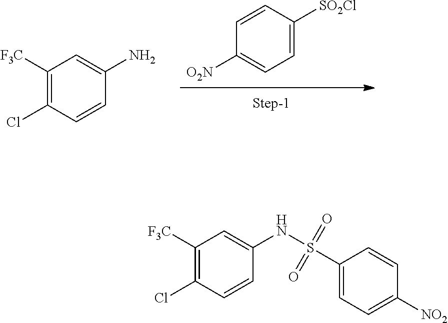 [SMILES: Nc1ccc(Cl)c(C(F)(F)F)c1 ; O=[N+]([O-])c1ccc(S(=O)(=O)Cl)cc1 ; O=[N+]([O-])c1ccc(S(=O)(=O)Nc2ccc(Cl)c(C(F)(F)F)c2)cc1]

Pyridine (4.03 g, 51.0 mmol, 2 eq.) was added to a solution of 4-chloro-3-(trifluoromethyl)aniline (5 g, 25.5 mmol, 1 eq.) and 4-nitrobenzene-1-sulfonyl chloride (6.77 g, 30.6 mmol, 1.2 eq.) in DCM (50 mL). The reaction was stirred overnight at rt. The reaction was quenched with water and extracted with dichloromethane three times. The organic extracts were combined, washed with brine, dried over anhydrous sodium sulfate, and concentrated. The resulting solid was washed with 5 mL DCM and the solid was filtered and dried to give N-(4-chloro-3-(trifluoromethyl)phenyl)-4-nitrobenzenesulfonamide (7.1 g, 53% yield) as a yellow solid. Neg. LC-MS: 378.72 (M−H)⁻, C₁₃H₈ClF₃N₂O₄S.

### Step 2: 4-amino-N-(4-chloro-3-(trifluoromethyl)phenyl)benzenesulfonamide

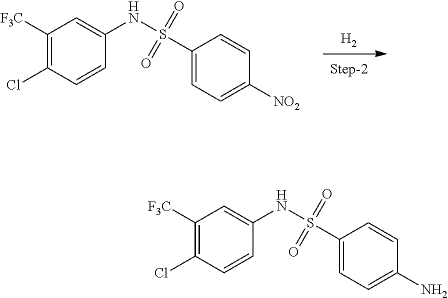 [SMILES: O=[N+]([O-])c1ccc(S(=O)(=O)Nc2ccc(Cl)c(C(F)(F)F)c2)cc1 ; "H2" ; Nc1ccc(S(=O)(=O)Nc2ccc(Cl)c(C(F)(F)F)c2)cc1]

A mixture of N-(4-chloro-3-(trifluoromethyl)phenyl)-4-nitrobenzenesulfonamide (2 g, 5.26 mmol, 1 eq.) and Pd/C (0.5 g) in MeOH (10 mL) was hydrogenated for 2 h. Pd/C was filtered off and the filtrate was concentrated in vacuo to give 4-amino-N-(4-chloro-3-(trifluoromethyl)phenyl)benzenesulfonamide (1.7 g, 92% yield). Neg. LC-MS: 348.85 (M−H)⁻, C₁₃H₁₀ClF₃N₂O₂S.

### Step 3: N-(4-chloro-3-(trifluoromethyl)phenyl)-4-isothiocyanatobenzenesulfonamide

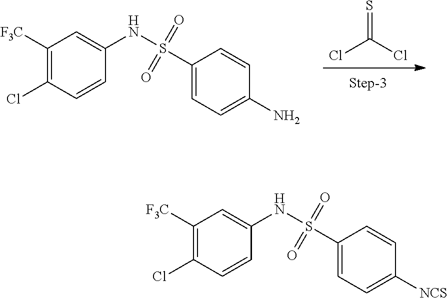

Thiophosgene (329 mg, 2.86 mmol, 2 eq.) was added to a mixture of 4-amino-N-(4-chloro-3-(trifluoromethyl)phenyl)benzenesulfonamide (500 mg, 1.43 mmol, 1 eq.) and saturated sodium bicarbonate-water solution (15 mL) in chloroform (15 mL). The reaction was stirred for 2 h at rt under nitrogen protection. The mixture was extracted with dichloromethane twice. The organic extracts were combined, washed with brine, dried over anhydrous sodium sulfate, and concentrated. The residue was purified by silica gel column chromatography (petroleum ether/ethyl acetate=100:1 to 10:1) to afford N-(4-chloro-3-(trifluoromethyl)phenyl)-4-isothiocyanatobenzenesulfonamide (503 mg, 90% yield).

### Step 4: N-(4-chloro-3-(trifluoromethyl)phenyl)-4-(5-thioxo-1H-1,2,4-triazol-4(5H)-yl)benzenesulfonamide

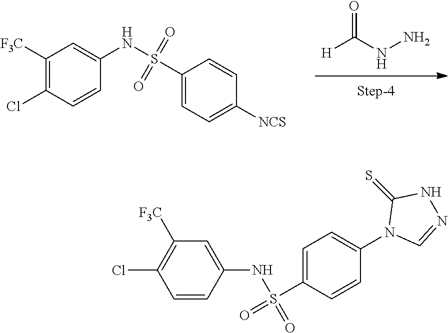

A solution of N-(4-chloro-3-(trifluoromethyl)phenyl)-4-isothiocyanatobenzenesulfonamide (200 mg, 0.51 mmol, 1 eq.) and formohydrazide (30.6 mg, 0.51 mmol, 1 eq.) in ethanol (10 mL) was refluxed for 30 min. The solvent was removed and the residue was dissolve in 2% NaOH (10 mL). The reaction was heated at 100° C. for another 2 h. The mixture was cooled to rt and acidified to pH=3-4 by HCl. The resulting precipitate was extracted with dichloromethane two times. The organic extracts were combined, washed with brine, dried over anhydrous sodium sulfate, and concentrated. The residue was purified by prep-TLC to give N-(4-chloro-3-(trifluoromethyl)phenyl)-4-(5-thioxo-1H-1,2,4-triazol-4(5H)-yl)benzenesulfonamide (62 mg, 0.14 mmol, 27% yield) as a white solid. Pos. LC-MS: 434.6 (M+H)⁺, C₁₅H₁₀ClF₃N₄O₂S₂. ¹H NMR (DMSO-d6, 400 MHz) δ: 14.05 (br, 1H), 11.09 (br, 1H), 8.77 (s, 1H), 7.97 (m, 4H), 7.64 (m, 1H), 7.52 (s, 1H), 7.43 (m, 1H).

### Example 23: 4-(4-((4-(trifluoromethyl)pyridin-2-yl)sulfonyl)phenyl)-2,4-dihydro-3H-1,2,4-triazole-3-thione (Compound 34)

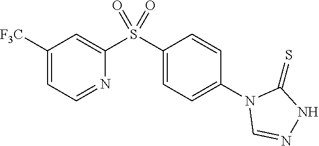 [SMILES: O=S(=O)(c1ccc(-n2cn[nH]c2=S)cc1)c1cc(C(F)(F)F)ccn1]

### Step 1: Synthesis of 2-(4-nitrophenylthio)-4-(trifluoromethyl)pyridine

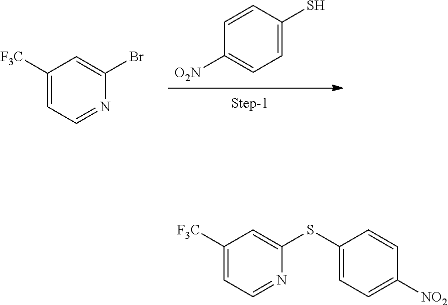 [SMILES: FC(F)(F)c1ccnc(Br)c1 ; O=[N+]([O-])c1ccc(S)cc1 ; O=[N+]([O-])c1ccc(Sc2cc(C(F)(F)F)ccn2)cc1]

A mixture of 2-bromo-4-(trifluoromethyl)pyridine (2 g, 8.85 mmol, 1 eq.), 4-nitrobenzenethiol (1.37 g, 8.85 mmol), and potassium carbonate (1.22 g, 8.85 mmol) in DMF (10 mL) was heated overnight at 100° C. The reaction was quenched with water and extracted with ethyl acetate three times. The organic extracts were combined, washed with brine, dried over anhydrous sodium sulfate, and concentrated. The residue was purified by silica gel column chromatography (petroleum ether/ethyl acetate=200:1 to 50:1) to give 2-(4-nitrophenylthio)-4-(trifluoromethyl)pyridine (2.3 g, 87% yield). Pos. LC-MS: 300.96 (M+H)⁺, C₁₂H₇F₃N₂O₂S.

### Step 2: Synthesis of 4-(4-(trifluoromethyl)pyridin-2-ylsulfonyl)aniline

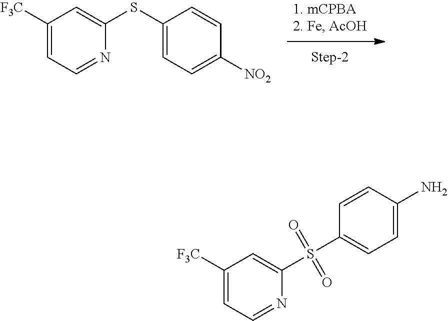 [SMILES: O=[N+]([O-])c1ccc(Sc2cc(C(F)(F)F)ccn2)cc1 ; Nc1ccc(S(=O)(=O)c2cc(C(F)(F)F)ccn2)cc1]

A mixture of 2-(4-nitrophenylthio)-4-(trifluoromethyl)pyridine (500 mg, 1.66 mmol, 1 eq.) and mCPBA (1.64 g, 6.67 mmol, 4 eq., 70% purity) in dichloromethane (16 mL) was stirred overnight at rt. Bis(pinacolato)diboron (1.7 g, 6.67 mmol, 4 eq.) was added. The reaction was stirred for another 30 min. The mixture was then poured into saturated sodium bicarbonate and extracted with dichloromethane. The organic extracts were combined, washed with brine, dried over anhydrous sodium sulfate, and concentrated. The residue was purified by silica gel column chromatography (petroleum ether/ethyl acetate=50:1 to 10:1) to give the nitro intermediate (550 mg, quantitative yield). The nitro intermediate (550 mg, 1.66 mmol, 1 eq.) was dissolved in acetic acid (15 mL) and Fe (928 mg, 16.6 mmol, 10 eq.) was added. The reaction was heated at 60° C. for 1 h. The mixture was cooled to rt, diluted with ethyl acetate, filtered, and the cake was washed with ethyl acetate. The volatile solvents were removed in vacuo and the water phase was neutralized to pH 7-8 with sodium bicarbonate. The resulting mixture was extracted with ethyl acetate for three times. The organic extracts were combined, washed with brine, dried over anhydrous sodium sulfate, and concentrated. The residue was purified by silica gel column chromatography (petroleum ether/ethyl acetate=20:1 to 5:1) to give 4-(4-(trifluoromethyl)pyridin-2-ylsulfonyl)aniline (310 mg, 62% yield). Pos. LC-MS: 302.8 (M+H)⁺, C₁₂H₉F₃N₂O₂S.

### Step 3: Synthesis of 2-(4-isothiocyanatophenylsulfonyl)-4-(trifluoromethyl)pyridine

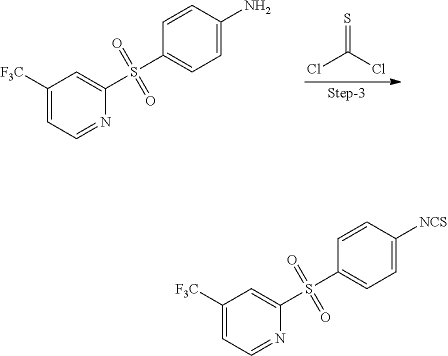

Thiophosgene (381 mg, 3.31 mmol, 2 eq.) was added to a mixture of 4-(4-(trifluoromethyl)pyridin-2-ylsulfonyl)aniline (500 mg, 1.66 mmol, 1 eq.) and saturated sodium bicarbonate-water solution (5 mL) in chloroform (5 mL). The reaction was stirred for 2 h at rt under nitrogen protection. The mixture was extracted with dichloromethane twice. The organic extracts were combined, washed with brine, dried over anhydrous sodium sulfate, and concentrated to afford crude 2-(4-isothiocyanatophenylsulfonyl)-4-(trifluoromethyl)pyridine (570 mg, quantitative yield).

### Step 4: Synthesis of 4-(4-((4-(trifluoromethyl)pyridin-2-yl)sulfonyl)phenyl)-2,4-dihydro-3H-1,2,4-triazole-3-thione

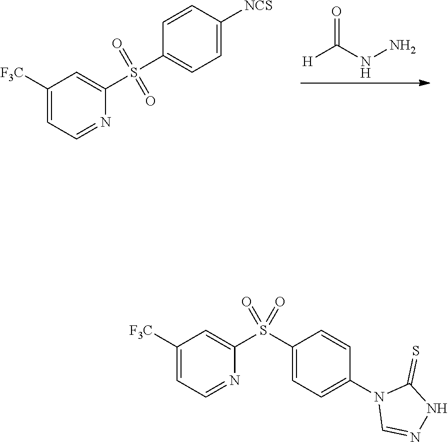

A solution of 2-(4-isothiocyanatophenylsulfonyl)-4-(trifluoromethyl)pyridine (300 mg, 0.87 mmol, 1 eq.), formohydrazide (52 mg, 0.87 mmol, 1 eq.), and TEA (264 mg, 2.62 mmol, 3 eq.) in ethanol (5 mL) was refluxed for 2 h. The solvent was removed in vacuo and the residue was diluted with water. The mixture was extracted with ethyl acetate three times. The organic extracts were combined, washed with brine, dried over anhydrous sodium sulfate, and concentrated. The residue was purified by silica gel column chromatography (DCM/MeOH=100:1) to give 4-(4-((4-(trifluoromethyl)pyridin-2-yl)sulfonyl)phenyl)-2,4-dihydro-3H-1,2,4-triazole-3-thione (110 mg, 0.14 mmol, 33% yield) as an off-white solid. Pos. LC-MS: 387.1 (M+H)⁺, C₁₄H₉F₃N₄O₂S₂. ¹H NMR (DMSO-d6, 400 MHz) δ: 14.11 (br, 1H), 9.04 (d, J=4.8 Hz, 1H), 8.81 (s, 1H), 8.55 (s, 1H), 8.18 (m, 3H), 8.04 (m, 2H).

### Example 24: 4-(4-(2-(trifluoromethyl)pyridin-4-ylsulfonyl)phenyl)-2,4-dihydro-3H-1,2,4-triazole-3-thione (Compound 35)

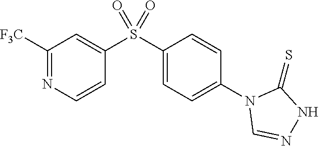 [SMILES: O=S(=O)(c1ccc(-n2cn[nH]c2=S)cc1)c1ccnc(C(F)(F)F)c1]

4-(4-((2-(trifluoromethyl)pyridin-4-yl)sulfonyl)phenyl)-2,4-dihydro-3H-1,2,4-triazole-3-thione was synthesized in a similar manner as described for Compound 34 of Example 23, 4-(4-(4-(trifluoromethyl)pyridin-2-ylsulfonyl)phenyl)-2,4-dihydro-3H-1,2,4-triazole-3-thione. Yield for final step: 51%, light yellow solid. Pos. LC-MS: 387.1 (M+H)⁺, C₁₄H₉F₃N₄O₂S₂. ¹H NMR (DMSO-d6, 400 MHz) δ: ¹H NMR (DMSO-d6, 400 MHz) δ: 14.11 (br, 1H), 9.12 (s, 1H), 8.80 (s, 1H), 8.47 (s, 1H), 8.35 (m, 3H), 8.07 (m, 2H).

### Example 25: 4-(5-(4-chloro-3-(trifluoromethyl)phenylsulfonyl)pyridin-2-yl)-2,4-dihydro-3H-1,2,4-triazole-3-thione (Compound 36)

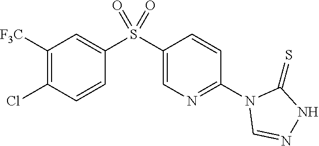 [SMILES: O=S(=O)(c1ccc(-n2cn[nH]c2=S)nc1)c1ccc(Cl)c(C(F)(F)F)c1]

### Step 1: 5-(4-chloro-3-(trifluoromethyl)phenylthio)-2-nitropyridine

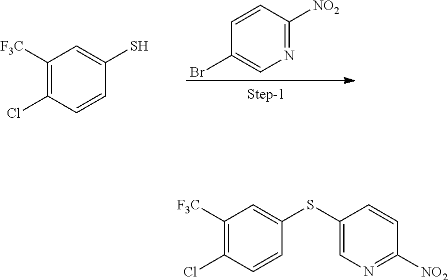 [SMILES: FC(F)(F)c1cc(S)ccc1Cl ; O=[N+]([O-])c1ccc(Br)cn1 ; O=[N+]([O-])c1ccc(Sc2ccc(Cl)c(C(F)(F)F)c2)cn1]

A solution of 4-chloro-3-(trifluoromethyl)benzenethiol (2.22 g, 10.0 mmol, 1 eq.) in THF (100 mL) was cooled to 0° C. before tBuOK (2.24 g, 20.0 mmol, 2 eq.) was added. The reaction was stirred for 30 min at 0° C. followed by addition of 5-bromo-2-nitropyridine (2.03 g, 10.0 mmol, 1 eq.). The reaction was then stirred at rt for 2 h. The reaction was quenched with sat. NH₄Cl solution and extracted with ethyl acetate three times. The organic extracts were combined, washed with brine, dried over anhydrous sodium sulfate, and concentrated. The residue was purified by silica gel column chromatography (petroleum ether/ethyl acetate=500:1 to 100:1) to give 5-(4-chloro-3-(trifluoromethyl)phenylthio)-2-nitropyridine (1.6 g, 48% yield). Pos. LC-MS: 334.74 (M+H)⁺, C₁₂H₆ClF₃N₂O₂S.

### Step 2: 5-(4-chloro-3-(trifluoromethyl)phenylsulfonyl)pyridin-2-amine

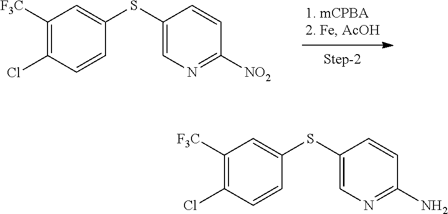 [SMILES: O=[N+]([O-])c1ccc(Sc2ccc(Cl)c(C(F)(F)F)c2)cn1 ; Nc1ccc(Sc2ccc(Cl)c(C(F)(F)F)c2)cn1]

A mixture of 5-(4-chloro-3-(trifluoromethyl)phenylthio)-2-nitropyridine (1.6 g, 4.78 mmol, 1 eq.) and mCPBA (3.54 g, 14.34 mmol, 3 eq., 70% purity) in dichloromethane (16 mL) was stirred overnight at rt. Bis(pinacolato)diboron (9.7 g, 38.24 mmol, 8 eq.) was added. The reaction was stirred for another 30 min. The mixture was then poured into saturated sodium bicarbonate and extracted with dichloromethane three times. The organic extracts were combined, washed with brine, dried over anhydrous sodium sulfate, and concentrated. The residue was purified by silica gel column chromatography (petroleum ether/ethyl acetate=100:1 to 20:1) to give 5-(4-chloro-3-(trifluoromethyl)phenylsulfonyl)-2-nitropyridine (710 mg, 40% yield). The 5-(4-Chloro-3-(trifluoromethyl)phenylsulfonyl)-2-nitropyridine (700 mg, 1.91 mmol, 1 eq.) was dissolved in acetic acid (15 mL) and Fe (1.07 g, 19.1 mmol, 10 eq.) was added. The reaction was heated at 60° C. for 3 h. The mixture was cooled to rt, diluted with ethyl acetate, filtered, and the cake was washed with ethyl acetate. The volatile solvents were removed in vacuo and the water phase was neutralized to pH 7-8 with sodium bicarbonate. The resulting mixture was extracted with ethyl acetate for three times. The organic extracts were combined, washed with brine, dried over anhydrous sodium sulfate, and concentrated to give 5-(4-chloro-3-(trifluoromethyl)phenylsulfonyl)pyridin-2-amine (503 mg, 78% yield).

### Step 3: 5-(4-chloro-3-(trifluoromethyl)phenylsulfonyl)-2-isothiocyanatopyridine

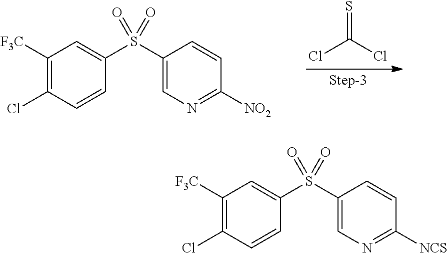

Thiophosgene (140 mg, 1.22 mmol, 1.1 eq.) was added to a mixture of 5-(4-chloro-3-(trifluoromethyl)phenylsulfonyl)pyridin-2-amine (370 mg, 1.10 mmol, 1 eq.) and saturated sodium bicarbonate-water solution (10 mL) in chloroform (10 mL). The reaction was stirred overnight at rt under nitrogen protection. The mixture was extracted with dichloromethane twice. The organic extracts were combined, washed with brine, dried over anhydrous sodium sulfate, and concentrated to afford crude 5-(4-chloro-3-(trifluoromethyl)phenylsulfonyl)-2-isothiocyanatopyridine, which was used for next reaction without further purification.

### Step 4: 4-(5-(4-chloro-3-(trifluoromethyl)phenylsulfonyl)pyridin-2-yl)-2,4-dihydro-3H-1,2,4-triazole-3-thione (Compound 36)

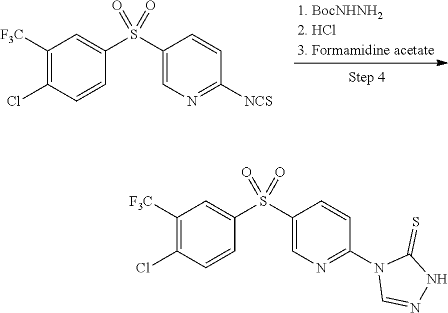

A solution of crude 5-(4-chloro-3-(trifluoromethyl)phenylsulfonyl)-2-isothiocyanatopyridine (200 mg, 0.53 mmol, 1 eq.), Boc-hydrazine (140 mg, 1.07 mmol, 2 eq.) and TEA (150 mg, 1.33 mmol, 2.5 eq.) in THF (5 mL) was refluxed for 1 h. The solvent was removed to give crude intermediate, which was treated with a dry 6 N HCl (gas)/ethyl acetate solution (10 mL) for 30 min at rt. The solvent was removed and co-evaporated with dichloromethane for two times to give hydrazine urea intermediate HCl salt (˜200 mg). The hydrazine urea intermediate HCl salt (˜200 mg) was dissolved in DMF (2 mL). Formamidine acetate (140 mg, 1.35 mmol, 3 eq.) and acetic acid (80 mg, 1.35 mmol, 3 eq.) were added. The reaction was heated at 80° C. for 1 h. The solution was cooled to rt, diluted with water, and extracted with ethyl acetate three times. The organic extracts were combined, washed with brine, dried over anhydrous sodium sulfate, and concentrated. The residue was purified by silica gel column chromatography (petroleum ether/ethyl acetate/dichloromethane=20:1:1 to 2:1:1) to give 4-(5-(4-chloro-3-(trifluoromethyl)phenylsulfonyl)pyridin-2-yl)-2,4-dihydro-3H-1,2,4-triazole-3-thione (30 mg, 0.07 mmol, 16% yield) as a light yellow solid. Pos. LC-MS: 420.8 (M+H)⁺, C₁₄H₈ClF₃N₄O₂S₂. ¹H NMR (DMSO-d6, 400 MHz) δ: 14.21 (br, 1H), 9.28 (s, 1H), 8.98 (d, J=8.8 Hz, 2H), 8.78 (d, J=8.8 Hz, 1H), 8.41 (m, 2H), 8.06 (d, J=8.4 Hz, 1H).

### Example 26: 4-(4-(4-chloro-3-(trifluoromethyl)phenylsulfonyl)phenyl)-1H-1,2,4-triazol-5(4H)-one (Compound 37)

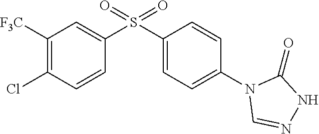 [SMILES: O=c1[nH]ncn1-c1ccc(S(=O)(=O)c2ccc(Cl)c(C(F)(F)F)c2)cc1]

### Step 1: Tert-butyl 2-(4-(4-chloro-3-(trifluoromethyl)phenylsulfonyl)phenylcarbamoyl)hydrazinecarboxylate

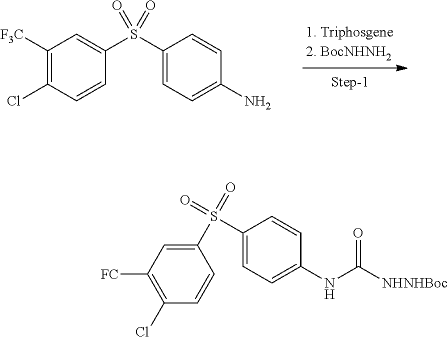

Triphosgene (470 mg, 1.58 mmol, 2 eq.) was added to a solution of 4-(4-chloro-3-(trifluoromethyl)phenylsulfonyl)aniline (250 mg, 0.75 mmol, 1 eq.) and TEA (240 mg, 2.38 mmol, 3 eq.) in DCM (20 mL). The reaction was stirred for 1 h at rt under nitrogen protection. The solvent was removed. The residue was dissolved in THF (5 mL) and Boc-hydrazine (213 mg, 1.50 mmol, 2 eq.) was added. The reaction was refluxed for 1 h. The solvent was removed in vacuo to give crude tert-butyl 2-(4-(4-chloro-3-(trifluoromethyl)phenylsulfonyl)phenylcarbamoyl)hydrazinecarboxylate, which was used for next reaction without further purification.

### Step 2: 4-(4-(4-chloro-3-(trifluoromethyl)phenylsulfonyl)phenyl)-1H-1,2,4-triazol-5(4H)-one (Compound 37)

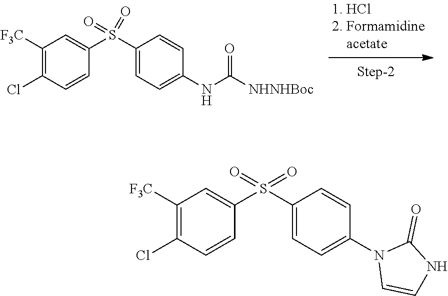

A solution of crude tert-butyl 2-(4-(4-chloro-3-(trifluoromethyl)phenylsulfonyl)phenylcarbamoyl) hydrazinecarboxylate (˜380 mg, 0.75 mmol, 1 eq.) in DCM (3 mL) was treated with a dry 6 N HCl (gas)/ethyl acetate solution (5 mL) for 1 h at rt. The solvent was removed in vacuo. The residue was dissolved in DMF (2 mL). Formamidine acetate (234 mg, 2.25 mmol, 3 eq.) and acetic acid (135 mg, 2.25 mmol, 3 eq.) were added. The reaction was heated at 80° C. for 1 h. The solution was cooled to rt, diluted with water, and extracted with ethyl acetate three times. The organic extracts were combined, washed with brine, dried over anhydrous sodium sulfate, and concentrated. The residue was purified by silica gel column chromatography (petroleum ether/ethyl acetate/dichloromethane=10:1:1 to 1:1:1) to give 4-(4-(4-chloro-3-(trifluoromethyl)phenylsulfonyl)phenyl)-1H-1,2,4-triazol-5(4H)-one (20 mg, 0.07 mmol, 7% yield) as a white solid. Pos. LC-MS: 403.8 (M+H)⁺, C₁₅H₉ClF₃N₃O₃S. ¹H NMR (DMSO-d6, 400 MHz) δ: 12.16 (br, 1H), 8.54 (s, 1H), 8.32 (m, 2H), 8.21 (d, J=9.2 Hz, 2H), 8.03 (m, 3H).

### Example 27: (4-chloro-3-(trifluoromethyl)phenyl)(4-(5-thioxo-1H-1,2,4-triazol-4(5H)-yl)phenyl)methanone (Compound 38)

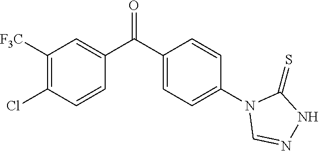 [SMILES: O=C(c1ccc(-n2cn[nH]c2=S)cc1)c1ccc(Cl)c(C(F)(F)F)c1]

### Step 1: Synthesis of tert-butyl 4-(4-chloro-3-(trifluoromethyl)benzoyl)phenylcarbamate

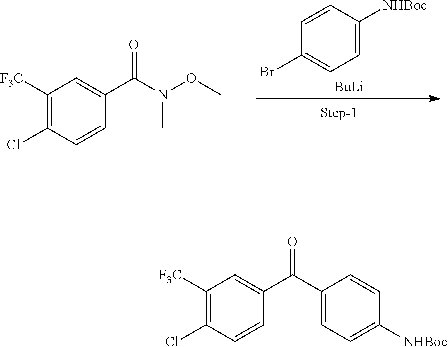

BuLi (2.90 mL, 1.6 M in hexane/THF, 4.65 mmol, 2.5 eq.) was added to a solution of tert-butyl 4-bromophenylcarbamate (0.5 g, 1.86 mmol, 1 eq.) in THF (10 mL) at −40° C. The reaction was stirred for 1 hour at −40° C. A solution of 4-chloro-N-methoxy-N-methyl-3-(trifluoromethyl)benzamide (0.49 g, 1.86 mmol, 1 eq.) in THF (3 mL) was added. The reaction was stirred for 1 h at rt. The solution was quenched with saturated ammonium chloride and extracted with ethyl acetate three times. The organic extracts were combined, washed with brine, dried over anhydrous sodium sulfate, and concentrated. The residue was purified by silica gel column chromatography (petroleum ether/ethyl acetate=100:1 to 10:1) to give tert-butyl 4-(4-chloro-3-(trifluoromethyl)benzoyl)phenylcarbamate as a white solid (0.51 g, 1.28 mmol, 68% yield).

### Step 2: Synthesis of (4-chloro-3-(trifluoromethyl)phenyl)(4-isothiocyanatophenyl)methanone

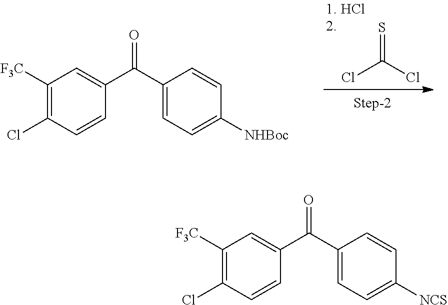

A solution of tert-butyl 4-(4-chloro-3-(trifluoromethyl)benzoyl)phenylcarbamate (0.5 g, 1.28 mmol, 1 eq.) in 6 N HCl(gas)/EA (10 mL) was stirred for 1 h. The solvent was removed and the residue was co-evaporated with DCM twice. The resulting amine (0.3 g, 1.00 mmol, 1 eq.) was dissolved in a mixture of saturated sodium bicarbonate-water solution (5 mL) and chloroform (5 mL). Thiophosgene (127 mg, 1.10 mmol, 1.1 eq.) was added. The reaction was stirred for 2 h at rt under nitrogen protection. The mixture was extracted with dichloromethane twice. The organic extracts were combined, washed with brine, dried over anhydrous sodium sulfate, and concentrated to afford crude (4-chloro-3-(trifluoromethyl)phenyl)(4-isothiocyanatophenyl)methanone, which was used for next reaction without further purification.

### Step 3: Synthesis of (4-chloro-3-(trifluoromethyl)phenyl)(4-(5-thioxo-1H-1,2,4-triazol-4(5H)-yl)phenyl)methanone

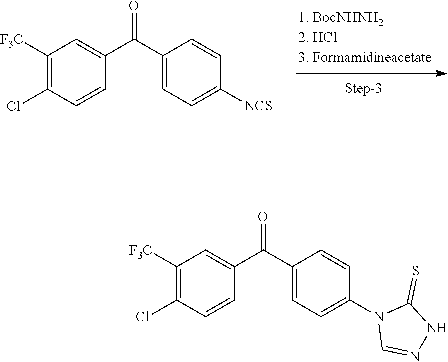

A solution of crude (4-chloro-3-(trifluoromethyl)phenyl)(4-isothiocyanatophenyl)methanone (0.2 g, 0.59 mmol, 1 eq.) and BocNHNH₂ (155 mg, 1.18 mmol, 2 eq.) in THF (6 mL) was refluxed for 40 min. The solvent was removed and the residue was suspended in sat. NaHCO₃ (5 mL). The resulting mixture was extracted with ethyl acetate three times. The organic extracts were combined, washed with brined, dried over anhydrous sodium sulfate, and concentrated. The residue was washed with a combination of petroleum ether/ethyl acetate (20:1) and then treated with 6 N HCl(gas)/ethyl acetate for 30 min and concentrated to give hydrazine urea. The urea was mixed with formamidineacetate (184 mg, 1.77 mmol, 3.0 eq.) and acetic acid (110 mg, 1.77 mmol, 3 eq.) in DMF (2.5 mL) and then was heated at 85° C. for 1 h. The solution was cooled to rt, diluted with saturated sodium bicarbonate, and extracted with ethyl acetate three times. The organic extracts were combined, washed with brine, dried over anhydrous sodium sulfate, and concentrated. The residue was purified by silica gel column chromatography with a gradient of petroleum ether/ethyl acetate (20:1 to 3:1) to give (4-chloro-3-(trifluoromethyl)phenyl)(4-(5-thioxo-1H-1,2,4-triazol-4(5H)-yl)phenyl)methanone (120 mg, 0.31 mmol, 53% yield) as a white solid. Neg. LC-MS: 382.0 (M−H)⁻, C₁₆H₉ClF₃N₃OS. ¹H NMR (DMSO-d6, 400 MHz) δ: 14.09 (br, 1H), 8.85 (s, 1H), 8.14 (s, 1H), 8.04 (m, 1H), 7.95 (m, 5H).

### Example 28: 4-(4-((4-chloro-3-(trifluoromethyl)phenyl)difluoromethyl)phenyl)-2,4-dihydro-3H-1,2,4-triazole-3-thione (Compound 40)

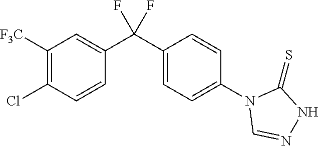 [SMILES: FC(F)(F)c1cc(C(F)(F)c2ccc(-n3cn[nH]c3=S)cc2)ccc1Cl]

### Step 1: Synthesis of (4-aminophenyl)(4-chloro-3-(trifluoromethyl)phenyl)methanone

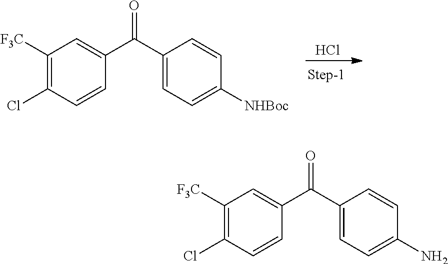 [SMILES: CC(C)(C)OC(=O)Nc1ccc(C(=O)c2ccc(Cl)c(C(F)(F)F)c2)cc1 ; Cl ; Nc1ccc(C(=O)c2ccc(Cl)c(C(F)(F)F)c2)cc1]

6 N HCl(gas)/dioxane (5 mL) was added to a solution of tert-butyl-4-(4-chloro-3-(trifluoromethyl) benzoyl)phenylcarbamate (500 mg, 1.25 mmol, 1 eq.) in dioxane (10 mL). The reaction was stirred for 30 min and the solvent was removed in vacuo to give (4-aminophenyl)(4-chloro-3-(trifluoromethyl) phenyl)methanone (380 mg, quantitative yield).

### Step 2: Synthesis of 4-(2-(4-chloro-3-(trifluoromethyl)phenyl)-1,3-dithian-2-yl)aniline

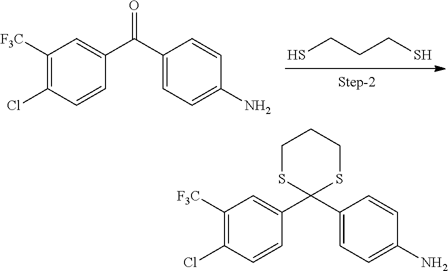 [SMILES: Nc1ccc(C(=O)c2ccc(Cl)c(C(F)(F)F)c2)cc1 ; SCCCS ; Nc1ccc(C2(c3ccc(Cl)c(C(F)(F)F)c3)SCCCS2)cc1]

BF₃.OEt₂ (284 mg, 2.0 mmol, 1.6 eq.) was added to a solution of crude (4-aminophenyl)(4-chloro-3-(trifluoromethyl)phenyl)methanone (380 mg, 1.25 mmol, 1 eq.) and propane-1,3-dithiol (203 mg, 1.88 mmol, 1.5 eq.) in dichloromethane (10 mL). The reaction was stirred overnight at rt. The solution was poured into saturated sodium bicarbonate and extracted with dichloromethane. The organic extracts were combined, washed with brine, dried over anhydrous sodium sulfate, and concentrated. The residue was purified by silica gel column chromatography (petroleum ether/ethyl acetate=50:1 to 10:1) to give 4-(2-(4-chloro-3-(trifluoromethyl)phenyl)-1,3-dithian-2-yl)aniline (370 mg, 76% yield). LC-MS: 390.1 (M+H)⁺, C₁₇H₁₅ClF₃NS₂.

### Step 3: Synthesis of 1-chloro-4-(difluoro(4-isothiocyanatophenyl)methyl)-2-(trifluoromethyl)benzene

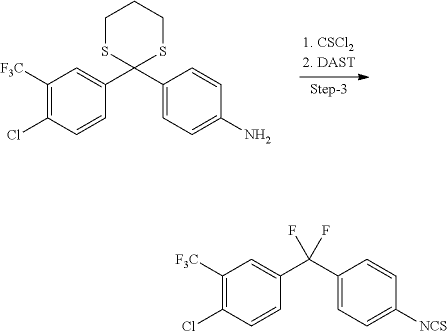

Thiophosgene (218.8 mg, 1.90 mmol, 2 eq.) was added to a mixture of 4-(4-(trifluoromethyl)pyridin-2-ylsulfonyl)aniline (370 mg, 0.95 mmol, 1 eq.) and saturated sodium bicarbonate-water solution (5 mL) in chloroform (5 mL). The reaction was stirred for 2 h at rt under nitrogen protection. The mixture was extracted with dichloromethane twice. The organic extracts were combined, washed with brine, dried over anhydrous sodium sulfate, and concentrated. The residue was purified by silica gel column chromatography (petroleum ether/ethyl acetate=100:1) to afford intermediate (310 mg, 76% yield). The intermediate (250 mg, 0.58 mmol, 1 eq.) was dissolved in DCM (5 mL) and DAST (234 mg, 1.45 mmol, 2.5 eq.) was added. The reaction was stirred for 5 h at rt. The mixture was poured into saturated sodium bicarbonate and extracted with DCM for three times. The organic extracts were combined, washed with brine, dried over anhydrous sodium sulfate, and concentrated at low temperature to give crude 1-chloro-4-(difluoro(4-isothiocyanatophenyl)methyl)-2-(trifluoromethyl)benzene (260 mg, quantitative yield).

### Step 4: Synthesis of 4-(4-((4-chloro-3-(trifluoromethyl)phenyl)difluoromethyl)phenyl)-2,4-dihydro-3H-1,2,4-triazole-3-thione

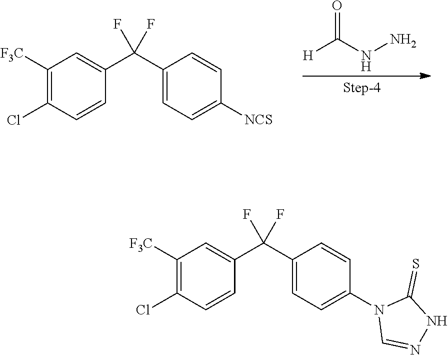

A solution of crude 1-chloro-4-(difluoro(4-isothiocyanatophenyl)methyl)-2-(trifluoromethyl)benzene (210 mg, 0.58 mmol, 1 eq.), formohydrazide (35 mg, 0.58 mmol, 1 eq.), and TEA (175 mg, 1.74 mmol, 3 eq.) in ethanol (5 mL) was refluxed for 2 h. The solvent was removed in vacuo, and the residue was diluted with water. The mixture was extracted with ethyl acetate three times. The organic extracts were combined, washed with brine, dried over anhydrous sodium sulfate, and concentrated. The residue was purified by silica gel column chromatography (petroleum ether/ethyl acetate=10:1 to 5:1) to give 4-(4-((4-chloro-3-(trifluoromethyl)phenyl)difluoromethyl)phenyl)-2,4-dihydro-3H-1,2,4-triazole-3-thione (43 mg, 0.11 mmol, 18% yield) as a light yellow solid. Pos. LC-MS: 406.1 (M+H)⁺, C₁₆H₉ClF₅N₃S. ¹H NMR (DMSO-d6, 400 MHz) δ: 14.03 (br, 1H), 8.75 (s, 1H), 8.03 (m, 1H), 7.94 (m, 2H), 7.84 (m, 4H).

In certain instances, the above processes further involving the step of forming a salt of a compound of the present disclosure. Embodiments are directed to the other processes described herein; and to the product prepared by any of the processes described herein.

In certain instances, the above processes further involving the step of forming a salt, including a pharmaceutically acceptable salt, of a compound of the present disclosure. Salt forms may be prepared using standard salt formation procedures known in the art. Embodiments are directed to the other processes described herein; and to the product prepared by any of the processes described herein.

### Example 29: Spray Dry Formulations

Formulations of Compound 1 were prepared using spray dry methods. Four spray solutions containing different polymers at a 3:1 polymer:compound ratio were prepared and sprayed onto a Buchi B-290 lab scale spray dryer. A summary of the spray parameters and results is shown in Table 3.

TABLE 3

| SDD | Polymer: | % | Approx. Flow | Average Outlet | Total Spray | |
|---|---|---|---|---|---|---|
| # | Compound | solids | Rate (g/min) | Temperature (° C.) | Time (min) | Yield |
| 1 | 3:1 PVP-VA 64: | 15% | 8 | 40 | 11 | 90.2% |
| | Cmpd 1 | | | | | |
| 2 | 3:1 Kollidon 30: | 15% | 8 | 41 | 11 | 87.7% |
| | Cmpd 1 | | | | | |
| 3 | 3:1 HPMC E5: | 10% | 8-10 | 40 | 14 | 76.5% |
| | Cmpd 1 | | | | | |
| 4 | 3:1 HPMC-AS: | 10% | 8 | 39 | 18 | 77.4% |
| | Cmpd 1 | | | | | |

A 80:20 DCM:methanol solution was used as the spray solvent for all solutions. Spray solutions containing PVP-VA 64 and Kollidon 30 contained 15% w/w solid content, which includes both the content of the polymer and the compound. Spray solutions containing HPMC E5 and HPMC-AS contained 10% w/w solid content. A total amount of 3.1 g of Compound 1 was used for each spray run.

All spray dry dispersions (SDDs) were dried overnight at 40° C. at −25 mmHg vacuum, with a nitrogen purge for 15-20 minutes prior to removing from the oven for storage under a nitrogen blanket in the primary container, and desiccated in the secondary container.

The compounds and SDDs were visualized using Polarized Light Microscopy (PLM) and analyzed by powder X-Ray Diffraction (PXRD), Differential Scanning Calorimetry (DSC) and Thermogravimetric Analysis (TGA).

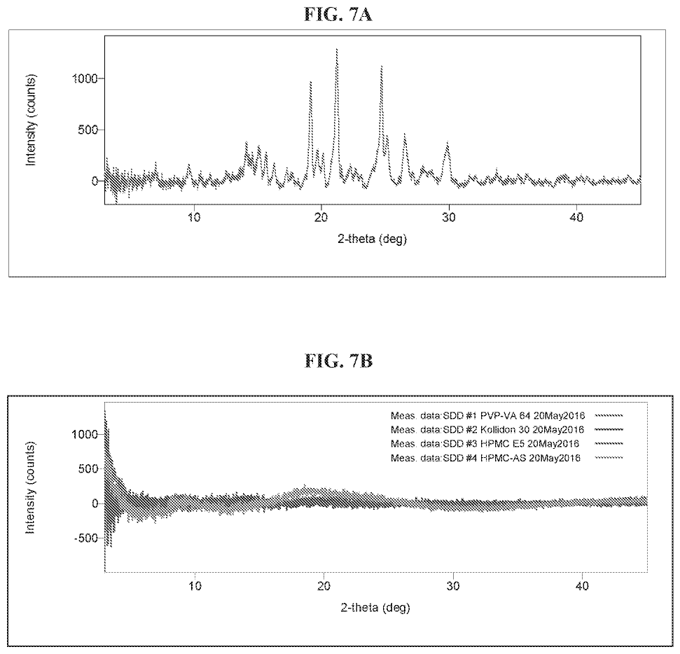

PXRD was performed using a Rigaku X-Ray Powder Diffractometer (MiniFlex 600 FAE-R PDXL-Version 2-0 Cu Kα radiation S/N BD63000375). FIG. 7A shows the PXRD (Powder X-Ray Diffraction) diffractogram of Compound 1. The PXRD diffractogram for Compound 1 indicates that the compound is mostly crystalline due to its sharply defined peaks. FIG. 7B shows the overlayed PXRD diffractogram of four different spray dried formulations of Compound 1. The PXRD diffractogram for spray dry dispersions (SDD #1-4) indicate that the spray dry dispersions are mostly amorphous material.

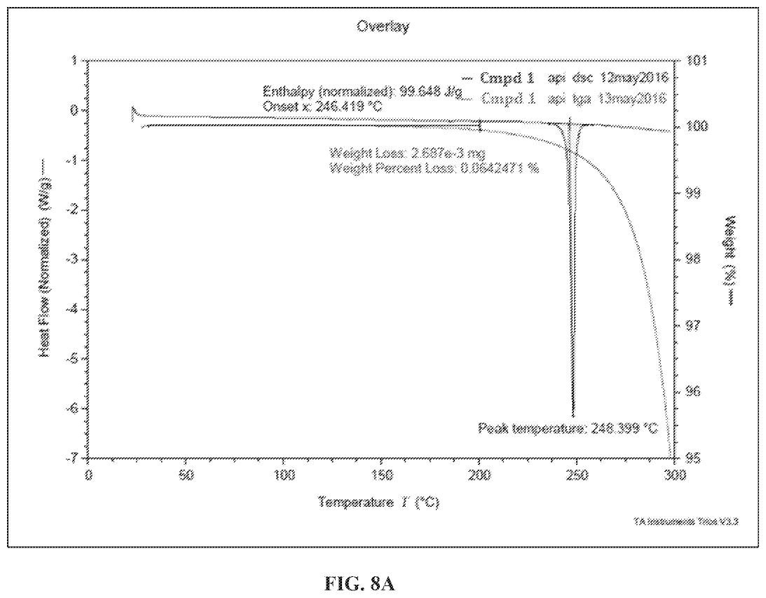

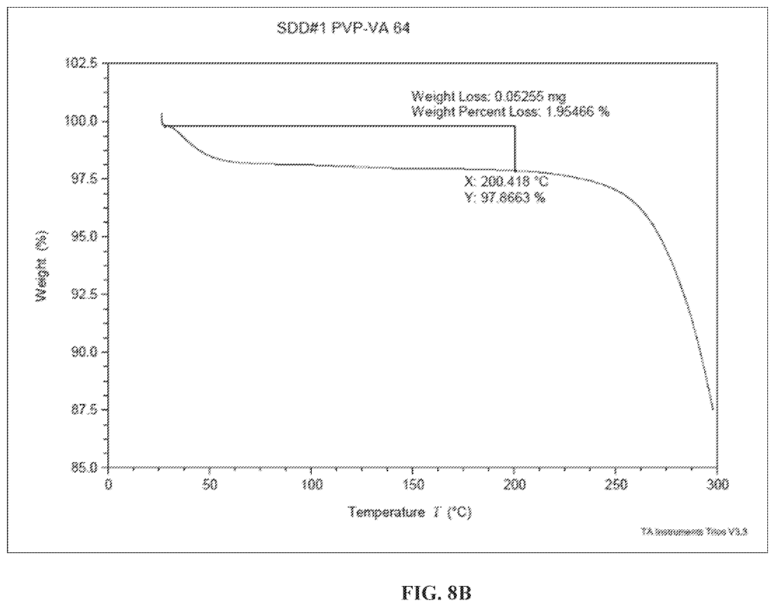

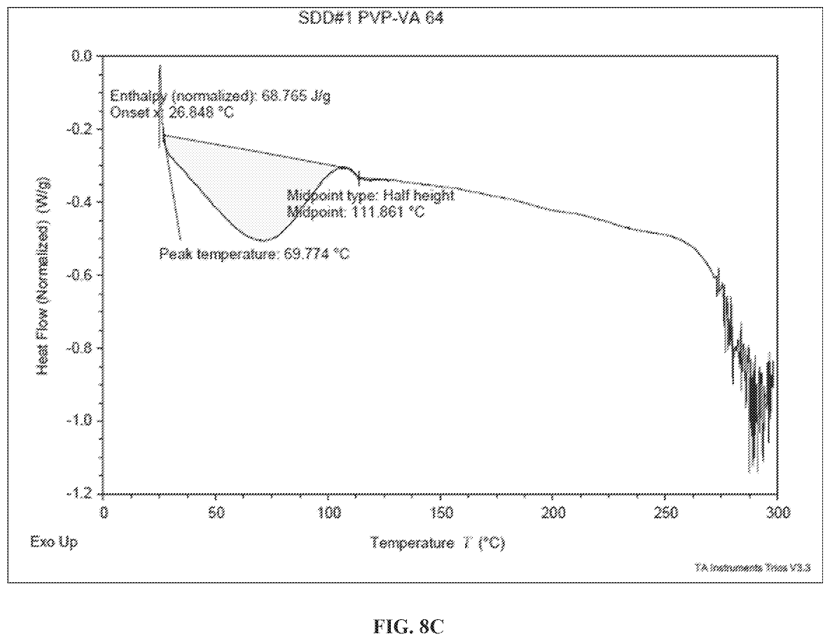

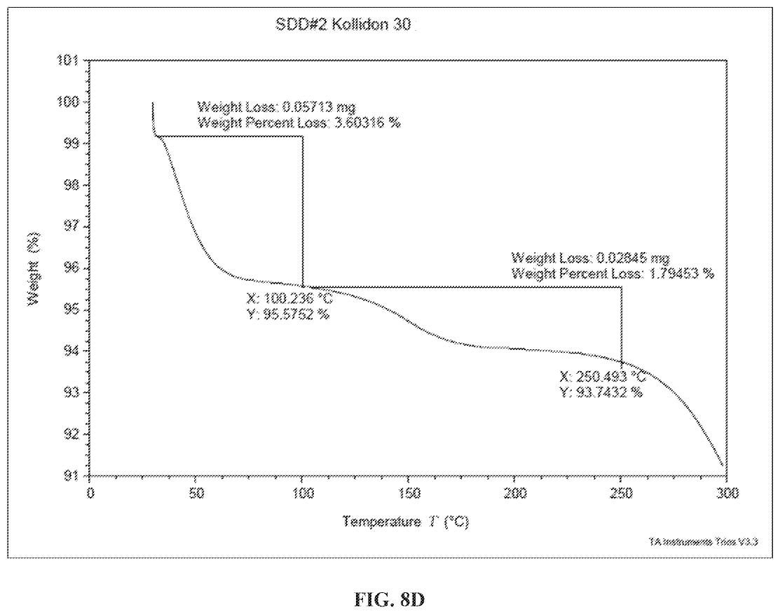

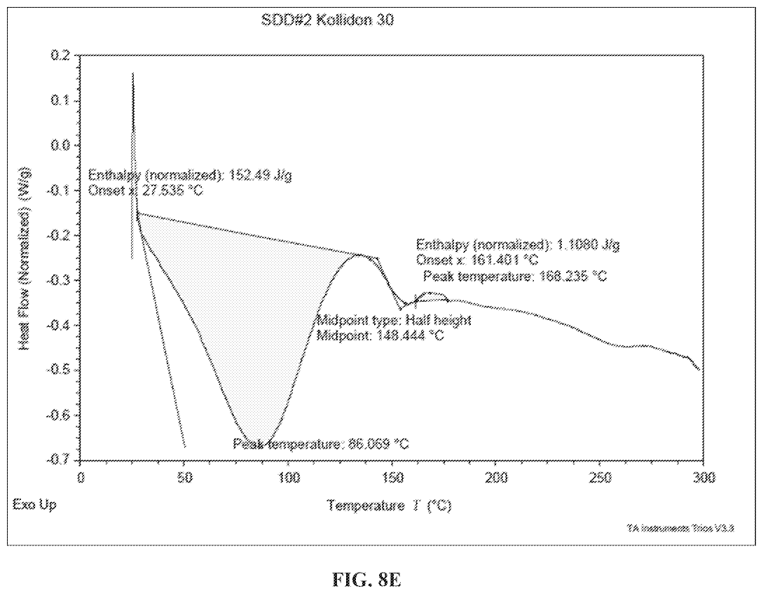

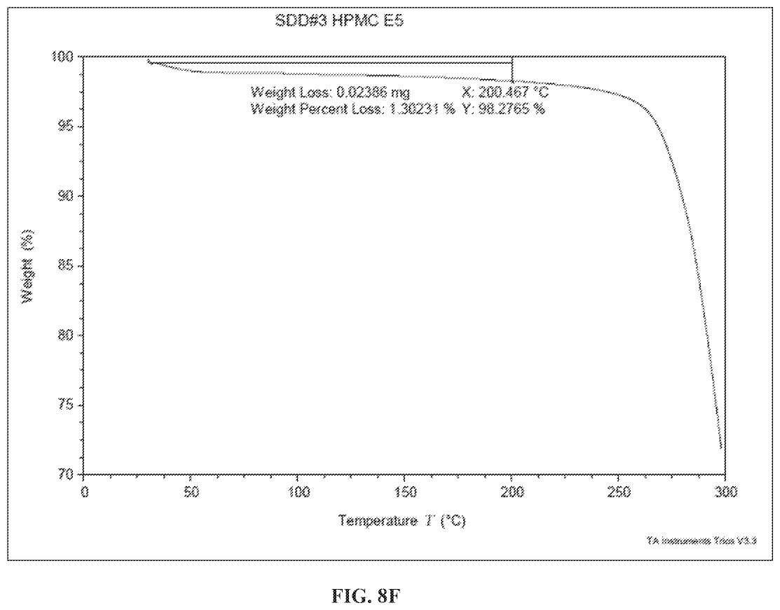

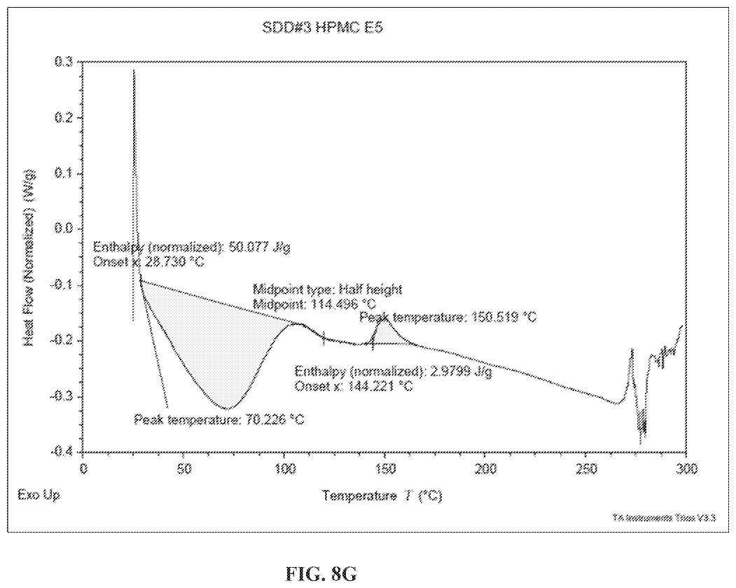

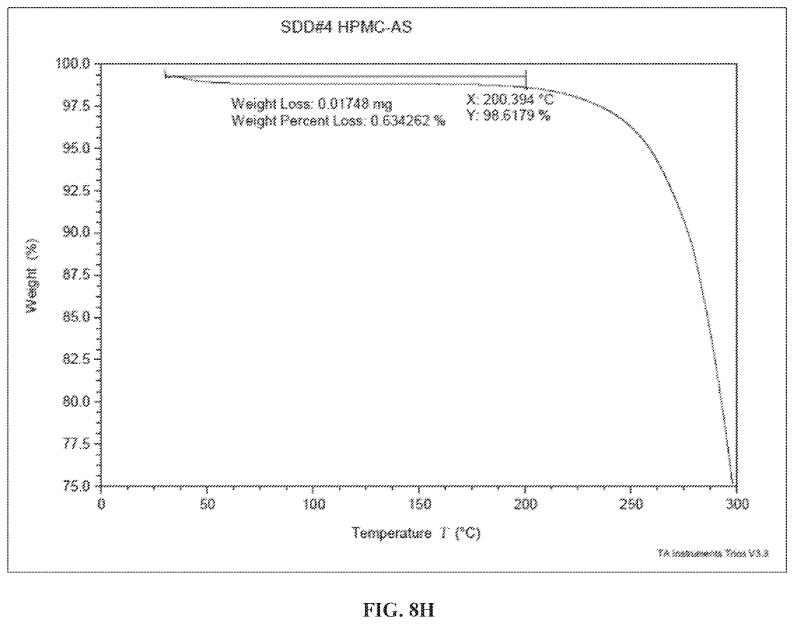

FIG. 8A shows the overlay of the DSC and TGA thermograms for Compound 1. FIGS. 8B, 8D, 8F, and 8H show the TGA thermographs of spray dry dispersions (SDD) #1-4, respectively. FIGS. 8C, 8E, 8G, and FIG. 8I show the DSC thermograms of spray dry dispersions (SDD) #1-4, respectively.

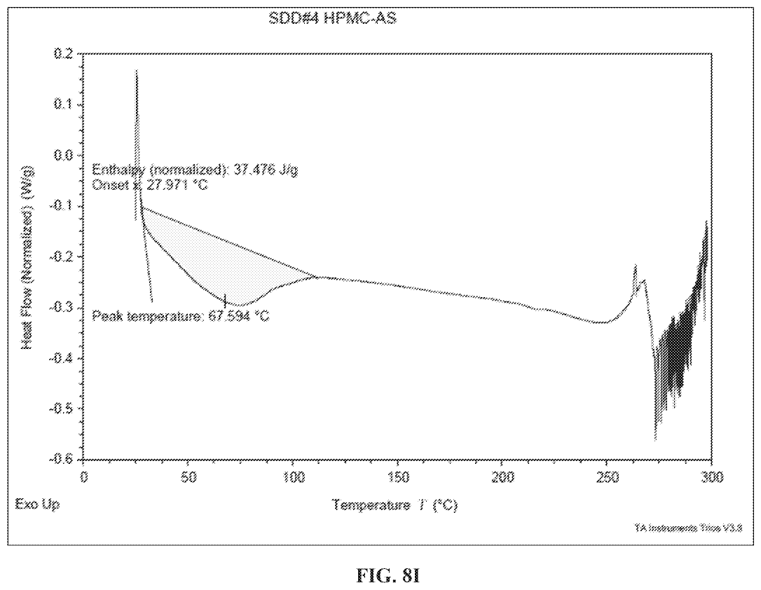

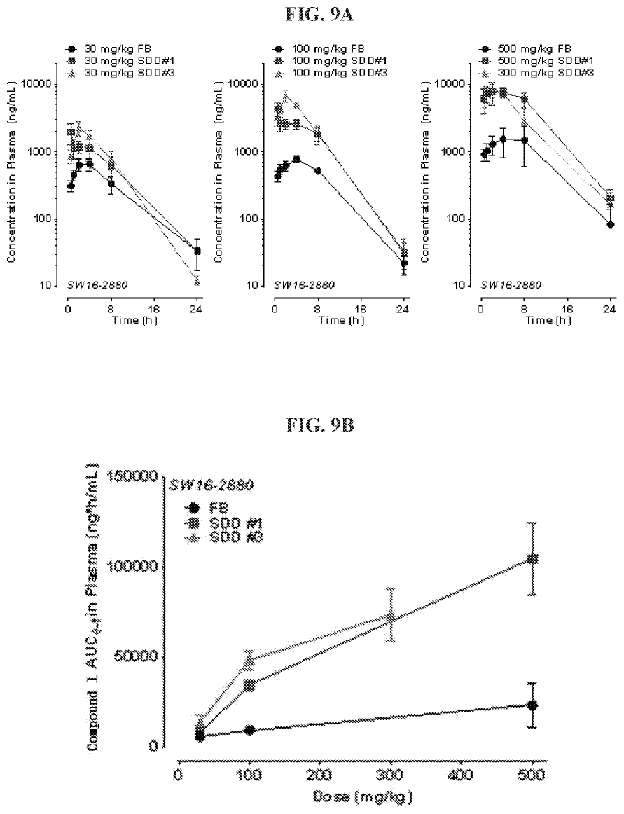

The pharmacokinetic (PK) properties of three separate formulations of Compound 1 (Free Base and two spray dry dispersions, SDD #1 and SDD#3) were evaluated in male Sprague Dawley (CD®IGS) rats following a single administration by oral (PO) gavage of 30, 100 or 500 mg/kg at a volume of 10 mL/kg. A total of 45 animals were used in this study (5 rats/dose×3 dose levels×3 formulations). The vehicle consisted of 0.75% hydroxypropyl methylcellulose (HPMC; w/v), 0.2% Tween 20 (v/v), and deionized water. FIG. 9A shows the PK curves of Compound 1 in free base form (FB) and two spray dry dispersions of Compound 1 (SDD #1 and SDD #3). FIG. 9B shows the AUC vs. dose for Compound 1 in free base form (FB) and two spray dry dispersions of Compound 1 (SDD #1 and SDD #3).

### Example 30. Single Crystal X-Ray Diffraction

Single crystal x-ray diffraction (SXRD) was carried out (Solid Form Solutions, Penicuik, Scotland, UK) to determine the structure of Compound 1, and the results are summarized in Tables 4 and 5. Single crystal X-ray analysis was conducted using an Agilent SuperNova dual source instrument, at 120 K using Mo Kα radiation (λ=0.71073 A) generated by a sealed tube. Data was corrected for absorption effects using an empirical correction with spherical harmonics. All data was reduced, solved and refined in the achiral triclinic space group P-1.

Compound 1 (approx. 10 mg) was dissolved in isopropyl acetate (500 μL) in a 2 ml clear glass HPLC vial and heptane slowly diffused into the solution of Compound 1 at ambient temperature. After standing at ambient temperature for several days, large block-like crystals were noted to have grown below the solution meniscus, that were suitable for interrogation by single crystal X-ray diffraction.

A colorless fragment of a lath (0.237×0.158×0.126 mm) was used in the single crystal diffraction study. The crystal was coated with Paratone oil and data collected on a Rigaku Oxford Diffraction (Dual Source) SuperNova diffractometer using graphite monochromated Mo Kα (λ=0.71073 A, 40 kV/40 mA) radiation at 120(1) K using an Oxford Cryosystems 700+ low temperature device and Atlas CCD plate detector (Rigaku Oxford Diffraction). A total of 2123 frames were collected for a hemisphere of reflections using a ω strategy calculated by CrysAlisPro (Rigaku Oxford Diffraction 1.171.38.43h, 2015) over the Θ range 3.02-31.25° with 1° step size and 20 sec/frame exposure. Frames were integrated using CrysAlisPro (Rigaku Oxford Diffraction 1.171.38.43h, 2015) to a triclinic cell using a moving average background, yielding a total of 106625 reflections, of which 10259 were independent (I>2σ(I)). Data were integrated to 2Θmax=62.5° (95.4% completeness). Absorption corrections were applied using SCALE3 ABSPACK (CrysAlisPro 1.171.38.43h, Rigaku Oxford Diffraction, 2015) using an empirical model using spherical harmonics coupled with gaussian integration over a multifaceted crystal model (absorption coefficient G=0.533 mm-1).

The OLEX2 (Dolomanov, O. V., Bourhis, L. J., Gildea, R. J., Howard, J. A. K., Puschmann, H. J Appl. Cryst. 2009, 42, 339-341) graphical software package was used as an interface for phase determination and structure refinement. Data were solved using Superflip (Palatinus, L. & Chapuis, G. (2007). J. Appl. Cryst., 40, 786-790; Palatinus, L. & van der Lee, A. (2008). J. Appl. Cryst. 41, 975-984; Palatinus, L., Prathapa, S. J. & van Smaalen, S. (2012). J. Appl. Cryst. 45, 575-580) and developed by full least squares refinement on F2 (Sheldrick, G. M. (2015). Acta Cryst. C71, 3-8) in the triclinic space-group P-1. A search for higher metric symmetry using the ADDSYMM (Le Page, Y. J. Appl. Cryst. 1987, 20, 264; Le Page, Y. J. Appl. Cryst. 1988, 21, 983) routine of PLATON (Spek A. L., Acta Cryst. 2009, D65, 148) was attempted, but failed to uncover any higher order symmetry. All non-hydrogen atoms were located in the Fourier map and their positions refined prior to describing their thermal movement of all non-hydrogen atoms anisotropically. Within the asymmetric unit, two complete, crystallographically independent Compound 1 formula units were found, where one of which (molecule ‘B’) was found to exhibit positional disorder over three positions. This disorder was refined using the SHELX compatible SUMP command with three parts to yield occupancies of 34.1:43.2:22.7%. Furthermore, the disordered rings C11B(C12B,C13B,C14B,C9B,C10B); C11C(C15D,C13C,C14C,C9C,C10C); C11D(C12D,C13D, C1-4D,C9D,C10D) were refined as rigid hexagons using the SHELX compatible command AFIX66. Furthermore, C15B-C13B was restrained to 1.49(2) A and C9B, C9D and C13D were restrained to give approximate isotropic thermal motion using the SHELX compatible command ISOR with sigma 0.01 and sigma 0.05 for terminal atoms. All hydrogen atoms were placed in calculated positions using a riding model with fixed Uiso at 1.2 times for all CH and NH groups. Highest peak: 0.76 e.A-3 at 0.19430.18000.0797 [0.42 A from S1B]. Deepest hole: −1.18 e.A-3 at 0.22030.15430.1130 [0.86 A from S1B].

Crystal Data for C₁₅H₉ClF₃N₃O₂S₂ (M=419.82 g/mol): triclinic, space group P-1 (no. 2), a=10.0426(2) A, b=12.6946(3) A, c=13.5882(3) A, α=89.219(2°), β=83.540(2°), γ=73.357(2)°, V=1648.89(6) A3, Z=4, T=120(1) K, (MoKα)=0.533 mm-1, Dcalc=1.691 g/cm³, 106625 reflections measured (6.04°<2Θ<62.5°), 10259 unique (Rint=0.0431, Rsigma=0.0252) which were used in all calculations. The final R₁ was 0.0700 (>2sigma(I)) and wR₂ was 0.1358 (all data).

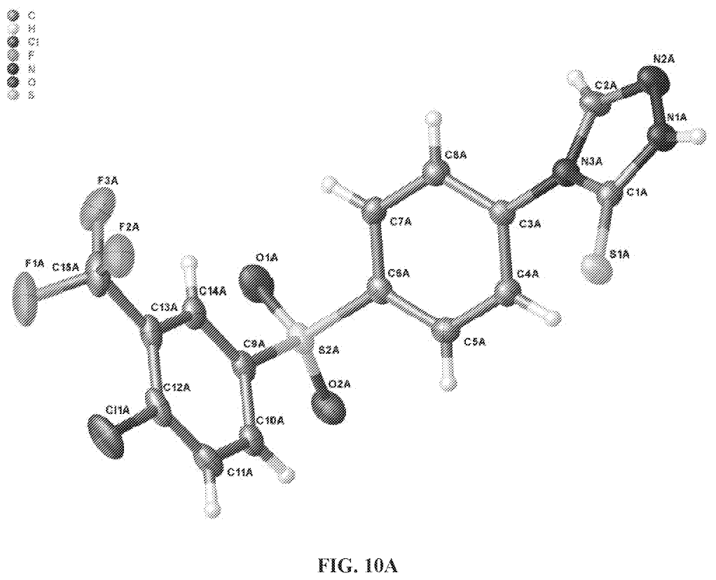

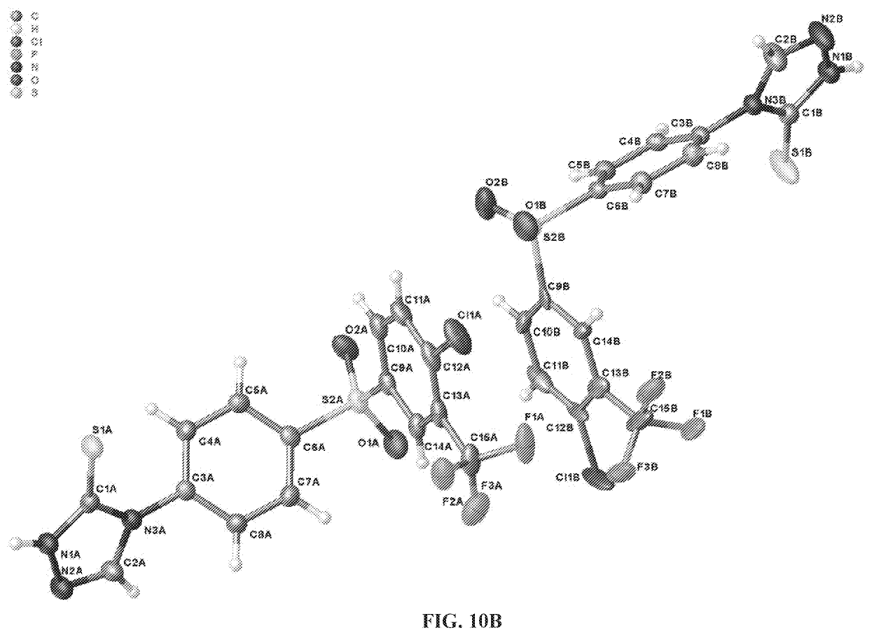

The single crystal structure analysis of Compound 1 is shown in FIG. 10A. FIG. 10B shows the single crystal structural analysis of an asymmetric unit of Compound 1. The asymmetric unit was found to contain two complete units of Compound 1 with 1-chloro-trifluorophenyl moiety of molecule ‘B’ refined occupancies of 34.1:43.2:22.7%. No further disorder was found within the overall model.

Table 4 shows the crystallographic refinement details of Compound 1 (Form 1).

TABLE 4

| Empirical formula | C₁₅H₉ClF₃N₃O₂S₂ |
|---|---|
| Formula weight | 419.82 |
| Temperature/K | 120(1) |
| Crystal system | triclinic |
| Space group | P-1 |
| a/Å | 10.0426(2) |
| b/Å | 12.6946(3) |
| c/Å | 13.5882(3) |
| α/° | 89.219(2) |
| β/° | 83.540(2) |
| γ/° | 73.357(2) |
| Volume/Å³ | 1648.89(6) |
| Z,Z′ | 4 |
| ρ_{calc} g/cm³ | 1.691 |
| μ/mm⁻¹ | 0.533 |
| F(000) | 848.0 |
| Crystal size/mm³ | 0.237 × 0.158 × 0.126 |
| Radiation | MoKα (λ = 0.71073) |
| 2Θ range for data | 6.04 to 62.5 |
| collection/° | |
| Index ranges | −14 ≤ h ≤ 14, −18 ≤ k ≤ 17, −19 ≤ l ≤ 19 |
| Reflections collected | 106625 |
| Independent reflections | 10259 [Rᵢₙₜ = 0.0431, R_{sigma} = 0.0252] |
| Data/restraints/ | 10259/20/634 |
| parameters | |
| S | 1.232 |
| Final R indexes | R₁ = 0.0700, wR₂ = 0.1327 |
| [F² > 2σ (F²)] | |
| Final R indexes | R₁ = 0.0787, wR₂ = 0.1358 |
| [all data] | |
| Δρmax, Δρmin/e Å⁻³ | 0.76/−1.18 |

R₁ = (Σ |Fₒ| − |F_{c}|)/Σ |Fₒ|)
wR₂ = {Σ [w(Fₒ² − F_{c}²)²]/Σ [w(Fₒ²)²]}^{1/2}
S = {Σ [w(Fₒ² − F_{c}²)²]/(n − p)}^{1/2}

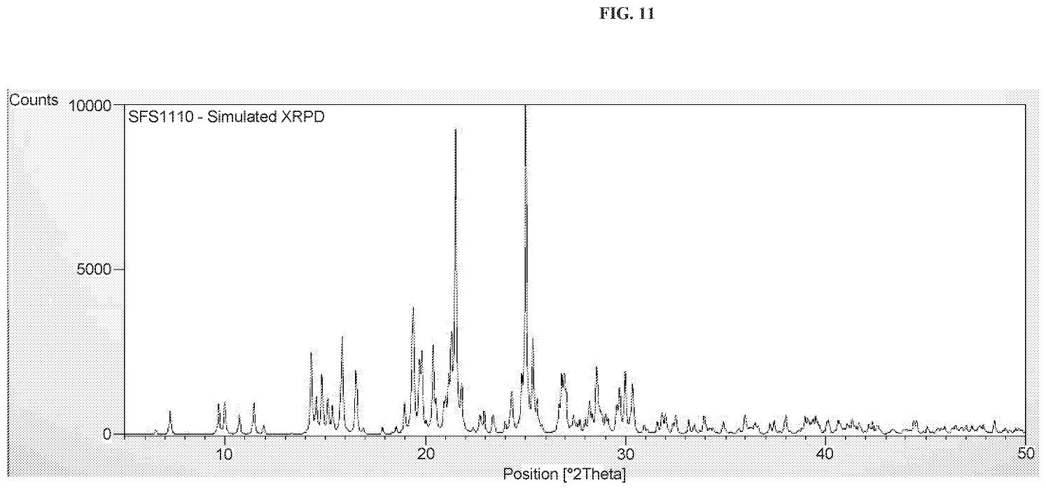

Table 5 shows the simulated 20 X-ray powder diffractogram (XRPD) of Compound 1 (Form 1). The XRPD is shown in FIG. 11.

TABLE 5

| | Pos. | FWHM | Area | d-spacing | Height | Rel. Int. |
|---|---|---|---|---|---|---|
| No. | [° 2Th.] | [° 2Th.] | [cts*° 2Th.] | [\acute{Å}] | [cts] | [%] |
| 1 | 7.2633 | 0.096 | 89.50 | 12.1610 | 699.26 | 6.94 |
| 2 | 9.6858 | 0.096 | 119.57 | 9.1242 | 934.16 | 9.27 |
| 3 | 9.9875 | 0.120 | 158.90 | 8.8492 | 993.15 | 9.85 |
| 4 | 10.7073 | 0.120 | 101.23 | 8.2559 | 632.68 | 6.28 |
| 5 | 11.4394 | 0.096 | 122.72 | 7.7291 | 958.77 | 9.51 |
| 6 | 11.9245 | 0.096 | 32.83 | 7.4157 | 256.50 | 2.54 |
| 7 | 14.3055 | 0.096 | 317.49 | 6.1864 | 2480.38 | 24.61 |
| 8 | 14.5557 | 0.096 | 149.08 | 6.0806 | 1164.67 | 11.55 |
| 9 | 14.8445 | 0.096 | 233.35 | 5.9630 | 1823.05 | 18.09 |
| 10 | 15.1296 | 0.120 | 174.44 | 5.8512 | 1090.27 | 10.82 |
| 11 | 15.3639 | 0.096 | 111.84 | 5.7625 | 873.74 | 8.67 |
| 12 | 15.8539 | 0.096 | 381.04 | 5.5855 | 2976.84 | 29.53 |
| 13 | 16.5457 | 0.096 | 250.15 | 5.3535 | 1954.28 | 19.39 |
| 14 | 18.9653 | 0.096 | 125.47 | 4.6756 | 980.23 | 9.72 |
| 15 | 19.3893 | 0.168 | 842.79 | 4.5743 | 3762.45 | 37.32 |
| 16 | 19.7223 | 0.072 | 219.15 | 4.4978 | 2282.80 | 22.65 |
| 17 | 19.8198 | 0.072 | 244.04 | 4.4759 | 2542.05 | 25.22 |
| 18 | 20.3840 | 0.096 | 347.46 | 4.3533 | 2714.51 | 26.93 |
| 19 | 20.5478 | 0.072 | 98.72 | 4.3189 | 1028.31 | 10.20 |
| 20 | 20.9792 | 0.144 | 197.96 | 4.2311 | 1031.04 | 10.23 |
| 21 | 21.3094 | 0.072 | 304.21 | 4.1663 | 3168.82 | 31.44 |
| 22 | 21.5096 | 0.120 | 1522.85 | 4.1279 | 9517.79 | 94.42 |
| 23 | 21.8210 | 0.096 | 200.37 | 4.0697 | 1565.37 | 15.53 |
| 24 | 22.7420 | 0.120 | 92.33 | 3.9070 | 577.05 | 5.72 |
| 25 | 22.9527 | 0.096 | 94.20 | 3.8716 | 735.93 | 7.30 |
| 26 | 23.3841 | 0.120 | 94.20 | 3.8011 | 588.74 | 5.84 |
| 27 | 24.3296 | 0.096 | 171.62 | 3.6555 | 1340.75 | 13.30 |
| 28 | 24.8115 | 0.072 | 167.72 | 3.5856 | 1747.10 | 17.33 |
| 29 | 25.0252 | 0.096 | 1290.30 | 3.5554 | 10080.44 | 100.00 |
| 30 | 25.3797 | 0.096 | 374.34 | 3.5066 | 2924.51 | 29.01 |
| 31 | 25.5790 | 0.072 | 103.60 | 3.4797 | 1079.11 | 10.71 |
| 32 | 26.6520 | 0.072 | 79.62 | 3.3420 | 829.39 | 8.23 |
| 33 | 26.8121 | 0.072 | 175.89 | 3.3224 | 1832.21 | 18.18 |
| 34 | 26.9266 | 0.072 | 180.08 | 3.3085 | 1875.81 | 18.61 |
| 35 | 27.4026 | 0.096 | 73.50 | 3.2521 | 574.20 | 5.70 |
| 36 | 27.9857 | 0.096 | 64.48 | 3.1857 | 503.75 | 5.00 |
| 37 | 28.2013 | 0.096 | 130.86 | 3.1618 | 1022.32 | 10.14 |
| 38 | 28.5619 | 0.096 | 264.26 | 3.1227 | 2064.57 | 20.48 |
| 39 | 29.0172 | 0.072 | 59.44 | 3.0747 | 619.21 | 6.14 |
| 40 | 29.5813 | 0.096 | 119.64 | 3.0174 | 934.67 | 9.27 |
| 41 | 29.7121 | 0.096 | 186.01 | 3.0044 | 1453.19 | 14.42 |
| 42 | 29.9864 | 0.120 | 308.97 | 2.9775 | 1931.07 | 19.16 |
| 43 | 30.3430 | 0.120 | 243.43 | 2.9433 | 1521.46 | 15.09 |
| 44 | 31.8318 | 0.096 | 89.32 | 2.8090 | 697.82 | 6.92 |
| 45 | 31.9921 | 0.096 | 84.06 | 2.7953 | 656.71 | 6.51 |
| 46 | 32.5089 | 0.096 | 76.75 | 2.7520 | 599.61 | 5.95 |
| 47 | 33.9457 | 0.168 | 124.88 | 2.6388 | 557.50 | 5.53 |
| 48 | 35.9745 | 0.120 | 96.42 | 2.4945 | 602.63 | 5.98 |
| 49 | 38.0146 | 0.192 | 150.07 | 2.3651 | 586.21 | 5.82 |
| 50 | 38.9727 | 0.072 | 54.51 | 2.3092 | 567.82 | 5.63 |

### Example 31: In Vivo Studies Using the Line 61 mThy1-Alpha-Synuclein Transgenic Mouse Model

Multiple in vivo administration studies of Compound 1 were carried out in the Line 61 (L61) mThy1-alpha-synuclein transgenic mouse model of Parkinson's disease (PD). The mThy1-alpha-synuclein transgenic mouse model overexpresses wild-type human ASYN under the Thy-1 promoter (commonly referred to as Line 61 transgenic mice; Rockenstein et al., 2002). This transgenic mouse develops extensive accumulation of alpha-synuclein (ASYN) in areas relevant to PD (Rockenstein et al., 2002; Chesselet et al., 2012; Games et al., 2013), neurodegeneration including dopaminergic neurodegeneration, reduced dopamine (DA) and TH loss in the striatum (Masliah et al., 2000; Lam et al., 2011), and motor deficits (Fleming et al., 2004). Male transgenic and non-transgenic littermates (3-3.5 mo) were used for all in vivo studies presented here.

i. Effects of Compound 1 on ASYN Pathology and a Marker of Neuroprotection and Autophagy

Alpha-synuclein (ASYN) is a neuronal protein whose dysregulation has been implicated in the pathogenesis of PD. The effects of Compound 1 on alpha-synuclein aggregation were assessed in both L61 ASYN transgenic and non-transgenic mice in a 1 month administration study. L61 ASYN transgenic mice (36 total mice, n=8-11 mice per treatment group) were injected (i.p.) daily with 1, 5, or 10 mg/kg of Compound 1 or a vehicle control (5% DMSO+20% Cremphor EL+0.9% normal saline) for 1 month. Non-transgenic mice (18 total mice, n=8-11 mice per treatment group) were used as a control and were injected daily (i.p.) with 10 mg/kg of Compound 1 or a vehicle control (5% DMSO+20% Cremphor EL+0.9% normal saline) per day for 1 month. At the end of one month, the mice were sacrificed, and immunohistochemical (IHC) detection of total alpha-synuclein deposits, insoluble alpha-synuclein deposits (PK+resistant), microtubule-associated protein 1A/1B-light chain 3 (LC3), and monomeric alpha-synuclein levels were assessed in the harvested brain tissues.

Data from the 1 month administration study show that Compound 1 at doses of 1, 5 and 10 mg/kg (i.p. injection, once daily) produced beneficial actions which included reductions in cortical hippocampal and striatal levels of monomeric, total and Proteinase K treatment-resistant (insoluble) ASYN as measured by immunohistochemistry (IHC) and/or biochemical methods. The data show that Compound 1 promotes the clearance of alpha-synuclein (ASYN), a neuronal protein whose dysregulation has been clearly implicated in the pathogenesis of PD. In addition to improvements in ASYN neuropathology, administration of Compound 1 increased levels of microtubule-associated protein 1A/1B-light chain 3 (LC3), a marker of autophagy and neuroprotective pathways. Finally, treatment using Compound 1 also produced functional improvements in the motor performance of L61 ASYN transgenic mice treated for 3 months.

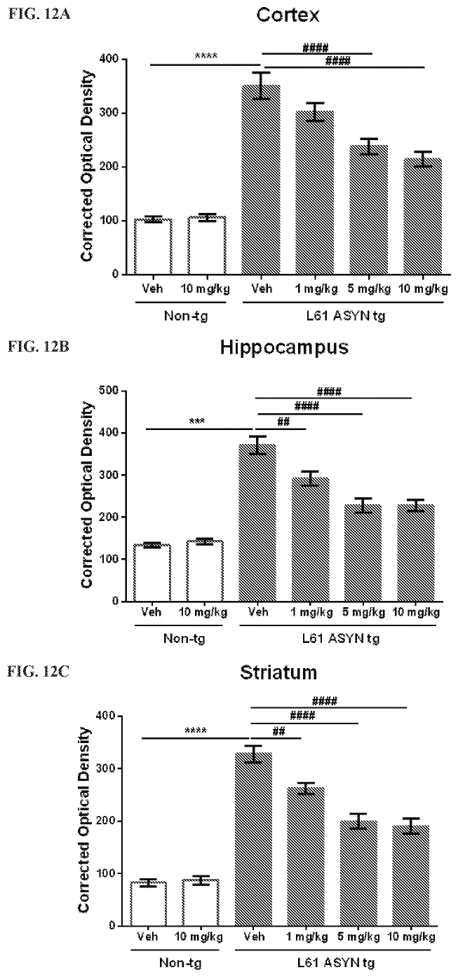

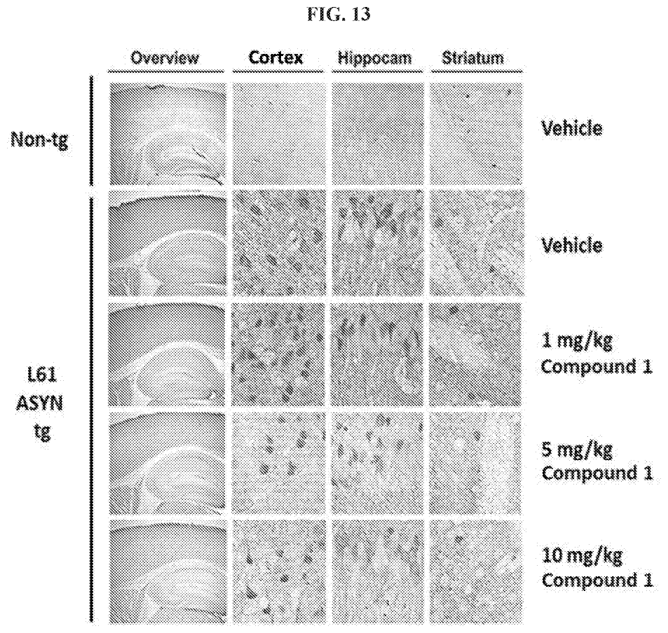

FIG. 12 shows the quantification of total alpha synuclein staining in cross-sections of the cortex, hippocampus, and striatum of L61 ASYN transgenic mice and control mice after i.p. administration of Compound 1 or vehicle for 1 month. FIG. 13 shows the IHC staining for total alpha-synuclein deposits in representative images of cross-sections of the cortex, hippocampus, and striatum of L61 ASYN transgenic mice and control mice after i.p. administration of Compound 1 or vehicle for 1 month. The quantification and IHC staining of total alpha-synuclein were performed using known techniques (Rockenstein et al., J Neurosci Res. 2002, 68(5):568-78; Tanji et al., Acta Neuropathol. 2010, 120, 145-154; Nuber et al., Brain. 2013 February; 136(Pt 2):412-32). FIG. 12 shows that administration of Compound 1 (1, 5 or 10 mg/kg per day i.p. for 1 month) reduced total ASYN in the neuropil of (A) cortex, (B) hippocampus, and (C) striatum of transgenic mice compared to the vehicle control, as assessed by quantitative immunocytochemistry. As shown in FIG. 12, the reductions of cortical, hippocampal, and striatal levels of total alpha-synuclein resulting from Compound 1 administration are statistically significant. In particular, the data in FIG. 12A shows that when administered daily at 1 mg/kg, 5 mg/kg and 10 mg/kg, Compound 1 reduces the total alpha-synuclein level in cortex by 13%, 32% and 38% respectively as compared to a vehicle control. This is also seen in FIG. 13, which shows the total alpha-synuclein deposits in representative images of cross-sections of the cortex, hippocampus, and striatum of the brain tissues harvested from these mice. The staining in FIG. 13 shows that Compound 1 produces beneficial actions in reducing cortical, hippocampal and striatal levels of total alpha-synuclein.

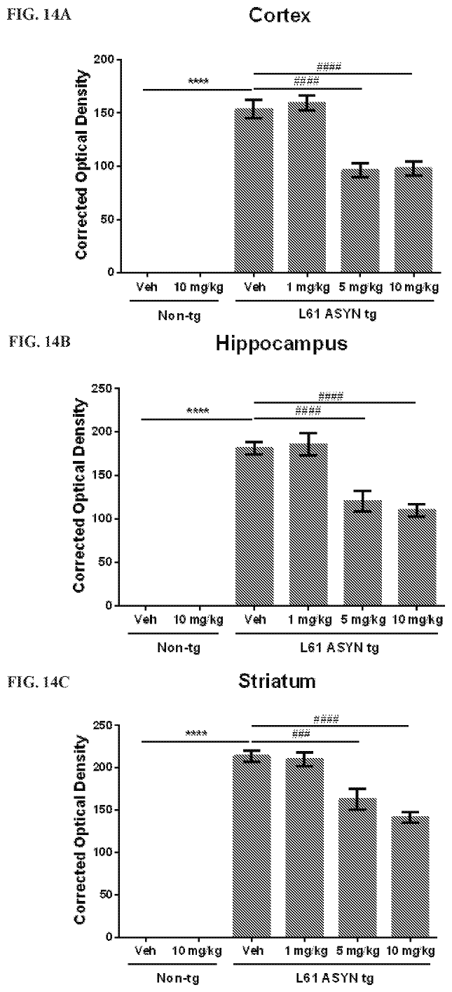

FIG. 14 shows the quantification of PK-resistant alpha synuclein staining in cross-sections of the cortex, hippocampus, and striatum of L61 ASYN transgenic mice and control mice after i.p. administration of Compound 1 or vehicle for 1 month. FIG. 15 shows the IHC staining for PK-resistant alpha-synuclein deposits in representative images of cross-sections of the cortex, hippocampus, and striatum of L61 ASYN transgenic mice and control mice after i.p. administration of Compound 1 or vehicle for 1 month. The quantification and IHC staining of PK-resistant alpha-synuclein were performed using known techniques (Rockenstein et al., J Neurosci Res. 2002, 68(5):568-78; Tanji et al., Acta Neuropathol. 2010, 120, 145-154; Nuber et al., Brain. 2013 February; 136(Pt 2):412-32). As shown in FIGS. 14 and 15, administration of Compound 1 (1, 5 or 10 mg/kg per day i.p. for 1 month) also reduced the insoluble alpha-synuclein deposits (PK+resistant) in the (A) cortex, (B) hippocampus, and (C) striatum of the transgenic mice. FIG. 14 shows that the reductions of cortical, hippocampal and striatal levels of PK-resistant alpha-synuclein resulting from Compound 1 administration are statistically significant. In particular, the data in FIG. 14A shows that Compound 1 when administered daily at 5 mg/kg and 10 mg/kg reduces the PK-resistant alpha synuclein levels in cortex by 37% and 36% respectively as compared to vehicle-treated mice. The staining in FIG. 15 shows that Compound 1 produces beneficial actions in reducing cortical, hippocampal and striatal levels of PK-resistant alpha-synuclein.

FIG. 16 shows that administration of Compound 1 (1, 5 or 10 mg/kg per day i.p. for 1 month) reduced the (A) cortical and (B) hippocampal levels of monomeric ASYN in the cytosolic fraction of brain homogenates from L61 ASYN transgenic mice. Biochemical evaluations were conducted using a ProteinSimple© western biochemical evaluation. Briefly, samples were mixed with pre-calculated volumes of 0.1× Sample Buffer and 5× Fluorescent Master Mix to make a final sample concentration of 0.4 mg/mL in 10 μL solution for signal optimization and evaporation reduction. Approximately 0.4 μL of sample was mixed with 2 μL of 5× fluorescent Master Mix and 7.8 μL of 0.1× Sample Buffer, vortexed, spun, and heated at 95° C. for 5 min. After brief cooling, the samples, blocking reagent, wash buffer, primary antibodies, secondary antibodies, and chemiluminescent substrate were dispensed into designated wells in the manufacturer provided plate (Kit#PS-MK14, ProteinSimple). Following plate loading the separation and immunodetection was performed automatically using default settings. The Compass software (ProteinSimple, version 2.6.7) was used to generate a report that included molecular weight, area, percent area and signal to noise for each protein detected. Data for the target protein of interest was normalized to beta-actin levels and further normalized between cartridges. Data are presented here as mean values±SEM.

It is shown in FIG. 16 that when administered daily at 1 mg/kg, 5 mg/kg or 10 mg/kg, Compound 1 reduces the levels of monomeric ASYN in the cortex as compared to vehicle treated L61 transgenic mice, in a statistically significant manner.

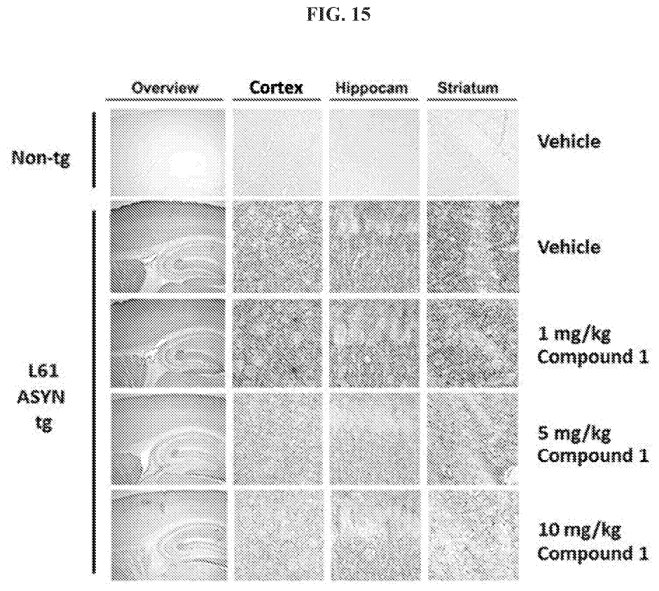

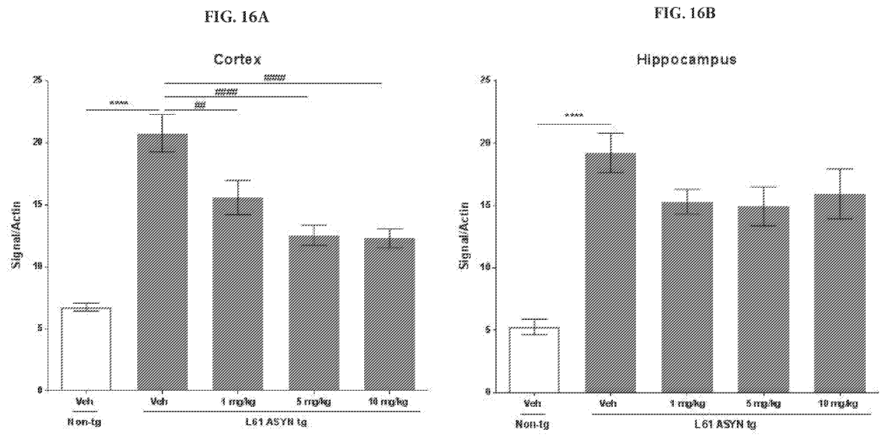

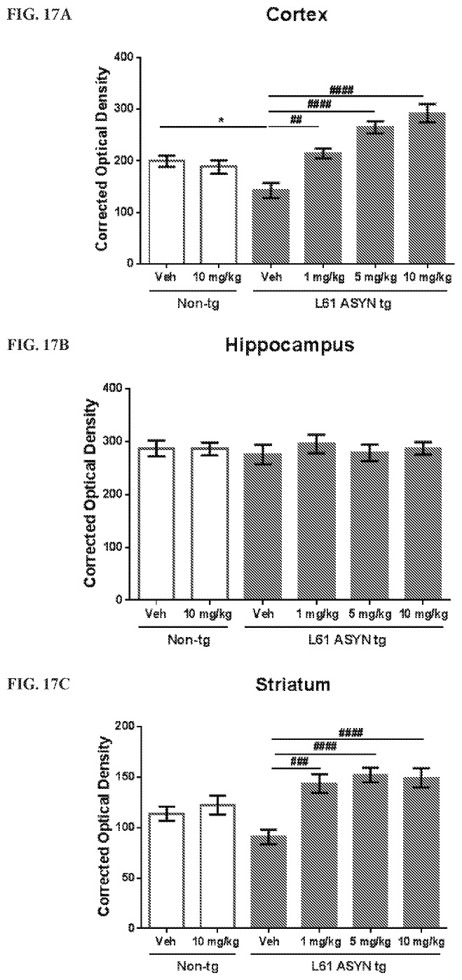

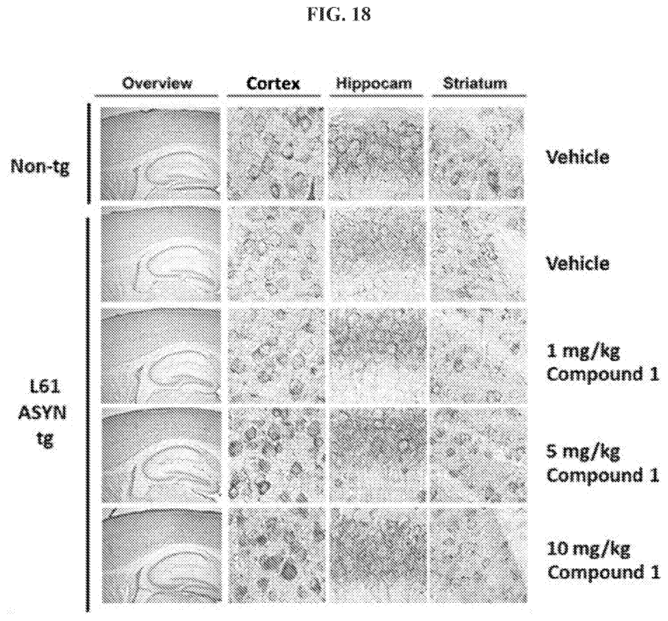

FIGS. 17 and 18 show that administration of Compound 1 (1, 5 or 10 mg/kg per day i.p. for 1 month) increased levels of microtubule-associated protein 1A/1B-light chain 3 (LC3) immunolabeling in the (A) cortex and (B) striatum, but not in the (B) hippocampus of the transgenic mice.

ii. Effects of Compound 1 on Motor Performance

The effects of Compound 1 on the motor performance deficits (grip strength) and a marker of neuroinflammation (Translocator Protein (18 kDa)) were assessed in both L61 ASYN transgenic and non-transgenic mice in a 3 month administration study.

Briefly, Compound 1 was injected into L61 ASYN transgenic mice and non-transgenic control mice (i.p., once daily) at doses of 5 and 10 mg/kg for 3 months (79 total mice, n=14-17 mice per treatment group). The vehicle control consisted of a solution containing 5% DMSO+20% Cremphor EL+0.9% normal saline. The baseline grip strength of mice was evaluated prior to starting treatments for the 3 month study, and then re-evaluated following 70 days of treatment with vehicle or Compound 1 (5 or 10 mg/kg, i.p. injection daily).

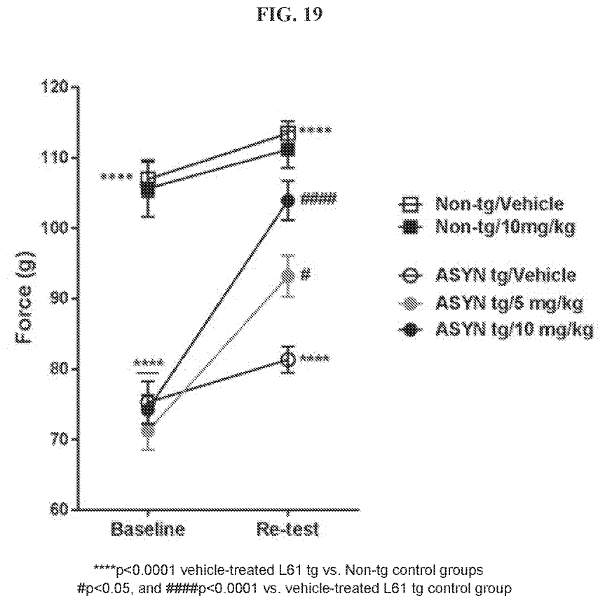

As shown in FIG. 19, administration of Compound 1 (5 or 10 mg/kg, i.p. daily) for the 3 month study produced beneficial effects on transgenic motor deficit phenotype present in L61 ASYN transgenic mice. At baseline, there was a statistically significant grip strength deficit in L61 ASYN transgenic mice compared to non-transgenic mice. Treatment with Compound 1 (5 & 10 mg/kg) improved L61 ASYN transgenic grip strength deficits. After 70 days of treatment, transgenic mice treated with Compound 1 at both 5 mg/kg and 10 mg/kg showed higher grip strengths than vehicle-treated transgenic mice in a statistically significant manner.

iii. Effects of Compound 1 on Neuroinflammation Marker TSPO

Neuroinflammation is associated with increased expression of the 18-kDa translocator protein (TSPO), which is a marker for inflammation and is present on the mitochondria of activated microglia, astroglia and macrophages (Crawshaw and Robertson 2017). The effects of Compound 1 on the levels of Translocator Protein (18 kDa) (TSPO) were assessed in both L61 ASYN transgenic and non-transgenic mice in the aforementioned 3 month administration study. At the end of the study, the mice were sacrificed, and immunofluoresence (IF) detection of TSPO were assessed in the harvested brain tissues.

FIG. 20 shows the levels of TSPO immunolabeling in representative cross-sections of the cortex of the mice. As shown in FIGS. 20A and 20B, administration of Compound 1 (5 and 10 mg/kg, i.p. daily) significantly decreased the levels of TSPO in L61 ASYN transgenic mice compared to the vehicle control. FIG. 20A shows representative TSPO immunostaining in the cortex of L61 transgenic mice injected daily with Compound 1 versus vehicle control. FIG. 20B shows the quantification of TPSO staining from representative cortical sections. The harvested brain tissues were fixed (drop fixed in 4% paraformaldehyde), sectioned using a vibratome, and representative sections were assessed for TSPO with standard immunofluorescence (IF) staining. Briefly, the right hemibrain was post-fixed in phosphate-buffered 4% PFA (pH 7.4) at 4° C. for 48 and then serially sectioned into 40 uM thick coronal sections using a vibratome. Sections were free-floated and incubated overnight at 4° C. Immunolabeling studies of TSPO were conducted using knockout validated rabbit monoclonal anti-TSPO antibody (1:500; ab199779; abeam, Temecula, Calif., USA) pre-conjugated to Alexa Fluor 488 secondary antibody. Immunolabeling, imaging and analysis was performed on blindcoded sections from Line 61 transgenic and non-transgenic mice. Slides were imaged using a EVOS Auto FL imaging system (ThermoFisher Scientific, Waltham, Mass., USA) with a 10× objective (EVOS PlanFL PH2 LWD; AMEP4681). Digitized images were analyzed using Halo (Indica Labs, Corrales, N. Mex., USA) image analysis software package by placing an ROI frame within the cortex (standardized frame placed on all images). A thresholding algorithm was defined and then applied equally to all images to determine the percentage of cortex ROI TSPO immunolabeled. The results of the analysis were then exported for graphing and statistical analysis.

Representative IF images in FIG. 20A show that when administered daily at 5 mg/kg or 10 mg/kg, Compound 1 produced beneficial actions in reducing cortical levels of TSPO, as visualized by reduced IF staining intensity. Furthermore, the quantification in FIG. 20B shows that Compound 1 at 5 mg/kg or 10 mg/kg reduces the TSPO level in a statistically significant manner as compared to vehicle-treated mice.

iv. Effects of Compound 1 on Neuroinflammation Marker GFAP

Neuroinflammation is also associated with increased expression of glial fibrillary acidic protein (GFAP) in activated astrocytes, which is induced by a variety of molecules including pro-inflammatory mediators released from activated microglia (Saijo et al. 2009). Increased expression of glial fibrillary acidic protein (GFAP) represents astroglial activation and gliosis during neurodegeneration. (Brahmachari et al., 2006). The effects of Compound 1 on GFAP expression were assessed in both L61 ASYN transgenic and non-transgenic mice in a 1 month administration study. After 30 days, the mice were sacrificed, and IHC detection of GFAP was assessed in the harvested brain tissues.

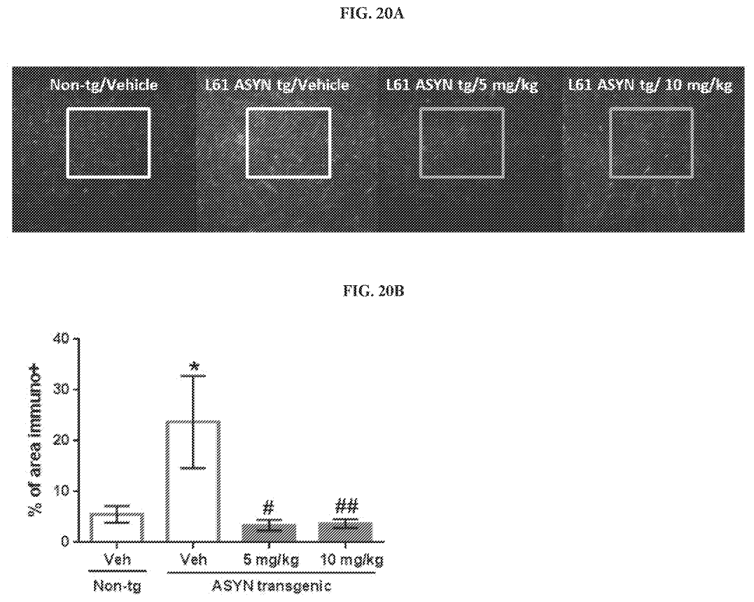

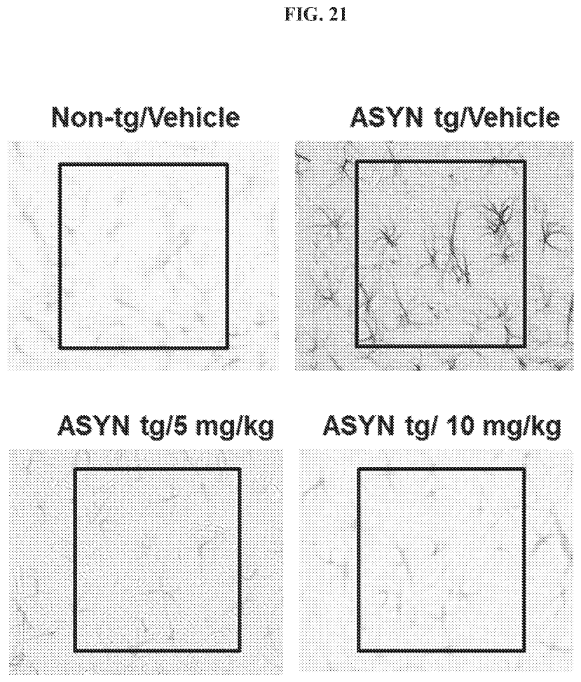

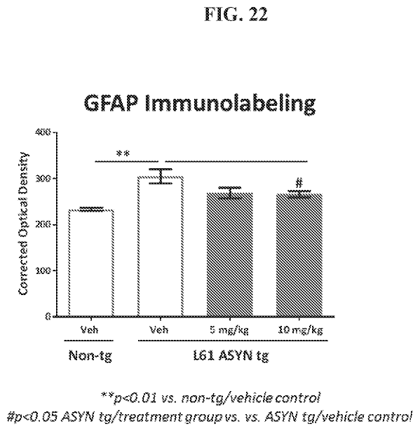

FIG. 21 shows representative GFAP immunostaining in sections containing hippocampus in L61 transgenic mice injected daily with Compound 1 versus vehicle control. FIG. 22 shows the quantification of the described GFAP staining from representative brain sections. The harvested brain tissues from treated mice were fixed (drop fixed in 4% PFA) and then sectioned into 40 micro thick sections using a vibratome. The representative sections containing the hippocampus were assessed for GFAP with standard immunohistochemistry staining. The general methods used for GFAP immunostaining follow those described in Rockenstein et al., J Neurosci Res. 2002, 68(5):568-78. Representative IHC images in FIG. 21 show that when administered daily at 5 mg/kg or 10 mg/kg, Compound 1 produces beneficial actions in reducing cortical levels of GFAP, as visualized by reduced IHC staining intensity. Furthermore, the quantification in FIG. 22 shows that at the 10 mg/kg dose, Compound 1 reduces the cortical GFAP levels in a statistically significant manner.

v. Effects of Compound 1 on Dopaminergic (DAT) Transporter Immunolabeling Levels

In Parkinson's disease, uncontrolled neuroinflammation caused by the synergic activation of microglia and astroyctes ultimately contributes to the enhanced death of DA neurons in striatum during neurodegeneration.

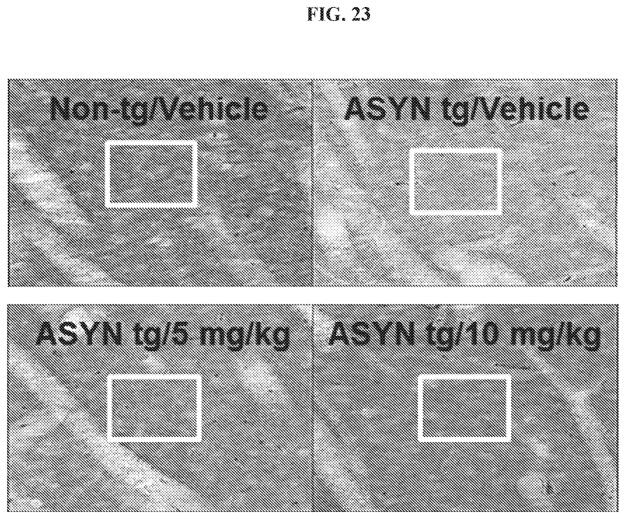

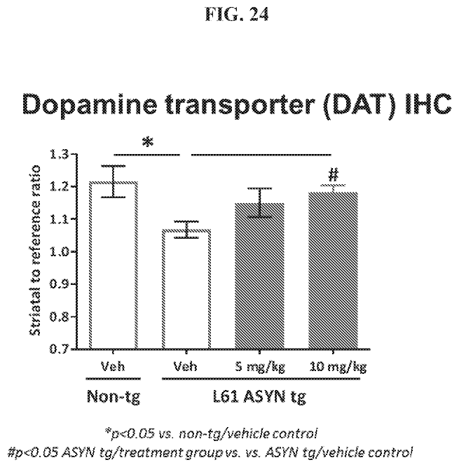

FIG. 23 shows representative dopaminergic (DAT) immunostaining in sections corresponding to the striatum in L61 transgenic mice injected daily with Compound 1 versus vehicle control. FIG. 24 shows the quantification of the described DAT staining from level matched sagittal sections containing striatum and of the cortex as a reference binding region. The harvested brain tissues were drop fixed using 4% PFA and sectioned on a vibratome, and representative sections corresponding to the striatum and cerebellum were assessed for DAT with IHC staining.

Immunolabeling studies of DAT were conducted using a monoclonal antibody (1:500; MAB369; Millipore, Temecula, Calif.) and a biotinylated secondary antibody (1:100; BA4000, Vector Labs) and analysis was performed on blindcoded sections from Line 61 transgenic and non-transgenic mice. Slides were digitized using a high resolution automated Nanozoomer slide scanner (Hamamatsu Corp.). Digitized images were analyzed using Halo (Indica Labs) image analysis software package by placing an ROI frame within the dorsal striatum and another within a separate reference brain region (for normalization of DAT signal). A thresholding algorithm was defined and then applied equally to all images to determine the average optical density of DAT immunolabeling across each ROI The results of the analysis were then exported for graphing and statistical analysis and the striatal DAT:cortical (reference region) DAT optical densities ratio was calculated for each subject.

Representative IF images in FIG. 23 show that when administered daily at 5 mg/kg or 10 mg/kg, Compound 1 produces beneficial actions in restoring striatal levels of DAT, as visualized by increased immunofluorescence intensity as compared to vehicle-treated L61 mice. Quantification of DAT density was carried out by calculating the immunofluorescence in striatal sections against that in cerebellum sections to derive a striatal-to-reference ratio. The quantification in FIG. 24 shows that Compound 1 at the 10 mg/kg dose reduces the GFAP levels in a statistically significant manner.

vi. Effects of Compound 1 on Neuroinflammation and Amyloid Beta Plaques

Neuroinflammation is associated with increased expression of the 18-kDa translocator protein (TSPO), which is present on the mitochondria of activated microglia, astroglia and macrophages (Crawshaw and Robertson 2017). The effects of Compound 1 on TSPO expression were assessed in both L41 APP transgenic and non-transgenic mice in a 1 month administration study. L41 APP transgenic mice (36 total mice, n=8-11 mice per treatment group) were injected (i.p.) daily with 5 mg/kg of Compound 1 or a vehicle control (5% DMSO+20% Cremphor EL+0.9% normal saline) for 3 months. Non-transgenic mice (18 total mice, n=8-11 mice per treatment group) were used as a control and were injected daily (i.p.) with 10 mg/kg of Compound 1 (data not shown) or a vehicle control (5% DMSO+20% Cremphor EL+0.9% normal saline) for 1 month. After 30 days, the mice were sacrificed, and immunofluoresence (IF) detection of TSPO were assessed in the harvested brain tissues.

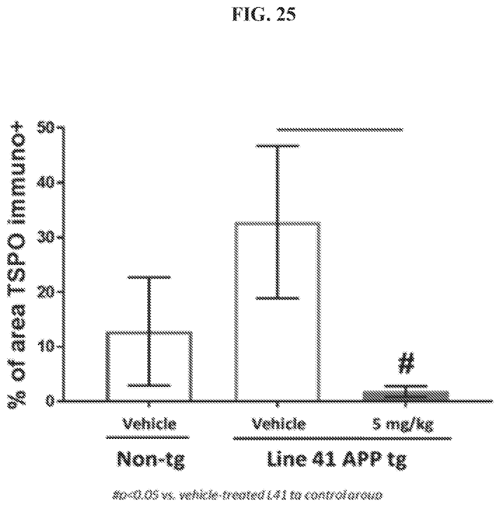

FIG. 25 shows the quantification of TPSO staining from representative brain sections. The harvested brain tissues were drop fixed using 4% PFA and sectioned on a vibratome, and representative sections corresponding to the neuropil of cortex were assessed for TSPO with standard immunofluorescence (IF) staining. The results show that when administered daily at 5 mg/kg, Compound 1 produced beneficial actions in reducing cortical levels of TSPO, as visualized by reduced IF staining intensity. Furthermore, the quantification in FIG. 25 shows that when administered daily at 5 mg/kg, Compound 1 reduces the TSPO level in a statistically significant manner as compared to vehicle-treated Line 41 mice.

vii. Effects of Compound 1 on Amyloid Beta Plaques

As described earlier, Line 41 transgenic mice express high levels of the mutant hAPP751 and develop mature plaques in the cortex, hippocampus, thalamus and olfactory region of mouse brain. The effects of Compound 1 on Amyloid beta plaque formation were assessed in both L41 APP transgenic and non-transgenic mice in a 1 month administration study. After 30 days, the mice were sacrificed, and immunofluorescence (IF) detection of amyloid beta were assessed in the harvested brain tissues.

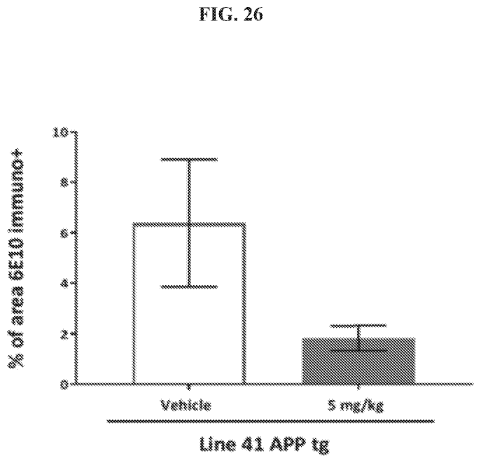

FIG. 26 shows the quantification of amyloid beta staining in L41 transgenic mice injected daily with Compound 1 versus vehicle control. The harvested brain tissues were drop fixed using 4% PFA and sectioned on a vibratome, and representative sections containing the neuropil of cortex, hippocampus and striatum were assessed for amyloid beta with standard IHC staining.

On approximately day 30, all subjects were euthanized within 2 hours of the last treatment and brain and other samples were collected. Brains were removed and divided sagitally. The right hemibrain was post-fixed in phosphate-buffered 4% PFA (pH 7.4) at 4° C. for 48 hours for neuropathological analysis. Drop fixed hemibrains were then serially sectioned into 40 uM thick coronal sections using a vibratome. Sections were free-floated and incubated overnight at 4° C. with primary antibodies. To confirm the specificity of primary antibodies, control experiments were performed in which sections were incubated overnight in the absence of primary antibody (deleted), preimmune serum, or primary antibody preadsorbed for 48 h with 20-fold excess of the corresponding peptide.

Immunolabeling studies of β-amyloid pathology were conducted using a purified anti-b-amyloid 1-16 antibody (1:500; 6E10 clone, reactive to amino acid residue 1-16 of β-amyloid and APP; #SIG-39320; Covance Research Products, Inc., Dedham, Mass., USA). Following incubations with primary antibodies, sections were then incubated with biotinylated secondary antibodies (1:200, Vector Laboratories, Burlingame, Calif.) and visualized using an avidin-biotin (ABC) kit (Vector Laboratories, Burlingame, Calif.) with diaminobenzidine tetrahydrochloride (DAB; Sigma-Aldrich, St. Louis, Mo.) as the chromogen.

Prepared slides were imaged at 40× using a high-resolution Hamamatsu Nanozoomer™ scanner located in the Microscopy Core in the UCSD Department of Neurosciences. Digital images were then transferred to Neuropore and analyzed using the Halo® imaging software package (Indica Labs, Corrales, N. Mex.). The same standardized regional mask (region of interest (ROI), with equal dimensions for equal analysis of area) was imported onto each image and positioned over the dorsal striatum. A window for thresholding was defined using representative images from the vehicle control groups, saved, and then applied to all images via a batch processing algorithm. Data are presented as percent (%) area of ROI immunopositive for each marker. Images were evaluated for specimen and imaging problems and any issues were noted prior to unblinding of samples and statistical analyses.

The results show that when administered daily at 5 mg/kg, Compound 1 produced beneficial actions in reducing cortical levels of amyloid beta, as visualized by reduced IF staining intensity. Furthermore, the quantification in FIG. 25 shows that when administered daily at 5 mg/kg, Compound 1 reduces the amyloid beta level in a statistically significant manner as compared to vehicle-treated Line 41 mice.

For all Figures, all data are presented as the group means±standard error of mean (****p<0.0001 or *p<0.05 denotes a statistically significant baseline or vehicle-treated phenotype compared to vehicle-treated non-transgenic control group; #p<0.05, ##p<0.01, ###p<0.001, or ####p<0.0001 denotes a statistically significant treatment effect in Compound 1-treated transgenic groups versus the vehicle-treated transgenic control group).

### Exemplary Embodiments

1. A method of treating a disease or condition associated with neurodegeneration or accumulation of proteins in the brain in a subject in need thereof, comprising administering to the subject an effective amount of an ion transporter inhibitor, wherein the ion transporter inhibitor modulates the efflux of one or more bioactive endogenous metabolites across the blood brain barrier (BBB) of the subject.

2. The method of embodiment 1, wherein the disease or condition associated with neurodegeneration is Alzheimer's Disease, Parkinson's Disease, fronto-temporal dementia, dementia with Lewy Bodies, PD dementia, multiple system atrophy, Huntington's disease, Amyotrophic lateral sclerosis, progressive supranuclear palsy, or neuroinflammation.

3. A method of modulating efflux of one or more bioactive endogenous metabolites across the blood brain barrier (BBB) in a subject in need thereof, comprising administering to the subject in need thereof an ion transporter inhibitor.

4. A method of improving neuroprotection in a subject in need thereof, comprising administering to the subject an effective amount of an ion transporter inhibitor that modulates the concentration of one or more bioactive endogenous metabolites in the brain interstitial space.

5. A method of decreasing neuroinflammation in a subject in need thereof, comprising administering to the subject an effective amount of an ion transporter inhibitor that modulates the concentration of one or more bioactive endogenous metabolites in the brain interstitial space.

6. The method of any one of embodiments 1-5, wherein the ion transporter inhibitor is an inhibitor of organic anion transporter 3 (OAT3).

7. The method of any one of embodiments 1-6, wherein the ion transporter inhibitor selectively inhibits OAT3 compared with other ion transporter proteins.

8. The method of any one of embodiments 1-7, wherein the ion transporter inhibitor has an IC₅₀ for OAT3 of about 1 μM or less.

9. The method of any one of embodiments 1-8, wherein the ion transporter inhibitor has an IC₅₀ for OAT3 that is at least about 10 fold lower compared with its IC₅₀ for organic anion transporter 1 (OAT1).

10. The method of any one of embodiments 1-9, wherein after administration of the ion transporter inhibitor, the efflux of the one or more bioactive endogenous metabolites across the BBB is reduced.

11. The method of any one of embodiments 1-10, wherein after administration of the ion transporter inhibitor, the local concentrations of the one or more bioactive endogenous metabolites in the brain interstitial space are increased.

12. The method of embodiment 11, wherein after administration of the ion transporter inhibitor, the levels of the one or more bioactive endogenous metabolites in the brain interstitial space are increased by about 50% or more.

13. The method of any one of embodiments 1-12, wherein after administration of the ion transporter inhibitor, the plasma levels of the one or more bioactive endogenous metabolites are decreased.

14. The method of embodiment 13, wherein the plasma levels of the bioactive endogenous metabolites are modulated by 50% or less.

15. The method of embodiment 11, wherein the plasma levels of the bioactive endogenous metabolites are decreased by 50% or less.

16. The method of any one of embodiments 1-15, wherein the one or more bioactive endogenous metabolites is an anionic neurotransmitter metabolite of epinephrine, norepinephrine, dopamine, and/or serotonin.

17. The method of any one of embodiments 1-15, wherein the one or more bioactive endogenous metabolites are selected from the group consisting of: uric acid, glutathione, dehydroepianodrosterone (DHEA), and DHEA sulfate (DHEAS).

18. The method of any one of embodiments 1-17, wherein the one or more bioactive endogenous metabolites have neuroprotective and/or anti-neuroinflammatory properties.

19. The method of embodiment 18, wherein the anti-neuroinflammatory properties include reduction of a pro-inflammatory response in the brain of the subject.

20. The method of embodiment 19, wherein the reduction of a pro-inflammatory response comprises reduction in gene expression of one or more of TNF, IL6, IL12/23p40 or MCP1.

21. The method of embodiment 19, wherein the reduction of a pro-inflammatory response is mediated by processes comprising activation of TrkA/Akt/CREB/Jmjd3 pathway in the brain of the subject.

22. The method of embodiment 21, wherein activation of the TrkA/Akt/CREB/Jmjd3 pathway comprises increase of pTrkA levels in the brain of the subject.

23. The method of embodiment 21 or embodiment 22, wherein activation of the TrkA/Akt/CREB/Jmjd3 pathway comprises increase of pAkt levels in the brain of the subject.

24. The method of any one of embodiments 21-23, wherein activation of the TrkA/Akt/CREB/Jmjd3 pathway comprises increase of pCREB levels in the brain of the subject.

25. The method of any one of embodiments 21-23, wherein activation of the TrkA/Akt/CREB/Jmjd3 pathway comprises an increase in Jmjd3 expression in the brain of the subject.

26. The method of embodiment 19, wherein the anti-neuroinflammatory properties comprises induction of an anti-inflammatory phenotype of microglia in the subject.

27. The method of embodiment 26, wherein the anti-inflammatory phenotype of microglia comprises increased gene expression of one or more of M2 polarization markers comprising one or more of arginase 1, Ym1 (chitinase-like protein 3), Fizz1, Klf4 (Kruppel like factor 4) or IL10.

28. The method of embodiment 26, wherein the anti-inflammatory phenotype of microglia comprises inhibition of a pro-inflammatory phenotype of microglia in the subject.

29. A method of preventing aggregation or accumulation or enhancing clearance of protease-resistant protein, comprising contacting the protease-resistant protein with an effective amount of a compound that is an inhibitor of organic anion transporter 3 (OAT3), wherein the contacting is in vitro, ex vivo, or in vivo.

30. The method of embodiment 29, wherein the protease-resistant protein is selected from alpha synuclein, a-beta, tau, Huntingtin, and TAR DNA binding protein 43 (TDP43) proteins.

31. The method of any one of embodiments 1-30, wherein the compound is a compound of Formula (I):

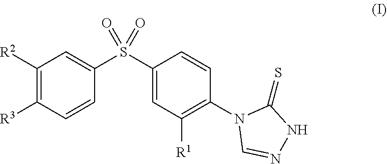

wherein
R¹, R², and R³ are each independently hydrogen, hydroxy, halogen, optionally substituted C₁₋₄ alkyl, optionally substituted C₁₋₄ alkoxy, —CN, —C(O)R^{x}, —C(O)OR^{x}, —S(O)₂R^{x}, or —NR^{y}R^{z};
R^{x}, R^{y}, and R^{z} are each independently H or optionally substituted C₁₋₄alkyl, or R^{y} and R^{z} taken together with the nitrogen to which they are attached form an optionally substituted monocyclic heterocycloalkyl ring;
   or a pharmaceutically acceptable salt thereof.

32. The method of embodiment 31, or a pharmaceutically acceptable salt thereof, wherein R¹ is hydrogen, optionally substituted C₁₋₄ alkoxy, or —NR^{y}R^{z}.

33. The method of embodiment 31 or embodiment 32, or a pharmaceutically acceptable salt thereof, wherein R¹ is hydrogen.

34. The method of embodiment 31 or embodiment 32, or a pharmaceutically acceptable salt thereof, wherein R¹ is C₁₋₄ alkoxy, which is unsubstituted or substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄ alkyl and R^{f} and R^{g} are each independently H, C₁₋₄alkyl, —C(O)C₁₋₄alkyl, —C(O)OC₁₋₄alkyl, or —S(O)₂C₁₋₄alkyl.

35. The method of embodiment 31 or embodiment 32, or a pharmaceutically acceptable salt thereof, wherein R¹ is —(OCH₂CH₂)ₚ—O—CH₂CH₃ or —(OCH₂CH₂)ₚ—O—CH₃, wherein p is 0-10.

36. The method of embodiment 35, or a pharmaceutically acceptable salt thereof, wherein R¹ is —OCH₂CH₂—O—CH₂CH₃ or —OCH₂CH₂OCH₃.

37. The method of embodiment 31 or embodiment 32, or a pharmaceutically acceptable salt thereof, wherein R¹ is —NR^{y}R^{z}, wherein R^{y} and R^{z} are each independently H or C₁₋₄alkyl, wherein the C₁₋₄alkyl is unsubstituted or substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄ alkyl.

38. The method of embodiment 37, wherein R¹ is —NHCH₂CH₂OH or —N(CH₂CH₃)₂.

39. The method of embodiment 31 or embodiment 32, or a pharmaceutically acceptable salt thereof, wherein R¹ is —NR^{y}R^{z}, and R^{y} and R^{z} taken together with the nitrogen to which they are attached form an optionally substituted monocyclic heterocycloalkyl ring.

40. The method of embodiment 39, or a pharmaceutically acceptable salt thereof, wherein R¹ is —NR^{y}R^{z}, and R^{y} and R^{z} taken together with the nitrogen to which they are attached form a monocyclic heterocycloalkyl ring selected from morpholinyl, piperazinyl, piperidinyl, and pyrrolidinyl, wherein the morpholinyl, piperazinyl, piperidinyl, and pyrrolidinyl are each unsubstituted or substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄alkyl.

41. The method of embodiment 39 or embodiment 40, wherein R¹ is morpholinyl, 4-methyl-piperazin-1-yl, piperidinyl, or pyrrolidinyl.

42. The method of any one of embodiments 31-41, or a pharmaceutically acceptable salt thereof, wherein R² is hydrogen, C₁₋₄ alkyl, or substituted C₁₋₄ alkyl.

43. The method of embodiment 42, or a pharmaceutically acceptable salt thereof, wherein R² is C₁₋₄ alkyl substituted with halogen.

44. The method of any one of embodiments 31-42, or a pharmaceutically acceptable salt thereof, wherein R² is CF₃.

45. The method of embodiment 42, or a pharmaceutically acceptable salt thereof, wherein R² is methyl.

46. The method of any one of embodiments 31-41, or a pharmaceutically acceptable salt thereof, wherein R² is optionally substituted C₁₋₄ alkoxy, —CN, or —NR^{y}R^{z}.

47. The method of embodiment 46, or a pharmaceutically acceptable salt thereof, wherein R² is —NR^{y}R^{z}, wherein R^{y} and R^{z} are each independently H or C₁₋₄alkyl, wherein the C₁₋₄alkyl is unsubstituted or substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄alkyl.

48. The method of embodiment 47, wherein R² is —N(CH₃)₂.

49. The method of embodiment 46, or a pharmaceutically acceptable salt thereof, wherein R² is —NR^{y}R^{z}, and R^{y} and R^{z} taken together with the nitrogen to which they are attached form an optionally substituted monocyclic heterocycloalkyl ring.

50. The method of embodiment 49, or a pharmaceutically acceptable salt thereof, wherein R² is —NR^{y}R^{z}, and R^{y} and R^{z} taken together with the nitrogen to which they are attached form a monocyclic heterocycloalkyl ring selected from morpholinyl, piperazinyl, piperidinyl, and pyrrolidinyl, wherein the morpholinyl, piperazinyl, piperidinyl, and pyrrolidinyl are each unsubstituted or substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄alkyl.

51. The method of any one of embodiments 46, 49 and 50, or a pharmaceutically acceptable salt thereof, wherein R² is morpholinyl.

52. The method of embodiment 46, or a pharmaceutically acceptable salt thereof, wherein R² is —CN.

53. The method of embodiment 46, or a pharmaceutically acceptable salt thereof, wherein R² is C₁₋₄ alkoxy, unsubstituted or substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄alkyl.

54. The method of any one of embodiments 31-41, or a pharmaceutically acceptable salt thereof, wherein R² is —(OCH₂CH₂)ₚ—O—CH₂CH₃ or —(OCH₂CH₂)ₚ—O—CH₃, wherein p is 0-10.

55. The method of embodiment 43, or a pharmaceutically acceptable salt thereof, wherein R² is methoxy or —OCH₂CH₂—O—CH₂CH₃ or —OCH₂CH₂OCH₃.

56. The method of any one of embodiments 31-55, or a pharmaceutically acceptable salt thereof, wherein R³ is halogen.

57. The method of any one of embodiments 31-55, or a pharmaceutically acceptable salt thereof, wherein R³ is chloro.

58. The method of any one of embodiments 31-55, or a pharmaceutically acceptable salt thereof, wherein R³ is hydrogen.

59. The method of any one of embodiments 31-55, or a pharmaceutically acceptable salt thereof, wherein R³ is C₁₋₄ alkyl or substituted C₁₋₄ alkyl.

60. The method of embodiment 59, or a pharmaceutically acceptable salt thereof, wherein R³ is methyl.

61. The method of embodiment 59, or a pharmaceutically acceptable salt thereof, wherein R³ is C₁₋₄ alkyl substituted with one or more halogen.

62. The method of embodiment 61, or a pharmaceutically acceptable salt thereof, wherein R³ is CF₃.

63. The method of any one of embodiments 31-55, or a pharmaceutically acceptable salt thereof, wherein R³ is —CN.

64. The method of any one of embodiments 31-55, or a pharmaceutically acceptable salt thereof, wherein R³ is —NR^{y}R^{z}, wherein R^{y} and R^{z} are each independently H or C₁₋₄alkyl, wherein the C₁₋₄alkyl is unsubstituted or substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄ alkyl.

65. The method of any one of embodiments 31-55, or a pharmaceutically acceptable salt thereof, wherein R³ is —NR^{y}R^{z}, and R^{y} and R^{z} taken together with the nitrogen to which they are attached form an optionally substituted monocyclic heterocycloalkyl ring.

66. The method of embodiment 55, or a pharmaceutically acceptable salt thereof, wherein R³ is —NR^{y}R^{z}, and R^{y} and R^{z} taken together with the nitrogen to which they are attached form a monocyclic heterocycloalkyl ring selected from morpholinyl, piperazinyl, piperidinyl, and pyrrolidinyl, wherein the morpholinyl, piperazinyl, piperidinyl, and pyrrolidinyl are each unsubstituted or substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄alkyl.

67. The method of embodiment 65 or embodiment 66, wherein R³ is morpholinyl.

68. The method of any one of embodiments 31-55, or a pharmaceutically acceptable salt thereof, wherein R³ is C₁₋₄ alkoxy, unsubstituted or substituted with one or more substituents selected from the group consisting of C₁-C₆ alkyl, C₂-C₆ alkenyl, C₂-C₆ alkynyl, hydroxyl, halogen, —NR^{f}R^{g}, cyano, nitro, C₁₋₄ alkoxy, C₁₋₄ haloalkoxy, —C(O)R⁴, —OC(O)R⁴, —C(O)OR⁴, —C(O)NR^{f}R^{g}, and —OC(O)NR^{f}R^{g}, wherein R⁴ is H or C₁₋₄alkyl and R^{f} and R^{g} are each independently H, C₁₋₄alkyl, —C(O)C₁₋₄alkyl, —C(O)OC₁₋₄alkyl, or —S(O)₂C₁₋₄ alkyl.

69. The method of any one of embodiments 31-55, or a pharmaceutically acceptable salt thereof, wherein R³ is —(OCH₂CH₂)ₚ—O—CH₂CH₃ or —(OCH₂CH₂)ₚ—O—CH₃, wherein p is 0-10.

70. The method of any one of embodiments 1-30, wherein the compound is

| Compound | | |
|---|---|---|
| No. | Structure | Name |
| 1 | 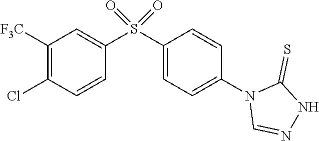 [SMILES: O=S(=O)(c1ccc(-n2cn[nH]c2=S)cc1)c1ccc(Cl)c(C(F)(F)F)c1] | 4-(4-((4-chloro-3- (trifluoromethyl)phenyl)sulfonyl)phenyl)- 2,4-dihydro-3H-1,2,4-triazole-3- thione; |
| 2 | 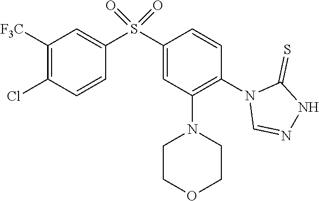 [SMILES: O=S(=O)(c1ccc(-n2cn[nH]c2=S)c(N2CCOCC2)c1)c1ccc(Cl)c(C(F)(F)F)c1] | 4-(4-((4-chloro-3- (trifluoromethyl)phenyl)sulfonyl)-2- morpholinophenyl)-2,4-dihydro-3H- 1,2,4-triazole-3-thione; |
| 3 | 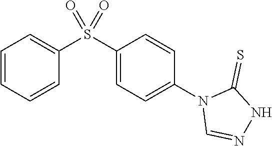 [SMILES: O=S(=O)(c1ccccc1)c1ccc(-n2cn[nH]c2=S)cc1] | 4-(4-(phenylsulfonyl)phenyl)-2,4- dihydro-3H-1,2,4-triazole-3-thione; |
| 4 | 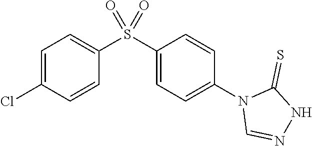 [SMILES: O=S(=O)(c1ccc(Cl)cc1)c1ccc(-n2cn[nH]c2=S)cc1] | 4-(4-((4-chlorophenyl)sulfonyl)phenyl)- 2,4-dihydro-3H-1,2,4-triazole-3-thione; |
| 5 | 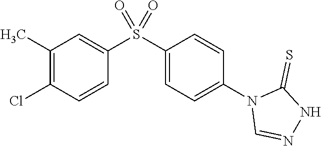 [SMILES: Cc1cc(S(=O)(=O)c2ccc(-n3cn[nH]c3=S)cc2)ccc1Cl] | 4-(4-((4-chloro-3- methylphenyl)sulfonyl)phenyl)-2,4- dihydro-3H-1,2,4-triazole-3-thione; |
| 6 | 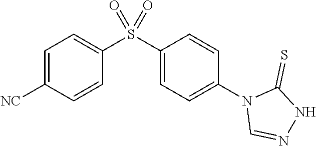 [SMILES: N#Cc1ccc(S(=O)(=O)c2ccc(-n3cn[nH]c3=S)cc2)cc1] | 4-((4-(5-thioxo-1,5-dihydro-4H-1,2,4- triazol-4- yl)phenyl)sulfonyl)benzonitrile; |
| 7 | 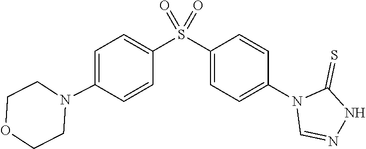 [SMILES: O=S(=O)(c1ccc(N2CCOCC2)cc1)c1ccc(-n2cn[nH]c2=S)cc1] | 4-(4-((4- morpholinophenyl)sulfonyl)phenyl)- 2,4-dihydro-3H-1,2,4-triazole-3-thione; |
| 8 | 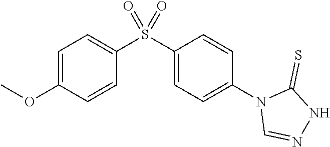 [SMILES: COc1ccc(S(=O)(=O)c2ccc(-n3cn[nH]c3=S)cc2)cc1] | 4-(4-((4- methoxyphenyl)sulfonyl)phenyl)-2,4- dihydro-3H-1,2,4-triazole-3-thione; |
| 9 | 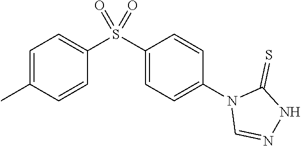 [SMILES: Cc1ccc(S(=O)(=O)c2ccc(-n3cn[nH]c3=S)cc2)cc1] | 4-(4-tosylphenyl)-2,4-dihydro-3H-1,2,4- triazole-3-thione; |
| 10 | 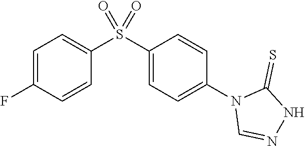 [SMILES: O=S(=O)(c1ccc(F)cc1)c1ccc(-n2cn[nH]c2=S)cc1] | 4-(4-((4-fluorophenyl)sulfonyl)phenyl)- 2,4-dihydro-3H-1,2,4-triazole-3-thione; |
| 11 | 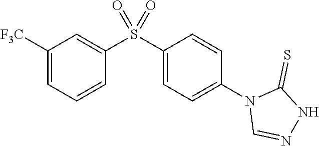 [SMILES: O=S(=O)(c1ccc(-n2cn[nH]c2=S)cc1)c1cccc(C(F)(F)F)c1] | 4-(4-((3- (trifluoromethyl)phenyl)sulfonyl)phenyl)- 2,4-dihydro-3H-1,2,4-triazole-3- thione; |
| 12 | 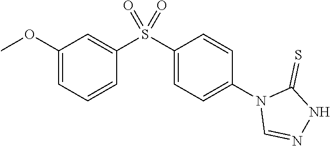 [SMILES: COc1cccc(S(=O)(=O)c2ccc(-n3cn[nH]c3=S)cc2)c1] | 4-(4-((3- methoxyphenyl)sulfonyl)phenyl)-2,4- dihydro-3H-1,2,4-triazole-3-thione; |
| 13 | 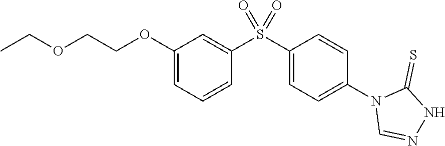 [SMILES: CCOCCOc1cccc(S(=O)(=O)c2ccc(-n3cn[nH]c3=S)cc2)c1] | 4-(4-((3-(2- ethoxyethoxy)phenyl)sulfonyl)phenyl)- 2,4-dihydro-3H-1,2,4-triazole-3-thione; |
| 14 | 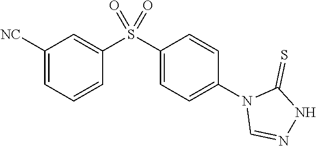 [SMILES: N#Cc1cccc(S(=O)(=O)c2ccc(-n3cn[nH]c3=S)cc2)c1] | 3-((4-(5-thioxo-1,5-dihydro-4H-1,2,4- triazol-4- yl)phenyl)sulfonyl)benzonitrile; |
| 15 | 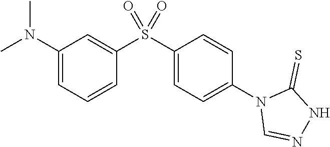 [SMILES: CN(C)c1cccc(S(=O)(=O)c2ccc(-n3cn[nH]c3=S)cc2)c1] | 4-(4-((3- (dimethylamino)phenyl)sulfonyl)phenyl)- 2,4-dihydro-3H-1,2,4-triazole-3- thione; |
| 16 | 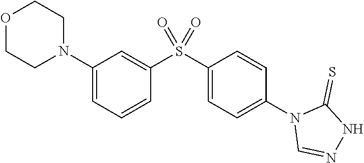 [SMILES: O=S(=O)(c1ccc(-n2cn[nH]c2=S)cc1)c1cccc(N2CCOCC2)c1] | 4-(4-((3- morpholinophenyl)sulfonyl)phenyl)- 2,4-dihydro-3H-1,2,4-triazole-3-thione; |
| 17 | 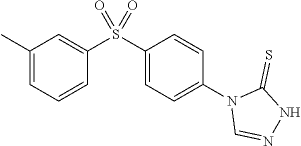 [SMILES: Cc1cccc(S(=O)(=O)c2ccc(-n3cn[nH]c3=S)cc2)c1] | 4-(4-(m-tolylsulfonyl)phenyl)-2,4- dihydro-3H-1,2,4-triazole-3-thione; |
| 18 | 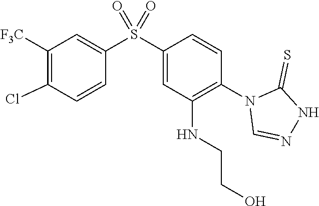 [SMILES: O=S(=O)(c1ccc(-n2cn[nH]c2=S)c(NCCO)c1)c1ccc(Cl)c(C(F)(F)F)c1] | 4-(4-((4-chloro-3- (trifluoromethyl)phenyl)sulfonyl)-2-((2- hydroxyethyl)amino)phenyl)-2,4- dihydro-3H-1,2,4-triazole-3-thione; |
| 19 | 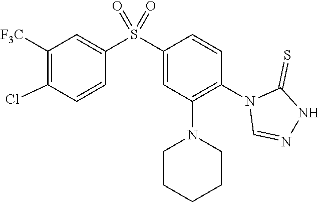 [SMILES: O=S(=O)(c1ccc(-n2cn[nH]c2=S)c(N2CCCCC2)c1)c1ccc(Cl)c(C(F)(F)F)c1] | 4-(4-((4-chloro-3- (trifluoromethyl)phenyl)sulfonyl)-2- (piperidin-1-yl)phenyl)-2,4-dihydro-3H- 1,2,4-triazole-3-thione; |
| 20 | 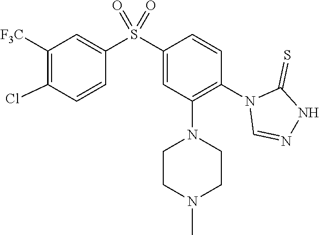 [SMILES: CN1CCN(c2cc(S(=O)(=O)c3ccc(Cl)c(C(F)(F)F)c3)ccc2-n2cn[nH]c2=S)CC1] | 4-(4-((4-chloro-3- (trifluoromethyl)phenyl)sulfonyl)-2-(4- methylpiperazin-1-yl)phenyl)-2,4- dihydro-3H-1,2,4-triazole-3-thione; |
| 21 | 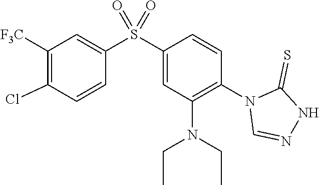 [SMILES: CCN(CC)c1cc(S(=O)(=O)c2ccc(Cl)c(C(F)(F)F)c2)ccc1-n1cn[nH]c1=S] | 4-(4-((4-chloro-3- (trifluoromethyl)phenyl)sulfonyl)-2- (diethylamino)phenyl)-2,4-dihydro-3H- 1,2,4-triazole-3-thione; |
| 22 | 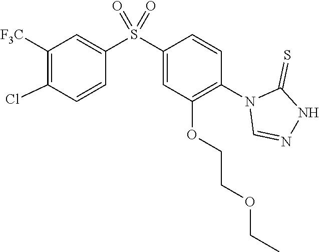 [SMILES: CCOCCOc1cc(S(=O)(=O)c2ccc(Cl)c(C(F)(F)F)c2)ccc1-n1cn[nH]c1=S] | 4-(4-((4-chloro-3- (trifluoromethyl)phenyl)sulfonyl)-2-(2- ethoxyethoxy)phenyl)-2,4-dihydro-3H- 1,2,4-triazole-3-thione; |
| 23 | 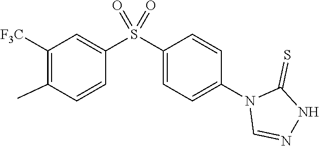 [SMILES: Cc1ccc(S(=O)(=O)c2ccc(-n3cn[nH]c3=S)cc2)cc1C(F)(F)F] | 4-(4-((4-methyl-3- (trifluoromethyl)phenyl)sulfonyl)phenyl)- 2,4-dihydro-3H-1,2,4-triazole-3- thione; |
| 24 | 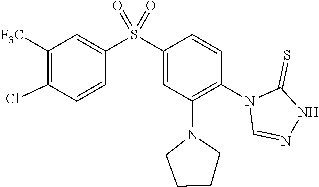 [SMILES: O=S(=O)(c1ccc(-n2cn[nH]c2=S)c(N2CCCC2)c1)c1ccc(Cl)c(C(F)(F)F)c1] | 4-(4-((4-chloro-3- (trifluoromethyl)phenyl)sulfonyl)-2- (pyrrolidin-1-yl)phenyl)-2,4-dihydro- 3H-1,2,4-triazole-3-thione; and |
| 25 | 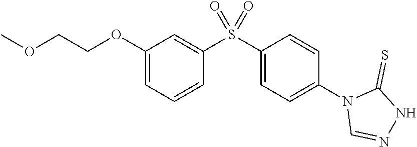 [SMILES: COCCOc1cccc(S(=O)(=O)c2ccc(-n3cn[nH]c3=S)cc2)c1] | 4-(4-((3-(2- methoxyethoxy)phenyl)sulfonyl)phenyl)- 2,4-dihydro-3H-1,2,4-triazole-3- thione, |

or a pharmaceutically acceptable salt thereof.

71. The method of any one of embodiments 1-30, wherein the compound is a compound having the following structure:

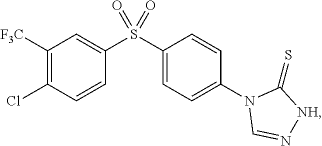 [SMILES: O=S(=O)(c1ccc(-n2cn[nH]c2=S)cc1)c1ccc(Cl)c(C(F)(F)F)c1]

or a pharmaceutically acceptable salt thereof.

72. The method of any one of embodiments 1-30, wherein the compound is a compound having the following structure:

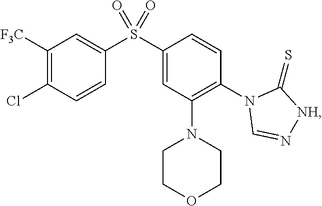 [SMILES: O=S(=O)(c1ccc(-n2cn[nH]c2=S)c(N2CCOCC2)c1)c1ccc(Cl)c(C(F)(F)F)c1]

or a pharmaceutically acceptable salt thereof.

73. The method of any one of embodiments 1-30, wherein the compound is not a

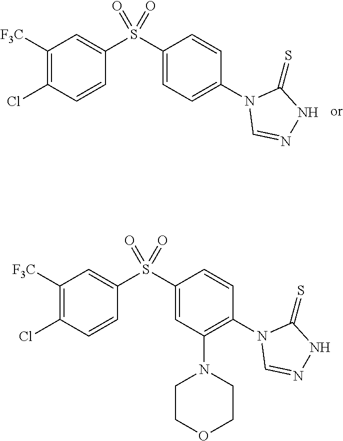 [SMILES: O=S(=O)(c1ccc(-n2cn[nH]c2=S)cc1)c1ccc(Cl)c(C(F)(F)F)c1 ; "or" ; O=S(=O)(c1ccc(-n2cn[nH]c2=S)c(N2CCOCC2)c1)c1ccc(Cl)c(C(F)(F)F)c1]

or a pharmaceutically acceptable salt thereof.

74. The method of any one of embodiments 1-30, wherein the compound is a compound of Formula (IIA):

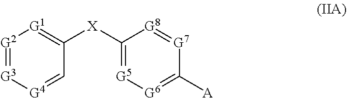

wherein

### G¹ is CH or N;

G² is CR^{2a} or N;
G³ is CR^{3a} or N;

### G⁴ is CH or N;

wherein no more than two of Gi, G², G³, and G⁴ are N;

### G⁵ is CH or N;

G⁶ is CR^{1a} or N;

### G⁷ is CH or N;

### G⁸ is CH or N;

wherein no more than one of G⁵, G⁶, G⁷, and G⁸ is N;
R^{1a}, R^{2a}, and R^{3a} are each independently hydrogen, hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, or an optionally substituted heterocyclyl;
   wherein R^{x} and R^{y} are each independently H or optionally substituted C₁₋₄alkyl;
or R^{1a} and R^{2a} are taken together with the carbons to which they are attached to form a 5- to 16-membered heterocyclyl ring;
   X is —CR^{4a}R^{5a}—, —O—, —S—, —S(O)—, —NR^{6a}—, —S(O)₂—, —NR^{6a}S(O)₂—, —CR^{4a}R^{5a}S(O)₂—, —C(O)—, —NR^{6a}C(O)—, or —NHNHC(O)—;

wherein R^{4a} and R^{5a} are independently hydrogen, hydroxy, halogen, substituted C₁₋₄ alkyl, C₁₋₄ alkoxy, or substituted C₁₋₄ alkoxy;

or R^{4a} and R^{5a} are taken together with the carbon to which they are attached to form a 3- to 6-membered cycloalkyl ring;

each R^{6a} is independently hydrogen or C₁₋₄ alkyl;

A is or

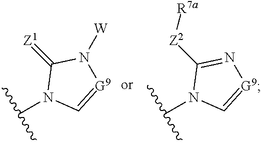

### G⁹ is CH or N;

Z¹ and Z² are independently S or O; and
W and R^{7a} are independently hydrogen or C₁₋₄ alkyl,

or a pharmaceutically acceptable salt thereof.

75. The method of any one of embodiments 1-30, wherein the compound is a compound of Formula (II):

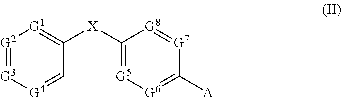

wherein

### G¹ is CH or N;

G² is CR^{2a} or N;
G³ is CR^{3a} or N;

### G⁴ is CH or N;

wherein no more than two of G¹, G², G³, and G⁴ are N;

### G⁵ is CH or N;

G⁶ is CR^{1a} or N;

### G⁷ is CH or N;

### G⁸ is CH or N;

wherein no more than one of G⁵, G⁶, G⁷, and G⁸ is N;
R^{1a}, R^{2a}, and R^{3a} are each independently hydrogen, hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, or an optionally substituted heterocyclyl;
   wherein R^{x} and R^{y} are each independently H or optionally substituted C₁₋₄alkyl;
or R^{1a} and R^{2a} are taken together with the carbons to which they are attached to form a 5- to 16-membered heterocyclyl ring;
   X is —CR^{4a}R^{5a}—, —O—, —S—, —S(O)—, —NR^{6a}—, —S(O)₂—, or —C(O)—;

wherein R^{4a} and R^{5a} are independently hydrogen, hydroxy, halogen, substituted C₁₋₄ alkyl, C₁₋₄ alkoxy, or substituted C₁₋₄ alkoxy;

or R^{4a} and R^{5a} are taken together with the carbon to which they are attached to form a 3- to 6-membered cycloalkyl ring;

R^{6a} is hydrogen or C₁₋₄ alkyl;

### A is

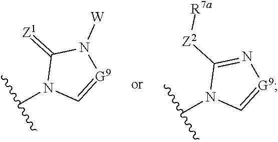

### G⁹ is CH or N;

Z¹ and Z² are independently S or O; and
W and R^{7a} are independently hydrogen or C₁₋₄ alkyl,
or a pharmaceutically acceptable salt thereof.

76. The method of embodiment 74 or 75, wherein G¹ is CH.

77. The method of embodiment 74 or 75, wherein G¹ is N.

78. The method of any one of embodiments 74-77, wherein G² is CR^{2a}.

79. The method of any one of embodiments 74-78, wherein G³ is CR^{3a}

80. The method of embodiment 78, wherein R^{2a} is hydrogen, hydroxy, halogen, C₁₋₄ alkyl, C₁₋₄ alkyl substituted with one or more halogen, C₁₋₄ alkoxy, C₁₋₄ alkoxy substituted with one or more halogen or C₁₋₄ alkoxy, —CN, —NR^{x}R^{y}, or an optionally substituted heterocyclyl.

81. The method of embodiment 79, wherein R^{3a} is hydrogen, hydroxy, halogen, C₁₋₄ alkyl, C₁₋₄ alkyl substituted with one or more halogen, C₁₋₄ alkoxy, C₁₋₄ alkoxy substituted with one or more halogen or C₁₋₄ alkoxy, —CN, —NR^{x}R^{y}, or an optionally substituted heterocyclyl.

82. The method of any one of embodiments 74-79, wherein R^{2a} and R^{3a} are taken together with the carbons to which they are attached to form a 5- to 16-membered heterocyclyl ring;

83. The method of any one of embodiments 74-77, 79, and 81, wherein G² is N.

84. The method of any one of embodiments 74-78, and 80, wherein G³ is N.

85. The method of any one of embodiments 74-84, wherein G⁴ is CH.

86. The method of any one of embodiments 74-84, wherein G⁴ is N.

87. The method of any one of embodiments 74-86, wherein X is —CR^{4a}R^{5a}—; wherein R^{4a} and R^{5a}, are each independently hydrogen, hydroxy, halogen, or R^{4a} and R^{5a} are taken together with the carbon to which they are attached to form a 3- to 6-membered cycloalkyl ring.

88. The method of any one of embodiments 74-86, wherein X is —S(O)₂—.

89. The method of any one of embodiments 74-86, wherein X is —O— or —S—.

90. The method of any one of embodiments 74-86, wherein X is —S(O)— or —C(O)—.

91. The method of any one of embodiments 74-88, wherein X is —NR^{6a}—, wherein R^{6a} is hydrogen or C₁₋₄ alkyl.

92. The method of any one of embodiments 74-91, wherein G⁵ is CH.

93. The method of any one of embodiments 74-91, wherein G⁵ is N.

94. The method of any one of embodiments 74-93, wherein G⁶ is CR^{1a}

95. The method of embodiment 94, wherein R^{1a} is hydrogen, hydroxy, halogen, C₁₋₄ alkyl, C₁₋₄ alkyl substituted with one or more halogen, C₁₋₄ alkoxy, C₁₋₄ alkoxy substituted with one or more halogen or C₁₋₄ alkoxy, —CN, —NR^{x}R^{y}, or an optionally substituted heterocyclyl.

96. The method of any one of embodiments 74-92, wherein G⁶ is N.

97. The method of any one of embodiments 74-96, wherein G⁷ is CH.

98. The method of any one of embodiments 74-92 and 94-95, wherein G⁷ is N.

99. The method of any one of embodiments 74-98, wherein G⁸ is CH.

100. The method of any one of embodiments 74-92, 94-95, and 97, wherein G⁸ is N.

101. The method of any one of embodiments 74-100, wherein A is

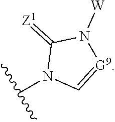

102. The method of embodiment 101, wherein Z¹ is S.

103. The method of embodiment 101, wherein Z¹ is O.

104. The method of any one of embodiments 101-103, wherein G⁹ is CH.

105. The method of any one of embodiments 101-103, wherein G⁹ is N.

106. The method of any one of embodiments 101-105, wherein W is hydrogen.

107. The method of any one of embodiments 101-105, wherein W is C₁₋₄ alkyl.

108. The method of any one of embodiments 74-100, wherein A is

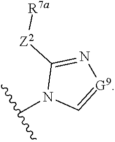

109. The method of embodiment 108, wherein Z² is S.

110. The method of embodiment 108, wherein Z² is O.

111. The method of any one of embodiments 108-110, wherein R^{7a} is hydrogen.

112. The method of any one of embodiments 108-110, wherein R^{7a} is C₁₋₄ alkyl.

113. The method of any one of embodiments 108-112, wherein G⁹ is CH.

114. The method of any one of embodiments 108-112, wherein G⁹ is N.

115. The method of any one of embodiments 1-30, wherein the compound is selected from the group consisting of

| Compound | | |
|---|---|---|
| No. | Structure | Name |
| 26 | 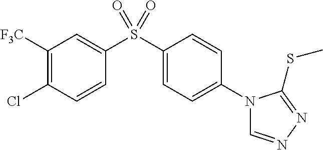 [SMILES: CSc1nncn1-c1ccc(S(=O)(=O)c2ccc(Cl)c(C(F)(F)F)c2)cc1] | 4-(4-((4-chloro-3- (trifluoromethyl)phenyl)sulfonyl)phenyl)- 3-(methylthio)-4H-1,2,4-triazole; |
| 27 | 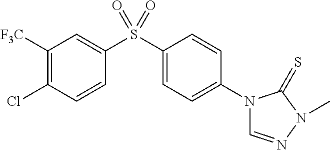 [SMILES: Cn1ncn(-c2ccc(S(=O)(=O)c3ccc(Cl)c(C(F)(F)F)c3)cc2)c1=S] | 4-(4-((4-chloro-3- (trifluoromethyl)phenyl)sulfonyl)phenyl)- 2-methyl-2,4-dihydro-3H-1,2,4-triazole- 3-thione; |
| 28 | 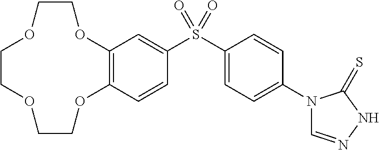 [SMILES: O=S(=O)(c1ccc(-n2cn[nH]c2=S)cc1)c1ccc2c(c1)OCCOCCOCCO2] | 4-(4-((2,3,5,6,8,9- hexahydrobenzo[b][1,4,7,10]tetraoxacyclo- dodecin-12-yl)sulfonyl)phenyl)-2,4- dihydro-3H-1,2,4-triazole-3-thione; |
| 29 | 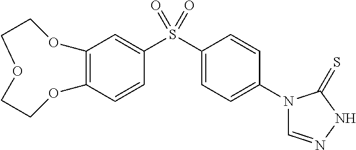 [SMILES: O=S(=O)(c1ccc(-n2cn[nH]c2=S)cc1)c1ccc2c(c1)OCCOCCO2] | 4-(4-((2,3,5,6- tetrahydrobenzo[b][1,4,7]trioxonin-9- yl)sulfonyl)phenyl)-2,4-dihydro-3H- 1,2,4-triazole-3-thione; |
| 30 | 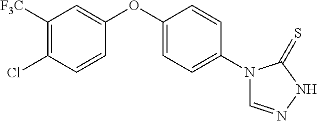 [SMILES: FC(F)(F)c1cc(Oc2ccc(-n3cn[nH]c3=S)cc2)ccc1Cl] | 4-(4-(4-chloro-3-(trifluoro-methyl)- phenoxy)-phenyl)-3,4-dihydro-2H-1,2,4- triazole-3-thione; |
| 31 | 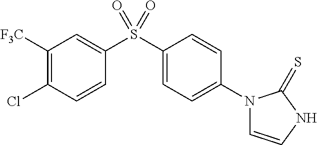 [SMILES: O=S(=O)(c1ccc(-n2cc[nH]c2=S)cc1)c1ccc(Cl)c(C(F)(F)F)c1] | 1-(4-(4-chloro-3-(trifluoro-methyl)- phenylsulfonyl)-phenyl)-2,3-dihydro- 1H-imidazole-2-thione; |
| 32 | 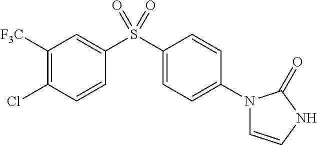 [SMILES: O=c1[nH]ccn1-c1ccc(S(=O)(=O)c2ccc(Cl)c(C(F)(F)F)c2)cc1] | 1-(4-(4-chloro-3-(trifluoro-methyl)- phenylsulfonyl)-phenyl)-2,3-dihydro- 1H-imidazol-2-one; |
| 33 | 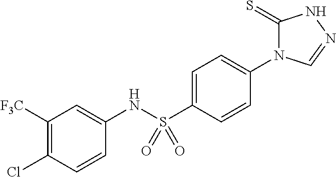 [SMILES: O=S(=O)(Nc1ccc(Cl)c(C(F)(F)F)c1)c1ccc(-n2cn[nH]c2=S)cc1] | N-(4-chloro-3-(trifluoromethyl)phenyl)- 4-(5-thioxo-1,5-dihydro-4H-1,2,4- triazol-4-yl)benzenesulfonamide; |
| 34 | 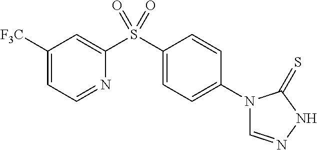 [SMILES: O=S(=O)(c1ccc(-n2cn[nH]c2=S)cc1)c1cc(C(F)(F)F)ccn1] | 4-(4-((2-(trifluoromethyl)pyridin-4- yl)sulfonyl)phenyl)-2,4-dihydro-3H- 1,2,4-triazole-3-thione; |
| 35 | 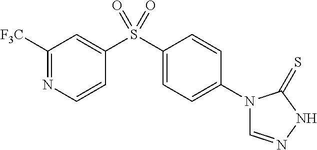 [SMILES: O=S(=O)(c1ccc(-n2cn[nH]c2=S)cc1)c1ccnc(C(F)(F)F)c1] | 4-(4-((4-(trifluoromethyl)pyridin-2- yl)sulfonyl)phenyl)-2,4-dihydro-3H- 1,2,4-triazole-3-thione; |
| 36 | 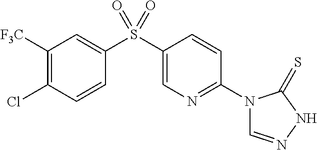 [SMILES: O=S(=O)(c1ccc(-n2cn[nH]c2=S)nc1)c1ccc(Cl)c(C(F)(F)F)c1] | 4-(5-((4-chloro-3- (trifluoromethyl)phenyl)sulfonyl)pyridin- 2-yl)-2,4-dihydro-3H-1,2,4-triazole-3- thione; |
| 37 | 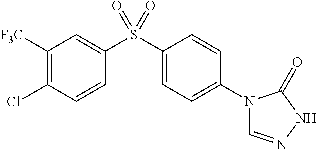 [SMILES: O=c1[nH]ncn1-c1ccc(S(=O)(=O)c2ccc(Cl)c(C(F)(F)F)c2)cc1] | 4-(4-((4-chloro-3- (trifluoromethyl)phenyl)sulfonyl)phenyl)- 2,4-dihydro-3H-1,2,4-triazol-3-one; |
| 38 | 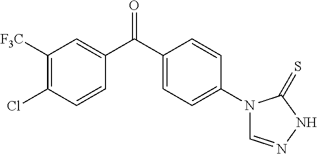 [SMILES: O=C(c1ccc(-n2cn[nH]c2=S)cc1)c1ccc(Cl)c(C(F)(F)F)c1] | (4-chloro-3-(trifluoromethyl)phenyl)(4- (5-thioxo-1,5-dihydro-4H-1,2,4-triazol- 4-yl)phenyl)methanone; |
| 39 | 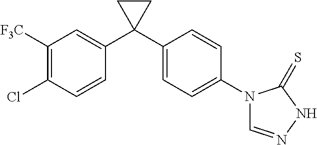 [SMILES: FC(F)(F)c1cc(C2(c3ccc(-n4cn[nH]c4=S)cc3)CC2)ccc1Cl] | 4-(4-(1-(4-chloro-3- (trifluoromethyl)phenyl)cyclopropyl)phe- nyl)-2,4-dihydro-3H-1,2,4-triazole-3- thione; |
| 40 | 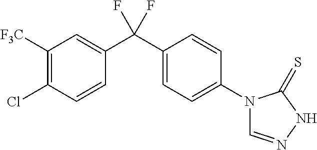 [SMILES: FC(F)(F)c1cc(C(F)(F)c2ccc(-n3cn[nH]c3=S)cc2)ccc1Cl] | 4-(4-((4-chloro-3- (trifluoromethyl)phenyl)difluoromethyl) phenyl)-2,4-dihydro-3H-1,2,4-triazole-3- thione; |
| 41 | 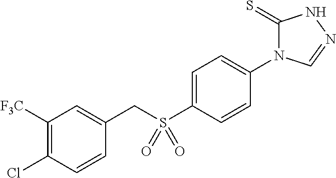 [SMILES: O=S(=O)(Cc1ccc(Cl)c(C(F)(F)F)c1)c1ccc(-n2cn[nH]c2=S)cc1] | 4-(4-((4-chloro-3- (trifluoromethyl)benzyl)sulfonyl)phenyl)- 2,4-dihydro-3H-1,2,4-triazole-3-thione; |
| 42 | 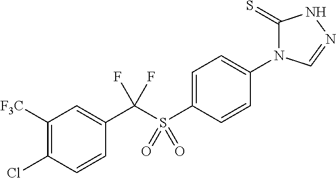 [SMILES: O=S(=O)(c1ccc(-n2cn[nH]c2=S)cc1)C(F)(F)c1ccc(Cl)c(C(F)(F)F)c1] | 4-(4-(((4-chloro-3- (trifluoromethyl)phenyl)difluoromethyl) sulfonyl)phenyl)-2,4-dihydro-3H-1,2,4- triazole-3-thione; |
| 43 | 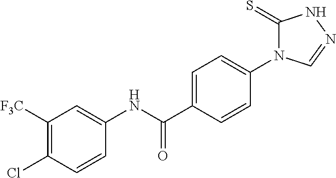 [SMILES: O=C(Nc1ccc(Cl)c(C(F)(F)F)c1)c1ccc(-n2cn[nH]c2=S)cc1] | N-(4-chloro-3-(trifluoromethyl)phenyl)- 4-(5-thioxo-1,5-dihydro-4H-1,2,4- triazol-4-yl)benzamide; and |
| 44 | 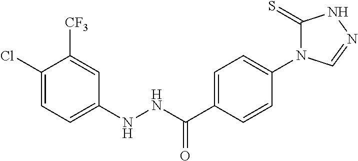 [SMILES: O=C(NNc1ccc(Cl)c(C(F)(F)F)c1)c1ccc(-n2cn[nH]c2=S)cc1] | N′-(4-chloro-3-(trifluoromethyl)phenyl)- 4-(5-thioxo-1,5-dihydro-4H-1,2,4- triazol-4-yl)benzohydrazide, |

or a pharmaceutically acceptable salt thereof.

116. A compound of Formula (IIA):

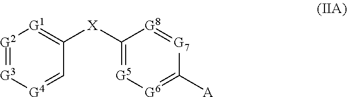

wherein

### G¹ is CH or N;

G² is CR^{2a} or N;
G³ is CR^{3a} or N;

### G⁴ is CH or N;

wherein no more than two of G¹, G², G³, and G⁴ are N;

### G⁵ is CH or N;

G⁶ is CR^{1a} or N;

### G⁷ is CH or N;

### G⁸ is CH or N;

wherein no more than one of G⁵, G⁶, G⁷, and G⁸ is N;
R^{1a}, R^{2a}, and R^{3a} are each independently hydrogen, hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, or an optionally substituted heterocyclyl;
   wherein R^{x} and R^{y} are each independently H or optionally substituted C₁₋₄alkyl;
or R^{1a} and R^{2a} are taken together with the carbons to which they are attached to form a 5- to 16-membered heterocyclyl ring;
   X is —CR^{4a}R^{5a}—, —O—, —S—, —S(O)—, —NR^{6a}—, —S(O)₂—, —NR^{6a}S(O)₂—, —CR^{4a}R^{5a}S(O)₂—, —C(O)—, —NR^{6a}C(O)—, or —NHNHC(O)—;

wherein R^{4a} and R^{5a} are independently hydrogen, hydroxy, halogen, substituted C₁₋₄ alkyl, C₁₋₄ alkoxy, or substituted C₁₋₄ alkoxy;

or R^{4a} and R^{5a} are taken together with the carbon to which they are attached to form a 3- to 6-membered cycloalkyl ring;

each R^{6a} is independently hydrogen or C₁₋₄ alkyl;

### A is

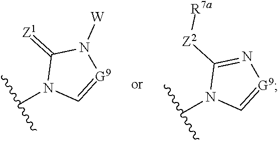

### G⁹ is CH or N;

Z¹ and Z² are independently S or O; and
W and R^{7a} are independently hydrogen or C₁₋₄ alkyl; and

one or more of the following apply:

(i) X is —CR^{4a}R^{5a}—, —O—, —S—, —S(O)—, —NR^{6a}—, —NR^{6a}S(O)₂—, —CR^{4a}R^{5a}S(O)₂—, —C(O)—, —NR^{6a}C(O)—, or —NHNHC(O)—;

(ii) one or two of G¹, G², G³, and G⁴ is N;

(iii) one of G⁵, G⁶, G⁷, and G⁸ is N;

(iv) R^{1a} is an optionally substituted heterocyclyl, wherein when the heterocyclyl is monocyclic, the point of connection is via a carbon atom;

(v) R^{2a} is an optionally substituted heterocyclyl, wherein when the heterocyclyl is monocyclic, the point of connection is via a carbon atom;

(vi) R^{3a} is an optionally substituted heterocyclyl, wherein when the heterocyclyl is monocyclic, the point of connection is via a carbon atom;

(vii) R^{2a} and R^{3a} are taken together with the carbons to which they are attached to form a 5- to 16-membered heterocyclyl ring;

(viii) A is

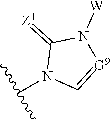

### and Z¹ is O;

(ix) A is

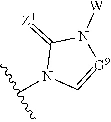

and W is C₁₋₄ alkyl;

(x) A is

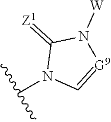

### and G⁹ is CH; and

(xii) A is

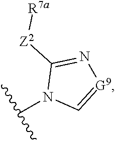

or a pharmaceutically acceptable salt thereof.

117. A compound of Formula (II):

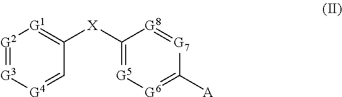

wherein

### G¹ is CH or N;

G² is CR^{2a} or N;
G³ is CR^{3a} or N;

### G⁴ is CH or N;

wherein no more than two of G¹, G², G³, and G⁴ are N;

### G⁵ is CH or N;

G⁶ is CR^{1a} or N;

### G⁷ is CH or N;

### G⁸ is CH or N;

wherein no more than one of G⁵, G⁶, G⁷, and G⁸ is N;
R^{1a}, R^{2a}, and R^{3a} are each independently hydrogen, hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, or an optionally substituted heterocyclyl;
   wherein R^{x} and R^{y} are each independently H or optionally substituted C₁₋₄alkyl;
or R^{1a} and R^{2a} are taken together with the carbons to which they are attached to form a 5- to 16-membered heterocyclyl ring;
   X is —CR^{4a}R^{5a}—, —O—, —S—, —S(O)—, —NR^{6a}—, —S(O)₂—, or —C(O)—;

wherein R^{4a} and R^{5a} are independently hydrogen, hydroxy, halogen, substituted C₁₋₄ alkyl, C₁₋₄ alkoxy, or substituted C₁₋₄ alkoxy;

or R^{4a} and R^{5a} are taken together with the carbon to which they are attached to form a 3- to 6-membered cycloalkyl ring;

R^{6a} is hydrogen or C₁₋₄ alkyl;

### A is

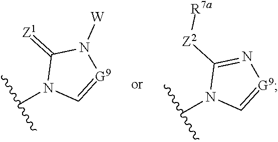

### G⁹ is CH or N;

Z¹ and Z² are independently S or O; and
W and R^{7a} are independently hydrogen or C₁₋₄ alkyl; and

one or more of the following apply:

(i) X is —CR^{4a}R^{5a}—, —O—, —S—, —S(O)—, —NR^{a}—, or —C(O)—;

(ii) one or two of G¹, G², G³, and G⁴ is N;

(iii) one of G⁵, G⁶, G⁷, and G⁸ is N;

(iv) R^{1a} is an optionally substituted heterocyclyl, wherein when the heterocyclyl is monocyclic, the point of connection is via a carbon atom;

(v) R^{2a} is an optionally substituted heterocyclyl, wherein when the heterocyclyl is monocyclic, the point of connection is via a carbon atom;

(vi) R^{3a} is an optionally substituted heterocyclyl, wherein when the heterocyclyl is monocyclic, the point of connection is via a carbon atom;

(vii) R^{2a} and R^{3a} are taken together with the carbons to which they are attached to form a 5- to 16-membered heterocyclyl ring;

(vii) A is

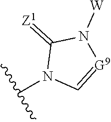

### and Z¹ is O;

(ix) A is

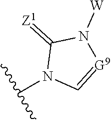

and W is C₁₋₄ alkyl;

(x) A is

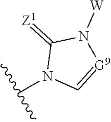

### and G⁹ is CH; and

(xii) A is

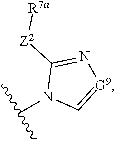

or a pharmaceutically acceptable salt thereof.

118. The compound of embodiment 117, or a pharmaceutically acceptable salt thereof, wherein G¹ is CH.

119. The compound of embodiment 117, or a pharmaceutically acceptable salt thereof, wherein G¹ is N.

120. The compound of any one of embodiments 117-119, or a pharmaceutically acceptable salt thereof, wherein G² is CR^{2a}.

121. The compound of any one of embodiments 117-120, or a pharmaceutically acceptable salt thereof, wherein G³ is CR^{3a},

122. The compound of embodiment 120, or a pharmaceutically acceptable salt thereof, wherein R^{2a} is hydrogen, hydroxy, halogen, C₁₋₄ alkyl, C₁₋₄ alkyl substituted with one or more halogen, C₁₋₄ alkoxy, C₁₋₄ alkoxy substituted with one or more halogen or C₁₋₄ alkoxy, —CN, —NR^{x}R^{y}, or an optionally substituted heterocyclyl.

123. The compound of embodiment 121, or a pharmaceutically acceptable salt thereof, wherein R^{3a} is hydrogen, hydroxy, halogen, C₁₋₄ alkyl, C₁₋₄ alkyl substituted with one or more halogen, C₁₋₄ alkoxy, C₁₋₄ alkoxy substituted with one or more halogen or C₁₋₄ alkoxy, —CN, —NR^{x}R^{y}, or an optionally substituted heterocyclyl.

124. The compound of any one of embodiments 117-121, or a pharmaceutically acceptable salt thereof, wherein R^{2a} and R^{3a} are taken together with the carbons to which they are attached to form a 5- to 16-membered heterocyclyl ring.

125. The compound of any one of embodiments 117-119, 121, and 123, or a pharmaceutically acceptable salt thereof, wherein G² is N.

126. The compound of any one of embodiments 117-120, and 122, or a pharmaceutically acceptable salt thereof, wherein G³ is N.

127. The compound of any one of embodiments 117-126, or a pharmaceutically acceptable salt thereof, wherein G⁴ is CH.

128. The compound of any one of embodiments 117-127, or a pharmaceutically acceptable salt thereof, wherein G⁴ is N.

129. The compound of any one of embodiments 117-128, or a pharmaceutically acceptable salt thereof, wherein X is —CR^{4a}R^{5a}—; wherein R^{4a} and R^{5a}, are each independently hydrogen, hydroxy, halogen, or R^{4a} and R^{5a} are taken together with the carbon to which they are attached to form a 3- to 6-membered cycloalkyl ring.

130. The compound of any one of embodiments 117-128, or a pharmaceutically acceptable salt thereof, wherein X is —S(O)₂—.

131. The compound of any one of embodiments 117-128, or a pharmaceutically acceptable salt thereof, wherein X is —O— or —S—.

132. The compound of any one of embodiments 117-128, or a pharmaceutically acceptable salt thereof, wherein X is —S(O)— or —C(O)—.

133. The compound of any one of embodiments 117-130, or a pharmaceutically acceptable salt thereof, wherein X is —NR^{6a}—, wherein R^{6a} is hydrogen or C₁₋₄ alkyl.

134. The compound of any one of embodiments 117-133, or a pharmaceutically acceptable salt thereof, wherein G⁵ is CH.

135. The compound of any one of embodiments 117-133, or a pharmaceutically acceptable salt thereof, wherein G⁵ is N.

136. The compound of any one of embodiments 117-135, or a pharmaceutically acceptable salt thereof, wherein G⁶ is CR^{1a},

137. The compound of embodiment 136, or a pharmaceutically acceptable salt thereof, wherein R^{1a} is hydrogen, hydroxy, halogen, C₁₋₄ alkyl, C₁₋₄ alkyl substituted with one or more halogen, C₁₋₄ alkoxy, C₁₋₄ alkoxy substituted with one or more halogen or C₁₋₄ alkoxy, —CN, —NR^{x}R^{y}, or an optionally substituted heterocyclyl.

138. The compound of any one of embodiments 117-134, or a pharmaceutically acceptable salt thereof, wherein G⁶ is N.

139. The compound of any one of embodiments 117-138, or a pharmaceutically acceptable salt thereof, wherein G⁷ is CH.

140. The compound of any one of embodiments 117-134 and 136-137, or a pharmaceutically acceptable salt thereof, wherein G⁷ is N.

1341. The compound of any one of embodiments 117-140, or a pharmaceutically acceptable salt thereof, wherein G⁸ is CH.

142. The compound of any one of embodiments 117-134, 136-137, and 139, or a pharmaceutically acceptable salt thereof, wherein G⁸ is N.

143. The compound of any one of embodiments 117-142, or a pharmaceutically acceptable salt thereof, wherein A is

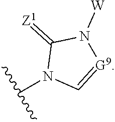

144. The compound of embodiment 143, or a pharmaceutically acceptable salt thereof, wherein Z¹ is S.

145. The compound of embodiment 143, or a pharmaceutically acceptable salt thereof, wherein Z¹ is O.

146. The compound of any one of embodiments 143-145, or a pharmaceutically acceptable salt thereof, wherein G⁹ is CH.

147. The compound of any one of embodiments 143-145, or a pharmaceutically acceptable salt thereof, wherein G⁹ is N.

148. The compound of any one of embodiments 143-147, or a pharmaceutically acceptable salt thereof, wherein W is hydrogen.

149. The compound of any one of embodiments 143-147, or a pharmaceutically acceptable salt thereof, wherein W is C₁₋₄ alkyl.

150. The compound of any one of embodiments 117-142, or a pharmaceutically

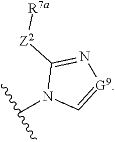

acceptable salt thereof, wherein A is

151. The compound of embodiment 150, or a pharmaceutically acceptable salt thereof, wherein Z² is S.

152. The compound of embodiment 160, or a pharmaceutically acceptable salt thereof, wherein Z² is O.

153. The compound of embodiment 150, or a pharmaceutically acceptable salt thereof, wherein R^{7a} is hydrogen.

154. The compound of embodiment 150, or a pharmaceutically acceptable salt thereof, wherein R^{7a} is C₁₋₄ alkyl.

155. The compound of any one of embodiments 150-154, or a pharmaceutically acceptable salt thereof, wherein G⁹ is CH.

156. The compound of any one of embodiments 150-154, or a pharmaceutically acceptable salt thereof, wherein G⁹ is N.

157. The compound of embodiment 116 or 117, or a pharmaceutically acceptable salt thereof, wherein the compound of Formula (IIA) or Formula (II) is a compound of a Formula (II-1):

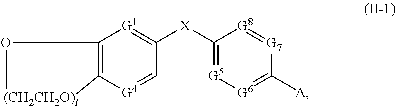

or a pharmaceutically acceptable salt thereof, wherein G¹, G⁴, G⁵, G⁶, G⁷, G^{g}, X and A are as defined for Formula (IIA) or Formula (II), and t is 1, 2, or 3.

158. The compound of embodiment 116 or 117, or a pharmaceutically acceptable salt thereof, wherein the compound of Formula (IIA) or Formula (II) is a compound of a Formula (II-2):

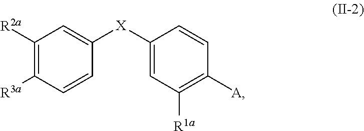

or a pharmaceutically acceptable salt thereof, wherein R^{1a}, R^{2a}, R^{3a}, X and A are as defined for Formula (IIA) or Formula (II).

159. The compound of embodiment 116 or 117, or a pharmaceutically acceptable salt thereof, wherein the compound of Formula (IIA) or Formula (II) is a compound of Formula (IIa), (IIb), (IIc), (IId), (IIe), (IIf), or (IIg):

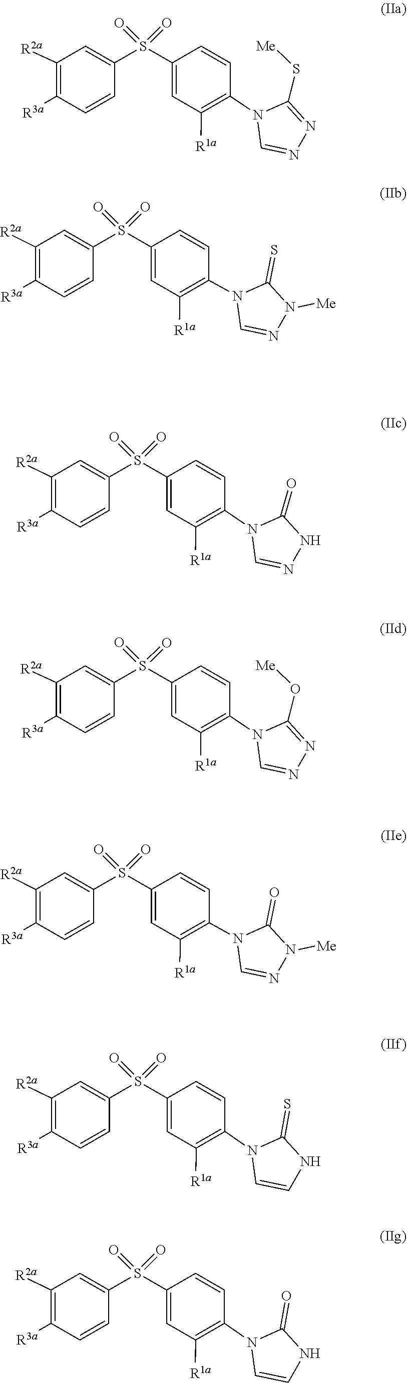

or a pharmaceutically acceptable salt thereof, wherein R^{1a}, R^{2a}, and R^{3a} are as defined for Formula (IIA) or Formula (II).

160. The compound of embodiment 116 or 117, or a pharmaceutically acceptable salt thereof, wherein the compound of Formula (IIA) or Formula (II) is a compound of Formula (IIh), (IIi), (IIj), (Ilk), (IIl), (IIm), (IIn), (IIo), or (IIp):

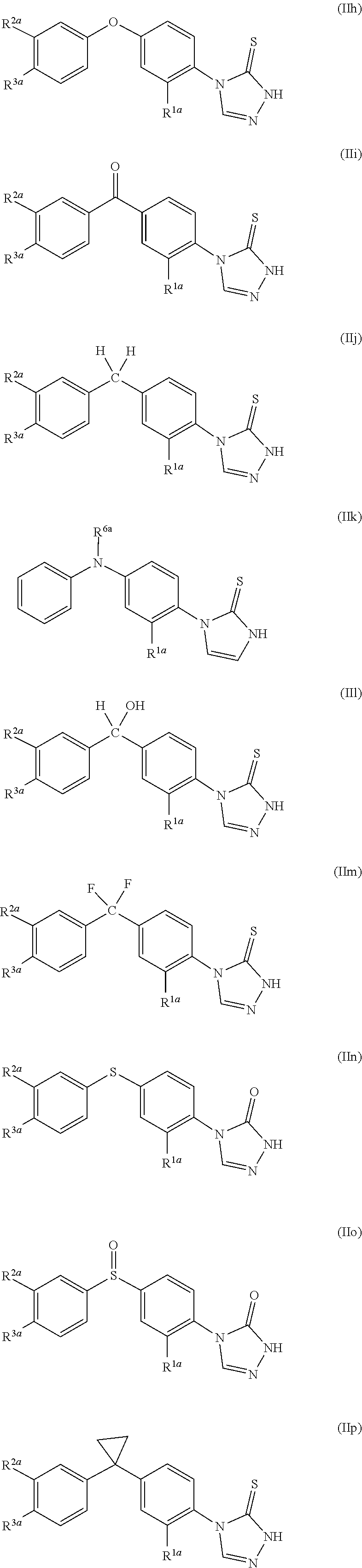

or a pharmaceutically acceptable salt thereof, wherein R^{1a}, R^{2a}, R^{3a}, and R^{6a} are as defined for Formula (A) or Formula (II).

161. A compound selected from the group consisting of

| Compound | | |
|---|---|---|
| No. | Structure | Name |
| 26 | 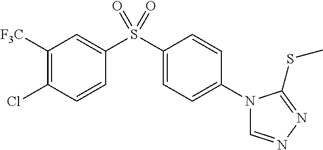 [SMILES: CSc1nncn1-c1ccc(S(=O)(=O)c2ccc(Cl)c(C(F)(F)F)c2)cc1] | 4-(4-((4-chloro-3- (trifluoromethyl)phenyl)sulfonyl)phenyl)- 3-(methylthio)-4H-1,2,4-triazole; |
| 27 | 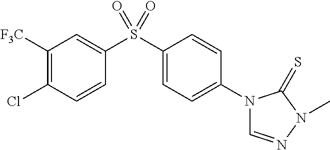 [SMILES: Cn1ncn(-c2ccc(S(=O)(=O)c3ccc(Cl)c(C(F)(F)F)c3)cc2)c1=S] | 4-(4-((4-chloro-3- (trifluoromethyl)phenyl)sulfonyl)phenyl)- 2-methyl-2,4-dihydro-3H-1,2,4-triazole- 3-thione; |
| 28 | 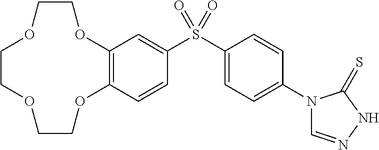 [SMILES: O=S(=O)(c1ccc(-n2cn[nH]c2=S)cc1)c1ccc2c(c1)OCCOCCOCCO2] | 4-(4-((2,3,5,6,8,9- hexahydrobenzo[b][1,4,7,10]tetraoxacyclo- dodecin-12-yl)sulfonyl)phenyl)-2,4- dihydro-3H-1,2,4-triazole-3-thione; |
| 29 | 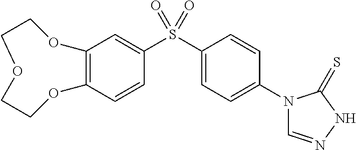 [SMILES: O=S(=O)(c1ccc(-n2cn[nH]c2=S)cc1)c1ccc2c(c1)OCCOCCO2] | 4-(4-((2,3,5,6- tetrahydrobenzo[b][1,4,7]trioxonin-9- yl)sulfonyl)phenyl)-2,4-dihydro-3H- 1,2,4-triazole-3-thione; |
| 30 | 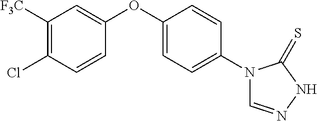 [SMILES: FC(F)(F)c1cc(Oc2ccc(-n3cn[nH]c3=S)cc2)ccc1Cl] | 4-(4-(4-chloro-3-(trifluoro-methyl)- phenoxy)-phenyl)-3,4-dihydro-2H-1,2,4- triazole-3-thione; |
| 31 | 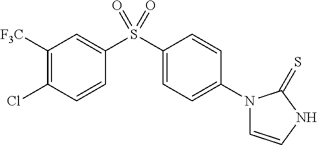 [SMILES: O=S(=O)(c1ccc(-n2cc[nH]c2=S)cc1)c1ccc(Cl)c(C(F)(F)F)c1] | 1-(4-(4-chloro-3-(trifluoro-methyl)- phenylsulfonyl)-phenyl)-2,3-dihydro- 1H-imidazole-2-thione; |
| 32 | 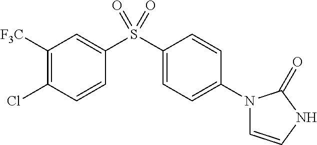 [SMILES: O=c1[nH]ccn1-c1ccc(S(=O)(=O)c2ccc(Cl)c(C(F)(F)F)c2)cc1] | 1-(4-(4-chloro-3-(trifluoro-methyl)- phenylsulfonyl)-phenyl)-2,3-dihydro- 1H-imidazol-2-one; |
| 33 | 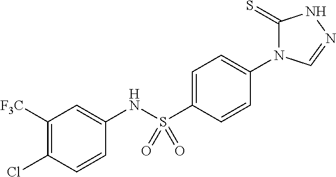 [SMILES: O=S(=O)(Nc1ccc(Cl)c(C(F)(F)F)c1)c1ccc(-n2cn[nH]c2=S)cc1] | N-(4-chloro-3-(trifluoromethyl)phenyl)- 4-(5-thioxo-1,5-dihydro-4H-1,2,4- triazol-4-yl)benzenesulfonamide; |
| 34 | 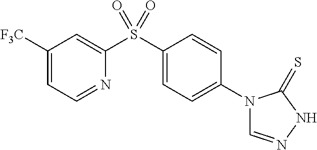 [SMILES: O=S(=O)(c1ccc(-n2cn[nH]c2=S)cc1)c1cc(C(F)(F)F)ccn1] | 4-(4-((2-(trifluoromethyl)pyridin-4- yl)sulfonyl)phenyl)-2,4-dihydro-3H- 1,2,4-triazole-3-thione; |
| 35 | 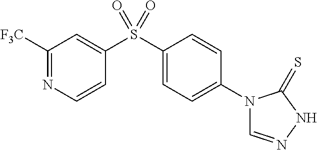 [SMILES: O=S(=O)(c1ccc(-n2cn[nH]c2=S)cc1)c1ccnc(C(F)(F)F)c1] | 4-(4-((4-(trifluoromethyl)pyridin-2- yl)sulfonyl)phenyl)-2,4-dihydro-3H- 1,2,4-triazole-3-thione; |
| 36 | 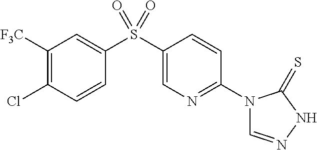 [SMILES: O=S(=O)(c1ccc(-n2cn[nH]c2=S)nc1)c1ccc(Cl)c(C(F)(F)F)c1] | 4-(5-((4-chloro-3- (trifluoromethyl)phenyl)sulfonyl)pyridin- 2-yl)-2,4-dihydro-3H-1,2,4-triazole-3- thione; |
| 37 | 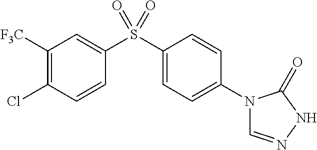 [SMILES: O=c1[nH]ncn1-c1ccc(S(=O)(=O)c2ccc(Cl)c(C(F)(F)F)c2)cc1] | 4-(4-((4-chloro-3- (trifluoromethyl)phenyl)sulfonyl)phenyl)- 2,4-dihydro-3H-1,2,4-triazol-3-one; |
| 38 | 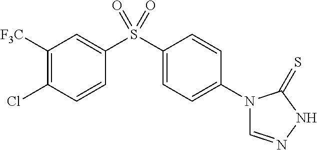 [SMILES: O=S(=O)(c1ccc(-n2cn[nH]c2=S)cc1)c1ccc(Cl)c(C(F)(F)F)c1] | (4-chloro-3-(trifluoromethyl)phenyl)(4- (5-thioxo-1,5-dihydro-4H-1,2,4-triazol- 4-yl)phenyl)methanone; |
| 39 | 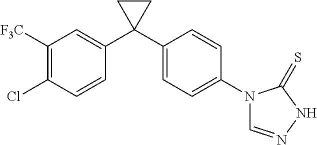 [SMILES: FC(F)(F)c1cc(C2(c3ccc(-n4cn[nH]c4=S)cc3)CC2)ccc1Cl] | 4-(4-(1-(4-chloro-3- (trifluoromethyl)phenyl)cyclopropyl)phe- nyl)-2,4-dihydro-3H-1,2,4-triazole-3- thione; |
| 40 | 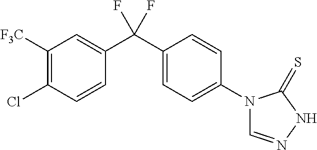 [SMILES: FC(F)(F)c1cc(C(F)(F)c2ccc(-n3cn[nH]c3=S)cc2)ccc1Cl] | 4-(4-((4-chloro-3- (trifluoromethyl)phenyl)difluoromethyl) phenyl)-2,4-dihydro-3H-1,2,4-triazole-3- thione; |
| 41 | 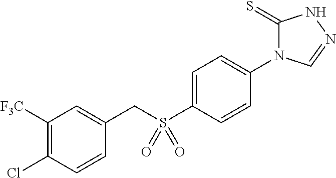 [SMILES: O=S(=O)(Cc1ccc(Cl)c(C(F)(F)F)c1)c1ccc(-n2cn[nH]c2=S)cc1] | 4-(4-((4-chloro-3- (trifluoromethyl)benzyl)sulfonyl)phenyl)- 2,4-dihydro-3H-1,2,4-triazole-3-thione; |
| 42 | 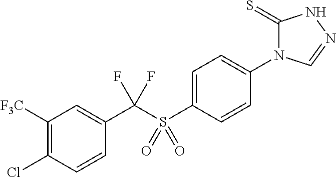 [SMILES: O=S(=O)(c1ccc(-n2cn[nH]c2=S)cc1)C(F)(F)c1ccc(Cl)c(C(F)(F)F)c1] | 4-(4-(((4-chloro-3- (trifluoromethyl)phenyl)difluoromethyl) sulfonyl)phenyl)-2,4-dihydro-3H-1,2,4- triazole-3-thione; |
| 43 | 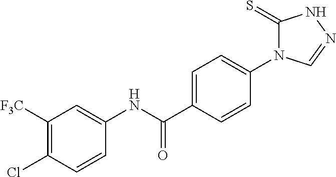 [SMILES: O=C(Nc1ccc(Cl)c(C(F)(F)F)c1)c1ccc(-n2cn[nH]c2=S)cc1] | N-(4-chloro-3-(trifluoromethyl)phenyl)- 4-(5-thioxo-1,5-dihydro-4H-1,2,4- triazol-4-yl)benzamide; and |
| 44 | 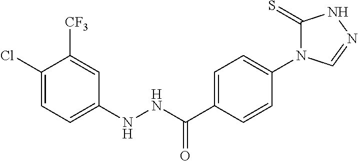 [SMILES: O=C(NNc1ccc(Cl)c(C(F)(F)F)c1)c1ccc(-n2cn[nH]c2=S)cc1] | N′-(4-chloro-3-(trifluoromethyl)phenyl)- 4-(5-thioxo-1,5-dihydro-4H-1,2,4- triazol-4-yl)benzohydrazide, |

or a pharmaceutically acceptable salt thereof.

162. A pharmaceutical composition comprising (a) at least one compound of any one of embodiments 117-161, or a pharmaceutically acceptable salt thereof, and (b) a pharmaceutically acceptable excipient.

163. The pharmaceutical composition of embodiment 162, wherein the pharmaceutically acceptable excipient is a polymeric agent.

164. The pharmaceutical composition of embodiment 162, wherein the pharmaceutically acceptable excipient is selected from the group consisting of carboxymethyl cellulose (CMC), hydroxypropyl cellulose (HPC), hydroxyethyl cellulose (HEC), hydroxypropylmethyl cellulose (HPMC), gelatin, gelatin hydrolysate, sucrose, dextrose, polyvinylpyrrolidone (PVP), polyethyleneglycol (PEG), vinyl pyrrolidone copolymers, pregelatinized starch, sorbitol, and glucose; and polyacrylates.

165. The pharmaceutical composition of embodiment 162, wherein the pharmaceutically acceptable excipient is selected from the group consisting of hydroxypropylmethyl cellulose (HPMC), polyvinylpyrrolidone (PVP), and Kollidon.

166. The pharmaceutical composition of any one of embodiments 162-165, wherein the pharmaceutical composition is in the form of a spray dry dispersion (SDD).

167. A method of treating a condition associated with neurodegeneration or accumulation of proteins, comprising administering to a subject in need of such treatment an effective amount of at least one compound of any one of embodiments 117-161, or a pharmaceutically acceptable salt thereof, or a pharmaceutical composition of any one of embodiments 162-166.

168. The method of embodiment 167, wherein the condition is Alzheimer's Disease, Parkinson's Disease, fronto-temporal dementia, dementia with Lewy Bodies, PD dementia, multiple system atrophy, Huntington's disease, Amyotrophic lateral sclerosis, progressive supranuclear palsy, cancer, infection, Crohn's disease, heart disease, aging, or traumatic brain injury (TBI).

169. A compound of any one of embodiments 117-161, or a pharmaceutically acceptable salt thereof, or a pharmaceutical composition of any one of embodiments 162-166, for use in the treatment of a condition associated with neurodegeneration or accumulation of proteins.

170. The compound or pharmaceutical composition of embodiment 169, wherein the condition is Alzheimer's Disease, Parkinson's Disease, fronto-temporal dementia, dementia with Lewy Bodies, PD dementia, multiple system atrophy, Huntington's disease, Amyotrophic lateral sclerosis, progressive supranuclear palsy, cancer, infection, Crohn's disease, heart disease, aging, or traumatic brain injury (TBI).

171. Use of at least one compound of any one of embodiments 117-161, or a pharmaceutically acceptable salt thereof, or a pharmaceutical composition of any one of embodiments 156-160, in the manufacture of a medicament for the treatment of a condition associated with neurodegeneration or accumulation of proteins.

172. The use of embodiment 171, wherein the condition is Alzheimer's Disease, Parkinson's Disease, fronto-temporal dementia, dementia with Lewy Bodies, PD dementia, multiple system atrophy, Huntington's disease, Amyotrophic lateral sclerosis, progressive supranuclear palsy, cancer, infection, Crohn's disease, heart disease, aging, or traumatic brain injury (TBI).

173. A method of preventing aggregation or accumulation or enhancing clearance of protease-resistant protein, comprising contacting the protease-resistant protein with an effective amount of at least one compound of any one of embodiments 117-161, or a salt thereof, or a pharmaceutical composition of any one of embodiments 162-166, wherein the contacting is in vitro, ex vivo, or in vivo.

174. The method of embodiment 173, wherein the protease-resistant protein is selected from alpha synuclein, a-beta, tau, Huntingtin, and TAR DNA binding protein 43 (TDP43) proteins.

175. A method of decreasing neuroinflammation in a subject, comprising administering to the subject an effective amount of at least one compound of any one of embodiments 117-161, or a pharmaceutically acceptable salt thereof, or a pharmaceutical composition of any one of embodiments 162-166.

176. A compound of any one of embodiments 117-161, or a pharmaceutically acceptable salt thereof, or a pharmaceutical composition of any one of embodiments 162-166, for use in decreasing neuroinflammation.

177. Use of at least one compound of any one of embodiments 117-161, or a pharmaceutically acceptable salt thereof, or a pharmaceutical composition of any one of embodiments 162-166, in the manufacture of a medicament for decreasing neuroinflammation.

178. A method of treating a disease or condition associated with neuroinflammation, comprising administering to a subject in need of such treatment an effective amount of at least one compound of any one of embodiments 117-161, or a pharmaceutically acceptable salt thereof, or a pharmaceutical composition of any one of embodiments 162-166.

179. A compound of any one of embodiments 117-161, or a pharmaceutically acceptable salt thereof, or a pharmaceutical composition of any one of embodiments 162-166, for use in the treatment of a disease or condition associated with neuroinflammation.

180. Use of at least one compound of any one of embodiments 117-161, or a pharmaceutically acceptable salt thereof, or a pharmaceutical composition of any one of embodiments 162-166, in the manufacture of a medicament for the treatment of a disease or condition associated with neuroinflammation.

1. A method of treating a disease or condition associated with neurodegeneration or accumulation of proteins in the brain in a subject in need thereof, comprising administering to the subject an effective amount of an ion transporter inhibitor, wherein the ion transporter inhibitor modulates the efflux of one or more bioactive endogenous metabolites across the blood brain barrier (BBB) of the subject.

2. The method of claim 1, wherein the disease or condition associated with neurodegeneration is Alzheimer's Disease, Parkinson's Disease, fronto-temporal dementia, dementia with Lewy Bodies, PD dementia, multiple system atrophy, Huntington's disease, Amyotrophic lateral sclerosis, progressive supranuclear palsy, or neuroinflammation.

3. A method of modulating efflux of one or more bioactive endogenous metabolites across the blood brain barrier (BBB) in a subject in need thereof, comprising administering to the subject in need thereof an ion transporter inhibitor.

4. A method of improving neuroprotection in a subject in need thereof, comprising administering to the subject an effective amount of an ion transporter inhibitor that modulates the concentration of one or more bioactive endogenous metabolites in the brain interstitial space.

5. A method of decreasing neuroinflammation in a subject in need thereof, comprising administering to the subject an effective amount of an ion transporter inhibitor that modulates the concentration of one or more bioactive endogenous metabolites in the brain interstitial space.

6. The method of any one of claims 1-5, wherein the ion transporter inhibitor is an inhibitor of organic anion transporter 3 (OAT3).

7. The method of any one of claims 1-6, wherein the ion transporter inhibitor selectively inhibits OAT3 compared with other ion transporter proteins.

8. The method of any one of claims 1-7, wherein the ion transporter inhibitor has an IC₅₀ for OAT3 of about 1 μM or less.

9. The method of any one of claims 1-8, wherein the ion transporter inhibitor has an IC₅₀ for OAT3 that is at least about 10 fold lower compared with its IC₅₀ for organic anion transporter 1 (OAT1).

10. The method of any one of claims 1-9, wherein after administration of the ion transporter inhibitor, the efflux of the one or more bioactive endogenous metabolites across the BBB is reduced.

11. The method of any one of claims 1-10, wherein after administration of the ion transporter inhibitor, the local concentrations of the one or more bioactive endogenous metabolites in the brain interstitial space are increased.

12. The method of any one of claims 1-11, wherein after administration of the ion transporter inhibitor, the plasma levels of the one or more bioactive endogenous metabolites are decreased.

13. The method of any one of claims 1-12, wherein the one or more bioactive endogenous metabolites is an anionic neurotransmitter metabolite of epinephrine, norepinephrine, dopamine, and/or serotonin.

14. The method of any one of claims 1-12, wherein the one or more bioactive endogenous metabolites are selected from the group consisting of: uric acid, glutathione, dehydroepianodrosterone (DHEA), and DHEA sulfate (DHEAS).

15. The method of any one of claims 1-14, wherein the one or more bioactive endogenous metabolites have neuroprotective and/or anti-neuroinflammatory properties.

16. The method of claim 15, wherein the anti-neuroinflammatory properties include reduction of a pro-inflammatory response in the brain of the subject.

17. A method of preventing aggregation or accumulation or enhancing clearance of protease-resistant protein, comprising contacting the protease-resistant protein with an effective amount of a compound that is an inhibitor of organic anion transporter 3 (OAT3), wherein the contacting is in vitro, ex vivo, or in vivo.

18. The method of any one of claims 1-17, wherein the compound is a compound of Formula (I): 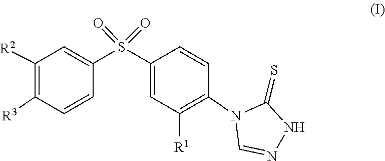
wherein
R¹, R², and R³ are each independently hydrogen, hydroxy, halogen, optionally substituted C₁₋₄ alkyl, optionally substituted C₁₋₄ alkoxy, —CN, —C(O)R^{x}, —C(O)OR^{x}, —S(O)₂R^{x}, or —NR^{y}R^{z};
R^{x}, R^{y}, and R^{z} are each independently H or optionally substituted C₁₋₄alkyl, or R^{y} and R^{z} taken together with the nitrogen to which they are attached form an optionally substituted monocyclic heterocycloalkyl ring;
or a pharmaceutically acceptable salt thereof.

19. The method of any one of claims 1-17, wherein the compound is

| Compound | | |
|---|---|---|
| No. | Structure | Name |
| 1 | 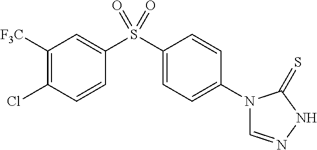 [SMILES: O=S(=O)(c1ccc(-n2cn[nH]c2=S)cc1)c1ccc(Cl)c(C(F)(F)F)c1] | 4-(4-((4-chloro-3- (trifluoromethyl)phenyl)sulfonyl)phenyl)- 2,4-dihydro-3H-1,2,4-triazole-3- thione; |
| 2 | 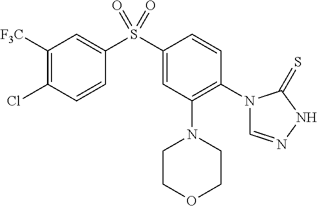 [SMILES: O=S(=O)(c1ccc(-n2cn[nH]c2=S)c(N2CCOCC2)c1)c1ccc(Cl)c(C(F)(F)F)c1] | 4-(4-((4-chloro-3- (trifluoromethyl)phenyl)sulfonyl)-2- morpholinophenyl)-2,4-dihydro-3H- 1,2,4-triazole-3-thione; |
| 3 | 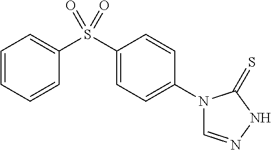 [SMILES: O=S(=O)(c1ccccc1)c1ccc(-n2cn[nH]c2=S)cc1] | 4-(4-(phenylsulfonyl)phenyl)-2,4- dihydro-3H-1,2,4-triazole-3-thione; |
| 4 | 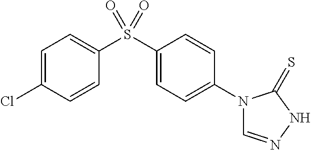 [SMILES: O=S(=O)(c1ccc(Cl)cc1)c1ccc(-n2cn[nH]c2=S)cc1] | 4-(4-((4-chlorophenyl)sulfonyl)phenyl)- 2,4-dihydro-3H-1,2,4-triazole-3-thione; |
| 5 | 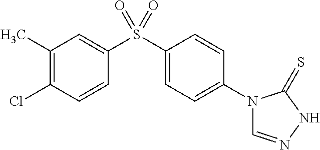 [SMILES: Cc1cc(S(=O)(=O)c2ccc(-n3cn[nH]c3=S)cc2)ccc1Cl] | 4-(4-((4-chloro-3- methylphenyl)sulfonyl)phenyl)-2,4- dihydro-3H-1,2,4-triazole-3-thione; |
| 6 | 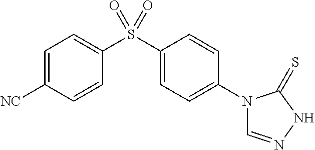 [SMILES: N#Cc1ccc(S(=O)(=O)c2ccc(-n3cn[nH]c3=S)cc2)cc1] | 4-((4-(5-thioxo-1,5-dihydro-4H-1,2,4- triazol-4- yl)phenyl)sulfonyl)benzonitrile; |
| 7 | 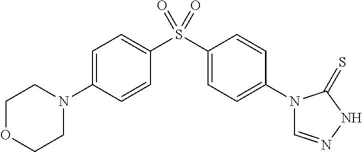 [SMILES: O=S(=O)(c1ccc(N2CCOCC2)cc1)c1ccc(-n2cn[nH]c2=S)cc1] | 4-(4-((4- morpholinophenyl)sulfonyl)phenyl)- 2,4-dihydro-3H-1,2,4-triazole-3-thione; |
| 8 | 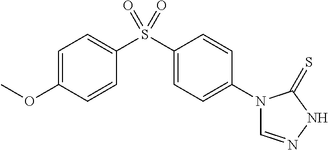 [SMILES: COc1ccc(S(=O)(=O)c2ccc(-n3cn[nH]c3=S)cc2)cc1] | 4-(4-((4- methoxyphenyl)sulfonyl)phenyl)-2,4- dihydro-3H-1,2,4-triazole-3-thione; |
| 9 | 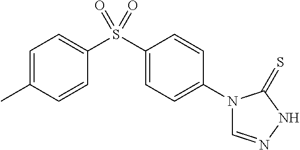 [SMILES: Cc1ccc(S(=O)(=O)c2ccc(-n3cn[nH]c3=S)cc2)cc1] | 4-(4-tosylphenyl)-2,4-dihydro-3H-1,2,4- triazole-3-thione; |
| 10 | 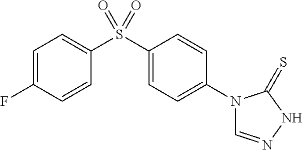 [SMILES: O=S(=O)(c1ccc(F)cc1)c1ccc(-n2cn[nH]c2=S)cc1] | 4-(4-((4-fluorophenyl)sulfonyl)phenyl)- 2,4-dihydro-3H-1,2,4-triazole-3-thione; |
| 11 | 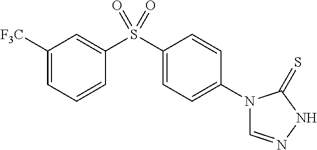 [SMILES: O=S(=O)(c1ccc(-n2cn[nH]c2=S)cc1)c1cccc(C(F)(F)F)c1] | 4-(4-((3- (trifluoromethyl)phenyl)sulfonyl)phenyl)- 2,4-dihydro-3H-1,2,4-triazole-3- thione; |
| 12 | 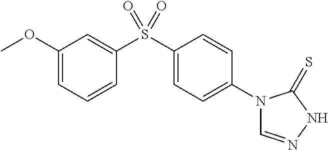 [SMILES: COc1cccc(S(=O)(=O)c2ccc(-n3cn[nH]c3=S)cc2)c1] | 4-(4-((3- methoxyphenyl)sulfonyl)phenyl)-2,4- dihydro-3H-1,2,4-triazole-3-thione; |
| 13 | 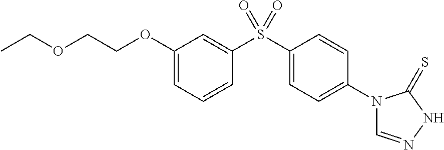 [SMILES: CCOCCOc1cccc(S(=O)(=O)c2ccc(-n3cn[nH]c3=S)cc2)c1] | 4-(4-((3-(2- ethoxyethoxy)phenyl)sulfonyl)phenyl)- 2,4-dihydro-3H-1,2,4-triazole-3-thione; |
| 14 | 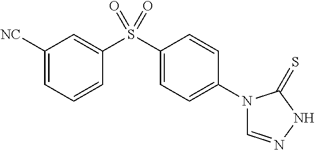 [SMILES: N#Cc1cccc(S(=O)(=O)c2ccc(-n3cn[nH]c3=S)cc2)c1] | 3-((4-(5-thioxo-1,5-dihydro-4H-1,2,4- triazol-4- yl)phenyl)sulfonyl)benzonitrile; |
| 15 | 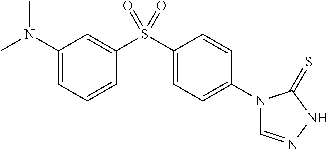 [SMILES: CN(C)c1cccc(S(=O)(=O)c2ccc(-n3cn[nH]c3=S)cc2)c1] | 4-(4-((3- (dimethylamino)phenyl)sulfonyl)phenyl)- 2,4-dihydro-3H-1,2,4-triazole-3- thione; |
| 16 | 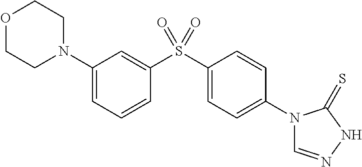 [SMILES: O=S(=O)(c1ccc(-n2cn[nH]c2=S)cc1)c1cccc(N2CCOCC2)c1] | 4-(4-((3- morpholinophenyl)sulfonyl)phenyl)- 2,4-dihydro-3H-1,2,4-triazole-3-thione; |
| 17 | 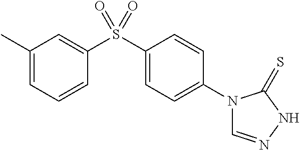 [SMILES: Cc1cccc(S(=O)(=O)c2ccc(-n3cn[nH]c3=S)cc2)c1] | 4-(4-(m-tolylsulfonyl)phenyl)-2,4- dihydro-3H-1,2,4-triazole-3-thione; |
| 18 | 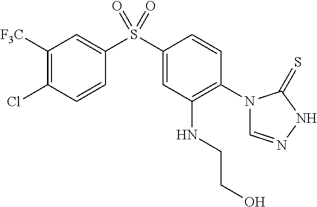 [SMILES: O=S(=O)(c1ccc(-n2cn[nH]c2=S)c(NCCO)c1)c1ccc(Cl)c(C(F)(F)F)c1] | 4-(4-((4-chloro-3- (trifluoromethyl)phenyl)sulfonyl)-2-((2- hydroxyethyl)amino)phenyl)-2,4- dihydro-3H-1,2,4-triazole-3-thione; |
| 19 | 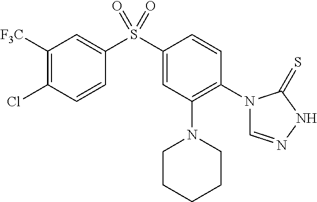 [SMILES: O=S(=O)(c1ccc(-n2cn[nH]c2=S)c(N2CCCCC2)c1)c1ccc(Cl)c(C(F)(F)F)c1] | 4-(4-((4-chloro-3- (trifluoromethyl)phenyl)sulfonyl)-2- (piperidin-1-yl)phenyl)-2,4-dihydro-3H- 1,2,4-triazole-3-thione; |
| 20 | 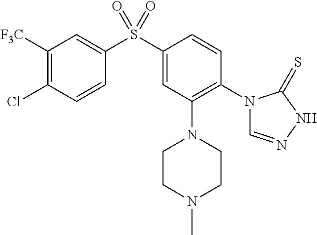 [SMILES: CN1CCN(c2cc(S(=O)(=O)c3ccc(Cl)c(C(F)(F)F)c3)ccc2-n2cn[nH]c2=S)CC1] | 4-(4-((4-chloro-3- (trifluoromethyl)phenyl)sulfonyl)-2-(4- methylpiperazin-1-yl)phenyl)-2,4- dihydro-3H-1,2,4-triazole-3-thione; |
| 21 | 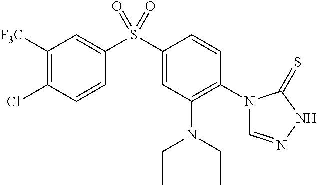 [SMILES: CCN(CC)c1cc(S(=O)(=O)c2ccc(Cl)c(C(F)(F)F)c2)ccc1-n1cn[nH]c1=S] | 4-(4-((4-chloro-3- (trifluoromethyl)phenyl)sulfonyl)-2- (diethylamino)phenyl)-2,4-dihydro-3H- 1,2,4-triazole-3-thione; |
| 22 | 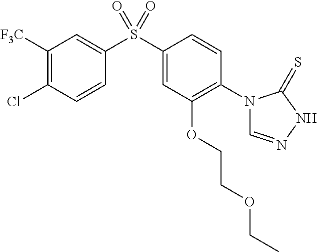 [SMILES: CCOCCOc1cc(S(=O)(=O)c2ccc(Cl)c(C(F)(F)F)c2)ccc1-n1cn[nH]c1=S] | 4-(4-((4-chloro-3- (trifluoromethyl)phenyl)sulfonyl)-2-(2- ethoxyethoxy)phenyl)-2,4-dihydro-3H- 1,2,4-triazole-3-thione; |
| 23 | 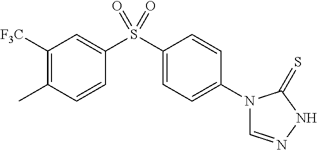 [SMILES: Cc1ccc(S(=O)(=O)c2ccc(-n3cn[nH]c3=S)cc2)cc1C(F)(F)F] | 4-(4-((4-methyl-3- (trifluoromethyl)phenyl)sulfonyl)phenyl)- 2,4-dihydro-3H-1,2,4-triazole-3- thione; |
| 24 | 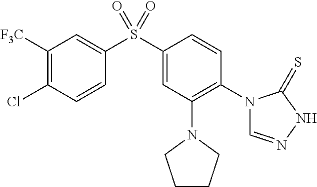 [SMILES: O=S(=O)(c1ccc(-n2cn[nH]c2=S)c(N2CCCC2)c1)c1ccc(Cl)c(C(F)(F)F)c1] | 4-(4-((4-chloro-3- (trifluoromethyl)phenyl)sulfonyl)-2- (pyrrolidin-1-yl)phenyl)-2,4-dihydro- 3H-1,2,4-triazole-3-thione; and |
| 25 | 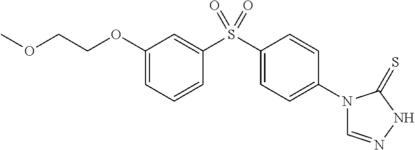 [SMILES: COCCOc1cccc(S(=O)(=O)c2ccc(-n3cn[nH]c3=S)cc2)c1] | 4-(4-((3-(2- methoxyethoxy)phenyl)sulfonyl)phenyl)- 2,4-dihydro-3H-1,2,4-triazole-3- thione, |

or a pharmaceutically acceptable salt thereof.

20. The method of any one of claims 1-17, wherein the compound is a compound of Formula (IIA): 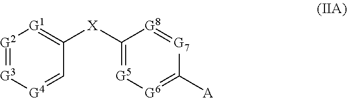
wherein
G¹ is CH or N;
G² is CR^{2a} or N;
G³ is CR^{3a} or N;
G⁴ is CH or N;
wherein no more than two of G¹, G², G³, and G⁴ are N;
G⁵ is CH or N;
G⁶ is CR^{1a} or N;
G⁷ is CH or N;
G⁸ is CH or N;
wherein no more than one of G⁵, G⁶, G⁷, and G⁸ is N;
R^{1a}, R^{2a}, and R^{3a} are each independently hydrogen, hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, or an optionally substituted heterocyclyl;
wherein R^{x} and R^{y} are each independently H or optionally substituted C₁₋₄alkyl;
or R^{1a} and R^{2a} are taken together with the carbons to which they are attached to form a 5- to 16-membered heterocyclyl ring;
X is —CR^{4a}R^{5a}—, —O—, —S—, —S(O)—, —NR^{6a}—, —S(O)₂—, —NR^{6a}S(O)₂—, —CR^{4a}R^{5a}S(O)₂—, —C(O)—, —NR^{6a}C(O)—, or —NHNHC(O)—;
wherein R^{4a} and R^{5a} are independently hydrogen, hydroxy, halogen, substituted C₁₋₄ alkyl, C₁₋₄ alkoxy, or substituted C₁₋₄ alkoxy;
or R^{4a} and R^{5a} are taken together with the carbon to which they are attached to form a 3- to 6-membered cycloalkyl ring;
each R^{6a} is independently hydrogen or C₁₋₄ alkyl;
A is 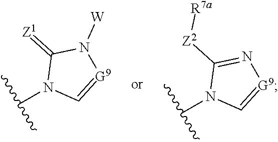
G⁹ is CH or N;
Z¹ and Z² are independently S or O; and
W and R^{7a} are independently hydrogen or C₁₋₄ alkyl,
or a pharmaceutically acceptable salt thereof.

21. The method of any one of claims 1-17, wherein the compound is selected from the group consisting of

| Compound | | |
|---|---|---|
| No. | Structure | Name |
| 26 | 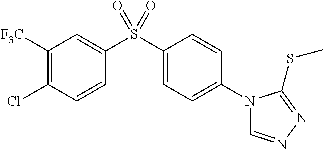 [SMILES: CSc1nncn1-c1ccc(S(=O)(=O)c2ccc(Cl)c(C(F)(F)F)c2)cc1] | 4-(4-((4-chloro-3- (trifluoromethyl)phenyl)sulfonyl)phenyl)- 3-(methylthio)-4H-1,2,4-triazole; |
| 27 | 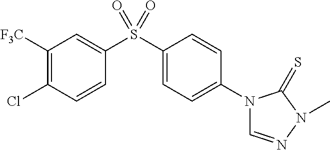 [SMILES: Cn1ncn(-c2ccc(S(=O)(=O)c3ccc(Cl)c(C(F)(F)F)c3)cc2)c1=S] | 4-(4-((4-chloro-3- (trifluoromethyl)phenyl)sulfonyl)phenyl)- 2-methyl-2,4-dihydro-3H-1,2,4-triazole- 3-thione; |
| 28 | 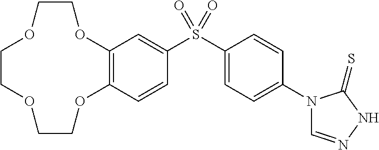 [SMILES: O=S(=O)(c1ccc(-n2cn[nH]c2=S)cc1)c1ccc2c(c1)OCCOCCOCCO2] | 4-(4-((2,3,5,6,8,9- hexahydrobenzo[b][1,4,7,10]tetraoxacyclo- dodecin-12-yl)sulfonyl)phenyl)-2,4- dihydro-3H-1,2,4-triazole-3-thione; |
| 29 | 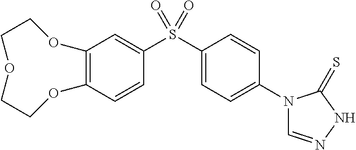 [SMILES: O=S(=O)(c1ccc(-n2cn[nH]c2=S)cc1)c1ccc2c(c1)OCCOCCO2] | 4-(4-((2,3,5,6- tetrahydrobenzo[b][1,4,7]trioxonin-9- yl)sulfonyl)phenyl)-2,4-dihydro-3H- 1,2,4-triazole-3-thione; |
| 30 | 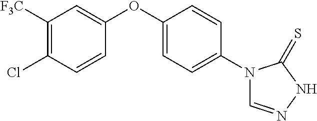 [SMILES: FC(F)(F)c1cc(Oc2ccc(-n3cn[nH]c3=S)cc2)ccc1Cl] | 4-(4-(4-chloro-3-(trifluoro-methyl)- phenoxy)-phenyl)-3,4-dihydro-2H-1,2,4- triazole-3-thione; |
| 31 | 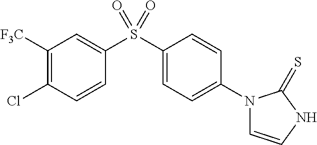 [SMILES: O=S(=O)(c1ccc(-n2cc[nH]c2=S)cc1)c1ccc(Cl)c(C(F)(F)F)c1] | 1-(4-(4-chloro-3-(trifluoro-methyl)- phenylsulfonyl)-phenyl)-2,3-dihydro- 1H-imidazole-2-thione; |
| 32 | 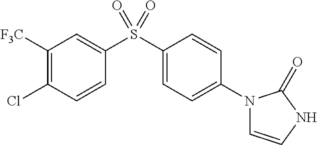 [SMILES: O=c1[nH]ccn1-c1ccc(S(=O)(=O)c2ccc(Cl)c(C(F)(F)F)c2)cc1] | 1-(4-(4-chloro-3-(trifluoro-methyl)- phenylsulfonyl)-phenyl)-2,3-dihydro- 1H-imidazol-2-one; |
| 33 | 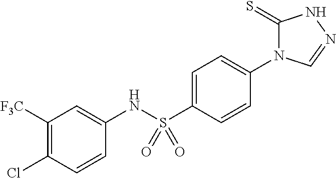 [SMILES: O=S(=O)(Nc1ccc(Cl)c(C(F)(F)F)c1)c1ccc(-n2cn[nH]c2=S)cc1] | N-(4-chloro-3-(trifluoromethyl)phenyl)- 4-(5-thioxo-1,5-dihydro-4H-1,2,4- triazol-4-yl)benzenesulfonamide; |
| 34 | 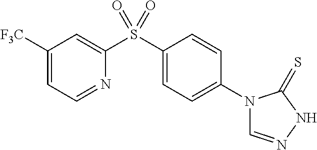 [SMILES: O=S(=O)(c1ccc(-n2cn[nH]c2=S)cc1)c1cc(C(F)(F)F)ccn1] | 4-(4-((2-(trifluoromethyl)pyridin-4- yl)sulfonyl)phenyl)-2,4-dihydro-3H- 1,2,4-triazole-3-thione; |
| 35 | 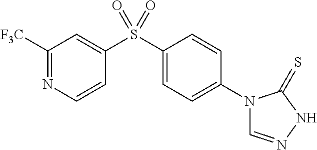 [SMILES: O=S(=O)(c1ccc(-n2cn[nH]c2=S)cc1)c1ccnc(C(F)(F)F)c1] | 4-(4-((4-(trifluoromethyl)pyridin-2- yl)sulfonyl)phenyl)-2,4-dihydro-3H- 1,2,4-triazole-3-thione; |
| 36 | 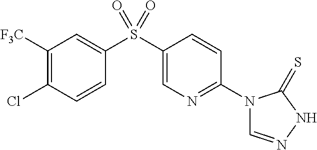 [SMILES: O=S(=O)(c1ccc(-n2cn[nH]c2=S)nc1)c1ccc(Cl)c(C(F)(F)F)c1] | 4-(5-((4-chloro-3- (trifluoromethyl)phenyl)sulfonyl)pyridin- 2-yl)-2,4-dihydro-3H-1,2,4-triazole-3- thione; |
| 37 | 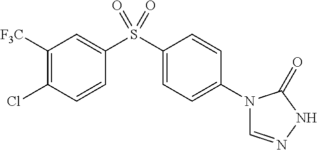 [SMILES: O=c1[nH]ncn1-c1ccc(S(=O)(=O)c2ccc(Cl)c(C(F)(F)F)c2)cc1] | 4-(4-((4-chloro-3- (trifluoromethyl)phenyl)sulfonyl)phenyl)- 2,4-dihydro-3H-1,2,4-triazole-3-one; |
| 38 | 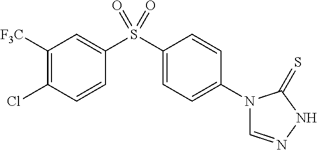 [SMILES: O=S(=O)(c1ccc(-n2cn[nH]c2=S)cc1)c1ccc(Cl)c(C(F)(F)F)c1] | (4-chloro-3-(trifluoromethyl)phenyl)(4- (5-thioxo-1,5-dihydro-4H-1,2,4-triazol- 4-yl)phenyl)methanone; |
| 39 | 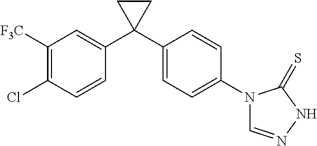 [SMILES: FC(F)(F)c1cc(C2(c3ccc(-n4cn[nH]c4=S)cc3)CC2)ccc1Cl] | 4-(4-(1-(4-chloro-3- (trifluoromethyl)phenyl)cyclopropyl)phe- nyl)-2,4-dihydro-3H-1,2,4-triazole-3- thione; |
| 40 | 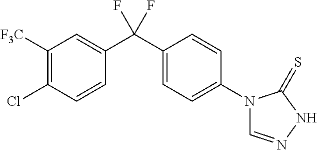 [SMILES: FC(F)(F)c1cc(C(F)(F)c2ccc(-n3cn[nH]c3=S)cc2)ccc1Cl] | 4-(4-((4-chlroo-3- (trifluoromethyl)phenyl)difluoromethyl) phenyl)-2,4-dihydro-3H-1,2,4-triazole-3- thione; |
| 41 | 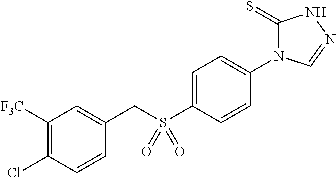 [SMILES: O=S(=O)(Cc1ccc(Cl)c(C(F)(F)F)c1)c1ccc(-n2cn[nH]c2=S)cc1] | 4-(4-((4-chloro-3- (trifluoromethyl)benzyl)sulfonyl)phenyl)- 2,4-dihydro-3H-1,2,4-triazole-3-thione; |
| 42 | 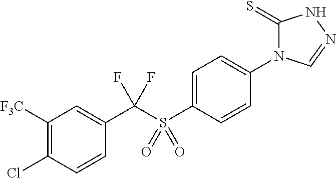 [SMILES: O=S(=O)(c1ccc(-n2cn[nH]c2=S)cc1)C(F)(F)c1ccc(Cl)c(C(F)(F)F)c1] | 4-(4-(((4-chloro-3- (trifluoromethyl)phenyl)difluoromethyl) sulfonyl)phenyl)-2,4-dihydro-3H-1,2,4- triazole-3-thione |
| 43 | 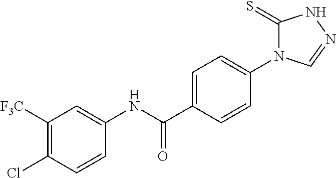 [SMILES: O=C(Nc1ccc(Cl)c(C(F)(F)F)c1)c1ccc(-n2cn[nH]c2=S)cc1] | N-(4-chloro-3-(trifluoromethyl)phenyl)- 4-(5-thioxo-1,5-dihydro-4H-1,2,4- triazol-4-yl)benzamide; and |
| 44 | 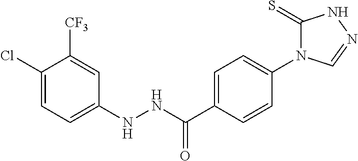 [SMILES: O=C(NNc1ccc(Cl)c(C(F)(F)F)c1)c1ccc(-n2cn[nH]c2=S)cc1] | N′-(4-chloro-3-(trifluoromethyl)phenyl)- 4-(5-thioxo-1,5-dihydro-4H-1,2,4- triazol-4-yl)benzohydrazide, |

or a pharmaceutically acceptable salt thereof.

22. A compound of Formula (IIA): 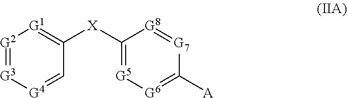
wherein
G¹ is CH or N;
G² is CR^{2a} or N;
G³ is CR^{3a} or N;
G⁴ is CH or N;
wherein no more than two of G¹, G², G³, and G⁴ are N;
G⁵ is CH or N;
G⁶ is CR^{1a} or N;
G⁷ is CH or N;
G⁸ is CH or N;
wherein no more than one of G⁵, G⁶, G⁷, and G⁸ is N;
R^{1a}, R^{2a}, and R^{3a} are each independently hydrogen, hydroxy, halogen, C₁₋₄ alkyl, substituted C₁₋₄ alkyl, C₁₋₄ alkoxy, substituted C₁₋₄ alkoxy, —CN, —C(O)R^{x}, —C(O)OR^{x}, —S(O)₂R^{x}, —NR^{x}R^{y}, or an optionally substituted heterocyclyl;
wherein R^{x} and R^{y} are each independently H or optionally substituted C₁₋₄alkyl;
or R^{1a} and R^{2a} are taken together with the carbons to which they are attached to form a 5- to 16-membered heterocyclyl ring;
X is —CR^{4a}R^{5a}—, —O—, —S—, —S(O)—, —NR^{6a}—, —S(O)₂—, —NR^{6a}S(O)₂—, —CR^{4a}R^{5a}S(O)₂—, —C(O)—, —NR^{6a}C(O)—, or —NHNHC(O)—;
wherein R^{4a} and R^{5a} are independently hydrogen, hydroxy, halogen, substituted C₁₋₄ alkyl, C₁₋₄ alkoxy, or substituted C₁₋₄ alkoxy;
or R^{4a} and R^{5a} are taken together with the carbon to which they are attached to form a 3- to 6-membered cycloalkyl ring;
each R^{6a} is independently hydrogen or C₁₋₄ alkyl;
A is 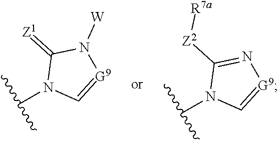
G⁹ is CH or N;
Z¹ and Z² are independently S or O; and
W and R^{7a} are independently hydrogen or C₁₋₄ alkyl; and
one or more of the following apply:
(i) X is —CR^{4a}R^{5a}—, —O—, —S—, —S(O)—, —NR^{6a}—, —NR^{6a}S(O)₂—, —CR^{4a}R^{5a}S(O)₂—, —C(O)—, —NR^{6a}C(O)—, or —NHNHC(O)—;
(ii) one or two of G¹, G², G³, and G⁴ is N;
(iii) one of G⁵, G⁶, G⁷, and G⁸ is N;
(iv) R^{1a} is an optionally substituted heterocyclyl, wherein when the heterocyclyl is monocyclic, the point of connection is via a carbon atom;
(v) R^{2a} is an optionally substituted heterocyclyl, wherein when the heterocyclyl is monocyclic, the point of connection is via a carbon atom;
(vi) R^{3a} is an optionally substituted heterocyclyl, wherein when the heterocyclyl is monocyclic, the point of connection is via a carbon atom;
(vii) R^{2a} and R^{3a} are taken together with the carbons to which they are attached to form a 5- to 16-membered heterocyclyl ring;
(viii) A is 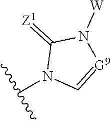
and Z¹ is O;
(ix) A is 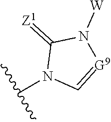
and W is C₁₋₄ alkyl;
(x) A is 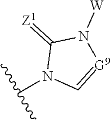
and G⁹ is CH; and
(xii) A is 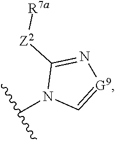
or a pharmaceutically acceptable salt thereof.

23. The compound of claim 22, or a pharmaceutically acceptable salt thereof, wherein the compound of Formula (IIA) is a compound of a Formula (II-1): 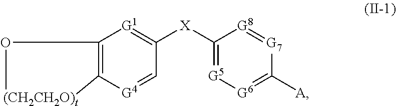
or a pharmaceutically acceptable salt thereof, wherein G¹, G⁴, G⁵, G⁶, G⁷, G^{g}, X and A are as defined for Formula (IIA), and t is 1, 2, or 3.

24. The compound of claim 22, or a pharmaceutically acceptable salt thereof, wherein the compound of Formula (IIA) is a compound of a Formula (II-2): 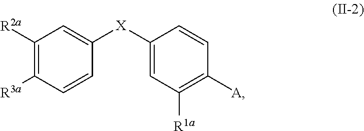
or a pharmaceutically acceptable salt thereof, wherein R^{1a}, R^{2a}, R^{3a}, X and A are as defined for Formula (IIA).

25. The compound of claim 22, or a pharmaceutically acceptable salt thereof, wherein the compound of Formula (IIA) is a compound of Formula (IIa), (IIb), (IIc), (IId), (IIe), (IIf), or (IIg): 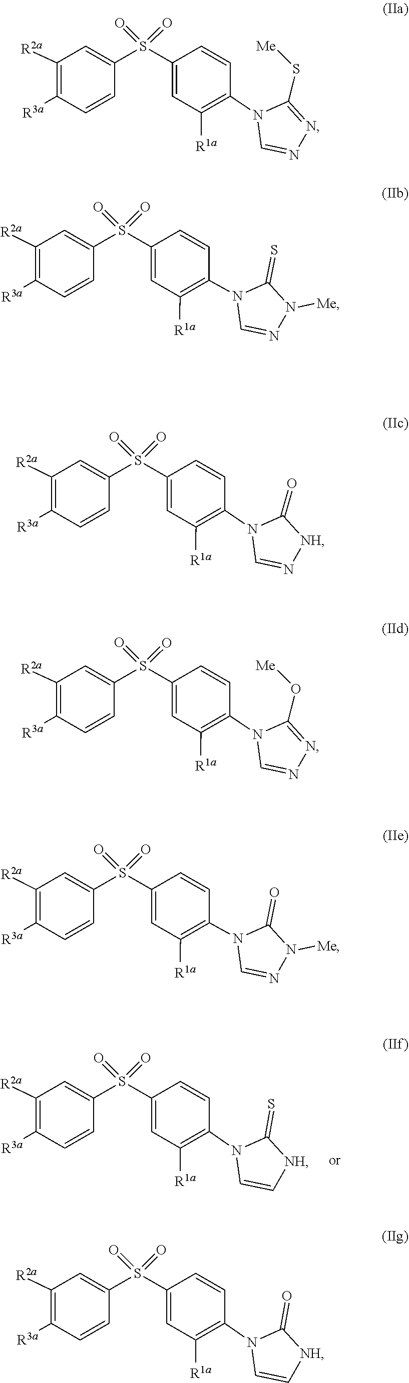
or a pharmaceutically acceptable salt thereof, wherein R^{1a}, R^{2a}, and R^{3a} are as defined for Formula (IIA).

26. The compound of claim 22, or a pharmaceutically acceptable salt thereof, wherein the compound of Formula (IIA) is a compound of Formula (IIh), (IIi), (IIj), (Ilk), (IIl), (IIm), (IIn), (IIo), or (IIp): 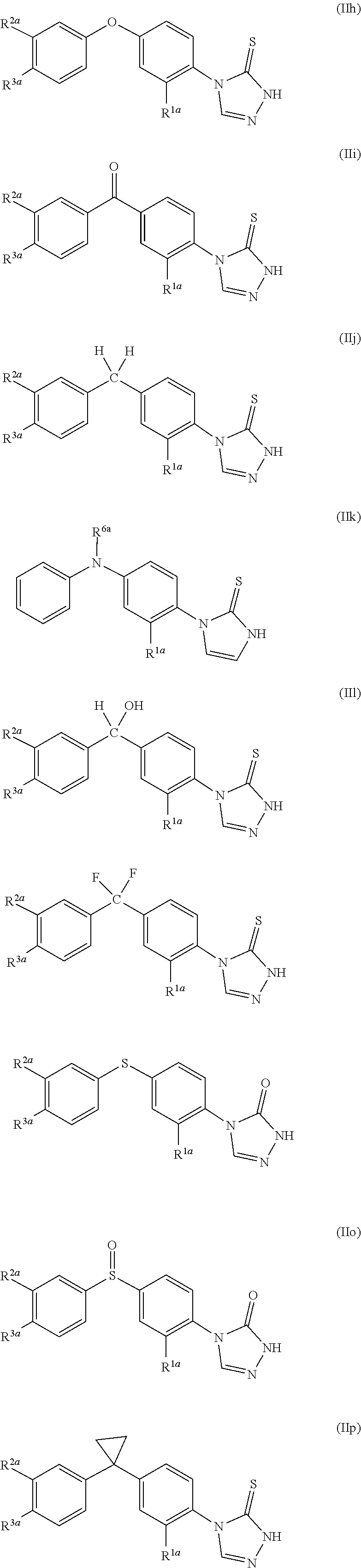
or a pharmaceutically acceptable salt thereof, wherein R^{1a}, R^{2a}, R^{3a}, and R^{6a} are as defined for Formula (IIA).

27. A compound selected from the group consisting of

| Compound | | |
|---|---|---|
| No. | Structure | Name |
| 26 | 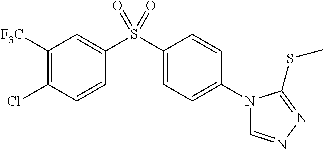 [SMILES: CSc1nncn1-c1ccc(S(=O)(=O)c2ccc(Cl)c(C(F)(F)F)c2)cc1] | 4-(4-((4-chloro-3- (trifluoromethyl)phenyl)sulfonyl)phenyl)- 3-(methylthio)-4H-1,2,4-triazole; |
| 27 | 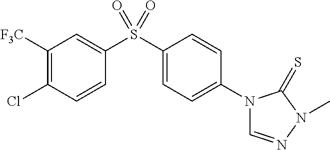 [SMILES: Cn1ncn(-c2ccc(S(=O)(=O)c3ccc(Cl)c(C(F)(F)F)c3)cc2)c1=S] | 4-(4-((4-chloro-3- (trifluoromethyl)phenyl)sulfonyl)phenyl)- 2-methyl-2,4-dihydro-3H-1,2,4-triazole- 3-thione; |
| 28 | 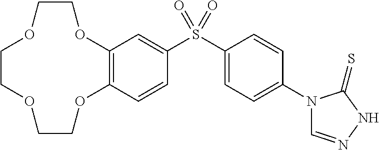 [SMILES: O=S(=O)(c1ccc(-n2cn[nH]c2=S)cc1)c1ccc2c(c1)OCCOCCOCCO2] | 4-(4-((2,3,5,6,8,9- hexahydrobenzo[b][1,4,7,10]tetraoxacyclo- dodecin-12-yl)sulfonyl)phenyl)-2,4- dihydro-3H-1,2,4-triazole-3-thione; |
| 29 | 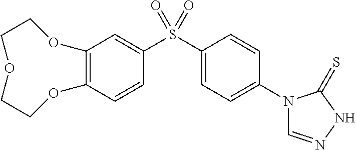 [SMILES: O=S(=O)(c1ccc(-n2cn[nH]c2=S)cc1)c1ccc2c(c1)OCCOCCO2] | 4-(4-((2,3,5,6- tetrahydrobenzo[b][1,4,7]trioxonin-9- yl)sulfonyl)phenyl)-2,4-dihydro-3H- 1,2,4-triazole-3-thione; |
| 30 | 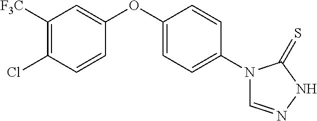 [SMILES: FC(F)(F)c1cc(Oc2ccc(-n3cn[nH]c3=S)cc2)ccc1Cl] | 4-(4-(4-chloro-3-(trifluoro-methyl)- phenoxy)-phenyl)-3,4-dihydro-2H-1,2,4- triazole-3-thione; |
| 31 | 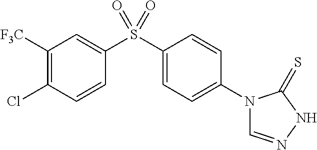 [SMILES: O=S(=O)(c1ccc(-n2cn[nH]c2=S)cc1)c1ccc(Cl)c(C(F)(F)F)c1] | 1-(4-(4-chloro-3-(trifluoro-methyl)- phenylsulfonyl)-phenyl)-2,3-dihydro- 1H-imidazole-2-thione; |
| 32 | 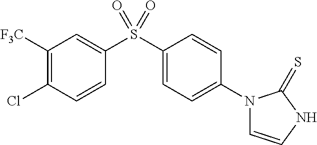 [SMILES: O=S(=O)(c1ccc(-n2cc[nH]c2=S)cc1)c1ccc(Cl)c(C(F)(F)F)c1] | 1-(4-(4-chloro-3-(trifluoro-methyl)- phenylsulfonyl)-phenyl)-2,3-dihydro- 1H-imidazol-2-one; |
| 33 | 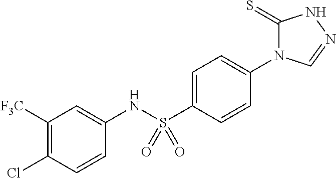 [SMILES: O=S(=O)(Nc1ccc(Cl)c(C(F)(F)F)c1)c1ccc(-n2cn[nH]c2=S)cc1] | N-(4-chloro-3-(trifluoromethyl)phenyl)- 4-(5-thioxo-1,5-dihydro-4H-1,2,4- triazol-4-yl)benzenesulfonamide; |
| 34 | 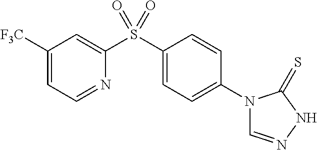 [SMILES: O=S(=O)(c1ccc(-n2cn[nH]c2=S)cc1)c1cc(C(F)(F)F)ccn1] | 4-(4-((2-(trifluoromethyl)pyridin-4- yl)sulfonyl)phenyl)-2,4-dihydro-3H- 1,2,4-triazole-3-thione; |
| 35 | 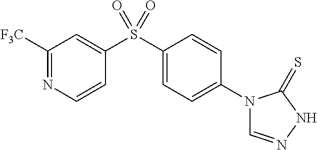 [SMILES: O=S(=O)(c1ccc(-n2cn[nH]c2=S)cc1)c1ccnc(C(F)(F)F)c1] | 4-(4-((4-(trifluoromethyl)pyridin-2- yl)sulfonyl)phenyl)-2,4-dihydro-3H- 1,2,4-triazole-3-thione; |
| 36 | 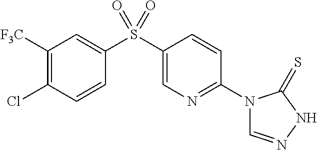 [SMILES: O=S(=O)(c1ccc(-n2cn[nH]c2=S)nc1)c1ccc(Cl)c(C(F)(F)F)c1] | 4-(5-((4-chloro-3- (trifluoromethyl)phenyl)sulfonyl)pyridin- 2-yl)-2,4-dihydro-3H-1,2,4-triazole-3- thione; |
| 37 | 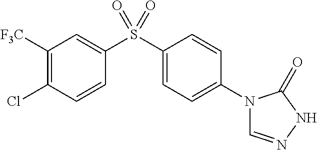 [SMILES: O=c1[nH]ncn1-c1ccc(S(=O)(=O)c2ccc(Cl)c(C(F)(F)F)c2)cc1] | 4-(4-((4-chloro-3- (trifluoromethyl)phenyl)sulfonyl)phenyl)- 2,4-dihydro-3H-1,2,4-triazol-3-one; |
| 38 | 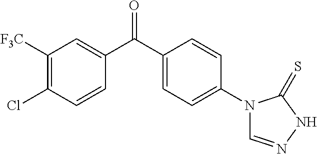 [SMILES: O=C(c1ccc(-n2cn[nH]c2=S)cc1)c1ccc(Cl)c(C(F)(F)F)c1] | (4-chloro-3-(trifluoromethyl)phenyl)(4- (5-thioxo-1,5-dihydro-4H-1,2,4-triazol- 4-yl)phenyl)methanone; |
| 39 | 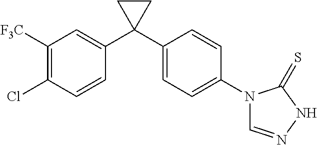 [SMILES: FC(F)(F)c1cc(C2(c3ccc(-n4cn[nH]c4=S)cc3)CC2)ccc1Cl] | 4-(4-(1-(4-chloro-3- (trifluoromethyl)phenyl)cyclopropyl)phe- nyl)-2,4-dihydro-3H-1,2,4-triazole-3- thione; |
| 40 | 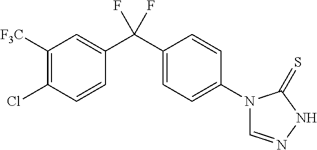 [SMILES: FC(F)(F)c1cc(C(F)(F)c2ccc(-n3cn[nH]c3=S)cc2)ccc1Cl] | 4-(4-((4-chloro-3- (trifluoromethyl)phenyl)difluoromethyl) phenyl)-2,4-dihydro-3H-1,2,4-triazole-3- thione; |
| 41 | 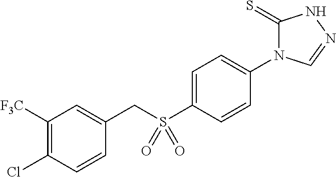 [SMILES: O=S(=O)(Cc1ccc(Cl)c(C(F)(F)F)c1)c1ccc(-n2cn[nH]c2=S)cc1] | 4-(4-((4-chloro-3- (trifluoromethyl)benzyl)sulfonyl)phenyl)- 2,4-dihydro-3H-1,2,4-triazole-3-thione; |
| 42 | 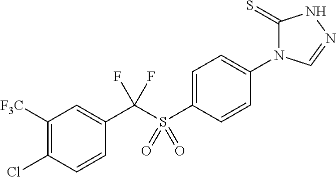 [SMILES: O=S(=O)(c1ccc(-n2cn[nH]c2=S)cc1)C(F)(F)c1ccc(Cl)c(C(F)(F)F)c1] | 4-(4-(((4-chloro-3- (trifluoromethyl)phenyl)difluoromethyl) sulfonyl)phenyl)-2,4-dihydro-3H-1,2,4- triazole-3-thione; |
| 43 | 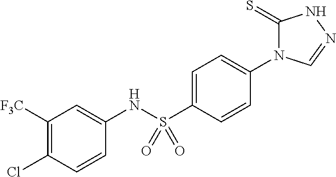 [SMILES: O=S(=O)(Nc1ccc(Cl)c(C(F)(F)F)c1)c1ccc(-n2cn[nH]c2=S)cc1] | N-(4-chloro-3-(trifluoromethyl)phenyl)- 4-(5-thioxo-1,5-dihydro-4H-1,2,4- triazol-4-yl)benzamide; and |
| 44 | 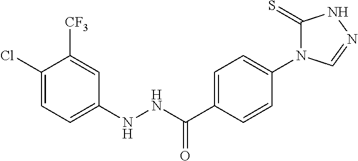 [SMILES: O=C(NNc1ccc(Cl)c(C(F)(F)F)c1)c1ccc(-n2cn[nH]c2=S)cc1] | N′-(4-chloro-3-(trifluoromethyl)phenyl)- 4-(5-thioxo-1,5-dihydro-4H-1,2,4- triazol-4-yl)benzohydrazide, |

or a pharmaceutically acceptable salt thereof.

28. A pharmaceutical composition comprising (a) at least one compound of any one of claims 22-27, or a pharmaceutically acceptable salt thereof, and (b) a pharmaceutically acceptable excipient.

29. A method of treating a condition associated with neurodegeneration or accumulation of proteins, comprising administering to a subject in need of such treatment an effective amount of at least one compound of any one of claims 22-27, or a pharmaceutically acceptable salt thereof, or a pharmaceutical composition of claim 28.

30. A compound of any one of claims 22-27, or a pharmaceutically acceptable salt thereof, or a pharmaceutical composition of claim 28, for use in the treatment of a condition associated with neurodegeneration or accumulation of proteins.

31. Use of at least one compound of any one of claims 22-27, or a pharmaceutically acceptable salt thereof, or a pharmaceutical composition of claim 28, in the manufacture of a medicament for the treatment of a condition associated with neurodegeneration or accumulation of proteins.

32. The method of claim 29, the compound of claim 30, or the use of claim 31, wherein the condition is Alzheimer's Disease, Parkinson's Disease, fronto-temporal dementia, dementia with Lewy Bodies, PD dementia, multiple system atrophy, Huntington's disease, Amyotrophic lateral sclerosis, progressive supranuclear palsy, cancer, infection, Crohn's disease, heart disease, aging, or traumatic brain injury (TBI).

33. A method of preventing aggregation or accumulation or enhancing clearance of protease-resistant protein, comprising contacting the protease-resistant protein with an effective amount of at least one compound of any one of claims 22-27, or a salt thereof, or a pharmaceutical composition of claim 28, wherein the contacting is in vitro, ex vivo, or in vivo.

34. The method of claim 33, wherein the protease-resistant protein is selected from alpha synuclein, a-beta, tau, Huntingtin, and TAR DNA binding protein 43 (TDP43) proteins.

35. A method of decreasing neuroinflammation in a subject, comprising administering to the subject an effective amount of at least one compound of any one of claims 22-27, or a pharmaceutically acceptable salt thereof, or a pharmaceutical composition of claim 28.

36. A compound of any one of claims 22-27, or a pharmaceutically acceptable salt thereof, or a pharmaceutical composition of claim 28, for use in decreasing neuroinflammation.

37. Use of at least one compound of any one of claims 22-27, or a pharmaceutically acceptable salt thereof, or a pharmaceutical composition of claim 28, in the manufacture of a medicament for decreasing neuroinflammation.

38. A method of treating a disease or condition associated with neuroinflammation, comprising administering to a subject in need of such treatment an effective amount of at least one compound of any one of claims 22-27, or a pharmaceutically acceptable salt thereof, or a pharmaceutical composition of claim 28.

39. A compound of any one of claims 22-27, or a pharmaceutically acceptable salt thereof, or a pharmaceutical composition of claim 28, for use in the treatment of a disease or condition associated with neuroinflammation.

40. Use of at least one compound of any one of claims 22-27, or a pharmaceutically acceptable salt thereof, or a pharmaceutical composition of claim 28, in the manufacture of a medicament for the treatment of a disease or condition associated with neuroinflammation.
